(19) United States
(12) Patent Application Publication
(10) Pub. No.: US 2026/0022096 A1
(43) Pub. Date: Jan. 22, 2026

(54) NOVEL SULFONAMIDES AND THEIR USE AS NEUROPROTECTIVE AND/OR NEURORESTORATIVE AGENTS

(71) Applicant: GENECODE, TALLINN (EE)

(72) Inventors: Delphine CHARVIN, GEX (FR); Serge MIGNANI, CHATENAY-MALABRY (FR); Frédéric MIEGE, LYON (FR); Vincent RODESCHINI, MIONS (FR); Yves RIBEILL, DURHAM, NC (US)

(73) Assignee: GENECODE, TALLINN (EE)

(21) Appl. No.: 18/995,561

(22) PCT Filed: Jul. 28, 2023
(86) PCT No.: PCT/EP2023/070968

§ 371 Date: Jan. 16, 2025

(30) Foreign Application Priority Data
Jul. 28, 2022 (EP) 22187646.9

(51) Int. Cl. C07D 211/16; A61K 31/495; A61K 31/496; A61K 31/4995; A61K 31/506; A61K 31/5377; A61K 31/5386; C07D 401/12; C07D 487/08; C07D 498/08

(52) U.S. Cl. CPC C07D 211/16; A61K 31/495; A61K 31/496; A61K 31/4995; A61K 31/506; A61K 31/5377; A61K 31/5386; C07D 401/12; C07D 487/08; C07D 498/08

(57) ABSTRACT

A compound of general formula (I)

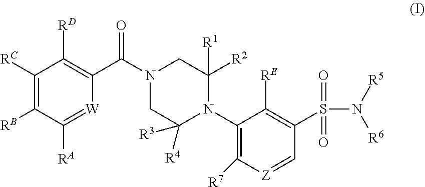

or a pharmaceutically acceptable salt and/or solvate thereof, and the process for manufacturing the compound of general formula (I). Also, a pharmaceutical composition that includes a compound of general formula (I), and the use of compounds of general formula (I) as neuroprotective and/or neurorestorative agents, in particular for use in the treatment of neurological disorders.

### FIELD OF INVENTION

The present invention relates to novel polycyclic sulfonamides comprising at least one substituted and/or bridged piperazine. The compounds of the invention are useful as neuroprotective and/or neurorestorative agents, in particular for use in the treatment of neurological disorders.

### BACKGROUND OF INVENTION

Neurological disorders (NDs) are heterogeneous diseases affecting the autonomic, peripheral and central nervous system of the body. Amongst the Central Nervous System (CNS) diseases, Alzheimer's disease (AD), Parkinson's disease (PD), Huntington's disease (HD), Amyotrophic Lateral Sclerosis (ALS), dementia, stroke, head trauma, brain tumor, pain and epilepsy are always the most challenging diseases to be addressed. Compounds actives for treating CNS might also be relevant to other diseases, for example diseases of the peripheral nervous system, eyes, spinal cord and enteric system.

The incidence of NDs is expected to increase dramatically in the 21^{st} century, in particular due to increased life expectancy and demographic changes. Some of these diseases are characterized by age-related gradual decline in neurological functions. In medicine, neurological diseases are the world's important and common cause of disability-adjusted life years, or years of healthy life lost due to death or disability. CNS diseases represent the largest and fastest growing therapeutic domain of unmet medical need, and it is being recognized as a global public health challenge and become a major global health priority. Adequate neurological diagnosis represents an immense challenge, and patients are more concerned about the development of new effective treatments to treat pathophysiology or symptoms. Neurological disorders affect millions of people worldwide and cause permanent damage. They are progressive diseases with symptoms that can degenerate overtime. Although there is generally no definitive cure, supporting treatments exist. The goal of these treatments is mainly to reduce symptoms and preserve the quality of life of the patient as long as possible.

Neurons are postmitotic cells that must live for a lifetime. While young neurons have proper functioning of self-healing protective mechanisms, aging or external or internal insults disturb them, eventually leading to neurodegeneration. These external/internal hazards are traumatic injuries or excitotoxic compounds, reactive oxygen species (ROS), protein aggregates, and other toxic molecules. Fortunately, cells have an intrinsic machinery that blocks death by activating resilience mechanisms or promoting regeneration pathways. Dysfunctionality or insufficiency of these self-healing mechanisms has also been described in neurodegenerative diseases.

Among natural self-healing agents, the glial cell line-derived neurotrophic factor (GDNF) acts as a potent neurotrophic factor, promoting survival in different neuronal populations such as spinal motor neurons, retinal cells, central noradrenergic neurons, or sympathetic neurons, among others. Likewise, GDNF (and other proteins of the GDNF family of neurotrophic factors such as neurturin, artemin and persephin) acts as a powerful trophic factor favoring, not only the survival and plasticity, but also the proliferation, differentiation, and protection of dopaminergic neurons, as well as the synthesis of dopamine and dopaminergic transmission in the developing and adult brain. GDNF can promote neuroprotection through MAP kinase/ERK, Src kinase and PI3 kinase/AKT pathways by inducing several neuroprotective signaling cascade, including the activation of the transcription factor Elk1 through the activation of the GFRal-RET receptor complex.

The field of applications for these proteins is vast. Pre-clinical and clinical trials have been carried out to evaluate the effect of neurotrophic factors of the GDNF family for the prevention, treatment or management of Parkinson's disease, chronic pain, Alzheimer's disease, amyotrophic lateral sclerosis, neuropathy, depression, stroke, and these proteins were even suggested as male contraceptives. However, the clinical application of GDNF is hampered by its poor pharmacokinetic properties, the fact that it does not cross the blood-brain barrier and thus the necessity for intracranial delivery via stereotaxic surgery, varying biological activity, and high price.

Blood-brain barrier penetrating small-molecule compounds that target the GDNF receptor complex and mimic GDNF biological effects in neurons may be an avenue to overcome these issues and translate to greater efficacy in the clinic. Superior tissue penetration of such compounds could promote survival in all affected neuronal pathways.

WO 2011/070177 A2 (BALTIC TECHNOLOGY DEV LTD) discloses polycyclic compounds for treating neurological disorders. Although these compounds represented a significant improvement at this time, they still have limitations in terms of activity on GFRα1-RET target, solubility, membrane permeability (PAMPA and CaCO2), intrinsic clearance microsomes and hepatocytes, plasma protein binding, and pharmacokinetic profile. In particular, these compounds are limited in terms of activity on GFRα1-RET target (as evidenced, for example, in luciferase assays).

It was surprisingly found out by the Applicants that novel polycyclic sulfonamides of formula (I) showed potent GFRα1-RET activity in a luciferase assay, thereby opening the way to overcoming the limitations of available therapeutic solutions.

### SUMMARY

An object of the present invention is a compound of formula (I)

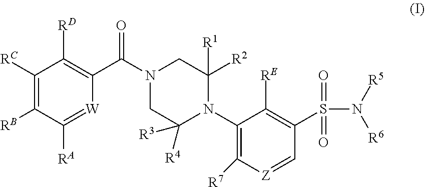

or a pharmaceutically acceptable salt and/or solvate thereof;
   wherein W, R^{A}-R^{D}, R¹-R⁴, Z, R⁵-R⁷ and R^{E} are as defined hereinafter and/or as defined in the claims.

According to one embodiment, the compound is selected from the compounds of Table 1 herein, and pharmaceutically acceptable salts and/or solvates thereof.

Another object of the present invention is a pharmaceutical composition comprising a compound according to the invention and at least one pharmaceutically acceptable carrier.

Another object of the present invention is a compound according to the invention or a pharmaceutical composition according to the invention for use as a medicament. According to one embodiment, the compound or the pharmaceutical composition is for use in the treatment of a neurological disorder.

Another object of the present invention is a process for manufacturing a compound according to the invention.

### Definitions

In the present invention, the following terms have the following meanings:

### Chemical Definitions

Where chemical substituents are combinations of chemical groups, the point of attachment of the substituent to the molecule is by the last chemical group recited on the right of the name of the substituent. For example, an arylalkyl substituent is linked to the rest of the molecule through the alkyl moiety and it may by represented as follows: “aryl-alkyl-”.

Unless otherwise indicated, the compounds were named using BIOVIA Draw 2021 (Dassault, France).

The definitions herein referring to the optional or mandatory substitution of a specified group apply both to the substituted group considered as such or to the same group comprised in another chemical moiety, which can both be substituted as set forth herein. For example, “R^{x}” represents hydrogen, (C₁-C₈) alkyl, (C₁-C₈) alkyl-O— or cycloalkyl-(C₁-C₈) alkyl-NH—; wherein the alkyl is optionally substituted by at least one F″ means that any alkyl group present in the structure of R^{x} may optionally substituted by at least one F, including said (C₁-C₈) alkyl as such (e.g., CF₃), the alkyl comprised in said (C₁-C₈) alkyl-O-(e.g., OCF₃) and the alkyl comprised in said cycloalkyl-(C₁-C₈) alkyl-NH— (e.g., cyclopropyl-CH₂—CHF—CH₂—NH—).

“Alkoxy” refers to an alkyl-O— group.

“Alkyl” refers to a saturated linear or branched hydrocarbon chain, typically comprising from 1 to 16 carbon atoms, preferably from 1 to 12 carbon atoms, more preferably from 1 to 8 carbon atoms, furthermore preferably from 1 to 6 carbon atoms. Alkyl groups may be monovalent or polyvalent (i.e., “alkylene” groups, which are divalent alkyl groups, are encompassed in “alkyl” definition). Non-limiting examples of alkyl groups include methyl, ethyl, n-propyl, i-propyl, n-butyl, i-butyl, s-butyl and t-butyl, pentyl and its isomers (e.g., n-pentyl, iso-pentyl), and hexyl and its isomers (e.g., n-hexyl, iso-hexyl). Particular examples of alkyl groups include methyl, ethyl, n-propyl, i-propyl, n-butyl, s-butyl and t-butyl (including methylene, ethylene, n-propylene, n-butylene and n-butylene).

“Amine” refers to derivatives of ammonia (NH₃), wherein one or more hydrogen atoms have been replaced by a substituent such as, for example, alkyl or aryl. “Amino” refers to the —NH₂ group.

“Aryl” refers to a cyclic, polyunsaturated, aromatic hydrocarbyl group comprising at least one aromatic ring and comprising from 5 to 12 carbon atoms, preferably from 6 to 10 carbon atoms. Aryl groups may be monovalent or polyvalent (e.g., divalent). Aryl groups may have a single ring (e.g., phenyl) or multiple aromatic rings fused together (e.g., naphthyl) or linked covalently. The aromatic ring may optionally include one to two additional rings (either cycloalkyl, heterocycloalkyl or heteroaryl) fused thereto. This definition of “aryl” encompasses the partially hydrogenated derivatives of the carbocyclic systems enumerated herein, as long as at least one ring is aromatic. Aryls may optionally be substituted by at least one group such as, for example, halogen (e.g., F or Cl), (C₁-C₈) alkyl (e.g., methyl) or nitrile (CN). Non-limiting examples of aryl groups include phenyl, biphenyl, biphenylenyl, 5- or 6-tetralinyl, naphthalen-1- or -2-yl, 4-, 5-, 6 or 7-indenyl, 1-2-, 3-, 4- or 5-acenaphthylenyl, 3-, 4- or 5-acenaphthenyl, 1- or 2-pentalenyl, 4- or 5-indanyl, 5-, 6-, 7- or 8-tetrahydronaphthyl, 1,2,3,4-tetrahydronaphthyl, 1,4-dihydronaphthyl, and 1-, 2-, 3-, 4- or 5-pyrenyl. A particular example of aryl group is phenyl.

“Benzylidene” refers to a phenyl group bond to a moiety through an exo carbon-carbon double bound, i.e., =CH—Ph bond to a carbon atom. The moiety is typically cyclic such as, for example, an heterocycloalkyl.

“Cycloalkyl” refers to a cyclic alkyl group, typically comprising from 3 to 15 carbon atoms, preferably from 3 to 12 carbon atoms, more preferably from 3 to 8 carbon atoms, further more preferably from 3 to 6 carbon atoms. Cycloalkyl groups may be monovalent or polyvalent (e.g., divalent). This definition of “cycloalkyl” encompasses polycyclic cycloalkyls (e.g., bicycles) and bridged cycloalkyl structures, including cycles bound together through one atom (“spiro”) or through two atoms. This definition of “cycloalkyl” encompasses cycloalkyls including a cyclic alkyl group substituted by at least one non-cyclic alkyl, such as, for example, a (C₁-C₈) alkyl (preferably, (C₁-C₄) alkyl, e.g., methyl). Non-limiting examples of cycloalkyl groups include cyclopropyl, cyclobutyl, cyclopentyl, cyclohexyl, cycoheptyl, cyclooctanyl, cyclononanyl, cyclodecanyl, norbornyl, adamantyl, bicyclo[2.2.2]octanyl, bicyclo[4.4.0]decanyl, bicyclo[3.2.1]octanyl, bicyclo[3.3.1]nonanyl, bicyclo[2.1.1]hexane, 2,3-dihydro-1H-indenyl, 1,2,3,4-tetrahydronaphthalenyl, decahydronaphthalenyl, 1,2,3,4-tetrahydronaphthalenyl, and octahydropentalenyl.

“Cₓ-C_{y}” or “(Cₓ-C_{y})” preceding the name of a group means that the group comprises from x to y carbon atoms, in accordance to common terminology in the chemistry field.

“Halogen” refers to a fluorine, chlorine, bromine or iodine atom.

“Heteroalkyl” refers to an alkyl group as defined herein, wherein one or more carbon atoms are replaced by a heteroatom selected from oxygen, nitrogen and sulfur, and wherein the resulting heteroalkyl group comprises at least one carbon atom. In heteroalkyl groups, the heteroatoms are bound along the alkyl chain only to carbon atoms, i.e., each heteroatom is separated from any other heteroatom by at least one carbon atom, typically by at least two carbon atoms. Heteroalkyl groups may be monovalent or polyvalent (e.g., divalent). The nitrogen and sulfur heteroatoms may optionally be oxidized and the nitrogen heteroatoms may optionally be quaternized (e.g., sulfur may be oxidized as SO or SO₂). Heteroalkyl groups may further include one or more ═O and/or ═S groups. In one embodiment, at least two carbon atoms are replaced by a heteroatom. In one embodiment, the heteroalkyl is bound to another group or molecule through a carbon atom, i.e., the binding atom is not selected among the heteroatoms included therein. In one embodiment, the heteroalkyl is bound to another group or molecule through one of the heteroatoms included therein. When substituted by one or more other group(s), an heteroalkyl may be substituted either through a carbon atom or through a heteroatom (e.g., nitrogen), unless otherwise specified. Non-limiting examples of heteroalkyl include alkoxy, ethers and polyethers (e.g., polyethylene glycol), secondary and tertiary amines and polyamines, thioethers and polythioethers, and combinations thereof.

“Heteroaryl” refers to aromatic rings or aromatic ring systems comprising from 5 to 15 carbon atoms, preferably from 4 to 12 carbon atoms, more preferably from 3 to 10 carbon atoms, having one or two rings that are fused together or linked covalently, wherein at least one ring is aromatic, and wherein one or more carbon atoms in one or more of these rings is replaced by oxygen, nitrogen and/or sulfur atoms. Heteroaryl groups may be monovalent or polyvalent (e.g., divalent). The nitrogen and sulfur heteroatoms may optionally be oxidized and the nitrogen heteroatoms may optionally be quaternized (e.g., the heteroatom is substituted by oxo (═O) for sulfur atom or (→O) for nitrogen atom). This definition of “heteroaryl” encompasses the partially hydrogenated derivatives of the carbocyclic systems enumerated herein, as well as ring systems including one or more fused non-aromatic cycloalkyl and/or heterocycloalkyl ring(s), as long as at least one ring is aromatic. In one embodiment, the heteroaryl is bound to another group or molecule through a carbon atom, i.e., the binding atom is not selected among the heteroatoms included therein. In one embodiment, the heteroaryl is bound to another group or molecule through one of the heteroatoms included therein. When substituted by one or more other group(s), an heteroaryl may be substituted either through a carbon atom or through a heteroatom (e.g., nitrogen), unless otherwise specified. Heteroaryls may optionally be substituted by at least one group such as, for example, halogen (e.g., F or Cl), (C₁-C₈) alkyl (preferably, (C₁-C₄) alkyl, e.g., methyl) or nitrile (CN). Non-limiting examples of heteroaryl groups include pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, tetrazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl, imidazo[1,2-a]pyridinyl, 6-oxo-pyridazin-1 (6H)-yl, 2-oxopyridin-1 (2H)-yl, 6-oxo-pyridazin-1 (6H)-yl, 2-oxopyridin-1 (2H)-yl, 1,3-benzodioxolyl, quinolinyl, isoquinolinyl, cinnolinyl, quinazolinyl and quinoxalinyl. Non-limiting examples of heteroaryl groups comprising at least one fused non-aromatic ring include 2,3-dihydrobenzofuranyl, benzo[d][1,3]dioxolyl, indolinyl, 2,3-dihydrobenzo[b][1,4]dioxinyl, 3,4-dihydro-2H-benzo[b][1,4]oxazinyl, 1,2,3,4-tetrahydroquinoxaline, 3,4-dihydro-2H-benzo[b][1,4]thiazine and 2,3-dihydrobenzo[b][1,4]oxathiine.

“Heteroarylidene” refers to a heteroaryl group bond to a moiety through an exo carbon-carbon double bound, i.e., ═CH-heteroaryl bond to a carbon atom. The moiety is typically cyclic such as, for example, an heterocycloalkyl.

“Heterocycloalkyl” refers to a cyclic heteroalkyl group, typically comprising from 2 to 15 carbon atoms, preferably from 2 to 11 carbon atoms, more preferably from 2 to 7 carbon atoms, furthermore preferably from 2 to 6 carbon atoms. Heterocycloalkyl groups may be monovalent or polyvalent (e.g., divalent). Heterocycloalkyl groups are typically 3- to 7-membered, preferably 5- or 6-membered. Heterocycloalkyl are typically monocyclic or bicyclic, preferably monocyclic. This definition encompasses polycyclic heterocycloalkyls (e.g., bicycles) and bridged heterocycloalkyl structures, including cycles bound together through one atom (“spiro”) or through two atoms. The nitrogen and sulfur heteroatoms may optionally be oxidized and the nitrogen heteroatoms may optionally be quaternized (e.g., the heteroatom is substituted by oxo (═O) for sulfur atom or (→O) for nitrogen atom). In one embodiment, the heterocycloalkyl is bound to another group or molecule through a carbon atom, i.e., the binding atom is not selected among the heteroatoms included therein. In one embodiment, the heterocycloalkyl is bound to another group or molecule through one of the heteroatoms included therein. When substituted by one or more other group(s), an heterocycloalkyl may be substituted either through a carbon atom or through a heteroatom (e.g., nitrogen), unless otherwise specified. Heterocycloalkyls may optionally be substituted by at least one group such as, for example, halogen (e.g., F or Cl), (C₁-C₈) alkyl (preferably, (C₁-C₄) alkyl, e.g., methyl), nitrile (CN) or ═O. Non-limiting examples of heterocycloalkyl include aziridine, pyrrolidine, piperidine, piperazine, morpholine, thiomorpholine, azepane, azocane, octahydro-1H-isoindole, decahydroisoquinoline, tetrahydrofuran, tetrahydropyran, tetrahydroisoquinoline (e.g., 1,2,3,4-tetrahydroisoquiline), hexahydropyridazine, hexahydropyrazine, hexahydropyrimidine, decahydroquinoline, octahydropyrrolo[3,4-c]pyrrole, isoindoline, 1,2,3,4-tetrahydroquinoline and oxetane.

“Isobutyronitrile” refers to a (NC) (CH₃)₂C-group of the following formula.

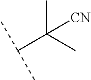

“Prodrug” refers to a pharmacologically acceptable derivative of a therapeutic agent (e.g., a compound according to the invention) whose in vivo biotransformation product is the therapeutic agent (active drug). Prodrugs are typically characterized by increased bioavailability and are readily metabolized in vivo into the active compounds. Non-limiting examples of prodrugs include amide prodrugs and carboxylic acid ester prodrugs.

“Solvate” refers to molecular complex comprising a compound along with stoichiometric or sub-stoichiometric amounts of one or more molecules of one or more solvents, typically the solvent is a pharmaceutically acceptable solvent such as, for example, ethanol. The term “hydrate” refers to a solvate when the solvent is water (H₂O).

“Ylidene” refers to CH group involved in an exo carbon-carbon double bound with another moiety. The moiety is typically cyclic such as, for example, an heterocycloalkyl.

### General Definitions

“About” is used herein to mean approximately, roughly, around, or in the region of. The term “about” preceding a figure means plus or less 10% of the value of the figure. When the term “about” is used in conjunction with a numerical range, it modifies that range by extending the boundaries above and below the numerical values set forth by 10%.

“Administration”, or a variant thereof (e.g., “administering”), means providing a therapeutic agent alone or as part of a pharmaceutically acceptable composition, to the patient in whom/which the condition, symptom, or disease is to be treated.

“Comprise” or a variant thereof (e.g., “comprises”, “comprising”) is used herein according to common patent application drafting terminology. Hence, “comprise” preceded by an object and followed by a constituent means that the presence of a constituent in the object is required (typically as a component of a composition), but without excluding the presence of any further constituent(s) in the object. Moreover, any occurrence of “comprise” or a variant thereof herein also encompasses narrower expression “substantially consist of”, further narrower expression “consist of” and any variants thereof (e.g., “consists of”, “consisting of”), and may be replaced thereby, unless otherwise stated.

“GDNF family receptor alpha-1”, “GFRα1”, or “GDNFRal”, also named “RET ligand 1” or “TGF-beta-related neurotrophic factor receptor 1”, is a protein from the GDNFR family that acts as a receptor for GDNF. It mediates the GDNF-induced autophosphorylation and activation of the RET receptor. In humans, GFRα1 is encoded by the GFRA1 gene. An exemplary amino acid sequence of human GFRα1 is given in SEQ ID NO: 1, in which amino acid residues 1-24 correspond to the signal peptide and amino acid residues 430-465 correspond to the propeptide which is removed in mature form.

SEQ ID NO: 1
MFLATLYFALPLLDLLLSAEVSGGDRLDCVKASDQCLKEQSCSTKYRTL
RQCVAGKETNFSLASGLEAKDECRSAMEALKOKSLYNCRCKRGMKKEKN
CLRIYWSMYQSLQGNDLLEDSPYEPVNSRLSDIFRVVPFISDVFQQVEH
IPKGNNCLDAAKACNLDDICKKYRSAYITPCTTSVSNDVCNRRKCHKAL
ROFFDKVPAKHSYGMLFCSCRDIACTERRRQTIVPVCSYEEREKPNCLN
LQDSCKTNYICRSRLADFFTNCQPESRSVSSCLKENYADCLLAYSGLIG
TVMTPNYIDSSSLSVAPWCDCSNSGNDLEECLKFLNFFKDNTCLKNAIQ
AFGNGSDVTVWQPAFPVQTTTATTTTALRVKNKPLGPAGSENEIPTHVL
PPCANLQAQKLKSNVSGNTHLCISNGNYEKEGLGASSHITTKSMAAPPS
CGLSPLLVLVVTALSTLLSLTETS

“Human” refers to a male or female human subject at any stage of development, including neonate, infant, juvenile, adolescent and adult.

“Neuroprotective” refers to the protection of a neuronal cell from insults, events, or conditions that would normally result in a loss of neuronal cell's functions, and ultimately, neuronal cell death. Such insults, events, or conditions include, without limitation, neuronal stress, for instance, caused by hypoxia or ischemia; traumatic injuries; and exposure to toxic molecules, for instance, to abnormal misfolded proteins, protein aggregates, excitotoxins, reactive oxygen species, endoplasmic reticulum stressors, mitochondrial stressors, Golgi apparatus antagonists and the like. The term also characterizes the detectable biological activity of a compound in reducing the amount or level of neuronal cell's loss of functions and/or neuronal cell death.

“Neurorestorative” refers to the restoration or rescue of a neuronal cell and in particular of its functions, from the effect of an insult, event, or condition that would normally result in a loss of neuronal cell's functions if not in neuronal cell death.

“Patient” refers to a subject who/which is awaiting the receipt of, or is receiving medical care or was/is/will be the object of a medical procedure, or is monitored for the development of the targeted disease or condition, such as, for example, a neurological disorder.

“Pharmaceutically acceptable” means that the ingredients of a composition are compatible with each other and not deleterious to the subject to which/whom it is administered.

“Pharmaceutically acceptable carrier” refers to an excipient that does not produce an adverse, allergic or other untoward reaction when administered to an animal, preferably a human. It includes any and all solvents, dispersion media, coatings, antibacterial and antifungal agents, isotonic and absorption delaying agents and the like. For human administration, preparations should meet sterility, pyrogenicity, general safety and purity standards as required by regulatory offices, such as, e.g., FDA Office or EMA. Examples of pharmaceutically acceptable carriers include, but are not limited to, ion exchangers, alumina, aluminium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

“Pharmaceutical composition” refers to a composition comprising at least one therapeutic agent (e.g., a compound according to the present invention) and at least one pharmaceutically acceptable carrier.

“Proto-oncogene tyrosine-protein kinase receptor Ret”, or in short “RET”, also named “cadherin family member 12”, is a receptor tyrosine kinase involved in numerous cellular mechanisms including cell proliferation, neuronal navigation, cell migration, and cell differentiation. RET is activated upon (i) binding of a neurotrophic factor of the GDNF family (e.g., GDNF, neurturin, artemin or persephin) to a receptor of the GDNFR family (e.g., GFRα1, GFRα2, GFRα3 or GFRα4), then (ii) complex formation between RET and the receptor of the GDNFR family, (iii) dimerization and (iv) trans-autophosphorylation. An exemplary amino acid sequence of human RET is given in SEQ ID NO: 2, in which amino acid residues 1-28 correspond to the signal peptide.

SEQ ID NO: 2
MAKATSGAAGLRLLLLLLLPLLGKVALGLYFSRDAYWEKLYVDQAAGTP
LLYVHALRDAPEEVPSFRLGQHLYGTYRTRLHENNWICIQEDTGLLYLN
RSLDHSSWEKLSVRNRGFPLLTVYLKVFLSPTSLREGECQWPGCARVYF
SFFNTSFPACSSLKPRELCFPETRPSFRIRENRPPGTFHQFRLLPVQFL
CPNISVAYRLLEGEGLPFRCAPDSLEVSTRWALDREQREKYELVAVCTV
HAGAREEVVMVPFPVTVYDEDDSAPTFPAGVDTASAVVEFKRKEDTVVA
TLRVFDADVVPASGELVRRYTSTLLPGDTWAQQTFRVEHWPNETSVQAN
GSFVRATVHDYRLVLNRNLSISENRTMQLAVLVNDSDFQGPGAGVLLLH
FNVSVLPVSLHLPSTYSLSVSRRARRFAQIGKVCVENCQAFSGINVQYK
LHSSGANCSTLGVVTSAEDTSGILFVNDTKALRRPKCAELHYMVVATDQ
QTSRQAQAQLLVTVEGSYVAEEAGCPLSCAVSKRRLECEECGGLGSPTG
RCEWRQGDGKGITRNFSTCSPSTKTCPDGHCDVVETQDINICPQDCLRG
SIVGGHEPGEPRGIKAGYGTCNCFPEEEKCFCEPEDIQDPLCDELCRTV
IAAAVLFSFIVSVLLSAFCIHCYHKFAHKPPISSAEMTFRRPAQAFPVS
YSSSGARRPSLDSMENQVSVDAFKILEDPKWEFPRKNLVLGKTLGEGEF
GKVVKATAFHLKGRAGYTTVAVKMLKENASPSELRDLLSEFNVLKQVNH
PHVIKLYGACSQDGPLLLIVEYAKYGSLRGFLRESRKVGPGYLGSGGSR
NSSSLDHPDERALTMGDLISFAWQISQGMQYLAEMKLVHRDLAARNILV
AEGRKMKISDFGLSRDVYEEDSYVKRSQGRIPVKWMAIESLFDHIYTTQ
SDVWSFGVLLWEIVTLGGNPYPGIPPERLFNLLKTGHRMERPDNCSEEM
YRLMLQCWKQEPDKRPVFADISKDLEKMMVKRRDYLDLAASTPSDSLIY
DDGLSEEETPLVDCNNAPLPRALPSTWIENKLYGMSDPNWPGESPVPLT
RADGTNTGFPRYPNDSVYANWMLSPSAAKLMDTFDS

“Selected from” is used herein according to common patent application drafting terminology, to introduce a list of elements among which one or more item(s) is (are) selected. Any occurrence of “selected from” in the specification may be replaced by “selected from the group comprising or consisting of” and reciprocally without changing the meaning thereof.

“Subject” refers to an animal, typically a warm-blooded animal, preferably a mammal, more preferably a primate, furthermore preferably a human. In one embodiment, the subject is a “patient” as defined herein. In one embodiment, the subject is affected, preferably is diagnosed, with a disease. In one embodiment, the subject is at risk of developing a disease. Examples of risks factor include, but are not limited to, genetic predisposition, or familial history of the disease.

“Therapeutic agent”, “active pharmaceutical ingredient” and “active ingredient” refer to a compound for therapeutic use and relating to health. Especially, a therapeutic agent (e.g., a compound according to the present invention) may be indicated for treating a disease (e.g., a neurological disorder). An active ingredient may also be indicated for improving the therapeutic activity of another therapeutic agent.

“Therapeutically effective amount” (in short “effective amount”) refers to the amount of a therapeutic agent (e.g., a compound according to the present invention) that is sufficient to achieve the desired therapeutic, prophylactic or preventative effect in the patient to which/whom it is administered, without causing significant negative or adverse side effects to said patient. A therapeutically effective amount may be administered prior to the onset of the disease for a prophylactic or preventive action. Alternatively, or additionally, the therapeutically effective amount may be administered after initiation of the disease for a therapeutic action.

“Treating”, “treatment” or “alleviation” refers to both therapeutic treatment and prophylactic or preventative measures, wherein the object is to prevent or slow down (lessen) the targeted pathologic condition or disorder (herein a “disease”) (e.g., a neurological disorder). Those in need of treatment include those already with the disease as well as those prone to have the disease or those in whom the condition or disease is to be prevented. A patient is successfully “treated” for a disease if, after receiving a therapeutic amount of a therapeutic agent (e.g., a compound according to the present invention), the patient shows observable and/or measurable reduction in or absence of one or more of the following: reduction in the number of pathogens; reduction in the percent of total cells that are pathogenic; and/or relief to some extent, one or more of the symptoms associated with the specific disease; reduced morbidity and mortality, and improvement in quality of life issues. The above parameters for assessing successful treatment and improvement in the disease are readily measurable by routine procedures familiar to a physician.

### DETAILED DESCRIPTION

### Compound

An object of the present invention is a compound of formula (I)

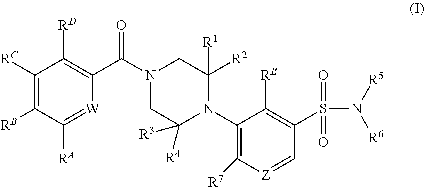

or a pharmaceutically acceptable salt and/or solvate thereof;
   wherein
   W represents CH or N;
   R^{A}, R^{B}, R^{C} and R^{D} each independently represents hydrogen, F, Cl, CH₃, CF₃, CHF₂ or CH₂F,
      provided that at least one among R^{A}, R^{B}, R^{C} and R^{D} does not represent hydrogen;
   R¹ represents (C₁-C₈) alkyl, wherein the alkyl is optionally substituted by at least one OH, (C₁-C₃) alkoxy or F; and R², R³ and R⁴ represents hydrogen;
   or R¹ and R⁴ form together —CH₂-O-CH₂- or —CH₂—CH₂—, wherein the —CH₂—CH₂— is optionally substituted by at least one F, OH or OCH₃; and R² and R³ each represents hydrogen;
   R⁷ represents hydrogen, OH, halogen, (C₁-C₅) alkyl, cycloalkyl, (C₁-C₈) alkyl-O—, cycloalkyl-O—, cycloalkyl-(C₁-C₈) alkyl-O—, heterocycloalkyl-O—, R¹¹O—(C₁-C₈) alkyl-O—, R¹¹R¹²N-(C₁-C₅) alkyl-O—, (R¹¹⁰) (R¹²)N—(C₁-C₅) alkyl-O—, R¹¹R¹²N—(C₁-C₅) alkyl-, R¹¹O—(C₁-C₅) alkyl-, NR¹¹R¹², CN, CO₂H, CO₂R¹¹, CONH₂, CON(R¹¹) H, heterocycloalkyl, or heteroaryl; wherein R¹¹ and R¹² each independently represents hydrogen or (C₁-C₅) alkyl;
      wherein the alkyl or cycloalkyl in R⁷ is optionally substituted by at least one F, Cl, OH, ═O, (C₁-C₈) alkyl, (C₁-C₅) alkyl-O—, heterocycloalkyl, aryl or heteroaryl;
         wherein the heterocycloalkyl, aryl or heteroaryl is optionally substituted by at least one F, Cl, (C₁-C₅) alkyl, CF₃, CHF₂, CH₂F, OCF₃, CN, OH, ═O, →O, (C₁-C₅) alkoxy, NR¹³R¹⁴, R¹³R¹⁴N—(C₁-C₅) alkyl-, R¹³O₂C—(C₁-C₅) alkyl-, CO₂H, R¹³R¹⁴N—C(O)—, R¹³⁰—NR¹⁴—, or (C₁-C₈) alkyl-CO₂—;wherein R¹³ and R¹⁴ each independently represents hydrogen or (C₁-C₈) alkyl;
   Z represents C—H, C—R⁸ or N;
      wherein R⁸ represents (C₁-C₄) alkyl, F, Cl, CF₃, CHF₂, CH₂F, OCF₃, CN, OH or (C₁-C₄) alkoxy;
   or Z represents C—R⁸ and R⁷ and R⁸ form together with the carbon atoms to which they are bound a cycloalkyl or heterocycloalkyl,
      wherein the cycloalkyl or heterocycloalkyl is optionally substituted by at least one F, OH, ═O, →O, (C₁-C₈) alkyl, CF₃, HO₂C—CH₂—, (C₁-C₄) alkyl-CO₂—CH₂—, R¹⁵R¹⁶N—CH₂—, aryl, or aryl-(C₁-C₅) alkyl-, wherein R¹⁵ and R¹⁶ each independently represents hydrogen or (C₁-C₈) alkyl;
   R⁵ represents hydrogen, (C₁-C₅) alkyl, CH₃ substituted by one to three (C₁-C₈) alkyl groups, cycloalkyl, heterocycloalkyl, aryl, or cycloalkyl-(C₁-C₈) alkyl;
      wherein the alkyl or cycloalkyl in R⁵ is optionally substituted by at least one F, Cl, (C₁-C₈) alkyl, CF₃, OCF₃, CN, OH, ═O, (C₁-C₈) alkoxy, NR¹⁷R¹⁸, CO₂H, R¹⁷R¹⁸N—C(O)—, R¹⁷O—NR¹⁸-, heterocycloalkyl, aryl or heteroaryl; wherein R¹⁷ and R¹⁸ each independently represents hydrogen or (C₁-C₅) alkyl;
      wherein the heterocycloalkyl, aryl or heteroaryl (i.e., any heterocycloalkyl, aryl or heteroaryl that is represented by R⁵ or that is part of any substituent thereof) is optionally substituted by at least one F, Cl, (C₁-C₅) alkyl, CF₃, CHF₂, CH₂F, OCF₃, CN, OH, ═O,→O, (C₁-C₅) alkoxy, NR¹⁹R²⁰, CO₂H, R¹⁹R 20N—C(O)—, R¹⁹O—NR²⁰, (C₁-C₈) alkyl-CO₂-, R¹⁹R²⁰N—(C₁-C₈) alkyl-, R¹⁹O₂C—(C₁-C₅) alkyl-, heterocycloalkyl, heteroaryl, aryl, or aryl-(C₁-C₅) alkyl-; wherein R¹⁹ and R²⁰ each independently represents hydrogen or (C₁-C₅) alkyl;
   R⁶ represents (C₁-C₅) alkyl, CH₃ substituted by one to three (C₁-C₅) alkyl groups, cycloalkyl, heterocycloalkyl, aryl, or cycloalkyl-(C₁-C₈) alkyl;
      wherein the alkyl or cycloalkyl in R⁶ is optionally substituted by at least one F, Cl, (C₁-C₅) alkyl, CF₃, OCF₃, CN, OH, ═O, (C₁-C₅) alkoxy, NR²¹R²², CO₂H, R²¹R²²N—C(O)—, R²¹⁰—NR²²-, heterocycloalkyl, aryl or heteroaryl; wherein R²¹ and R²² each independently represents hydrogen or (C₁-C₅) alkyl;
      wherein the heterocycloalkyl, aryl or heteroaryl (i.e., any heterocycloalkyl, aryl or heteroaryl that is represented by R⁶ or that is part of any substituent thereof) is optionally substituted by at least one F, Cl, (C₁-C₅) alkyl, CF₃, CHF₂, CH₂F, OCF₃, CN, OH, ═O,→O, (C₁-C₈) alkoxy, NR²³R²⁴, CO₂H, R²³R²⁴N—C(O)—, R²³⁰—NR²⁴—, (C₁-C₅) alkyl-CO₂—, R²³R²⁴N—(C₁-C₈) alkyl-, R²³O₂C—(C₁-C₈) alkyl-, heterocycloalkyl, heteroaryl, aryl, or aryl-(C₁-C₈) alkyl-; wherein R²³ and R²⁴ each independently represents hydrogen or (C₁-C₈) alkyl;
   or R⁵ and R⁶ form together with the nitrogen atom to which they are bound an heterocycloalkyl,
      wherein the heterocycloalkyl (i.e., the heterocycloalkyl that is formed by R⁵, R⁶ and the nitrogen atom to which they are bound) is optionally substituted by at least one F, Cl, (C₁-C₈) alkyl, CF₃, OCF₃, CN, OH, ═O, →O, (C₁-C₈) alkoxy, NR²⁵R²⁶, CO₂H, (C₁-C₈) alkyl-CO₂—, R²⁵R²⁶N—C(O)—, R²⁵⁰—NR²⁶, R²⁵R²⁶N—(C₁-C₈) alkyl-, R²⁵⁰²C—(C₁-C₈) alkyl-, cycloalkyl, heterocycloalkyl, aryl, heteroaryl, cycloalkyl-(C₁-C₈) alkyl-, heterocycloalkyl-(C₁-C₈) alkyl-, aryl-(C₁-C₈) alkyl-, heteroaryl-(C₁-C₈) alkyl-, aryl-cycloalkyl-, cycloalkyl-O—, heterocycloalkyl-O—, aryl-O—, heteroaryl-O—, cycloalkyl-(C₁-C₈) alkyl-O—, heterocycloalkyl-(C₁-C₈) alkyl-O—, aryl-(C₁-C₈) alkyl-O—, heteroaryl-(C₁-C₈) alkyl-O—, cycloalkyl-NR²⁵-, heterocycloalkyl-NR²⁵, aryl-NR²⁵, heteroaryl-NR²⁵-, cycloalkyl-(C₁-C₈) alkyl-NR²⁵, heterocycloalkyl-(C₁-C₈) alkyl-NR²⁵-, aryl-(C₁-C₈) alkyl-NR²⁵-, heteroaryl-(C₁-C₈) alkyl-NR²⁵-, benzylidene, heteroarylidene, aryl-(C₁-C₈) alkyl-ylidene- or heteroaryl-(C₁-C₈) alkyl-ylidene-;
         wherein R²⁵ and R²⁶ each independently represents hydrogen or (C₁-C₈) alkyl;
         wherein the heterocycloalkyl, aryl, heteroaryl, benzylidene or heteroarylidene (i.e., any heterocycloalkyl, aryl, heteroaryl, benzylidene or heteroarylidene that is part of any substituent of the heterocycloalkyl formed by R⁵, R⁶ and the nitrogen atom to which they are bound) is optionally substituted at least one F, Cl, (C₁-C₈) alkyl, CF₃, CHF₂, CH₂F, OCF₃, CN, OH, ═O,→O, (C₁-C₈) alkoxy, NR²⁷R²⁸, CO₂H, R²⁷R²⁸N—C(O)—, R²⁷O —NR²⁸-, (C₁-C₈) alkyl-CO₂—, R²⁷R²⁸N—(C₁-C₅) alkyl-, R²⁷⁰²C—(C₁-C₈) alkyl-, heterocycloalkyl, heteroaryl, aryl, or aryl-(C₁-C₈) alkyl-; wherein R²⁷ and R²⁸ each independently represents hydrogen or (C₁-C₈) alkyl;
   R^{E} represents hydrogen, (C₁-C₃) alkyl or halogen.

In any one of the following embodiments directed to specific limitations to the structure of the compounds of formula (I), any alkyl, heteroalkyl, cycloalkyl, heterocycloalkyl, aryl, heteroaryl, benzylidene or heteroarylidene may independently be optionally substituted as indicated under formula (I) herein, unless otherwise specified.

According to one preferred embodiment, the compound of formula (I) is a compound of formula (I′)

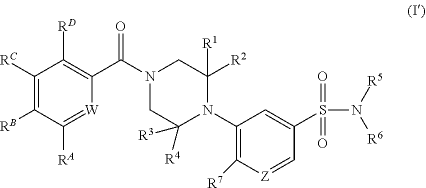

or a pharmaceutically acceptable salt and/or solvate thereof;
   wherein
   W represents CH or N;
   R^{A}, R^{B}, R^{C} and R^{D} each independently represents hydrogen, F, Cl, CH₃, CF₃, CHF₂ or CH₂F,
      provided that at least one among R^{A}, R^{B}, R^{C} and R^{D} does not represent hydrogen;
   R¹ represents (C₁-C₈) alkyl, wherein the alkyl is optionally substituted by at least one OH, (C₁-C₃) alkoxy or F; and R², R³ and R⁴ represents hydrogen;
   or R¹ and R⁴ form together —CH₂-O-CH₂- or —CH₂—CH₂—, wherein the —CH₂—CH₂— is optionally substituted by at least one F, OH or OCH₃; and R² and R³ each represents hydrogen;
   R⁷ represents hydrogen, OH, halogen, (C₁-C₈) alkyl, cycloalkyl, (C₁-C₈) alkyl-O—, cycloalkyl-O—, cycloalkyl-(C₁-C₈) alkyl-O—, heterocycloalkyl-O—, R¹¹O—(C₁-C₈) alkyl-O—, R¹¹R¹²N—(C₁-C₈) alkyl-O— or (R¹¹O) (R¹²)N—(C₁-C₈) alkyl-O—; wherein R¹¹ and R¹² each independently represents hydrogen or (C₁-C₈) alkyl;
      wherein the alkyl or cycloalkyl is optionally substituted by at least one F, Cl, heterocycloalkyl, aryl or heteroaryl;
         wherein the heterocycloalkyl, aryl or heteroaryl is optionally substituted by at least one F, Cl, CF₃, CHF₂, CH₂F, OCF₃, CN, OH, (C₁-C₈) alkoxy, NR¹³R¹⁴, CO₂H, R¹³R¹⁴N—C(O)—, or R¹³⁰—NR¹⁴—; wherein R¹³ and R¹⁴ each independently represents hydrogen or (C₁-C₈) alkyl;
      Z represents C—H or N;
      or Z represents C-R⁸ and R⁷ and R⁸ form together with the carbon atoms to which they are bound a cycloalkyl or heterocycloalkyl,
         wherein the cycloalkyl or heterocycloalkyl is optionally substituted by at least one F, OH, CF₃, HO₂C—CH₂—, (C₁-C₄) alkyl-CO₂—CH₂—, or R¹⁵R¹⁶N—CH₂—, wherein R¹⁵ and R¹⁶ each independently represents hydrogen or (C₁-C₈) alkyl;
      R⁵ represents hydrogen, (C₁-C₈) alkyl, CH₃ substituted by one to three (C₁-C₈) alkyl groups, cycloalkyl or cycloalkyl-(C₁-C₈) alkyl;
         wherein the alkyl or cycloalkyl is optionally substituted by at least one F, Cl, CF₃, OCF₃, CN, OH, (C₁-C₈) alkoxy, NR¹⁷R¹⁸, CO₂H, R¹⁷R¹⁸N—C(O)—, R¹⁷O—NR¹⁸—, heterocycloalkyl, aryl or heteroaryl; wherein R¹⁷ and R¹⁸ each independently represents hydrogen or (C₁-C₈) alkyl;
         wherein the heterocycloalkyl, aryl or heteroaryl is optionally substituted by at least one F, Cl, CF₃, CHF₂, CH₂F, OCF₃, CN, OH, (C₁-C₈) alkoxy, NR¹⁹R²⁰, CO₂H, R¹⁹R²⁰N—C(O)—, or R¹⁹O—NR²⁰ —; wherein R¹⁹ and R²⁰ each independently represents hydrogen or (C₁-C₈) alkyl;
      R⁶ represents (C₁-C₈) alkyl, CH₃ substituted by one to three (C₁-C₈) alkyl groups, cycloalkyl or cycloalkyl-(C₁-C₈) alkyl;
         wherein the alkyl or cycloalkyl is optionally substituted by at least one F, Cl, CF₃, OCF₃, CN, OH, (C₁-C₈) alkoxy, NR²¹R²², CO₂H, R²¹R²²N—C(O)—, R²¹⁰—NR²²-, heterocycloalkyl, aryl or heteroaryl; wherein R²¹ and R²² each independently represents hydrogen or (C₁-C₈) alkyl;
         wherein the heterocycloalkyl, aryl or heteroaryl is optionally substituted by at least one F, Cl, CF₃, CHF₂, CH₂F, OCF₃, CN, OH, (C₁-C₈) alkoxy, NR²³R²⁴, CO₂H, R²³R²⁴N—C(O)—, or R²³⁰—NR²⁴; wherein R²³ and R²⁴ each independently represents hydrogen or (C₁-C₈) alkyl;
      or R⁵ and R⁶ form together with the nitrogen atom to which they are bound an heterocycloalkyl,
         wherein the heterocycloalkyl is optionally substituted by at least one F, Cl, CF₃, OCF₃, CN, OH, (C₁-C₈) alkoxy, NR²⁵R²⁶, CO₂H, R²⁵R²⁶N—C(O)—, R²⁵⁰—NR²⁶-, heterocycloalkyl, aryl, heteroaryl, heterocycloalkyl-(C₁-C₈) alkyl-, aryl-(C₁-C₈) alkyl-, heteroaryl-(C₁-C₈) alkyl-, cycloalkyl-(C₁-C₈) alkyl-O—, aryl-(C₁-C₈) alkyl-O—, heteroaryl-(C₁-C₈) alkyl-O—, cycloalkyl-(C₁-C₈) alkyl-NR²⁵_, aryl-(C₁-C₈) alkyl-NR²⁵-, heteroaryl-(C₁--C₈) alkyl-NR²⁵-, benzylidene, heteroarylidene, aryl-(C₁-C₈) alkyl-ylidene- or heteroaryl-(C₁-C₈) alkyl-ylidene-;
         wherein R²⁵ and R²⁶ each independently represents hydrogen or (C₁-C₈) alkyl;
         wherein the heterocycloalkyl, aryl, heteroaryl, benzylidene or heteroarylidene is optionally substituted at least one F, Cl, CF₃, CHF₂, CH₂F, OCF₃, CN, OH, (C₁-C₈) alkoxy, NR²⁷R²⁸, CO₂H, R²⁷R²⁸N—C(O)—, or R²⁷O —NR²⁸—; wherein R²⁷ and R²⁸ each independently represents hydrogen or (C₁-C₈) alkyl.

According to one embodiment, W represents CH.

According to one embodiment, at least one among R^{A}, R^{B}, R^{C} and R^{D} represents hydrogen. In one embodiment, exactly one among R^{A}, R^{B}, R^{C} and R^{D} represents hydrogen. In one embodiment, exactly two among R^{A}, R^{B}, R^{C} and R^{D} represents hydrogen. In one embodiment, exactly three among R^{A}, R^{B}, R^{C} and R^{D} represents hydrogen.

In one embodiment, at least one among R^{A} and R^{C} represents hydrogen. In one particular embodiment, R^{A} represents hydrogen. In one particular embodiment, R^{C} represents hydrogen.

According to one embodiment, at least one among R^{A}, R^{B} and R^{D} represents F or Cl. In one embodiment, at least one among R^{B} and R^{D} represents F or Cl. In one particular embodiment, R^{B} and R^{D} each independently represents F or Cl. In one particular embodiment, R^{B} represents F. In one particular embodiment, R^{B} represents Cl. In one particular embodiment, R^{D} represents F. In one particular embodiment, R^{D} represents Cl. In one preferred embodiment, R^{B} represents F. In one preferred embodiment, R^{D} represents Cl. In one further preferred embodiment, R^{B} represents F and R^{D} represents Cl.

As defined under formula (I) hereinabove, R¹ may represents (C₁-C₈) alkyl, wherein the alkyl is optionally substituted by at least one OH, (C₁-C₃) alkoxy or F, i.e., the (C₁-C₈) alkyl in R¹ is optionally substituted by at least one group, and the group may be OH, (C₁-C₃) alkoxy or F. According to one embodiment, R¹ represents (C₁-C₈) alkyl, wherein the alkyl is optionally substituted by at least one group selected from OH, (C₁-C₃) alkoxy and F.

According to one embodiment, R¹ and R⁴ form together —CH₂—O—CH₂— or —CH₂—CH₂. In one embodiment, R¹ and R⁴ form together —CH₂—CH₂—. In one embodiment, the —CH₂—CH₂— is optionally substituted by at least one F, OH or OCH₃. In one embodiment, the —CH₂—CH₂— is not substituted.

According to one embodiment, R¹ represents methyl, ethyl, CF₃ or methoxymethyl (CH₃OCH₂—). In one embodiment, R¹ represents methyl, ethyl or CF₃. In one embodiment, R¹ represents methoxymethyl (CH₃OCH₂—).

According to one embodiment, R⁷ represents hydrogen, OH, halogen, (C₁-C₈) alkyl, cycloalkyl, (C₁-C₈) alkyl-O—, cycloalkyl-O—, cycloalkyl-(C₁-C₈) alkyl-O—, heterocycloalkyl-O—, R¹¹O—(C₁-C₈) alkyl-O—, R¹¹R¹²N—(C₁-C₈) alkyl-O— or (R¹¹O) (R¹²)N—(C₁-C₈) alkyl-O—; wherein R¹¹ and R¹² each independently represents hydrogen or (C₁-C₈) alkyl; wherein the alkyl, cycloalkyl, heterocycloalkyl, aryl or heteroaryl is optionally substituted as defined under formula (I) hereinabove.

According to one embodiment, R⁷ represents hydrogen, OH or halogen. In one embodiment, R⁷ represents hydrogen. In one preferred embodiment, R⁷ represents hydroxyl (OH). In one embodiment, R⁷ represents halogen. In one particular preferred embodiment, R⁷ represents F or Cl. In one particular embodiment, R⁷ represents F. In one particular embodiment, R⁷ represents Cl.

In one preferred embodiment, R⁷ does not represent hydrogen.

According to one embodiment, R⁷ represents (C₁-C₈) alkyl-O— (i.e., (C₁-C₈) alkoxy), or cycloalkyl-O—. In one embodiment, the alkyl or cycloalkyl is optionally substituted by at least one F, Cl, OH, (C₁-C₈) alkoxy or aryl. In one particular embodiment, R⁷ represents OCH₃ (methoxy), OCH₂CH₃, OCF₃, cyclobutyl-O—, HO-CH₂— CH₂—O—, CH₃₀—CH₂—CH₂—O— or phenyl-CH₂—O—.

According to one embodiment, R⁷ represents (C₁-C₈) alkyl-O— (i.e., (C₁-C₈) alkoxy). In one embodiment, the alkyl is optionally substituted by at least one F or Cl. In one particular embodiment, the halogen is F. In one embodiment, R⁷ represents OCH₃ (methoxy), OCH₂CH₃ or OCF₃.

According to one embodiment, R⁷ represents cycloalkyl-O—. In one embodiment, R⁷ represents cyclobutyl-O—.

According to one embodiment, R⁷ represents (C₁-C₈) alkyl-O— (i.e., (C₁-C₈) alkoxy), wherein the alkyl is optionally substituted by at least one OH or (C₁-C₈) alkoxy, i.e., RO—(C₁-C₈) alkyl-O— wherein R represents H or (C₁-C₈) alkyl. In one embodiment, R⁷ represents HO—CH₂—CH₂—O— or CH₃O—CH₂—CH₂—O—.

According to one embodiment, R⁷ represents (C₁-C₈) alkyl-O— (i.e., (C₁-C₈) alkoxy), wherein the alkyl is optionally substituted by at least one aryl. In one embodiment, the aryl is not substituted. In one embodiment, R⁷ represents phenyl-CH₂—O—.

According to one embodiment, R⁷ represents (C₁-C₈) alkyl-O— (i.e., (C₁-C₈) alkoxy), wherein the alkyl is optionally substituted by at least one heteroaryl. In one embodiment, the heteroaryl is not substituted. In one embodiment, R⁷ represents heteroaryl-CH₂—O—.

According to one embodiment, R⁷ represents (C₁-C₈) alkyl-O— (i.e., (C₁-C₈) alkoxy), wherein the alkyl is optionally substituted by at least one heterocycloalkyl. In one embodiment, R⁷ represents heterocycloalkyl-(C₁-C₈) alkyl-O—, i.e., the alkyl is substituted by exactly one heterocycloalkyl. In one embodiment, the heterocycloalkyl is not substituted.

In one preferred embodiment, R⁷ represents F, Cl, OCH₃ (methoxy), OCH₂CH₃, OCF₃, cyclobutyl-O—, HO—CH₂—CH₂—O—, CH₃₀—CH₂—CH₂—O—, phenyl-CH₂—O—, 1H-imidazole-4-yl-, 1-methyl-imidazole-4-yl-, CH₃, CN, CO₂H, Cl, F, CH₂OH, C(CH₃)₂OH, CH₂N(CH₃) 2, cyclopropyl, cyclobutyl-O—, (4-pyridine) —CH₂—O—, (3-pyridine) —CH₂—O— or benzyl-O—. In one particular preferred embodiment, R⁷ represents 1H-imidazole-4-yl-, 1-methyl-imidazole-4-yl-, CH₃, CN, CO₂H, CH₂OH, C(CH₃)₂OH, CH₂N(CH₃) 2, cyclopropyl, cyclobutyl-O—, (4-pyridine) —CH₂—O—, (3-pyridine) —CH₂—O— or benzyl-O—.

According to one embodiment, Z represents C—H or N; or Z represents C-R⁸ and R⁷ and R⁸ form together with the carbon atoms to which they are bound a cycloalkyl or heterocycloalkyl; wherein the cycloalkyl or heterocycloalkyl is optionally substituted as defined under formula (I) hereinabove. In other words, in this embodiment, R⁸ represent hydrogen, except where R⁷ and R⁸ form together with the carbon atoms to which they are bound a cycloalkyl or heterocycloalkyl.

According to one embodiment, Z represents C—H or N. In one embodiment, Z represents C—H. In one embodiment, Z represents N.

According to one preferred embodiment, Z represents C-R⁸; wherein R⁸ represents (C₁-C₄) alkyl, F, Cl, CF₃, CHF₂, CH₂F, OCF₃, CN, OH or (C₁-C₄) alkoxy. In one preferred embodiment, R⁸ represents methyl, ethyl, F, Cl, CF₃, CN or OH.

According to another embodiment, Z represents C-R⁸ and R⁷ and R⁸ form together with the carbon atoms to which they are bound a cycloalkyl or heterocycloalkyl; wherein the cycloalkyl or heterocycloalkyl is optionally substituted as defined under formula (I) herein.

In one preferred embodiment, Z represents C-R⁸ and R⁷ and R⁸ form together with the carbon atoms to which they are bound an heterocycloalkyl; wherein the heterocycloalkyl is optionally substituted as defined under formula (I) herein. In one particular embodiment, the heterocycloalkyl comprises at least one oxygen atom, i.e., the heterocycloalkyl is a cyclic ether. In one particular embodiment, the heterocycloalkyl comprises at least one nitrogen atom, i.e., the heterocycloalkyl is a cyclic amine. In one particular embodiment, the heterocycloalkyl is six-membered or five-membered. In one particular embodiment, the heterocycloalkyl comprises exactly one oxygen atom, two oxygen atoms, or one nitrogen atom.

According to one embodiment, R⁵ represents hydrogen or (C₁-C₈) alkyl. In one embodiment, R⁵ represents hydrogen. In one embodiment, R⁵ represents (C₁-C₈) alkyl. In one preferred embodiment, R⁵ represents hydrogen, methyl or ethyl. In one particular embodiment, R⁵ represents hydrogen. In one particular embodiment, R⁵ represents methyl or ethyl.

According to one embodiment, R⁶ represents (C₁-C₈) alkyl, cycloalkyl or cycloalkyl-(C₁-C₈) alkyl-. In one embodiment, the alkyl or cycloalkyl is optionally substituted by at least one F. In one preferred embodiment, the alkyl or cycloalkyl is not substituted. In one embodiment, R⁶ represents methyl, ethyl, n-propyl, 1-fluoro-n-propane, tert-butyl, cyclopropyl, cyclobutyl or cyclopropyl-CH₂—.

According to one embodiment, R⁶ represents (C₁-C₈) alkyl, cycloalkyl or cycloalkyl-(C₁-C₈) alkyl- or heterocycloalkyl. In one embodiment, the alkyl or cycloalkyl is optionally substituted by at least one F, CN or CF₃. In one preferred embodiment, the alkyl or cycloalkyl is not substituted. In one embodiment, R⁶ represents methyl, ethyl, i-propyl, isobutyronitrile, n-propyl, 3-fluoro-n-propane, tert-butyl, cyclopropyl, 1-methylcyclopropyl, cyclobutyl, 1-methylcyclobutyl, cyclopropyl-CH₂—, (F3C) C (Me) 2-, 3-methyloxetane-3-yl or oxetane-3-yl.

According to one embodiment, R⁶ represents (C₁-C₈) alkyl. In one embodiment, the alkyl is optionally substituted by at least one F. In one embodiment, the alkyl is not substituted. In one particular embodiment, R⁵ represents methyl, ethyl, n-propyl, 1-fluoro-n-propane or tert-butyl.

According to one embodiment, R⁶ represents cycloalkyl. In one embodiment, R⁶ represents cyclopropyl or cyclobutyl.

According to one embodiment, R⁶ represents cycloalkyl-(C₁-C₈) alkyl. In one particular embodiment, R⁶ represents cyclopropylmethyl (cyclopropyl-CH₂—).

According to one embodiment, R⁵ and R⁶ form together with the nitrogen atom to which they are bound an heterocycloalkyl. In one embodiment, the heterocycloalkyl is optionally substituted by at least one F. In one preferred embodiment, R⁵ and R⁶ form together with the nitrogen atom to which they are bound a pyrrolidine, piperidine, 4-fluoropiperidine, 3-fluoropyrrolidine, 4-trifluoromethylpiperidine, 4-benzyl-piperidine, 3-benzylpyrrolidine, 3-benzyl-piperidine, 4-phenylpiperidine, 4-benzylidenepiperidine, octahydro-1H-isoindole, 2-benzyloctahydropyrrolo[3,4-c]pyrrole, 3-(benzyloxy) pyrrole, 3-(methoxymethyl) azetidine, 2-azabicyclo[2.2.1]heptane, 5-(4-methylpiperazin-1-yl) pyrimidine and 4-phenethylpiperidine. In one particular embodiment, R⁵ and R⁶ form together with the nitrogen atom to which they are bound a pyrrolidine, 4-fluoropiperidine, 3-fluoropyrrolidine, 4-trifluoromethylpiperidine, 4-benzyl-piperidine, 3-benzylpyrrolidine, 3-benzyl-piperidine, 4-phenylpiperidine, 4-benzylidenepiperidine, octahydro-1H-isoindole, 3-(benzyloxy) pyrrole, 2-azabicyclo[2.2.1]heptane, 5-(4-methylpiperazin-1-yl)pyrimidine or 4-phenethylpiperidine.

In one preferred embodiment, R⁵ and R⁶ form together with the nitrogen atom to which they are bound a pyrrolidine, 3,3-dimethylmorpholine, 4-fluoropiperidine, 3-fluoropyrrolidine, 4-trifluoromethylpiperidine, 4-benzyl-piperidine, 3-benzylpyrrolidine, 3-benzyl-piperidine, 4-phenylpiperidine, 4-(4-fluorophenyl) piperidine, 4-benzylidenepiperidine, octahydro-1H-isoindole, 3-(benzyloxy) pyrrolidine, 3-phenoxypyrrolidine, N-methyl-N-phenyl-pyrrolidin-3-amine, 2-azabicyclo[2.2.1]heptane, 5-(4-methylpiperazin-1-yl)pyrimidine, 4-phenethylpiperidine, phenylpiperazine, 4-benzyl-piperazine, 3-(4-piperidyl)benzonitrile, methyl 3-(4-piperidyl)benzoate, methyl 4-(4-piperidyl)benzoate, 4-(3-pyrazol-1-ylphenyl) piperidine, 1-[(1R,2S)-2-(4-fluorophenyl)cyclopropyl]piperazine, 1-[2-(4-fluorophenyl) propyl]piperazine, 1-[2-(4-fluorophenyl)ethyl]piperazine 1-[2-(4-chlorophenyl)ethyl]piperazine, 1-(2-phenylpropyl) piperazine, or 1-(4-fluorophenyl)-2-piperazin-1-yl-ethanol.

According to one embodiment, at least one heterocycloalkyl present in the compound of formula (I) is a water-solubilizing group, i.e., the presence of this group in the molecule increases the solubility thereof in water, compared with the same molecule not comprising the heterocycloalkyl.

According to one embodiment, R^{E} represents hydrogen or halogen. In one preferred embodiment, R^{E} represents hydrogen or F. In one further preferred embodiment, R^{E} represents hydrogen. According to one embodiment, R^{E} represents (C₁-C₃) alkyl. According to one embodiment, R^{E} represents halogen.

According to one embodiment, the compound of formula (I) is a compound of formula (I-a)

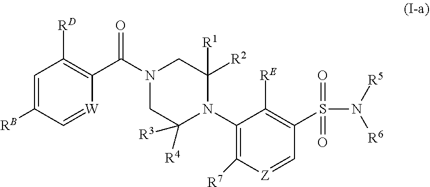

or a pharmaceutically acceptable salt and/or solvate thereof;
   wherein W, R^{B}, R^{D}, Z, R¹-R⁴, R⁵, R⁶, R⁷ and R^{E} are as defined under formula (I) herein.

In one embodiment, W in formula (I-a) represents CH.

In one embodiment, R^{B} and R^{D} in formula (I-a) each independently represents F or Cl. In one particular embodiment, R^{B} represents F, R^{D} represents Cl.

In one embodiment, R^{E} in formula (I-a) represents hydrogen.

5 According to one embodiment, the compound of formula (I) is selected from the compounds of Table 1 below, and pharmaceutically acceptable salts and/or solvates thereof

TABLE 1

| Cpd | Structure | Name |
|---|---|---|
| 001 | 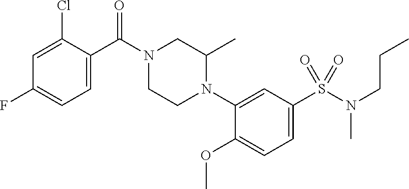 [SMILES: CCCN(C)S(=O)(=O)c1ccc(OC)c(N2CCN(C(=O)c3ccc(F)cc3Cl)CC2C)c1] | 4-methoxy-N-methyl-N- propyl-3-[rac-(2S)-4-(2- chloro-4-fluoro-benzoyl)- 2-methyl-piperazin-1- yl]benzenesulfonamide |
| 002 | 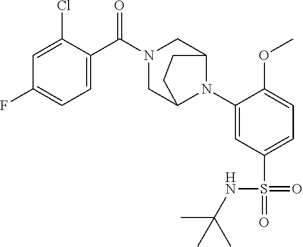 [SMILES: COc1ccc(S(=O)(=O)NC(C)(C)C)cc1N1C2CCC1CN(C(=O)c1ccc(F)cc1Cl)C2] | N-tert-butyl-3-[(1S,5R)-3- (2-chloro-4-fluoro- benzoyl)-3,8- diazabicyclo[3.2.1]]octan- 8-yl]-4-methoxy- benzenesulfonamide |
| 003 | 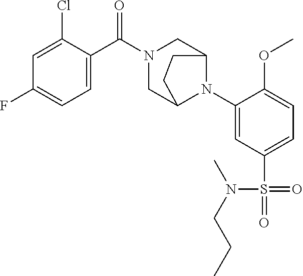 [SMILES: CCCN(C)S(=O)(=O)c1ccc(OC)c(N2C3CCC2CN(C(=O)c2ccc(F)cc2Cl)C3)c1] | 3-[(1S,5R)-3-(2-chloro-4- fluoro-benzoyl)-3,8- diazabicyclo[3.2.1]]octan- 8-yl]-4-methoxy-N- methyl-N-propyl- benzenesulfonamide |
| 004 | 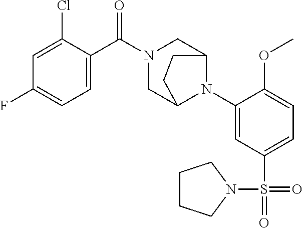 [SMILES: COc1ccc(S(=O)(=O)N2CCCC2)cc1N1C2CCC1CN(C(=O)c1ccc(F)cc1Cl)C2] | (2-chloro-4-fluoro- phenyl)-[(1S,5R)-8-(2- methoxy-5-pyrrolidin-1- ylsulfonyl-phenyl)-3,8- diazabicyclo[3.2.1]octan- 3-yl]methanone |
| 005 | 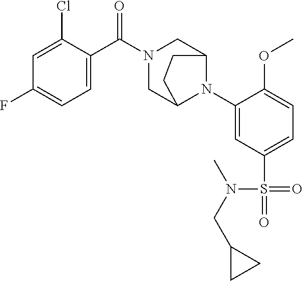 [SMILES: COc1ccc(S(=O)(=O)N(C)CC2CC2)cc1N1C2CCC1CN(C(=O)c1ccc(F)cc1Cl)C2] | 3-[(1S,5R)-3-(2-chloro-4- fluoro-benzoyl)-3,8- diazabicyclo[3.2.1]octan- 8-yl]-N- (cyclopropylmethyl)-4- methoxy-N-methyl- benzenesulfonamide |
| 006 | 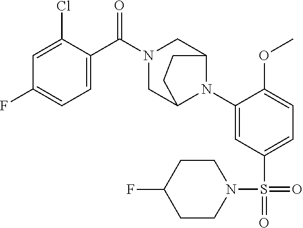 [SMILES: COc1ccc(S(=O)(=O)N2CCC(F)CC2)cc1N1C2CCC1CN(C(=O)c1ccc(F)cc1Cl)C2] | (2-chloro-4-fluoro- phenyl)-[(1S,5R)-8-[5-[(4- fluoro-1- piperidyl)sulfonyl]-2- methoxy-phenyl]-3,8- diazabicyclo[3.2.1]]octan- 3-yl]methanone |
| 007 | 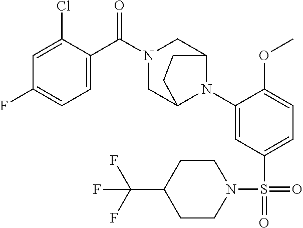 [SMILES: COc1ccc(S(=O)(=O)N2CCC(C(F)(F)F)CC2)cc1N1C2CCC1CN(C(=O)c1ccc(F)cc1Cl)C2] | (2-chloro-4-fluoro- phenyl)-[(1S,5R)-8-[2- methoxy-5-[[4- (trifluoromethyl)-1- piperidyl]sulfonyl]phenyl]- 3,8- diazabicyclo[3.2.1]octan- 3-yl]methanone |
| 008 | 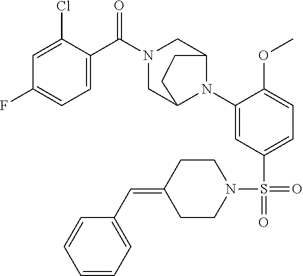 [SMILES: COc1ccc(S(=O)(=O)N2CCC(=Cc3ccccc3)CC2)cc1N1C2CCC1CN(C(=O)c1ccc(F)cc1Cl)C2] | [(1S,5R)-8-[5-[(4- benzylidene-1- piperidyl)sulfonyl]-2- methoxy-phenyl]-3,8- diazabicyclo[3.2.1]octan- 3-yl]-(2-chloro-4-fluoro- phenyl)methanone |
| 009 | 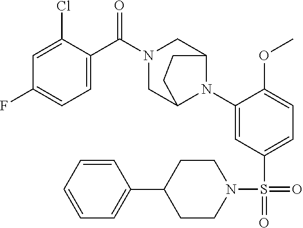 [SMILES: COc1ccc(S(=O)(=O)N2CCC(c3ccccc3)CC2)cc1N1C2CCC1CN(C(=O)c1ccc(F)cc1Cl)C2] | (2-chloro-4-fluoro- phenyl)-[(1S,5R)-8-[2- methoxy-5-[(4-phenyl-1- piperidyl)sulfonyl]phenyl]- 3,8- diazabicyclo[3.2.1]octan- 3-yl]methanone |
| 010 | 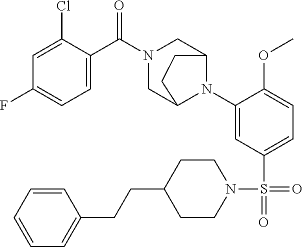 [SMILES: COc1ccc(S(=O)(=O)N2CCC(CCc3ccccc3)CC2)cc1N1C2CCC1CN(C(=O)c1ccc(F)cc1Cl)C2] | (2-chloro-4-fluoro- phenyl)-[(1S,5R)-8-[2- methoxy-5-[[4-(2- phenylethyl)-1- piperidyl]sulfonyl]phenyl]- 3,8- diazabicyclo[3.2.1]octan- 3-yl]methanone |
| 011 | 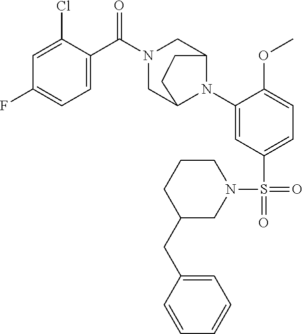 [SMILES: COc1ccc(S(=O)(=O)N2CCCC(Cc3ccccc3)C2)cc1N1C2CCC1CN(C(=O)c1ccc(F)cc1Cl)C2] | [(1S,5R)-8-[5-[[(3RS)-3- benzyl-1- piperidyl]sulfonyl]-2- methoxy-phenyl]-3,8- diazabicyclo[3.2.1]]octan- 3-yl]-(2-chloro-4-fluoro- phenyl)methanone |
| 012 | 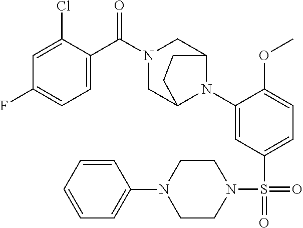 [SMILES: COc1ccc(S(=O)(=O)N2CCN(c3ccccc3)CC2)cc1N1C2CCC1CN(C(=O)c1ccc(F)cc1Cl)C2] | (2-chloro-4-fluoro- phenyl)-[(1S,5R)-8-[2- methoxy-5-(4- phenylpiperazin-1- yl)sulfonyl-phenyl]-3,8- diazabicyclo[3.2.1]octan- 3-yl]methanone |
| 013 | 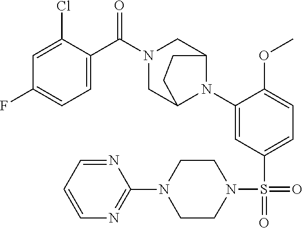 [SMILES: COc1ccc(S(=O)(=O)N2CCN(c3ncccn3)CC2)cc1N1C2CCC1CN(C(=O)c1ccc(F)cc1Cl)C2] | (2-chloro-4-fluoro- phenyl)-[(1S,5R)-8-[2- methoxy-5-(4-pyrimidin- 2-ylpiperazin-1- yl)sulfonyl-phenyl]-3,8- diazabicyclo[3.2.1]]octan- 3-yl]methanone |
| 014 | 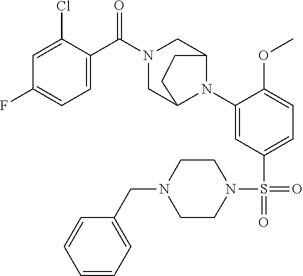 [SMILES: COc1ccc(S(=O)(=O)N2CCN(Cc3ccccc3)CC2)cc1N1C2CCC1CN(C(=O)c1ccc(F)cc1Cl)C2] | [(1S,5R)-8-[5-(4- benzylpiperazin-1- yl)sulfonyl-2-methoxy- phenyl]-3,8- diazabicyclo[3.2.1]]octan- 3-yl]-(2-chloro-4-fluoro- phenyl)methanone;ethane |
| 015 | 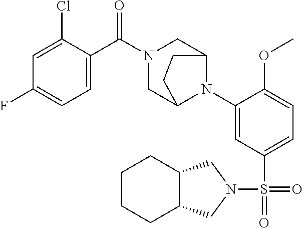 [SMILES: COc1ccc(S(=O)(=O)N2C[C@H]3CCCC[C@H]3C2)cc1N1C2CCC1CN(C(=O)c1ccc(F)cc1Cl)C2] | [(1S,5R)-8-[5-[[(3aS,7aR)- 1,3,3a,4,5,6,7,7a- octahydroisoindol-2- yl]sulfonyl]-2-methoxy- phenyl]-3,8- diazabicyclo[3.2.1]octan- 3-yl]-(2-chloro-4-fluoro- phenyl)methanone |
| 016 | 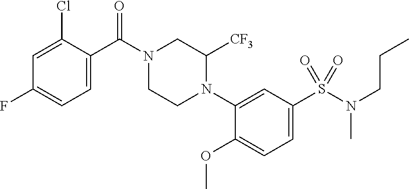 [SMILES: CCCN(C)S(=O)(=O)c1ccc(OC)c(N2CCN(C(=O)c3ccc(F)cc3Cl)CC2C(F)(F)F)c1] | 4-methoxy-N-methyl-N- propyl-3-[rac-(2S)-4-[4- fluoro-2- (trifluoromethyl)benzoyl]- 2- (trifluoromethyl)piperazin- 1-yl]benzenesulfonamide |
| 017 | 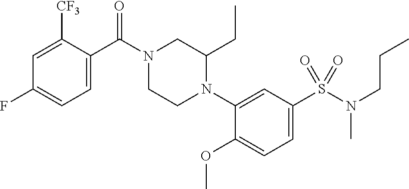 [SMILES: CCCN(C)S(=O)(=O)c1ccc(OC)c(N2CCN(C(=O)c3ccc(F)cc3C(F)(F)F)CC2CC)c1] | 4-methoxy-N-methyl-N- propyl-3-[rac-(2S)-2-ethyl- 4-[4-fluoro-2- (trifluoromethyl)benzoyl] piperazin-1- yl]benzenesulfonamide |
| 018 | 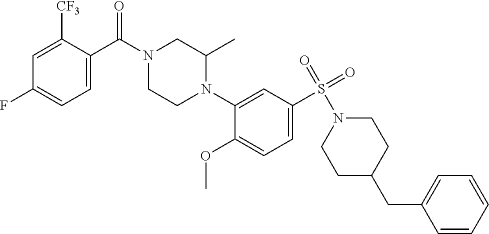 [SMILES: COc1ccc(S(=O)(=O)N2CCC(Cc3ccccc3)CC2)cc1N1CCN(C(=O)c2ccc(F)cc2C(F)(F)F)CC1C] | [4-fluoro-2- (trifluoromethyl)phenyl]- [rac-(3S)-4-[5-[(4-benzyl- 1-piperidyl)sulfonyl]-2- methoxy-phenyl]-3- methyl-piperazin-1- yl]methanone |
| 019 | 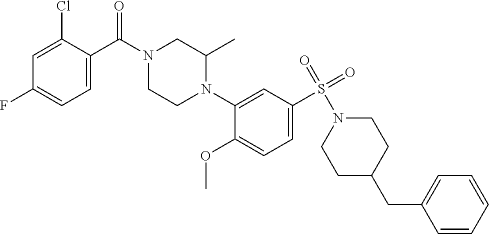 [SMILES: COc1ccc(S(=O)(=O)N2CCC(Cc3ccccc3)CC2)cc1N1CCN(C(=O)c2ccc(F)cc2Cl)CC1C] | (2-chloro-4-fluoro- phenyl)-[rac-(3S)-4-[5-[(4- benzyl-1- piperidyl)sulfonyl]-2- methoxy-phenyl]-3- methyl-piperazin-1- yl]methanone |
| 020 | 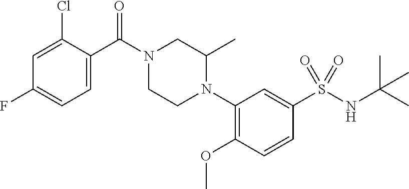 [SMILES: COc1ccc(S(=O)(=O)NC(C)(C)C)cc1N1CCN(C(=O)c2ccc(F)cc2Cl)CC1C] | N-tert-butyl-4-methoxy-3- [rac-(2S)-4-(2-chloro-4- fluoro-benzoyl)-2-methyl- piperazin-1- yl]benzenesulfonamide |
| 021 | 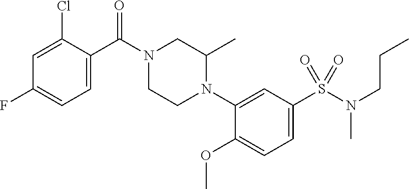 [SMILES: CCCN(C)S(=O)(=O)c1ccc(OC)c(N2CCN(C(=O)c3ccc(F)cc3Cl)CC2C)c1] | 4-methoxy-N-methyl-N- propyl-3-[rac-(2S)-4-[4- fluoro-2- (trifluoromethyl)benzoyl]- 2-methyl-piperazin-1- yl]benzenesulfonamide |
| 022 | 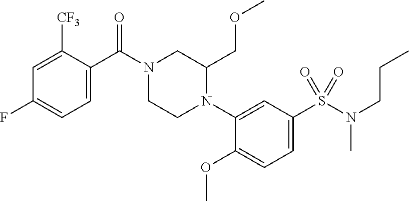 [SMILES: CCCN(C)S(=O)(=O)c1ccc(OC)c(N2CCN(C(=O)c3ccc(F)cc3C(F)(F)F)CC2COC)c1] | 4-methoxy-N-methyl-N- propyl-3-[rac-(2R)-4-[4- fluoro-2- (trifluoromethyl)benzoyl]- 2- (methoxymethyl)piperazin- 1-yl]benzenesulfonamide |
| 023 | 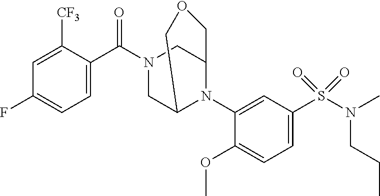 [SMILES: CCCN(C)S(=O)(=O)c1ccc(OC)c(N2C3COCC2CN(C(=O)c2ccc(F)cc2C(F)(F)F)C3)c1] | 3-[(1S,5R)-7-[4-fluoro-2- (trifluoromethyl)benzoyl]- 3-oxa-7,9- diazabicyclo[3.3.1]nonan- 9-yl]-4-methoxy-N- methyl-N-propyl- benzenesulfonamide |
| 024 | 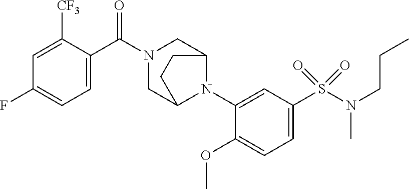 [SMILES: CCCN(C)S(=O)(=O)c1ccc(OC)c(N2C3CCC2CN(C(=O)c2ccc(F)cc2C(F)(F)F)C3)c1] | 3-[3-[4-fluoro-2- (trifluoromethyl)benzoyl]- 3,8- diazabicyclo[3.2.1]octan- 8-yl]-4-methoxy-N- methyl-N-propyl- benzenesulfonamide |
| 025 | 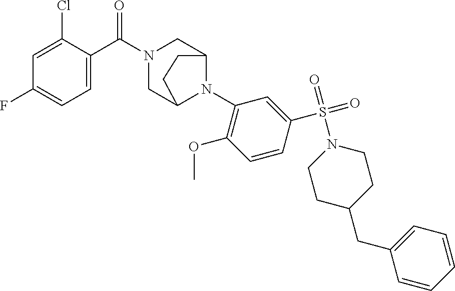 [SMILES: COc1ccc(S(=O)(=O)N2CCC(Cc3ccccc3)CC2)cc1N1C2CCC1CN(C(=O)c1ccc(F)cc1Cl)C2] | (2-chloro-4-fluoro- phenyl)-[rac-(1S,5S)-8-[5- [(4-benzyl-1- piperidyl)sulfonyl]-2- methoxy-phenyl]-3,8- diazabicyclo[3.2.1]]octan- 3-yl]methanone |
| 026 | 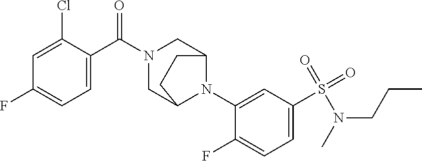 [SMILES: CCCN(C)S(=O)(=O)c1ccc(F)c(N2C3CCC2CN(C(=O)c2ccc(F)cc2Cl)C3)c1] | 3-[(1S,5R)-3-(2-chloro-4- fluoro-benzoyl)-3,8- diazabicyclo[3.2.1]]octan- 8-yl]-4-fluoro-N-methyl- N-propyl- benzenesulfonamide |
| 027 | 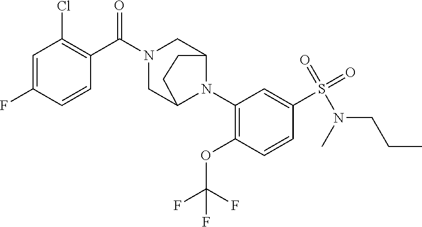 [SMILES: CCCN(C)S(=O)(=O)c1ccc(OC(F)(F)F)c(N2C3CCC2CN(C(=O)c2ccc(F)cc2Cl)C3)c1] | 3-[(1S,5R)-3-(2-chloro-4- fluoro-benzoyl)-3,8- diazabicyclo[3.2.1]octan- 8-yl]-N-methyl-N-propyl- 4- (trifluoromethoxy)benzene sulfonamide |
| 028 | 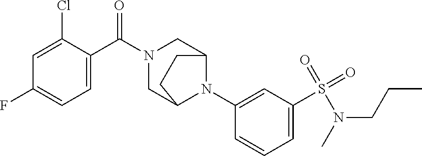 [SMILES: CCCN(C)S(=O)(=O)c1cccc(N2C3CCC2CN(C(=O)c2ccc(F)cc2Cl)C3)c1] | 3-[(1S,5R)-3-(2-chloro-4- fluoro-benzoyl)-3,8- diazabicyclo[3.2.1]octan- 8-yl]-N-methyl-N-propyl- benzenesulfonamide |
| 029 | 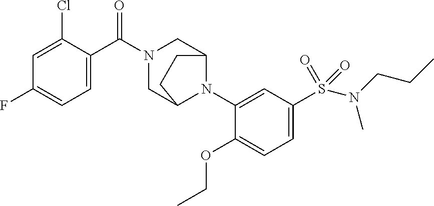 [SMILES: CCCN(C)S(=O)(=O)c1ccc(OCC)c(N2C3CCC2CN(C(=O)c2ccc(F)cc2Cl)C3)c1] | 3-[(1S,5R)-3-(2-chloro-4- fluoro-benzoyl)-3,8- diazabicyclo[3.2.1]]octan- 8-yl]-4-ethoxy-N-methyl- N-propyl- benzenesulfonamide |
| 030 | 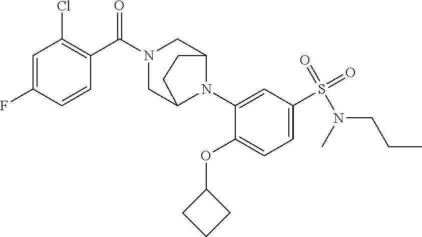 [SMILES: CCCN(C)S(=O)(=O)c1ccc(OC2CCC2)c(N2C3CCC2CN(C(=O)c2ccc(F)cc2Cl)C3)c1] | 3-[(1S,5R)-3-(2-chloro-4- fluoro-benzoyl)-3,8- diazabicyclo[3.2.1]octan- 8-yl]-4-(cyclobutoxy)-N- methyl-N-propyl- benzenesulfonamide |
| 031 | 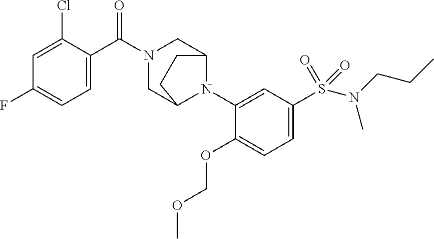 [SMILES: CCCN(C)S(=O)(=O)c1ccc(OCOC)c(N2C3CCC2CN(C(=O)c2ccc(F)cc2Cl)C3)c1] | 3-[(1S,5R)-3-(2-chloro-4- fluoro-benzoyl)-3,8- diazabicyclo[3.2.1]octan- 8-yl]-4- (methoxymethoxy)-N- methyl-N-propyl- benzenesulfonamide |
| 032 | 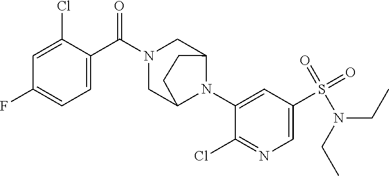 [SMILES: CCN(CC)S(=O)(=O)c1cnc(Cl)c(N2C3CCC2CN(C(=O)c2ccc(F)cc2Cl)C3)c1] | 6-chloro-5-[(1S,5R)-3-(2- chloro-4-fluoro-benzoyl)- 3,8- diazabicyclo[3.2.1]octan- 8-yl]-N,N-diethyl- pyridine-3-sulfonamide |
| 033 | 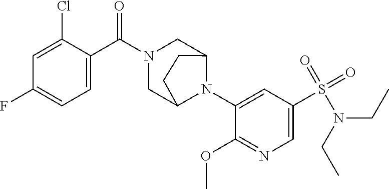 [SMILES: CCN(CC)S(=O)(=O)c1cnc(OC)c(N2C3CCC2CN(C(=O)c2ccc(F)cc2Cl)C3)c1] | 5-[(1S,5R)-3-(2-chloro-4- fluoro-benzoyl)-3,8- diazabicyclo[3.2.1]octan- 8-yl]-N,N-diethyl-6- methoxy-pyridine-3- sulfonamide |
| 034 | 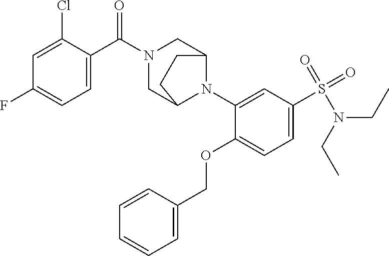 [SMILES: CCN(CC)S(=O)(=O)c1ccc(OCc2ccccc2)c(N2C3CCC2CN(C(=O)c2ccc(F)cc2Cl)C3)c1] | 6-benzyloxy-5-[(1S,5R)-3- (2-chloro-4-fluoro- benzoyl)-3,8- diazabicyclo[3.2.1]octan- 8-yl]-N,N-diethyl- pyridine-3-sulfonamide |
| 035 | 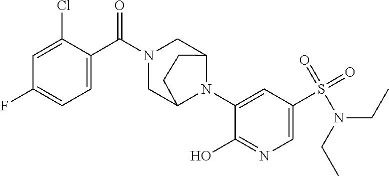 [SMILES: CCN(CC)S(=O)(=O)c1cnc(O)c(N2C3CCC2CN(C(=O)c2ccc(F)cc2Cl)C3)c1] | 5-[(1S,5R)-3-(2-chloro-4- fluoro-benzoyl)-3,8- diazabicyclo[3.2.1]]octan- 8-yl]-N,N-diethyl-6- hydroxy-pyridine-3- sulfonamide |
| 036 | 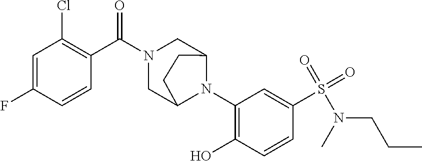 [SMILES: CCCN(C)S(=O)(=O)c1ccc(O)c(N2C3CCC2CN(C(=O)c2ccc(F)cc2Cl)C3)c1] | 3-[(1S,5R)-3-(2-chloro-4- fluoro-benzoyl)-3,8- diazabicyclo[3.2.1]octan- 8-yl]-4-hydroxy-N- methyl-N-propyl- benzenesulfonamide |
| 037 | 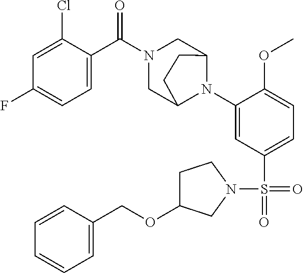 [SMILES: COc1ccc(S(=O)(=O)N2CCC(OCc3ccccc3)C2)cc1N1C2CCC1CN(C(=O)c1ccc(F)cc1Cl)C2] | [(1S,5R)-8-[5-[(3SR)-3- benzyloxypyrrolidin-1- yl]sulfonyl-2-methoxy- phenyl]-3,8- diazabicyclo[3.2.1]octan- 3-yl]-(2-chloro-4-fluoro- phenyl)methanone |
| 038 | 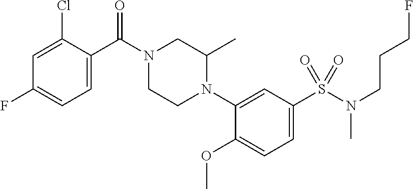 [SMILES: COc1ccc(S(=O)(=O)N(C)CCCF)cc1N1CCN(C(=O)c2ccc(F)cc2Cl)CC1C] | N-(3-fluoropropyl)-4- methoxy-N-methyl-3-[rac- (2S)-4-(2-chloro-4-fluoro- benzoyl)-2-methyl- piperazin-1- yl]benzenesulfonamide |
| 039 | 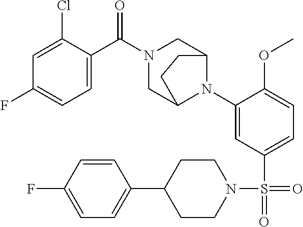 [SMILES: COc1ccc(S(=O)(=O)N2CCC(c3ccc(F)cc3)CC2)cc1N1C2CCC1CN(C(=O)c1ccc(F)cc1Cl)C2] | (2-chloro-4-fluoro- phenyl)-[(1S,5R)-8-[5-[[4- (4-fluorophenyl)-1- piperidyl]sulfonyl]-2- methoxy-phenyl]-3,8- diazabicyclo[3.2.1]octan- 3-yl]methanone |
| 040 | 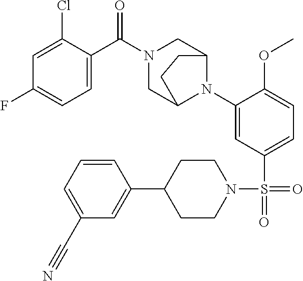 [SMILES: COc1ccc(S(=O)(=O)N2CCC(c3cccc(C#N)c3)CC2)cc1N1C2CCC1CN(C(=O)c1ccc(F)cc1Cl)C2] | 3-[1-[3-[(1S,5R)-3-(2- chloro-4-fluoro-benzoyl)- 3,8- diazabicyclo[3.2.1]octan- 8-yl]-4-methoxy- phenyl]sulfonyl-4- piperidyl]benzonitrile |
| 041 | 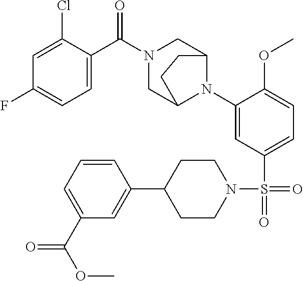 [SMILES: COC(=O)c1cccc(C2CCN(S(=O)(=O)c3ccc(OC)c(N4C5CCC4CN(C(=O)c4ccc(F)cc4Cl)C5)c3)CC2)c1] | methyl 3-[1-[3-[(1S,5R)-3- (2-chloro-4-fluoro- benzoyl)-3,8- diazabicyclo[3.2.1]octan- 8-yl]-4-methoxy- phenyl]sulfonyl-4- piperidyl]benzoate |
| 042 | 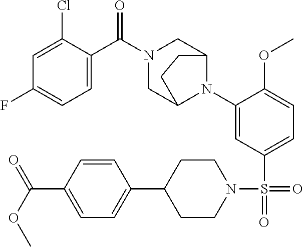 [SMILES: COC(=O)c1ccc(C2CCN(S(=O)(=O)c3ccc(OC)c(N4C5CCC4CN(C(=O)c4ccc(F)cc4Cl)C5)c3)CC2)cc1] | methyl 4-[1-[3-[(1S,5R)-3- (2-chloro-4-fluoro- benzoyl)-3,8- diazabicyclo[3.2.1]octan- 8-yl]-4-methoxy- phenyl]sulfonyl-4- piperidyl]benzoate |
| 043 | 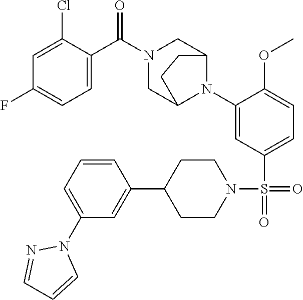 [SMILES: COc1ccc(S(=O)(=O)N2CCC(c3cccc(-n4cccn4)c3)CC2)cc1N1C2CCC1CN(C(=O)c1ccc(F)cc1Cl)C2] | (2-chloro-4-fluoro- phenyl)-[(1S,5R)-8-[2- methoxy-5-[[4-(3-pyrazol- 1-ylphenyl)-1- piperidyl]sulfonyl]phenyl]- 3,8- diazabicyclo[3.2.1]octan- 3-yl]methanone |
| 044 | 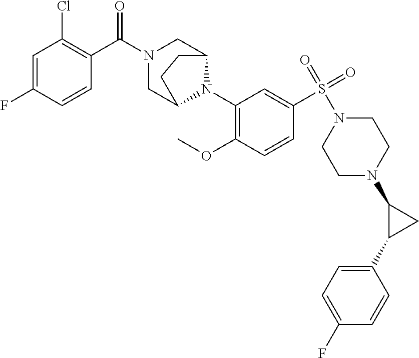 [SMILES: COc1ccc(S(=O)(=O)N2CCN([C@H]3C[C@@H]3c3ccc(F)cc3)CC2)cc1N1[C@@H]2CC[C@H]1CN(C(=O)c1ccc(F)cc1Cl)C2] | (2-chloro-4-fluoro- phenyl)-[(1S,5R)-8-[5-[4- [(1SR,2RS)-2-(4- fluorophenyl)cyclopropyl] piperazin-1-yl]sulfonyl-2- methoxy-phenyl]-3,8- diazabicyclo[3.2.1]octan- 3-yl]methanone |
| 045 | 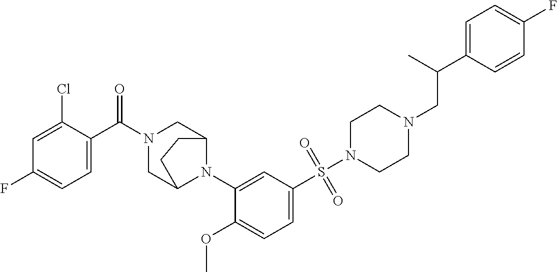 [SMILES: COc1ccc(S(=O)(=O)N2CCN(CC(C)c3ccc(F)cc3)CC2)cc1N1C2CCC1CN(C(=O)c1ccc(F)cc1Cl)C2] | (2-chloro-4-fluoro- phenyl)-[(1S,5R)-8-[5-[4- [(2RS)-2-(4- fluorophenyl)propyl]piper azin-1-yl]sulfonyl-2- methoxy-phenyl]-3,8- diazabicyclo[3.2.1]octan- 3-yl]methanone |
| 046 | 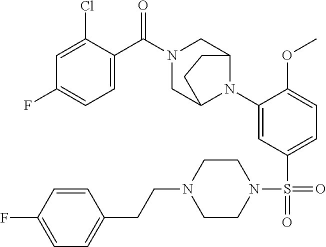 [SMILES: COc1ccc(S(=O)(=O)N2CCN(CCc3ccc(F)cc3)CC2)cc1N1C2CCC1CN(C(=O)c1ccc(F)cc1Cl)C2] | (2-chloro-4-fluoro- phenyl)-[(1S,5R)-8-[5-[4- [2-(4- fluorophenyl)ethyl]piperazin- 1-yl]sulfonyl-2- methoxy-phenyl]-3,8- diazabicyclo[3.2.1]octan- 3-yl]methanone |
| 047 | 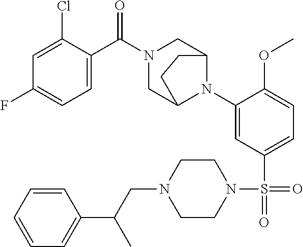 [SMILES: COc1ccc(S(=O)(=O)N2CCN(CC(C)c3ccccc3)CC2)cc1N1C2CCC1CN(C(=O)c1ccc(F)cc1Cl)C2] | (2-chloro-4-fluoro- phenyl)-[(1S,5R)-8-[2- methoxy-5-[4-[(2RS)-2- phenylpropyl]piperazin-1- yl]sulfonyl-phenyl]-3,8- diazabicyclo[3.2.1]octan- 3-yl]methanone |
| 048 | 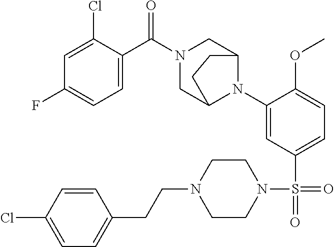 [SMILES: COc1ccc(S(=O)(=O)N2CCN(CCc3ccc(Cl)cc3)CC2)cc1N1C2CCC1CN(C(=O)c1ccc(F)cc1Cl)C2] | (2-chloro-4-fluoro- phenyl)-[(1S,5R)-8-[5-[4- [2-(4- chlorophenyl)ethyl]piperazin- 1-yl]sulfonyl-2- methoxy-phenyl]-3,8- diazabicyclo[3.2.1]octan- 3-yl]methanone |
| 049 | 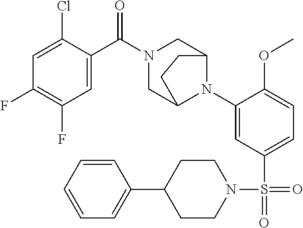 [SMILES: COc1ccc(S(=O)(=O)N2CCC(c3ccccc3)CC2)cc1N1C2CCC1CN(C(=O)c1cc(F)c(F)cc1Cl)C2] | (2-chloro-4,5-difluoro- phenyl)-[(1S,5R)-8-[2- methoxy-5-[(4-phenyl-1- piperidyl)sulfonyl]phenyl]- 3,8- diazabicyclo[3.2.1]octan- 3-yl]methanone |
| 050 | 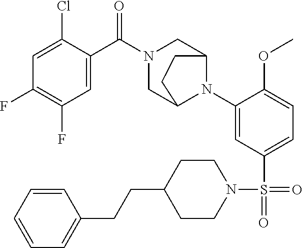 [SMILES: COc1ccc(S(=O)(=O)N2CCC(CCc3ccccc3)CC2)cc1N1C2CCC1CN(C(=O)c1cc(F)c(F)cc1Cl)C2] | (2-chloro-4,5-difluoro- phenyl)-[(1S,5R)-8-[2- methoxy-5-[[4-(2- phenylethyl)-1- piperidyl]sulfonyl]phenyl]- 3,8- diazabicyclo[3.2.1]octan- 3-yl]methanone |
| 051 | 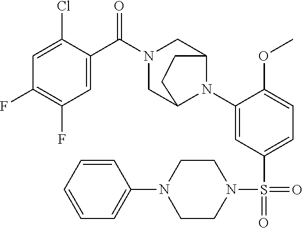 [SMILES: COc1ccc(S(=O)(=O)N2CCN(c3ccccc3)CC2)cc1N1C2CCC1CN(C(=O)c1cc(F)c(F)cc1Cl)C2] | (2-chloro-4,5-difluoro- phenyl)-[(1S,5R)-8-[2- methoxy-5-(4- phenylpiperazin-1- yl)sulfonyl-phenyl]-3,8- diazabicyclo[3.2.1]]octan- 3-yl]methanone |
| 052 | 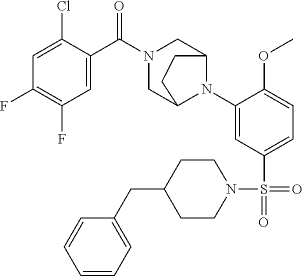 [SMILES: COc1ccc(S(=O)(=O)N2CCC(Cc3ccccc3)CC2)cc1N1C2CCC1CN(C(=O)c1cc(F)c(F)cc1Cl)C2] | [(1S,5R)-8-[5-[(3-benzyl- 1-piperidyl)sulfonyl]-2- methoxy-phenyl]-3,8- diazabicyclo[3.2.1]octan- 3-yl]-(2-chloro-4,5- difluoro- phenyl)methanone |
| 053 | 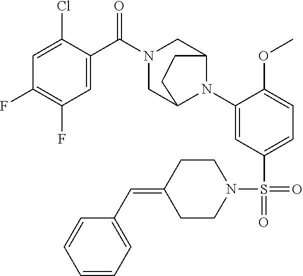 [SMILES: COc1ccc(S(=O)(=O)N2CCC(=Cc3ccccc3)CC2)cc1N1C2CCC1CN(C(=O)c1cc(F)c(F)cc1Cl)C2] | [(1S,5R)-8-[5-[(4- benzylidene-1- piperidyl)sulfonyl]-2- methoxy-phenyl]-3,8- diazabicyclo[3.2.1]octan- 3-yl]-(2-chloro-4,5- difluoro- phenyl)methanone |
| 054 | 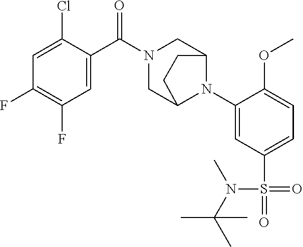 [SMILES: COc1ccc(S(=O)(=O)N(C)C(C)(C)C)cc1N1C2CCC1CN(C(=O)c1cc(F)c(F)cc1Cl)C2] | N-tert-butyl-3-[(1S,5R)-3- (2-chloro-4,5-difluoro- benzoyl)-3,8- diazabicyclo[3.2.1]octan- 8-yl]-4-methoxy-N- methyl- benzenesulfonamide |
| 055 | 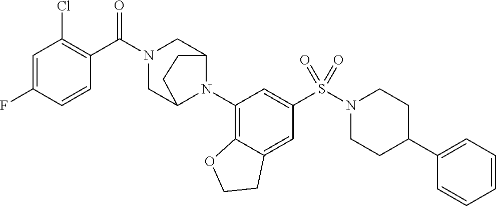 [SMILES: O=C(c1ccc(F)cc1Cl)N1CC2CCC(C1)N2c1cc(S(=O)(=O)N2CCC(c3ccccc3)CC2)cc2c1OCC2] | (2-chloro-4-fluoro- phenyl)-[(1S,5R)-8-[5-[(4- phenyl-1- piperidyl)sulfonyl]-2,3- dihydrobenzofuran-7-yl]- 3,8- diazabicyclo[3.2.1]octan- 3-yl]methanone |
| 056 | 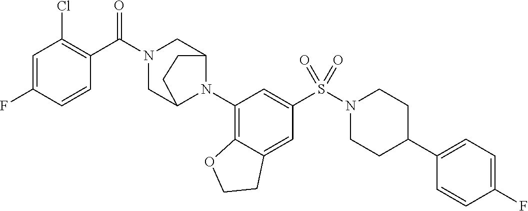 [SMILES: O=C(c1ccc(F)cc1Cl)N1CC2CCC(C1)N2c1cc(S(=O)(=O)N2CCC(c3ccc(F)cc3)CC2)cc2c1OCC2] | (2-chloro-4-fluoro- phenyl)-[(1S,5R)-8-[5-[[4- (4-fluorophenyl)-1- piperidyl]sulfonyl]-2,3- dihydrobenzofuran-7-yl]- 3,8- diazabicyclo[3.2.1]octan- 3-yl]methanone |
| 057 | 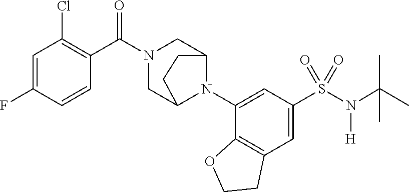 [SMILES: CC(C)(C)NS(=O)(=O)c1cc2c(c(N3C4CCC3CN(C(=O)c3ccc(F)cc3Cl)C4)c1)OCC2] | N-tert-butyl-7-[(1S,5R)-3- (2-chloro-4-fluoro- benzoyl)-3,8- diazabicyclo[3.2.1]octan- 8-yl]-2,3- dihydrobenzofuran-5- sulfonamide |
| 058 | 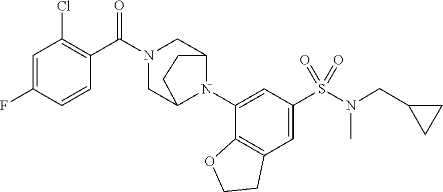 [SMILES: CN(CC1CC1)S(=O)(=O)c1cc2c(c(N3C4CCC3CN(C(=O)c3ccc(F)cc3Cl)C4)c1)OCC2] | 7-[(1S,5R)-3-(2-chloro-4- fluoro-benzoyl)-3,8- diazabicyclo[3.2.1]octan- 8-yl]-N- (cyclopropylmethyl)-N- methyl-2,3- dihydrobenzofuran-5- sulfonamide |
| 059 | 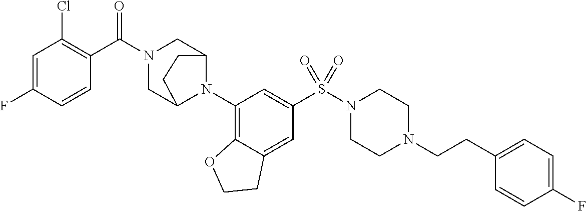 [SMILES: O=C(c1ccc(F)cc1Cl)N1CC2CCC(C1)N2c1cc(S(=O)(=O)N2CCN(CCc3ccc(F)cc3)CC2)cc2c1OCC2] | (2-chloro-4-fluoro- phenyl)-[(1S,5R)-8-[5-[4- [2-(4- fluorophenyl)ethyl]piperazin- 1-yl]sulfonyl-2,3- dihydrobenzofuran-7-yl]- 3,8- diazabicyclo[3.2.1]octan- 3-yl]methanone |
| 060 | 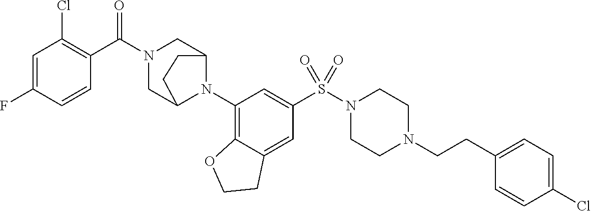 [SMILES: O=C(c1ccc(F)cc1Cl)N1CC2CCC(C1)N2c1cc(S(=O)(=O)N2CCN(CCc3ccc(Cl)cc3)CC2)cc2c1OCC2] | (2-chloro-4-fluoro- phenyl)-[(1S,5R)-8-[5-[4- [2-(4- chlorophenyl)ethyl]piperazin- 1-yl]sulfonyl-2,3- dihydrobenzofuran-7-yl]- 3,8- diazabicyclo[3.2.1]octan- 3-yl]methanone |
| 061 | 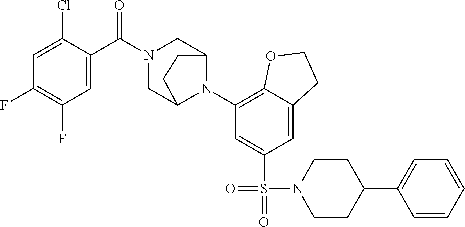 [SMILES: O=C(c1cc(F)c(F)cc1Cl)N1CC2CCC(C1)N2c1cc(S(=O)(=O)N2CCC(c3ccccc3)CC2)cc2c1OCC2] | (2-chloro-4,5-difluoro- phenyl)-[(1S,5R)-8-[5-[(4- phenyl-1- piperidyl)sulfonyl]-2,3- dihydrobenzofuran-7-yl]- 3,8- diazabicyclo[3.2.1]octan- 3-yl]methanone |
| 062 | 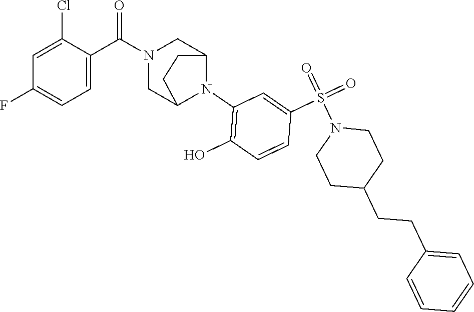 [SMILES: O=C(c1ccc(F)cc1Cl)N1CC2CCC(C1)N2c1cc(S(=O)(=O)N2CCC(CCc3ccccc3)CC2)ccc1O] | (2-chloro-4-fluoro- phenyl)-[(1S,5R)-8-[2- hydroxy-5-[[4-(2- phenylethyl)-1- piperidyl]sulfonyl]phenyl]- 3,8- diazabicyclo[3.2.1]]octan- 3-yl]methanone |
| 063 | 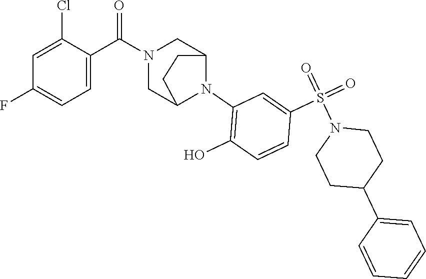 [SMILES: O=C(c1ccc(F)cc1Cl)N1CC2CCC(C1)N2c1cc(S(=O)(=O)N2CCC(c3ccccc3)CC2)ccc1O] | (2-chloro-4-fluoro- phenyl)-[(1S,5R)-8-[2- hydroxy-5-[(4-phenyl-1- piperidyl)sulfonyl]phenyl]- 3,8- diazabicyclo[3.2.1]octan- 3-yl]methanone |
| 064 | 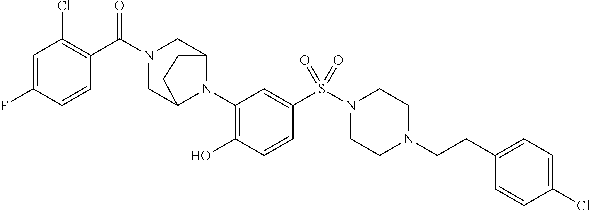 [SMILES: O=C(c1ccc(F)cc1Cl)N1CC2CCC(C1)N2c1cc(S(=O)(=O)N2CCN(CCc3ccc(Cl)cc3)CC2)ccc1O] | (2-chloro-4-fluoro- phenyl)-[(1S,5R)-8-[5-[4- [2-(4- chlorophenyl)ethyl]piperazin- 1-yl]sulfonyl-2- hydroxy-phenyl]-3,8- diazabicyclo[3.2.1]octan- 3-yl]methanone |
| 065 | 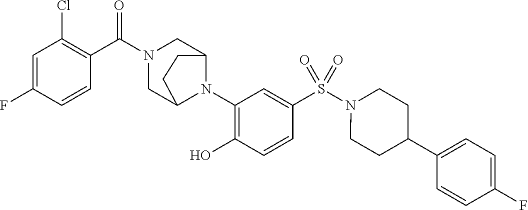 [SMILES: O=C(c1ccc(F)cc1Cl)N1CC2CCC(C1)N2c1cc(S(=O)(=O)N2CCC(c3ccc(F)cc3)CC2)ccc1O] | (2-chloro-4-fluoro- phenyl)-[(1S,5R)-8-[5-[[4- (4-fluorophenyl)-1- piperidyl]sulfonyl]-2- hydroxy-phenyl]-3,8- diazabicyclo[3.2.1]octan- 3-yl]methanone |
| 066 | 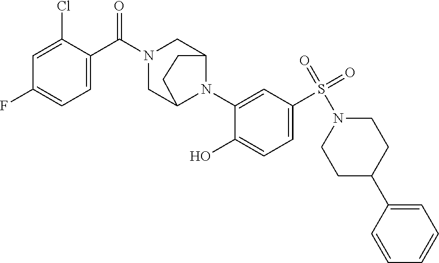 [SMILES: O=C(c1ccc(F)cc1Cl)N1CC2CCC(C1)N2c1cc(S(=O)(=O)N2CCC(c3ccccc3)CC2)ccc1O] | (2-chloro-4-fluoro- phenyl)-[(1S,5R)-8-[2- chloro-5-[(4-phenyl-1- piperidyl)sulfonyl]phenyl]- 3,8- diazabicyclo[3.2.1]octan- 3-yl]methanone |
| 067 | 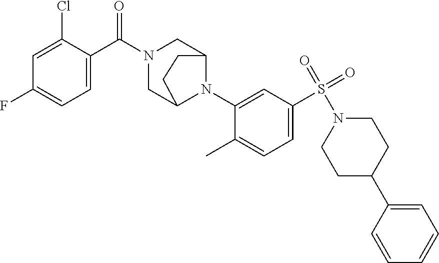 [SMILES: Cc1ccc(S(=O)(=O)N2CCC(c3ccccc3)CC2)cc1N1C2CCC1CN(C(=O)c1ccc(F)cc1Cl)C2] | (2-chloro-4-fluoro- phenyl)-[(1S,5R)-8-[2- methyl-5-[(4-phenyl-1- piperidyl)sulfonyl]phenyl]- 3,8- diazabicyclo[3.2.1]octan- 3-yl]methanone |
| 068 | 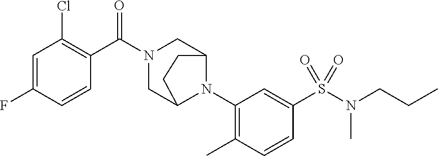 [SMILES: CCCN(C)S(=O)(=O)c1ccc(C)c(N2C3CCC2CN(C(=O)c2ccc(F)cc2Cl)C3)c1] | 3-[(1S,5R)-3-(2-chloro-4- fluoro-benzoyl)-3,8- diazabicyclo[3.2.1]octan- 8-yl]-N,4-dimethyl-N- propyl- benzenesulfonamide |
| 069 | 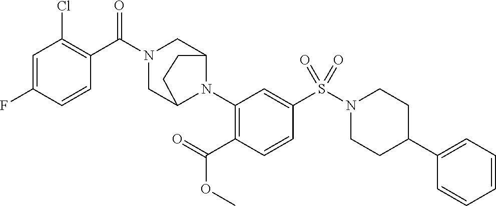 [SMILES: COC(=O)c1ccc(S(=O)(=O)N2CCC(c3ccccc3)CC2)cc1N1C2CCC1CN(C(=O)c1ccc(F)cc1Cl)C2] | methyl 2-[(1S,5R)-3-(2- chloro-4-fluoro-benzoyl)- 3,8- diazabicyclo[3.2.1]octan- 8-yl]-4-[(4-phenyl-1- piperidyl)sulfonyl]benzoate |
| 070 | 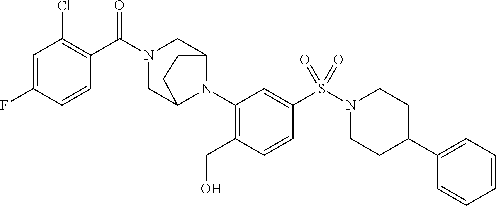 [SMILES: O=C(c1ccc(F)cc1Cl)N1CC2CCC(C1)N2c1cc(S(=O)(=O)N2CCC(c3ccccc3)CC2)ccc1CO] | (2-chloro-4-fluoro- phenyl)-[(1S,5R)-8-[2- (hydroxymethyl)-5-[(4- phenyl-1- piperidyl)sulfonyl]phenyl]- 3,8- diazabicyclo[3.2.1]octan- 3-yl]methanone |
| 071 | 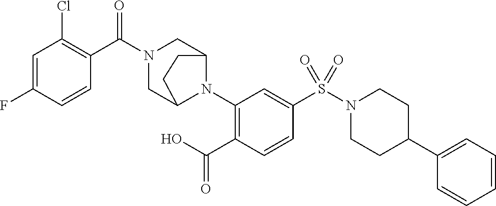 [SMILES: O=C(O)c1ccc(S(=O)(=O)N2CCC(c3ccccc3)CC2)cc1N1C2CCC1CN(C(=O)c1ccc(F)cc1Cl)C2] | 2-[(1S,5R)-3-(2-chloro-4- fluoro-benzoyl)-3,8- diazabicyclo[3.2.1]octan- 8-yl]-4-[(4-phenyl-1- piperidyl)sulfonyl]benzoic acid |
| 072 | 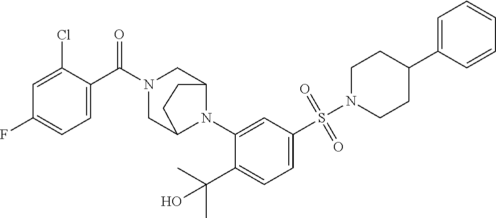 [SMILES: CC(C)(O)c1ccc(S(=O)(=O)N2CCC(c3ccccc3)CC2)cc1N1C2CCC1CN(C(=O)c1ccc(F)cc1Cl)C2] | (2-chloro-4-fluoro- phenyl)-[(1S,5R)-8-[2-(1- hydroxy-1-methyl-ethyl)- 5-[(4-phenyl-1- piperidyl)sulfonyl]phenyl]- 3,8- diazabicyclo[3.2.1]octan- 3-yl]methanone |
| 073 | 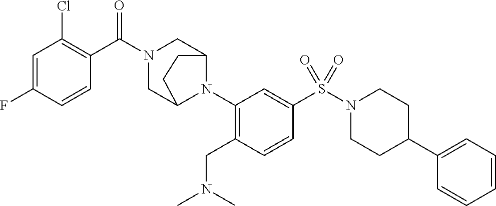 [SMILES: CN(C)Cc1ccc(S(=O)(=O)N2CCC(c3ccccc3)CC2)cc1N1C2CCC1CN(C(=O)c1ccc(F)cc1Cl)C2] | (2-chloro-4-fluoro- phenyl)-[(1S,5R)-8-[2- [(dimethylamino)methyl]- 5-[(4-phenyl-1- piperidyl)sulfonyl]phenyl]- 3,8- diazabicyclo[3.2.1]octan- 3-yl]methanone |
| 074 | 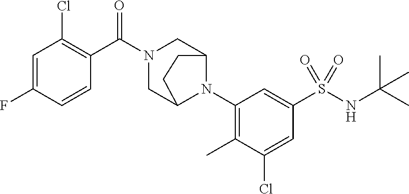 [SMILES: Cc1c(Cl)cc(S(=O)(=O)NC(C)(C)C)cc1N1C2CCC1CN(C(=O)c1ccc(F)cc1Cl)C2] | N-tert-butyl-3-chloro-5- [(1S,5R)-3-(2-chloro-4- fluoro-benzoyl)-3,8- diazabicyclo[3.2.1]octan- 8-yl]-4-methyl- benzenesulfonamide |
| 075 | 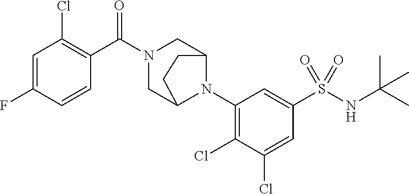 [SMILES: CC(C)(C)NS(=O)(=O)c1cc(Cl)c(Cl)c(N2C3CCC2CN(C(=O)c2ccc(F)cc2Cl)C3)c1] | N-tert-butyl-3,4-dichloro- 5-[(1S,5R)-3-(2-chloro-4- fluoro-benzoyl)-3,8- diazabicyclo[3.2.1]octan- 8-yl]benzenesulfonamide |
| 076 | 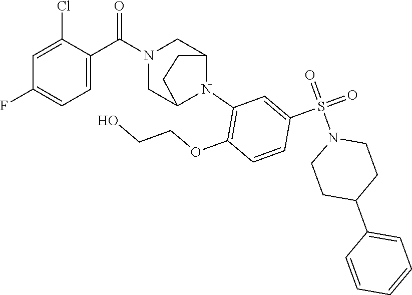 [SMILES: O=C(c1ccc(F)cc1Cl)N1CC2CCC(C1)N2c1cc(S(=O)(=O)N2CCC(c3ccccc3)CC2)ccc1OCCO] | (2-chloro-4-fluoro- phenyl)-[(1S,5R)-8-[2-(2- hydroxyethoxy)-5-[(4- phenyl-1- piperidyl)sulfonyl]phenyl]- 3,8- diazabicyclo[3.2.1]]octan- 3-yl]methanone |
| 077 | 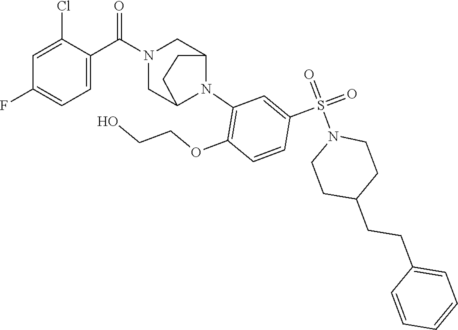 [SMILES: O=C(c1ccc(F)cc1Cl)N1CC2CCC(C1)N2c1cc(S(=O)(=O)N2CCC(CCc3ccccc3)CC2)ccc1OCCO] | (2-chloro-4-fluoro- phenyl)-[(1S,5R)-8-[2-(2- hydroxyethoxy)-5-[[4-(2- phenylethyl)-1- piperidyl]sulfonyl]phenyl]- 3,8- diazabicyclo[3.2.1]octan- 3-yl]methanone |
| 078 | 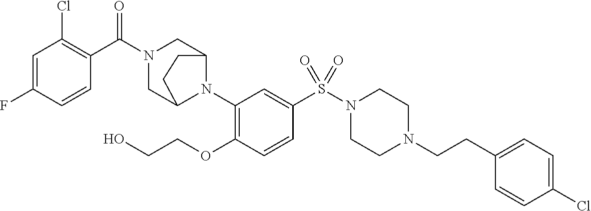 [SMILES: O=C(c1ccc(F)cc1Cl)N1CC2CCC(C1)N2c1cc(S(=O)(=O)N2CCN(CCc3ccc(Cl)cc3)CC2)ccc1OCCO] | (2-chloro-4-fluoro- phenyl)-[(1S,5R)-8-[5-[4- [2-(4- chlorophenyl)ethyl]piperazin- 1-yl]sulfonyl-2-(2- hydroxyethoxy)phenyl]- 3,8- diazabicyclo[3.2.1]octan- 3-yl]methanone |
| 079 | 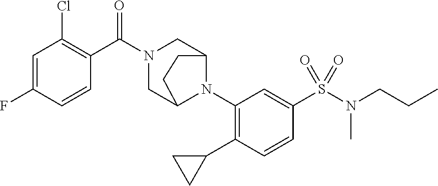 [SMILES: CCCN(C)S(=O)(=O)c1ccc(C2CC2)c(N2C3CCC2CN(C(=O)c2ccc(F)cc2Cl)C3)c1] | 3-[(1S,5R)-3-(2-chloro-4- fluoro-benzoyl)-3,8- diazabicyclo[3.2.1]]octan- 8-yl]-4-cyclopropyl-N- methyl-N-propyl- benzenesulfonamide |
| 080 | 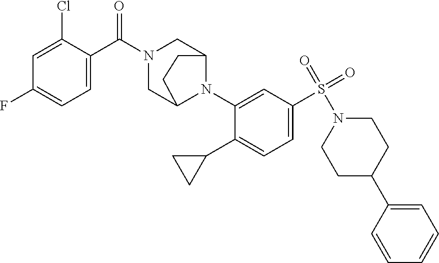 [SMILES: O=C(c1ccc(F)cc1Cl)N1CC2CCC(C1)N2c1cc(S(=O)(=O)N2CCC(c3ccccc3)CC2)ccc1C1CC1] | (2-chloro-4-fluoro- phenyl)-[(1S,5R)-8-[2- cyclopropyl-5-[(4-phenyl- 1- piperidyl)sulfonyl]phenyl]- 3,8- diazabicyclo[3.2.1]octan- 3-yl]methanone |
| 081 | 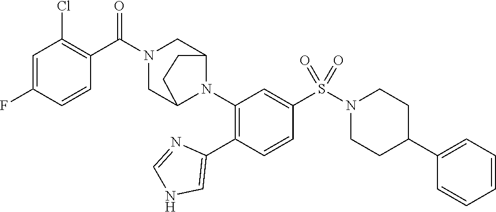 [SMILES: O=C(c1ccc(F)cc1Cl)N1CC2CCC(C1)N2c1cc(S(=O)(=O)N2CCC(c3ccccc3)CC2)ccc1-c1c[nH]cn1] | (2-chloro-4-fluoro- phenyl)-[(1S,5R)-8-[2- (1H-imidazol-4-yl)-5-[(4- phenyl-1- piperidyl)sulfonyl]phenyl]- 3,8- diazabicyclo[3.2.1]]octan- 3-yl]methanone |
| 082 | 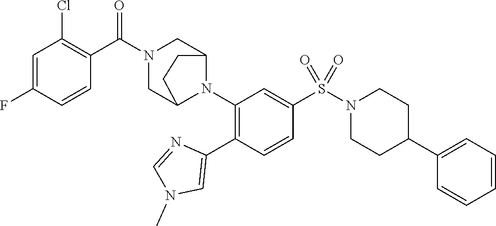 [SMILES: Cn1cnc(-c2ccc(S(=O)(=O)N3CCC(c4ccccc4)CC3)cc2N2C3CCC2CN(C(=O)c2ccc(F)cc2Cl)C3)c1] | (2-chloro-4-fluoro- phenyl)-[(1S,5R)-8-[2-(1- methylimidazol-4-yl)-5- [(4-phenyl-1- piperidyl)sulfonyl]phenyl]- 3,8- diazabicyclo[3.2.1]octan- 3-yl]methanone |
| 083 | 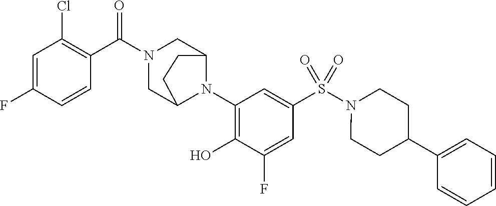 [SMILES: O=C(c1ccc(F)cc1Cl)N1CC2CCC(C1)N2c1cc(S(=O)(=O)N2CCC(c3ccccc3)CC2)cc(F)c1O] | (2-chloro-4-fluoro- phenyl)-[(1S,5R)-8-[3- fluoro-2-hydroxy-5-[(4- phenyl-1- piperidyl)sulfonyl]phenyl]- 3,8- diazabicyclo[3.2.1]octan- 3-yl]methanone |
| 084 | 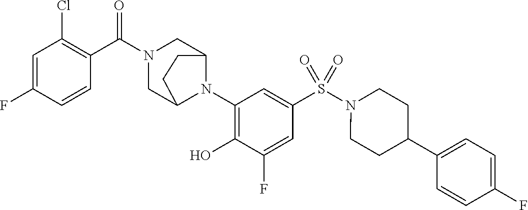 [SMILES: O=C(c1ccc(F)cc1Cl)N1CC2CCC(C1)N2c1cc(S(=O)(=O)N2CCC(c3ccc(F)cc3)CC2)cc(F)c1O] | (2-chloro-4-fluoro- phenyl)-[(1S,5R)-8-[3- fluoro-5-[[4-(4- fluorophenyl)-1- piperidyl]sulfonyl]-2- hydroxy-phenyl]-3,8- diazabicyclo[3.2.1]octan- 3-yl]methanone |
| 085 | 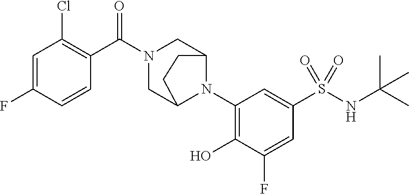 [SMILES: CC(C)(C)NS(=O)(=O)c1cc(F)c(O)c(N2C3CCC2CN(C(=O)c2ccc(F)cc2Cl)C3)c1] | N-tert-butyl-3-[(1S,5R)-3- (2-chloro-4-fluoro- benzoyl)-3,8- diazabicyclo[3.2.1]octan- 8-yl]-5-fluoro-4-hydroxy- benzenesulfonamide |
| 086 | 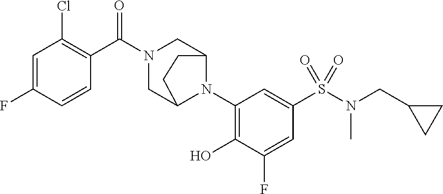 [SMILES: CN(CC1CC1)S(=O)(=O)c1cc(F)c(O)c(N2C3CCC2CN(C(=O)c2ccc(F)cc2Cl)C3)c1] | 3-[(1S,5R)-3-(2-chloro-4- fluoro-benzoyl)-3,8- diazabicyclo[3.2.1]octan- 8-yl]-N- (cyclopropylmethyl)-5- fluoro-4-hydroxy-N- methyl- benzenesulfonamide |
| 087 | 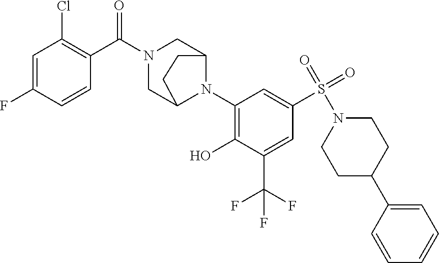 [SMILES: O=C(c1ccc(F)cc1Cl)N1CC2CCC(C1)N2c1cc(S(=O)(=O)N2CCC(c3ccccc3)CC2)cc(C(F)(F)F)c1O] | (2-chloro-4-fluoro- phenyl)-[(1S,5R)-8-[2- hydroxy-5-[(4-phenyl-1- piperidyl)sulfonyl]-3- (trifluoromethyl)phenyl]- 3,8- diazabicyclo[3.2.1]]octan- 3-yl]methanone |
| 088 | 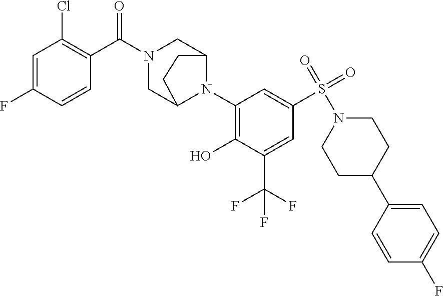 [SMILES: O=C(c1ccc(F)cc1Cl)N1CC2CCC(C1)N2c1cc(S(=O)(=O)N2CCC(c3ccc(F)cc3)CC2)cc(C(F)(F)F)c1O] | (2-chloro-4-fluoro- phenyl)-[(1S,5R)-8-[5-[[4- (4-fluorophenyl)-1- piperidyl]sulfonyl]-2- hydroxy-3- (trifluoromethyl)phenyl]- 3,8- diazabicyclo[3.2.1]octan- 3-yl]methanone |
| 089 | 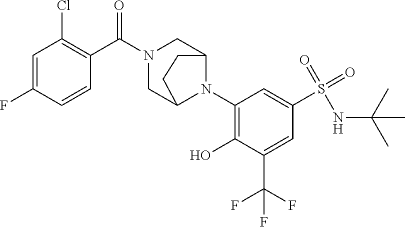 [SMILES: CC(C)(C)NS(=O)(=O)c1cc(N2C3CCC2CN(C(=O)c2ccc(F)cc2Cl)C3)c(O)c(C(F)(F)F)c1] | N-tert-butyl-3-[(1S,5R)-3- (2-chloro-4-fluoro- benzoyl)-3,8- diazabicyclo[3.2.1]]octan- 8-yl]-4-hydroxy-5- (trifluoromethyl) benzenesulfonamide |
| 090 | 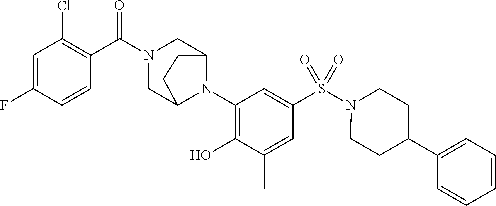 [SMILES: Cc1cc(S(=O)(=O)N2CCC(c3ccccc3)CC2)cc(N2C3CCC2CN(C(=O)c2ccc(F)cc2Cl)C3)c1O] | (2-chloro-4-fluoro- phenyl)-[(1S,5R)-8-[2- hydroxy-3-methyl-5-[(4- phenyl-1- piperidyl)sulfonyl]phenyl]- 3,8- diazabicyclo[3.2.1]octan- 3-yl]methanone |
| 091 | 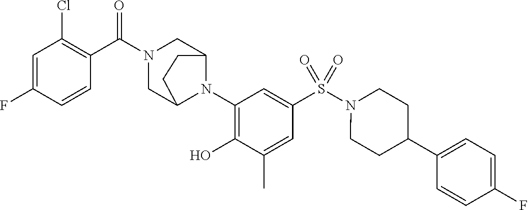 [SMILES: Cc1cc(S(=O)(=O)N2CCC(c3ccc(F)cc3)CC2)cc(N2C3CCC2CN(C(=O)c2ccc(F)cc2Cl)C3)c1O] | (2-chloro-4-fluoro- phenyl)-[(1S,5R)-8-[5-[[4- (4-fluorophenyl)-1- piperidyl]sulfonyl]-2- hydroxy-3-methyl- phenyl]-3,8- diazabicyclo[3.2.1]octan- 3-yl]methanone |
| 092 | 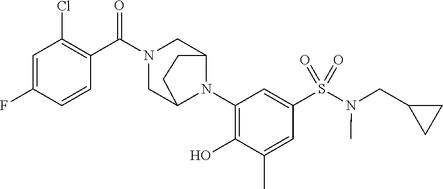 [SMILES: Cc1cc(S(=O)(=O)N(C)CC2CC2)cc(N2C3CCC2CN(C(=O)c2ccc(F)cc2Cl)C3)c1O] | 3-[(1S,5R)-3-(2-chloro-4- fluoro-benzoyl)-3,8- diazabicyclo[3.2.1]octan- 8-yl]-N- (cyclopropylmethyl)-4- hydroxy-N,5-dimethyl- benzenesulfonamide |
| 093 | 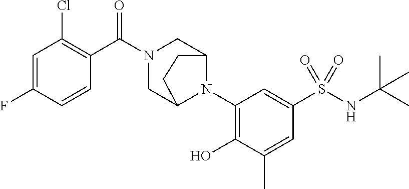 [SMILES: Cc1cc(S(=O)(=O)NC(C)(C)C)cc(N2C3CCC2CN(C(=O)c2ccc(F)cc2Cl)C3)c1O] | N-tert-butyl-3-[(1S,5R)-3- (2-chloro-4-fluoro- benzoyl)-3,8- diazabicyclo[3.2.1]]octan- 8-yl]-4-hydroxy-5-methyl- benzenesulfonamide |
| 094 | 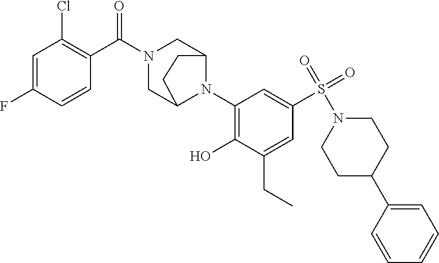 [SMILES: CCc1cc(S(=O)(=O)N2CCC(c3ccccc3)CC2)cc(N2C3CCC2CN(C(=O)c2ccc(F)cc2Cl)C3)c1O] | (2-chloro-4-fluoro- phenyl)-[(1S,5R)-8-[3- ethyl-2-hydroxy-5-[(4- phenyl-1- piperidyl)sulfonyl]phenyl]- 3,8- diazabicyclo[3.2.1]octan- 3-yl]methanone |
| 095 | 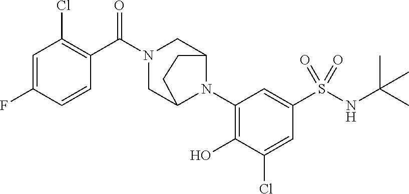 [SMILES: CC(C)(C)NS(=O)(=O)c1cc(Cl)c(O)c(N2C3CCC2CN(C(=O)c2ccc(F)cc2Cl)C3)c1] | N-tert-butyl-3-chloro-5- [(1S,5R)-3-(2-chloro-4- fluoro-benzoyl)-3,8- diazabicyclo[3.2.1]octan- 8-yl]-4-hydroxy- benzenesulfonamide |
| 096 | 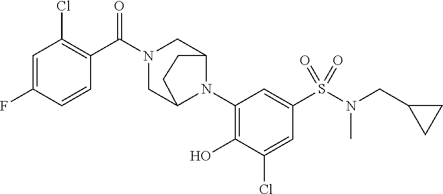 [SMILES: CN(CC1CC1)S(=O)(=O)c1cc(Cl)c(O)c(N2C3CCC2CN(C(=O)c2ccc(F)cc2Cl)C3)c1] | 3-chloro-5-[(1S,5R)-3-(2- chloro-4-fluoro-benzoyl)- 3,8- diazabicyclo[3.2.1]octan- 8-yl]-N- (cyclopropylmethyl)-4- hydroxy-N-methyl- benzenesulfonamide |
| 097 | 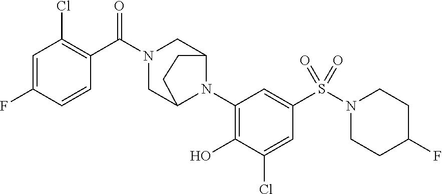 [SMILES: O=C(c1ccc(F)cc1Cl)N1CC2CCC(C1)N2c1cc(S(=O)(=O)N2CCC(F)CC2)cc(Cl)c1O] | (2-chloro-4-fluoro- phenyl)-[(1S,5R)-8-[3- chloro-5-[(4-fluoro-1- piperidyl)sulfonyl]-2- hydroxy-phenyl]-3,8- diazabicyclo[3.2.1]octan- 3-yl]methanone |
| 098 | 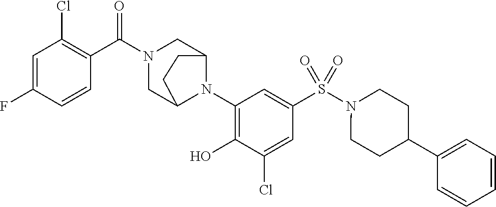 [SMILES: O=C(c1ccc(F)cc1Cl)N1CC2CCC(C1)N2c1cc(S(=O)(=O)N2CCC(c3ccccc3)CC2)cc(Cl)c1O] | (2-chloro-4-fluoro- phenyl)-[(1S,5R)-8-[3- chloro-2-hydroxy-5-[(4- phenyl-1- piperidyl)sulfonyl]phenyl]- 3,8- diazabicyclo[3.2.1]]octan- 3-yl]methanone |
| 099 | 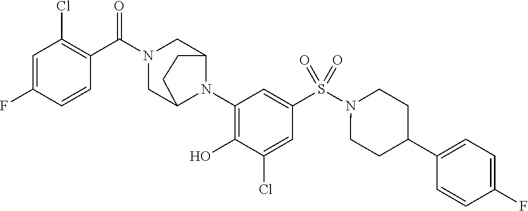 [SMILES: O=C(c1ccc(F)cc1Cl)N1CC2CCC(C1)N2c1cc(S(=O)(=O)N2CCC(c3ccc(F)cc3)CC2)cc(Cl)c1O] | (2-chloro-4-fluoro- phenyl)-[(1S,5R)-8-[3- chloro-5-[[4-(4- fluorophenyl)-1- piperidyl]sulfonyl]-2- hydroxy-phenyl]-3,8- diazabicyclo[3.2.1]octan- 3-yl]methanone |
| 100 | 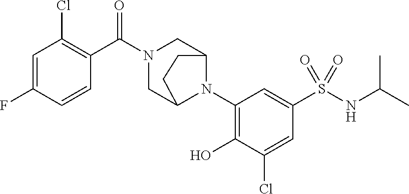 [SMILES: CC(C)NS(=O)(=O)c1cc(Cl)c(O)c(N2C3CCC2CN(C(=O)c2ccc(F)cc2Cl)C3)c1] | 3-chloro-5-[(1S,5R)-3-(2- chloro-4-fluoro-benzoyl)- 3,8- diazabicyclo[3.2.1]]octan- 8-yl]-4-hydroxy-N- isopropyl- benzenesulfonamide |
| 101 | 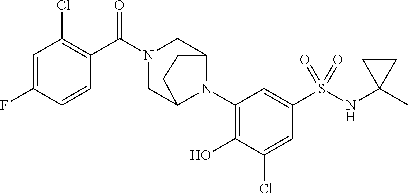 [SMILES: CC1(NS(=O)(=O)c2cc(Cl)c(O)c(N3C4CCC3CN(C(=O)c3ccc(F)cc3Cl)C4)c2)CC1] | 3-chloro-5-[(1S,5R)-3-(2- chloro-4-fluoro-benzoyl)- 3,8- diazabicyclo[3.2.1]octan- 8-yl]-4-hydroxy-N-(1- methylcyclopropyl) benzenesulfonamide |
| 102 | 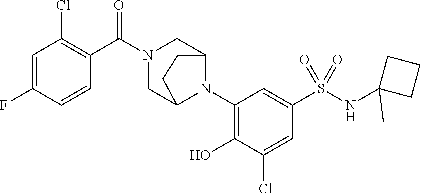 [SMILES: CC1(NS(=O)(=O)c2cc(Cl)c(O)c(N3C4CCC3CN(C(=O)c3ccc(F)cc3Cl)C4)c2)CCC1] | 3-chloro-5-[(1S,5R)-3-(2- chloro-4-fluoro-benzoyl)- 3,8- diazabicyclo[3.2.1]octan- 8-yl]-4-hydroxy-N-(1- methylcyclobutyl) benzenesulfonamide |
| 103 | 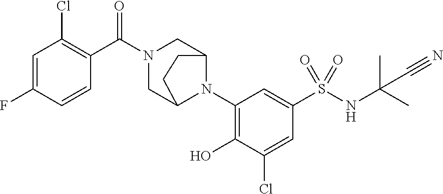 [SMILES: CC(C)(C#N)NS(=O)(=O)c1cc(Cl)c(O)c(N2C3CCC2CN(C(=O)c2ccc(F)cc2Cl)C3)c1] | 3-chloro-5-[(1S,5R)-3-(2- chloro-4-fluoro-benzoyl)- 3,8- diazabicyclo[3.2.1]]octan- 8-yl]-N-(1-cyano-1- methyl-ethyl)-4-hydroxy- benzenesulfonamide |
| 104 | 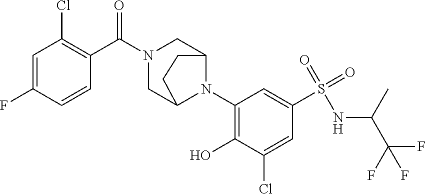 [SMILES: CC(NS(=O)(=O)c1cc(Cl)c(O)c(N2C3CCC2CN(C(=O)c2ccc(F)cc2Cl)C3)c1)C(F)(F)F] | 3-chloro-5-[(1S,5R)-3-(2- chloro-4-fluoro-benzoyl)- 3,8- diazabicyclo[3.2.1]octan- 8-yl]-4-hydroxy-N-[(1SR)- 2,2,2-trifluoro-1-methyl- ethyl]benzenesulfonamide |
| 105 | 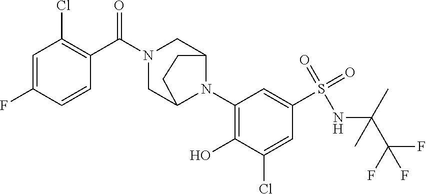 [SMILES: CC(C)(NS(=O)(=O)c1cc(Cl)c(O)c(N2C3CCC2CN(C(=O)c2ccc(F)cc2Cl)C3)c1)C(F)(F)F] | 3-chloro-5-[(1S,5R)-3-(2- chloro-4-fluoro-benzoyl)- 3,8- diazabicyclo[3.2.1]octan- 8-yl]-4-hydroxy-N-(2,2,2- trifluoro-1,1-dimethyl- ethyl)benzenesulfonamide |
| 106 | 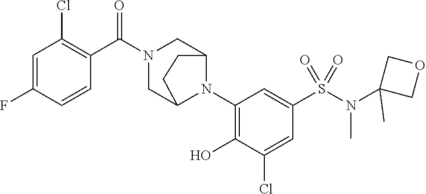 [SMILES: CN(C1(C)COC1)S(=O)(=O)c1cc(Cl)c(O)c(N2C3CCC2CN(C(=O)c2ccc(F)cc2Cl)C3)c1] | 3-chloro-5-[(1S,5R)-3-(2- chloro-4-fluoro-benzoyl)- 3,8- diazabicyclo[3.2.1]octan- 8-yl]-4-hydroxy-N- methyl-N-(3- methyloxetan-3- yl)benzenesulfonamide |
| 107 | 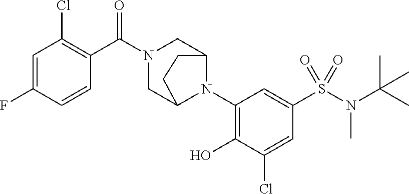 [SMILES: CN(C(C)(C)C)S(=O)(=O)c1cc(Cl)c(O)c(N2C3CCC2CN(C(=O)c2ccc(F)cc2Cl)C3)c1] | N-tert-butyl-3-chloro-5- [(1S,5R)-3-(2-chloro-4- fluoro-benzoyl)-3,8- diazabicyclo[3.2.1]]octan- 8-yl]-4-hydroxy-N- methyl- benzenesulfonamide |
| 108 | 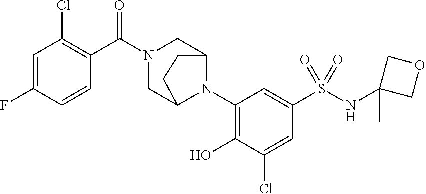 [SMILES: CC1(NS(=O)(=O)c2cc(Cl)c(O)c(N3C4CCC3CN(C(=O)c3ccc(F)cc3Cl)C4)c2)COC1] | 3-chloro-5-[(1S,5R)-3-(2- chloro-4-fluoro-benzoyl)- 3,8- diazabicyclo[3.2.1]]octan- 8-yl]-4-hydroxy-N-(3- methyloxetan-3- yl)benzenesulfonamide |
| 109 | 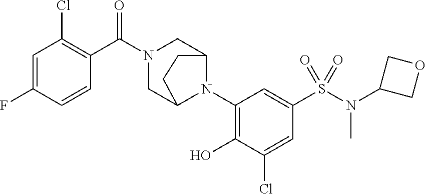 [SMILES: CN(C1COC1)S(=O)(=O)c1cc(Cl)c(O)c(N2C3CCC2CN(C(=O)c2ccc(F)cc2Cl)C3)c1] | 3-chloro-5-[(1S,5R)-3-(2- chloro-4-fluoro-benzoyl)- 3,8- diazabicyclo[3.2.1]octan- 8-yl]-4-hydroxy-N- methyl-N-(oxetan-3- yl)benzenesulfonamide |
| 110 | 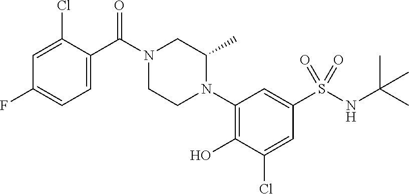 [SMILES: C[C@H]1CN(C(=O)c2ccc(F)cc2Cl)CCN1c1cc(S(=O)(=O)NC(C)(C)C)cc(Cl)c1O] | N-tert-butyl-3-chloro-5- [(2S)-4-(2-chloro-4-fluoro- benzoyl)-2-methyl- piperazin-1-yl]-4-hydroxy- benzenesulfonamide |
| 111 | 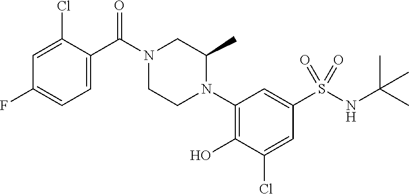 [SMILES: C[C@@H]1CN(C(=O)c2ccc(F)cc2Cl)CCN1c1cc(S(=O)(=O)NC(C)(C)C)cc(Cl)c1O] | N-tert-butyl-3-chloro-5- [(2R)-4-(2-chloro-4- fluoro-benzoyl)-2-methyl- piperazin-1-yl]-4-hydroxy- benzenesulfonamide |
| 112 | 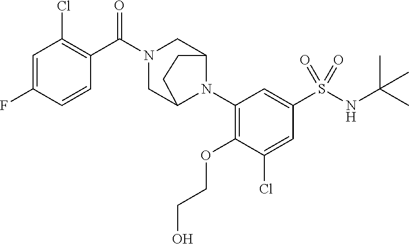 [SMILES: CC(C)(C)NS(=O)(=O)c1cc(Cl)c(OCCO)c(N2C3CCC2CN(C(=O)c2ccc(F)cc2Cl)C3)c1] | N-tert-butyl-3-chloro-5- [(1S,5R)-3-(2-chloro-4- fluoro-benzoyl)-3,8- diazabicyclo[3.2.1]]octan- 8-yl]-4-(2- hydroxyethoxy) benzenesulfonamide |
| 113 | 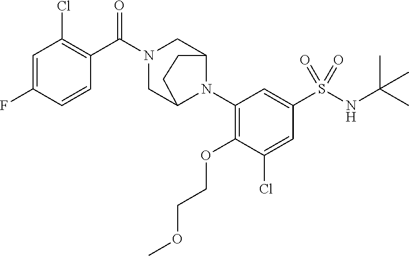 [SMILES: COCCOc1c(Cl)cc(S(=O)(=O)NC(C)(C)C)cc1N1C2CCC1CN(C(=O)c1ccc(F)cc1Cl)C2] | N-tert-butyl-3-chloro-5- [(1S,5R)-3-(2-chloro-4- fluoro-benzoyl)-3,8- diazabicyclo[3.2.1]]octan- 8-yl]-4-(2- methoxyethoxy) benzenesulfonamide |
| 114 | 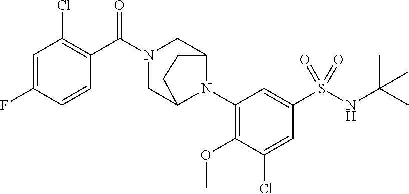 [SMILES: COc1c(Cl)cc(S(=O)(=O)NC(C)(C)C)cc1N1C2CCC1CN(C(=O)c1ccc(F)cc1Cl)C2] | N-tert-butyl-3-chloro-5- [(1S,5R)-3-(2-chloro-4- fluoro-benzoyl)-3,8- diazabicyclo[3.2.1]octan- 8-yl]-4-methoxy- benzenesulfonamide |
| 115 | 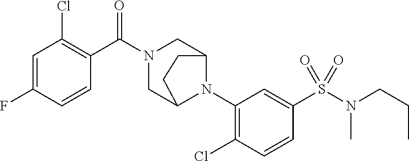 [SMILES: CCCN(C)S(=O)(=O)c1ccc(Cl)c(N2C3CCC2CN(C(=O)c2ccc(F)cc2Cl)C3)c1] | 4-chloro-3-[(1S,5R)-3-(2- chloro-4-fluoro-benzoyl)- 3,8- diazabicyclo[3.2.1]octan- 8-yl]-N-methyl-N-propyl- benzenesulfonamide |
| 116 | 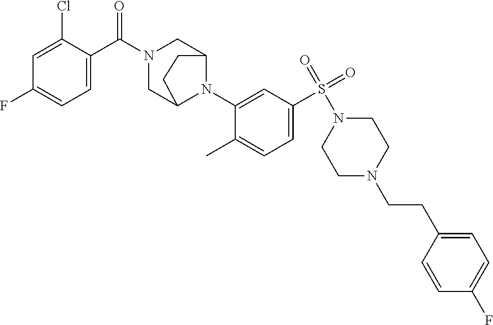 [SMILES: Cc1ccc(S(=O)(=O)N2CCN(CCc3ccc(F)cc3)CC2)cc1N1C2CCC1CN(C(=O)c1ccc(F)cc1Cl)C2] | (2-chloro-4-fluoro- phenyl)-[(1S,5R)-8-[5-[4- [2-(4- fluorophenyl)ethyl] piperazin-1-yl]sulfonyl- 2-methyl- phenyl]-3,8- diazabicyclo[3.2.1]octan- 3-yl]methanone |
| 117 | 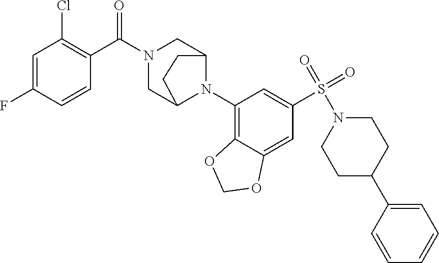 [SMILES: O=C(c1ccc(F)cc1Cl)N1CC2CCC(C1)N2c1cc(S(=O)(=O)N2CCC(c3ccccc3)CC2)cc2c1OCO2] | (2-chloro-4-fluoro- phenyl)-[(1S,5R)-8-[6-[(4- phenyl-1- piperidyl)sulfonyl]-1,3- benzodioxol-4-yl]-3,8- diazabicyclo[3.2.1]octan- 3-yl]methanone |
| 118 | 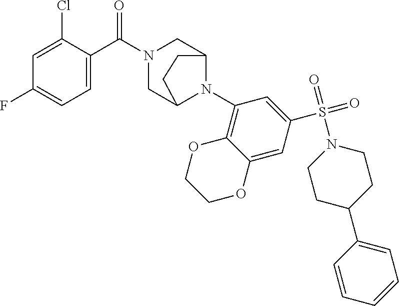 [SMILES: O=C(c1ccc(F)cc1Cl)N1CC2CCC(C1)N2c1cc(S(=O)(=O)N2CCC(c3ccccc3)CC2)cc2c1OCCO2] | (2-chloro-4-fluoro- phenyl)-[(1S,5R)-8-[7-[(4- phenyl-1- piperidyl)sulfonyl]-2,3- dihydro-1,4-benzodioxin- 5-yl]-3,8- diazabicyclo[3.2.1]octan- 3-yl]methanone |
| 119 | 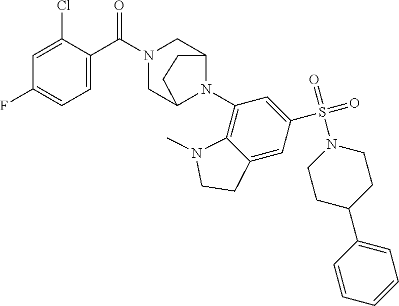 [SMILES: CN1CCc2cc(S(=O)(=O)N3CCC(c4ccccc4)CC3)cc(N3C4CCC3CN(C(=O)c3ccc(F)cc3Cl)C4)c21] | (2-chloro-4-fluoro- phenyl)-[(1S,5R)-8-[1- methyl-5-[(4-phenyl-1- piperidyl)sulfonyl]indolin- 7-yl]-3,8- diazabicyclo[3.2.1]octan- 3-yl]methanone |
| 120 | 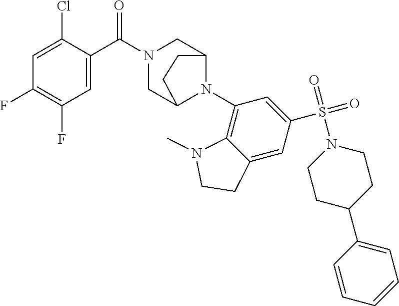 [SMILES: CN1CCc2cc(S(=O)(=O)N3CCC(c4ccccc4)CC3)cc(N3C4CCC3CN(C(=O)c3cc(F)c(F)cc3Cl)C4)c21] | (2-chloro-4,5-difluoro- phenyl)-[(1S,5R)-8-[1- methyl-5-[(4-phenyl-1- piperidyl)sulfonyl]indolin- 7-yl]-3,8- diazabicyclo[3.2.1]octan- 3-yl]methanone |
| 121 | 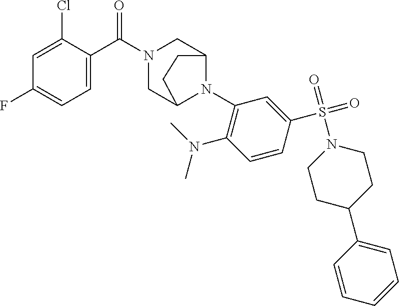 [SMILES: CN(C)c1ccc(S(=O)(=O)N2CCC(c3ccccc3)CC2)cc1N1C2CCC1CN(C(=O)c1ccc(F)cc1Cl)C2] | (2-chloro-4-fluoro- phenyl)-[(1S,5R)-8-[2- (dimethylamino)-5-[(4- phenyl-1- piperidyl)sulfonyl]phenyl]- 3,8- diazabicyclo[3.2.1]octan- 3-yl]methanone |
| 122 | 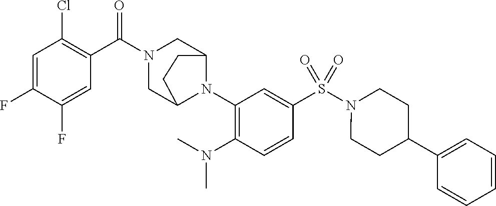 [SMILES: CN(C)c1ccc(S(=O)(=O)N2CCC(c3ccccc3)CC2)cc1N1C2CCC1CN(C(=O)c1cc(F)c(F)cc1Cl)C2] | (2-chloro-4,5-difluoro- phenyl)-[(1S,5R)-8-[2- (dimethylamino)-5-[(4- phenyl-1- piperidyl)sulfonyl]phenyl]- 3,8- diazabicyclo[3.2.1]octan- 3-yl]methanone |
| 123 | 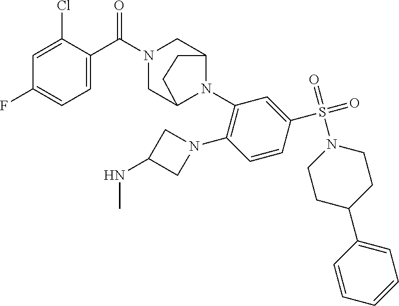 [SMILES: CNC1CN(c2ccc(S(=O)(=O)N3CCC(c4ccccc4)CC3)cc2N2C3CCC2CN(C(=O)c2ccc(F)cc2Cl)C3)C1] | (2-chloro-4-fluoro- phenyl)-[(1S,5R)-8-[2-[3- (methylamino)azetidin-1- yl]-5-[(4-phenyl-1- piperidyl)sulfonyl]phenyl]- 3,8- diazabicyclo[3.2.1]octan- 3-yl]methanone |
| 124 | 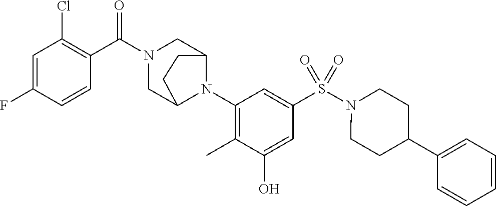 [SMILES: Cc1c(O)cc(S(=O)(=O)N2CCC(c3ccccc3)CC2)cc1N1C2CCC1CN(C(=O)c1ccc(F)cc1Cl)C2] | (2-chloro-4-fluoro- phenyl)-[(1S,5R)-8-[3- hydroxy-2-methyl-5-[(4- phenyl-1- piperidyl)sulfonyl]phenyl]- 3,8- diazabicyclo[3.2.1]octan- 3-yl]methanone |
| 125 | 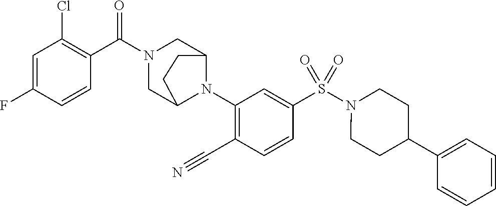 [SMILES: N#Cc1ccc(S(=O)(=O)N2CCC(c3ccccc3)CC2)cc1N1C2CCC1CN(C(=O)c1ccc(F)cc1Cl)C2] | 2-[(1R,5S)-3-(2-chloro-4- fluoro-benzoyl)-3,8- diazabicyclo[3.2.1]octan- 8-yl]-4-(4- phenylcyclohexyl)sulfonyl- benzonitrile |
| 126 | 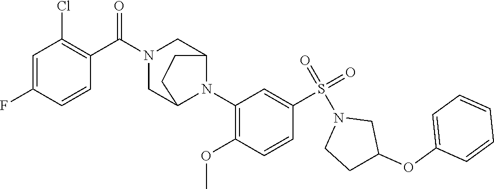 [SMILES: COc1ccc(S(=O)(=O)N2CCC(Oc3ccccc3)C2)cc1N1C2CCC1CN(C(=O)c1ccc(F)cc1Cl)C2] | (2-chloro-4-fluoro- phenyl)-[(1S,5R)-8-[2- methoxy-5-[(3SR)-3- phenoxypyrrolidin-1- yl]sulfonyl-phenyl]-3,8- diazabicyclo[3.2.1]octan- 3-yl]methanone |
| 127 | 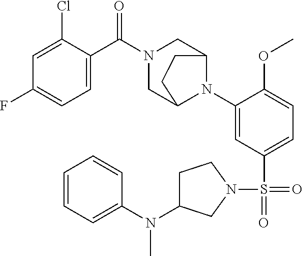 [SMILES: COc1ccc(S(=O)(=O)N2CCC(N(C)c3ccccc3)C2)cc1N1C2CCC1CN(C(=O)c1ccc(F)cc1Cl)C2] | (2-chloro-4-fluoro- phenyl)-[(1S,5R)-8-[2- methoxy-5-[(3SR)-3-(N- methylanilino)pyrrolidin- 1-yl]sulfonyl-phenyl]-3,8- diazabicyclo[3.2.1]octan- 3-yl]methanone |
| 128 | 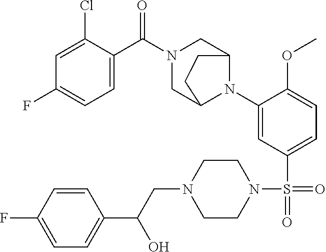 [SMILES: COc1ccc(S(=O)(=O)N2CCN(CC(O)c3ccc(F)cc3)CC2)cc1N1C2CCC1CN(C(=O)c1ccc(F)cc1Cl)C2] | (2-chloro-4-fluoro- phenyl)-[(1S,5R)-8-[5-[4- [(2RS)-2-(4-fluorophenyl)- 2-hydroxy-ethyl]piperazin- 1-yl]sulfonyl-2-methoxy- phenyl]-3,8- diazabicyclo[3.2.1]octan- 3-yl]methanone |
| 129 | 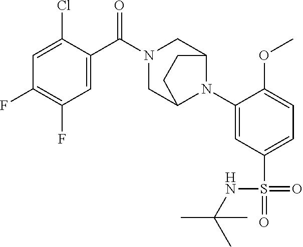 [SMILES: COc1ccc(S(=O)(=O)NC(C)(C)C)cc1N1C2CCC1CN(C(=O)c1cc(F)c(F)cc1Cl)C2] | N-tert-butyl-3-[(1S,5R)-3- (2-chloro-4,5-difluoro- benzoyl)-3,8- diazabicyclo[3.2.1]octan- 8-yl]-4-methoxy- benzenesulfonamide |
| 130 | 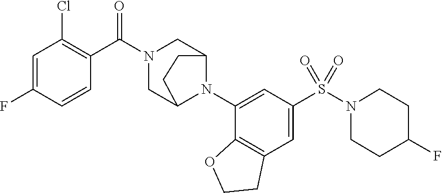 [SMILES: O=C(c1ccc(F)cc1Cl)N1CC2CCC(C1)N2c1cc(S(=O)(=O)N2CCC(F)CC2)cc2c1OCC2] | (2-chloro-4-fluoro- phenyl)-[(1S,5R)-8-[5-[(4- fluoro-1- piperidyl)sulfonyl]-2,3- dihydrobenzofuran-7-yl]- 3,8- diazabicyclo[3.2.1]octan- 3-yl]methanone |
| 131 | 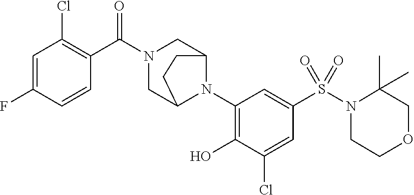 [SMILES: CC1(C)COCCN1S(=O)(=O)c1cc(Cl)c(O)c(N2C3CCC2CN(C(=O)c2ccc(F)cc2Cl)C3)c1] | [(1S,5R)-8-[3-chloro-5- (3,3-dimethylmorpholin-4- yl)sulfonyl-2-hydroxy- phenyl]-3,8- diazabicyclo[3.2.1]octan- 3-yl]-(2-chloro-4-fluoro- phenyl)methanone |
| 132 | 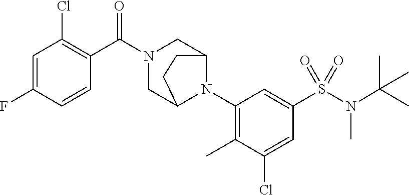 [SMILES: Cc1c(Cl)cc(S(=O)(=O)N(C)C(C)(C)C)cc1N1C2CCC1CN(C(=O)c1ccc(F)cc1Cl)C2] | N-tert-butyl-3-chloro-5- [(1S,5R)-3-(2-chloro-4- fluoro-benzoyl)-3,8- diazabicyclo[3.2.1]octan- 8-yl]-N,4-dimethyl- benzenesulfonamide |
| 133 | 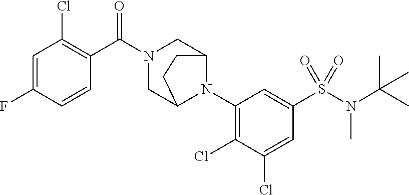 [SMILES: CN(C(C)(C)C)S(=O)(=O)c1cc(Cl)c(Cl)c(N2C3CCC2CN(C(=O)c2ccc(F)cc2Cl)C3)c1] | N-tert-butyl-3,4-dichloro- 5-[(1S,5R)-3-(2-chloro-4- fluoro-benzoyl)-3,8- diazabicyclo[3.2.1]octan- 8-yl]-N-methyl- benzenesulfonamide |
| 134 | 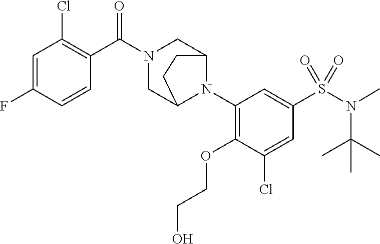 [SMILES: CN(C(C)(C)C)S(=O)(=O)c1cc(Cl)c(OCCO)c(N2C3CCC2CN(C(=O)c2ccc(F)cc2Cl)C3)c1] | N-tert-butyl-3-chloro-5- [(1S,5R)-3-(2-chloro-4- fluoro-benzoyl)-3,8- diazabicyclo[3.2.1]]octan- 8-yl]-4-(2- hydroxyethoxy)-N-methyl- benzenesulfonamide |
| 135 | 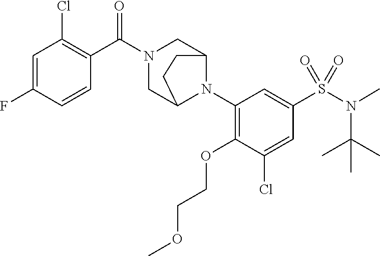 [SMILES: COCCOc1c(Cl)cc(S(=O)(=O)N(C)C(C)(C)C)cc1N1C2CCC1CN(C(=O)c1ccc(F)cc1Cl)C2] | N-tert-butyl-3-chloro-5- [(1S,5R)-3-(2-chloro-4- fluoro-benzoyl)-3,8- diazabicyclo[3.2.1]octan- 8-yl]-4-(2- methoxyethoxy)-N- methyl- benzenesulfonamide |
| 136 | 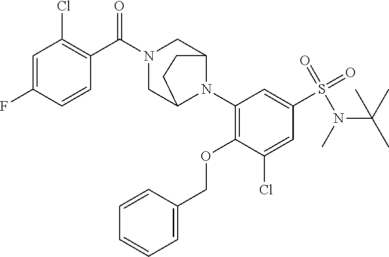 [SMILES: CN(C(C)(C)C)S(=O)(=O)c1cc(Cl)c(OCc2ccccc2)c(N2C3CCC2CN(C(=O)c2ccc(F)cc2Cl)C3)c1] | 4-benzyloxy-N-tert-butyl- 3-chloro-5-[(1S,5R)-3-(2- chloro-4-fluoro-benzoyl)- 3,8- diazabicyclo[3.2.1]octan- 8-yl]-N-methyl- benzenesulfonamide |
| 137 | 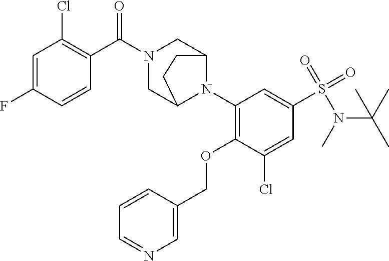 [SMILES: CN(C(C)(C)C)S(=O)(=O)c1cc(Cl)c(OCc2cccnc2)c(N2C3CCC2CN(C(=O)c2ccc(F)cc2Cl)C3)c1] | N-tert-butyl-3-chloro-5- [(1S,5R)-3-(2-chloro-4- fluoro-benzoyl)-3,8- diazabicyclo[3.2.1]]octan- 8-yl]-N-methyl-4-(3- pyridylmethoxy)benzenesu lfonamide |
| 138 | 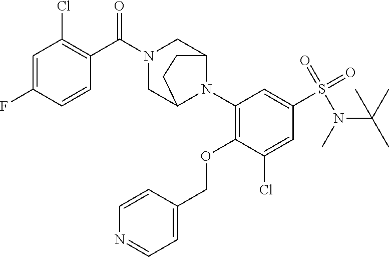 [SMILES: CN(C(C)(C)C)S(=O)(=O)c1cc(Cl)c(OCc2ccncc2)c(N2C3CCC2CN(C(=O)c2ccc(F)cc2Cl)C3)c1] | N-tert-butyl-3-chloro-5- [(1S,5R)-3-(2-chloro-4- fluoro-benzoyl)-3,8- diazabicyclo[3.2.1]octan- 8-yl]-N-methyl-4-(4- pyridylmethoxy) benzenesulfonamide |
| 139 | 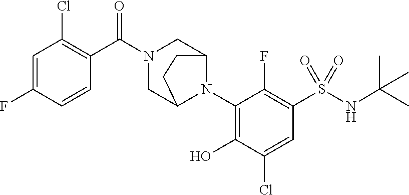 [SMILES: CC(C)(C)NS(=O)(=O)c1cc(Cl)c(O)c(N2C3CCC2CN(C(=O)c2ccc(F)cc2Cl)C3)c1F] | N-tert-butyl-5-chloro-3- [(1S,5R)-3-(2-chloro-4- fluoro-benzoyl)-3,8- diazabicyclo[3.2.1]octan- 8-yl]-2-fluoro-4-hydroxy- benzenesulfonamide |

All references herein to a compound of the invention (e.g., “compound of formula (I)”) include references to salts, solvates, multi component complexes and liquid crystals thereof. All references herein to a compound of the invention include references to polymorphs and crystal habits thereof. All references herein to a compound of the invention include references to isotopically-labelled compounds thereof, including deuterated compounds thereof. All references herein to a compound of the invention include references to stereoisomers thereof. All references herein to a compound of the invention include references to pharmaceutically acceptable prodrugs thereof.

In particular, the compounds of the invention may be in the form of pharmaceutically acceptable salts. According to one embodiment, the compound of the invention is a pharmaceutically acceptable salt.

Pharmaceutically acceptable salts include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinafoate salts. Suitable base salts are formed from bases which form non-toxic salts. Examples include the aluminium, arginine, benzathine, calcium, choline, diethylamine, 2-(diethylamino) ethanol, diolamine, ethanolamine, glycine, 4-(2-hydroxyethyl)-morpholine, lysine, magnesium, meglumine, morpholine, olamine, potassium, sodium, tromethamine and zinc salts. Hemisalts of acids and bases may also be formed, for example, hemisulphate and hemicalcium salts. When a compound contains an acidic group as well as a basic group the compound may also form internal salts, and such compounds are within the scope of the invention. When a compound contains a hydrogen-donating heteroatom (e.g., NH), the invention also encompasses salts and/or isomers formed by transfer of said hydrogen atom to a basic group or atom within the molecule.

Pharmaceutically acceptable salts of compounds of the invention may be prepared by one or more of these methods: (i) by reacting the compound with the desired acid; (ii) by reacting the compound with the desired base; (iii) by removing an acid- or base-labile protecting group from a suitable precursor of the compound or by ring-opening a suitable cyclic precursor, e.g., a lactone or lactam, using the desired acid; and/or (iv) by converting one salt of the compound to another by reaction with an appropriate acid or by means of a suitable ion exchange column. All these reactions are typically carried out in solution. The salt may precipitate from solution and be collected by filtration or may be recovered by evaporation of the solvent. The degree of ionization in the salt may vary from completely ionized to almost non-ionized.

In particular, the compounds of the invention may be in the form of pharmaceutically acceptable solvates. According to one embodiment, the compound of the invention is a pharmaceutically acceptable solvate. According to one embodiment, the compound of the invention is a pharmaceutically acceptable salt and solvate.

In particular, the compounds of the invention may include at least one asymmetric center(s) and thus may exist as different stereoisomeric forms. Accordingly, all references herein to a compound of the invention include all possible stereoisomers and include not only the racemic compounds, but the individual enantiomers and their non-racemic mixtures as well. Non-racemic mixtures may comprise any amounts of each distinct stereoisomer, for example one stereoisomer may be preponderant (e.g., a 90/10 or 80/20 mixture), or the enantiomeric ratio may be close to a racemic mixture (e.g., a 40/60 mixture). When a compound is desired as a single enantiomer, such single enantiomer may be obtained by stereospecific synthesis, by resolution of the final product or any convenient intermediate, or by chiral chromatographic methods as each are known in the art. Resolution of the final product, an intermediate, or a starting material may be carried out by any suitable method known in the art.

### Pharmaceutical Composition

Another object of the present invention is a composition comprising a compound according to the invention, as described herein. According to one embodiment, the composition further comprises at least one pharmaceutically acceptable carrier, so that the composition is a “pharmaceutical composition” as defined herein.

In a first embodiment, the pharmaceutical composition comprises the compound according to the invention as sole therapeutic agent. In a second embodiment, the pharmaceutical composition further comprises at least another therapeutic agent such as, for example, a therapeutic agent suitable for treating a neurological disorder.

Another object of the present invention is a medicament comprising a compound according to the invention, as described herein.

### Kit

Another object of the present invention is a kit of parts (in short “kit”) comprising a compound or composition according to the invention, as described herein. According to one embodiment, the kit comprises a manufacture such as, for example, a package or a container. According to one embodiment, the kit comprises instructions for use. The kit may be promoted, distributed, or sold as a unit for performing the methods of the present invention.

According to one embodiment, the kit comprises: a pharmaceutical composition comprising the compound according to the invention, and another separate pharmaceutical composition comprising at least another therapeutic agent such as, for example, a therapeutic agent suitable for treating a neurological disorder.

### Medical Use of the Compound

Another object of the present invention is a compound or a composition according to the invention, as described herein, for use as a medicament.

Another object of the present invention is a compound or a composition according to the invention, as described herein, for use in the treatment of a neurological disorder.

Another object of the present invention is a method for treating a neurological disorder in a subject in need thereof. Another object of the present invention is the use of a compound or a composition according to the invention, as described herein, in the manufacture of a medicament for the treatment of a neurological disorder. Another object of the present invention is the use of a compound or a composition according to the invention, as described herein, for treating a neurological disorder.

According to one embodiment, the method or the use comprises a step of administering to a subject a therapeutically effective amount of a compound, a composition or a medicament according to the invention, as described herein.

According to one embodiment, the neurological disorder to be treated by the method or the use of the invention is:
a disease or a disorder associated with defective neurogenesis such as, for example, Hirschsprung disease, schizophrenia, Ataxia telangiectasia, age-related decline of nervous system performance, or a neurodevelopmental disorder;
a neurodegenerative disease or disorder, such as, for example, Alzheimer's disease, Parkinson's disease, Huntington's disease, Amyotrophic Lateral Sclerosis, Fronto-temporal dementia, retinal neurodegenerative diseases, neuro-ophthalmic diseases, neurotrophic keratitis, Charcot-Marie-Tooth disease, Spinal Muscular Atrophy, epilepsy (e.g., an epilepsy disorder, or a seizure disorder, or a chronic neurological disorder presenting with epilepsy), dementia, age-related decline of nervous system performance, a prion disease, Creutzfeldt-Jacob disease, Multiple System Atrophy (Shy Drager Syndrome), Multiple sclerosis, or Guillain-Barre syndrome;
a disease or a disorder associated with nerve injury or neurotoxicity, such as, for example, head injury, brain damage, traumatic brain injury, peripheral nerve injury, traumatic peripheral nerve injury, peripheral neuropathy, nerve transplantation complications, spinal cord injury, traumatic spinal cord injury, severance of nerves or nerve damage, severance of cerebrospinal nerve cord, a damage to brain or nerve cells, syringomyelia, optic neuropathy, trauma, stroke, ischemia, stroke, ischemic stroke, neurotoxicity caused by alcohol or substance abuse (e.g., ecstasy, methamphetamine etc.), or aphasia;
a neurodevelopmental disorder, such as, for example, Rett syndrome, X-linked mental retardation, fragile X syndrome, Down's syndrome, Autism Spectrum Disorder, Hirschsprung's disease, Tourette syndrome, childhood learning disorder, an attention deficit disorder, Attention Deficit Hyperactivity Disorder (ADHD), Angelman syndrome, micropreemie, schizophrenia, Language disorder, preterm birth, perinatal arterial ischemic stroke, spina bifida, mental retardation, nonsyndromic X-linked mental retardation, Ondine syndrome, or WAGR syndrome;
a neuropsychiatric disorder such as, for example, depression, Major Depressive Disorder, schizophrenia, schizophrenic form disorder, schizoaffective disorder, delusional disorder, anxiety, anxiety disorders, panic disorder, phobias, an obsessive-compulsive disorder, a post-traumatic stress disorder, a bipolar disorder, anorexia nervosa, bulimia nervosa, anhedonia, apathy, dementia, substance-induced dementia, a motor and/or tic disorder characterized by motor and/or vocal tics (e.g., Tourette's disorder), a substance use behavior, addiction, mood disorders, suicidality, cancer-related psychiatric symptoms, Alzheimer's disease, Huntington's disease, Fronto-temporal dementia, or a reward deficiency syndrome (RDS);
a movement disorder, such as, for example, Parkinson's disease, Huntington's disease, Amyotrophic Lateral Sclerosis, a motor and tic disorder characterized by motor and/or vocal tics (e.g., Tourette's disorder), an ataxia, ataxias muscular rigidity (spasticity), Charcot-Marie-Tooth disease, Spinal Muscular Atrophy, Werdnig-Hoffmann disease, or chronic proximal spinal muscular atrophy;
a pain disorder, such as, for example, neuralgia, trigeminal neuralgia, chronic pain, inflammatory pain, pain associated with arthritis, fibromyalgia, back pain, cancer-associated pain, pain associated with digestive disease, pain associated with Crohn's disease, pain associated with autoimmune disease, pain associated with endocrine disease, pain associated with diabetic neuropathy, phantom limb pain, spontaneous pain, chronic post-surgical pain, chronic, temporomandibular pain, causalgia, post-herpetic neuralgia, AIDS-related pain, complex regional pain syndromes type I and II, trigeminal neuralgia, chronic back pain, pain associated with spinal cord injury, pain associated with drug intake and recurrent acute pain, neuropathic pain, or inappropriate neuronal activity resulting in neurodysthesia in a disease such as diabetes, an MS and a motor neuron disease;
an ophthalmic disease or an ocular disorder such as, for example, a retinal disorder, retinal neurodegenerative diseases, Retinitis Pigmentosa, Non-Arthritic Anterior

Ischemic Optic Neuropathy (NAION), macular degeneration, age-related macular degeneration, glaucoma, diabetic retinopathy, optic neuropathy and retinal degeneration, neuro-ophthalmic diseases, age-related cataract, primary open-angle glaucoma (POAG), retinal ganglion cell damage, ocular hypertension, ischemic optic neuropathy, macular telangiectasia, cystoid macular edema, Macular Telangiectasia Type 2, neurotrophic keratitis, or strabismus;
a disorder of the enteric system or a gastro-intestinal disorder, such as, for example, a disorder of intestinal motility, constipation, Hirschsprung's disease, Inflammatory Bowel Disease, Intestinal Neuronal Dysplasia, ulcerative colitis, Achalasia, Esophageal spasm, duodenal ulcer, Zollinger-Ellison Syndrome, hypersecretion of gastric acid, malabsorptive disorder or intestinal inflammation;
a progressive muscular dystrophy, such as, for example, Duchenne, Becker, Emery-Dreifuss, Landowy-Dejerine, scapulohumeral, limb-girdle, Von Graefe-Fuchs, oculopharyngeal, myotonic and congenital, a congenital or acquired myopathy, Charcot-Marie-Tooth disease, Werdnig-Hoffmann disease, or chronic proximal spinal muscular atrophy;
a disease or a disorder associated with defective long-term or short-term memory, such as, for example, memory loss, benign forgetfulness, or Alzheimer's disease;
an autoimmune disorder such as, for example, multiple sclerosis, autoimmune encephalomyelitis, autoimmune encephalitis, autoimmune hemolytic anemia, chronic lymphocytic leukemia, Churg-Strauss syndrome, anti-N-methyl-D-aspartate receptor (NMDAR) encephalitis, Thyroid-associated orbitopathy, autoimmune thyroiditis, Guillain-Barré syndrome, or autoimmune thrombocytopenia purpura;
a neuro-ontological disease or disorder such as, for example, cochlear sensory cell damage, defective auditory perception, hearing loss, or tinnitus;
a sleep disorder, such as, for example, narcolepsy, restless leg syndrome, Obstructive sleep apnea, chronic insomnia disorder, paradoxical sleep deprivation or REM sleep deprivation;
a cerebrovascular disease or a neurovascular disease, such as, for example, early brain injury (EBI) after subarachnoid hemorrhage (SAH), cerebral ischemia, stroke, hypoxic-ischemic brain injury, perinatal arterial ischemic stroke, or neovascular Age-related macular degeneration (nvAMD);
a substance abuse disorder, such as, for example, substance dependence, substance abuse and the sequalae of substance abuse dependence, substance-induced psychological disorder, substance withdrawal and substance-induced dementia or amnestic disorder;
a neuronal reaction to viral infection, Trypanosoma infection, a neurological deficit associated with AIDS, obesity, temporomandibular joint dysfunction, aphasia, Bell's palsy, encephalitis, a kidney disease or renal dysfunction, phaeochromocytoma, a metabolic syndrome, cancer, eczema, thrombocytopenia; hypoplasia; disseminated intravascular coagulation (DIC); myelodysplasia; immune thrombocytopenia purpura (ITP), HIV induced ITP, a neuro-oncological disease or disorder, neuro-immunological disease or disorder, multiple endocrine neoplasia type 2, von Hippel-Lindau disease (VHL), type I neurofibromatosis, Scleroderma, an epidermal and stromal wound healing disorder and/or a scarring disorder; or
a disease or disorder associated with aging and/or senescence.

According to one embodiment, the neurological disorder is epilepsy, such as, for example, Dravet syndrome, benign Rolandic epilepsy, frontal lobe epilepsy, infantile spasms, juvenile myoclonic epilepsy (JME), juvenile absence epilepsy, childhood absence epilepsy (e.g. pyknolepsy), febrile seizures, progressive myoclonus epilepsy of Lafora, Lennox-Gastaut syndrome, Landau-Kleffner syndrome, Generalized Epilepsy with Febrile Seizures (GEFS+), Severe Myoclonic, Epilepsy of Infancy (SMEI), Benign Neonatal Familial Convulsions (BFNC), West Syndrome, Ohtahara Syndrome, early myoclonic encephalopathies, migrating partial epilepsy, infantile epileptic encephalopathies, Tuberous Sclerosis Complex (TSC), focal cortical dysplasia, Type I Lissencephaly, Miller-Dieker Syndrome, Angelman's syndrome, Fragile X syndrome, epilepsy in autism spectrum disorders, epilepsy in subcortical band heterotopia, epilepsy in Walker-Warburg syndrome, epilepsy in Alzheimer's disease, post-traumatic epilepsy, progressive myoclonus epilepsies, reflex epilepsy, Rasmussen's syndrome, temporal lobe epilepsy, limbic epilepsy, status epilepticus, abdominal epilepsy, massive bilateral myoclonus, catamenial epilepsy, Jacksonian seizure disorder, Unverricht-Lundborg disease, or photosensitive epilepsy.

According to one embodiment, the composition or the medicament according to the invention, as described herein, is to be administered to a subject, and may be formulated using methods well-known in the art. Non-limiting examples of forms adapted for administration include solutions (such as, for example, sterile aqueous solutions), gels, dispersions, emulsions, suspensions and solid forms suitable for using to prepare solutions or suspensions upon the addition of a liquid prior to use (such as, for example, powder or liposomal forms).

The composition or the medicament according to the invention, as described herein, may be administered using administration route well-known in the art such as, for example, parenterally, orally, by inhalation, spray, rectally, nasally, or via an implanted reservoir.

It will be however understood that the total daily usage of the compound, the composition or the medicament will be decided by the attending physician within the scope of sound medical judgment. The specific therapeutically effective dose level for any particular patient will depend upon a variety of factors including the disease being treated and the severity of the disease; activity of the compound employed; the age, body weight, general health, sex and diet of the subject; the time of administration, route of administration, and rate of excretion of the specific therapeutic agent employed; the duration of the treatment; drugs used in combination or coincidental with the specific therapeutic agent employed; and like factors well known in the medical arts. For example, it is well within the skill of the art to start doses of the compound at levels lower than those required to achieve the desired therapeutic effect and to gradually increase the dosage until the desired effect is achieved. The total dose required for each treatment may be administered by multiple doses or in a single dose.

In one embodiment, the dosage of the compound is about 0.01 to 500 mg per kg patient body weight per day, which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be about 0.05 to 0.5, about 0.5 to 5 or about 5 to 50 mg/kg per day. For oral administration, the compositions are preferably provided in the form of tablets containing from about 1.0 to 1000 milligrams of the active ingredient, particularly about 1.0, about 5.0, about 10.0, about 15.0, about 20.0, about 25.0, about 50.0, about 75.0, about 100.0, about 150.0, about 200.0, about 250.0, about 300.0, about 400.0, about 500.0, about 600.0, about 750.0, about 800.0, about 900.0, and about 1000.0 milligrams of the active ingredient for the symptomatic adjustment of the dosage to the patient to be treated. The compounds may be administered on a regimen of 1 to 4 times per day, preferably once or twice per day. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

According to a first embodiment, the composition or the medicament according to the invention, as described herein, is to be administered as sole therapeutic agent. According to a second embodiment, the composition or the medicament according to the invention, as described herein, is to be administered before at least another therapeutic agent, concomitantly with at least another therapeutic agent, and/or after at least another therapeutic agent. In one embodiment, the other therapeutic agent is suitable for treating a neurological disorder.

Another object of the present invention is a method of promoting neuronal cell's survival and/or neuronal cell's functions. Another object of the present invention is a compound or a composition according to the invention, as described herein, for use in promoting neuronal cell's survival and/or neuronal cell's functions. According to one embodiment, the method or the use comprises a step of contacting a neuronal cell with a therapeutically effective amount of a compound, a composition or a medicament according to the invention, as described herein. The method or the use may be in vitro, ex vivo or in vivo.

Another object of the present invention is a method of rescuing neuronal cell's functions after the neuronal cell has been subjected to insults, events, or conditions detrimental to neuronal cell's functions. Another object of the present invention is a compound or a composition according to the invention, as described herein, for use in rescuing neuronal cell's functions after the neuronal cell has been subjected to insults, events, or conditions detrimental to neuronal cell's functions. Such insults, events, or conditions include, without limitation, neuronal stress, for instance, caused by hypoxia or ischemia; traumatic injuries; and exposure to toxic molecules, for instance, to abnormal misfolded proteins, protein aggregates, excitotoxins, reactive oxygen species, endoplasmic reticulum stressors, mitochondrial stressors, Golgi apparatus antagonists and the like. According to one embodiment, the method or the use comprises a step of contacting a neuronal cell with a therapeutically effective amount of a compound, a composition or a medicament according to the invention, as described herein. The method or the use may be in vitro, ex vivo or in vivo.

Another object of the present invention is a method of binding or modulating GFRα1 using a compound or a composition according to the invention, as described herein. Another object of the present invention is a compound or a composition according to the invention, as described herein, for binding or modulating GFRα1. According to one preferred embodiment, the compound or a composition is for activating GFRα1. In one embodiment, GFRα1 is human GFRα1, preferably with SEQ ID NO: 1.

Another object of the present invention is a method of activating the GFRα1/RET signaling pathway using a compound or a composition according to the invention, as described herein. Another object of the present invention is a compound or a composition according to the invention, as described herein, for activating the GFRα1/RET signaling pathway.

Another object of the present invention is a method of detecting GFRα1 in a sample, using a compound or a composition according to the invention, as described herein. Another object of the present invention is a compound or a composition according to the invention, as described herein, for detecting GFRα1 in a sample. In one embodiment, GFRα1 is human GFRα1, preferably with SEQ ID NO: 1. In one embodiment, the compound according to the invention may be fused to a detectable label, such as, e.g., a fluorophore or any other moiety that can re-emit light upon light excitation, a radiolabel, a contrast agent and the like.

### Manufacturing Process

Synthesis of the compound

The compound according to the invention, as described herein, may be manufactured by means of synthetic methods well-known in the art.

Another object of the present invention is a process for manufacturing a compound of the invention, as described herein. According to one embodiment, the process is a Buchwald-Hartwig amination.

According to one embodiment, the process comprises a step of reaction of:
a compound of formula (II)

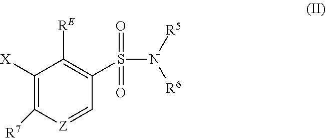

wherein Z, R⁵, R⁶, R⁷ and R^{E} are as defined under formula (I) herein and X represents halide or —CF₃SO₃,
with a compound of formula (III)

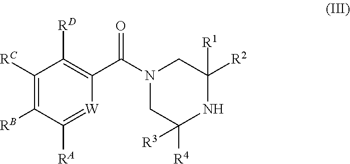

wherein W, R^{A}-R^{D} and R¹-R⁴ are as defined under formula (I) herein, in presence of a base and a metal catalyst; thereby obtaining the compound of the invention.

In one embodiment, the base is cesium carbonate (Cs₂CO₃), sodium carbonate (Na₂CO₃) or potassium carbonate (K₂CO₃). In one particular embodiment, the base is cesium carbonate (Cs₂CO₃). In one embodiment, the base is sodium tert-butanoate (t-BuONa), potassium tert-butanoate (t-BuOK) or potassium phosphate. In one particular embodiment, the base is sodium tert-butanoate (t-BuONa).

In one embodiment, the catalyst is a palladium catalyst. In one particular embodiment, the catalyst is a Pd (Oac) 2 and rac-BINAP (2,2′-bis(diphenylphosphino)-1,1′-binaphtyle) system. In one particular embodiment, the catalyst is Xphos-Pd-G3.

In one embodiment, X represents halide. In one particular embodiment, X represents Br.

In one embodiment, the reaction is carried out in a solvent. In one particular embodiment, the solvent is toluene. In one embodiment, the reaction is carried out at reflux.

According to another embodiment, the process comprises:
(a′-1) a step of reaction of
a compound of formula (II)

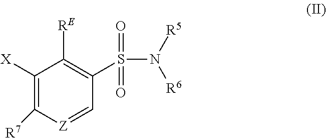

wherein Z, R⁵, R⁶, R⁷ and R^{E} are as defined under formula (I) herein and X represents halide or —CF₃SO₃,
with a mono-protected piperazine (i.e., a piperazine wherein only one of the NH groups is protected by means of a protecting group RP) of the following formula (MPP),

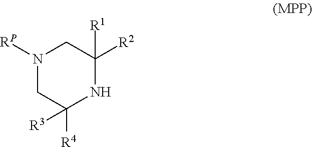

wherein R¹-R⁴ are as defined under formula (I) herein;
in presence of a base and a metal catalyst;
thereby obtaining a compound of formula (IV)

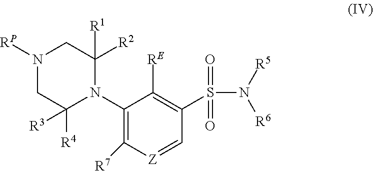

wherein Z, R¹-R⁴, R⁵, R⁶, R⁷ and R^{E} are as defined under formula (I) herein and RP is the protecting group; then (a′-2) a step of deprotection of the compound of formula (IV);
thereby obtaining a compound of formula (V)

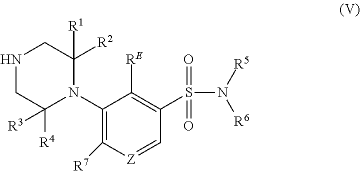

wherein Z, R¹-R⁴, R⁵, R⁶, R⁷ and R^{E} are as defined under formula (I) herein; and
(a′-3) a step of reaction of the compound of formula (V) with a compound of formula (VI)

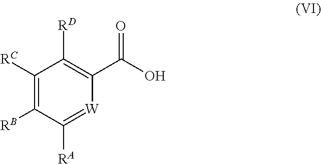

wherein W and R^{A}-R^{D} are as defined under formula (I) herein,
in presence of a peptide coupling agent and a base;
thereby obtaining the compound of the invention.

In one embodiment, the base and/or the catalyst at step (a′-1) are as described hereinabove under step (a-1). In one embodiment, X represents halide. In one particular embodiment, X represents Br.

The protecting group may be any protecting group known in the art such as, for example, tert-butyloxycarbonyl (Boc). The protective group may be removed at step (a′-2) by any method known in the art appropriate to the nature of the protective group, such as, for example, addition of a strong Bronsted acid (e.g., hydrochloric acid [HCl]).

The peptide coupling agent at step (a′-3) may be any peptide coupling agent known in the art such as, for example, 2-(1H-benzotriazole-1-yl)-1,1,3,3-tetramethylaminium tetrafluoroborate (TBTU).

The base at step (a′-3) may be any base known in the art such as, for example, an amine base. According to one embodiment, the amine is triethylamine (Et₃N) or diisopropylethylamine (iPr₂Net).

In one embodiment, the reaction at step (a′-3) is carried out in a solvent. In one particular embodiment, the solvent is dimethylformamide (DMF) and/or tetrahydrofuran (THF). In one particular embodiment, the solvent is dichloroethane (DCE) and/or acetonitrile (MeCN). In one embodiment, the reaction is carried out at room temperature (RT).

According to another embodiment, the process comprises:
(a″-1) a step of reaction of:
a compound of formula (III)

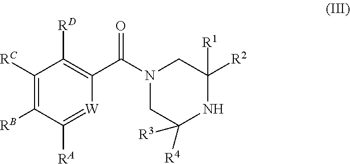

wherein W, R¹-R⁴, and R^{A}-R^{D} are as defined under formula (I) herein, with a compound of formula (VII)

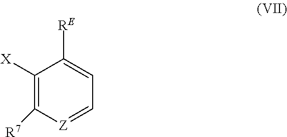

wherein Z, R⁷ and R^{E} are as defined under formula (I) herein, except that Z does not represent N, and X represents halide or —CF₃SO₃,
in presence of a base and a metal catalyst;
thereby obtaining a compound of formula (VIII)

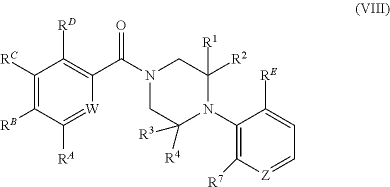

wherein W, R^{A}-R^{D}, R¹-R⁴, R⁷ and R^{E} are as defined under formula (I) herein and Z is as defined under formula (VII) herein;
(a″-2) a step of reaction of the compound of formula (VIII) with chlorosulfonic acid (HSO₃Cl);
thereby obtaining a compound of formula (IX);

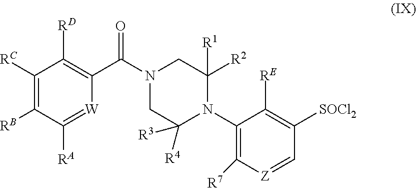

wherein W, R^{A}-R^{D}, R¹-R⁴, R⁷ and R^{E} are as defined under formula (I) herein and Z is as defined under formula (VII) herein;
(a″-3) a step of reaction of the compound of formula (IX) with a primary or secondary amine of formula (X)

NHR⁵R⁶ (X)
wherein R⁵ and R⁶ are as defined under formula (I) herein, in presence of a base;
thereby obtaining the compound of the invention.

According to an alternative embodiment, the process comprises a step (a″-1) of reaction of a compound of formula (III) with a compound of formula (VII) as described hereinabove, thereby obtaining a compound of formula (VIII); but no step (a″-2) of reaction of the compound of formula (VIII) with chlorosulfonic acid (HSO₃Cl); and Z may represent N.

In one embodiment, the base and/or the catalyst at step (a″-1) are as described hereinabove under step (a-1). In one embodiment, X represents halide. In one particular embodiment, X represents Br.

The base at step (a″-3) may be any base known in the art such as, for example, an amine base. According to one embodiment, the amine is triethylamine (Et₃N) or diisopropylethylamine (iPr₂Net).

In one embodiment, the reaction at step (a″-3) is carried out in a solvent. In one particular embodiment, the solvent is dichloromethane (DCM).

In one embodiment, the process further comprises a work-up step (b). In one embodiment, the work-up step (b) comprises a step of extraction by a solvent. In one particular embodiment, the solvent is ethyl acetate (EtOAc). In one particular embodiment, the solvent is water or a 1N HCl solution. In one particular embodiment, the solvent is dichloromethane (DCM). In one embodiment, the work-up step (b) comprises a step of filtration. In one particular embodiment, the filtration is over Celite® In one embodiment, the work-up step (b) comprises a step of concentration under reduced pressure.

In one embodiment, the process further comprises a purificationstep (c). In one embodiment, the purification step (c) comprises a purification by chromatography. In one particular embodiment, the chromatography is flash chromatography (FC) (e.g., cHex/EtOAc gradient), preparative thin-layer chromatography (PTLC) or semi-preparative high-performance liquid chromatography (HPLC).

### Synthetic Intermediates

Another object of the present invention is a compound of formula (II)

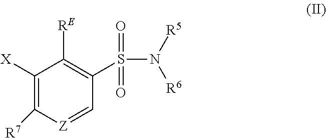

wherein Z, R⁵, R⁶, R⁷ and R^{E} are as defined under formula (I) herein and X represents halide or —CF₃SO₃.

Another object of the present invention is a compound of formula (III)

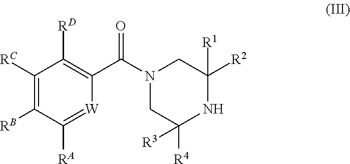

wherein W, R^{A}-R^{D} and R¹-R⁴ are as defined under formula (I) herein.

Another object of the present invention is a compound of formula (IV)

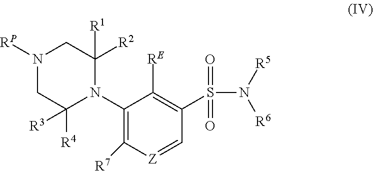

wherein Z, R¹-R⁴, R⁵, R′, R⁷ and R^{E} are as defined under formula (I) herein and RP is a protecting group (e.g., Boc).

Another object of the present invention is a compound of formula (V)

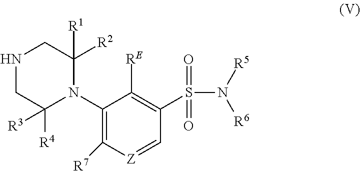

wherein Z, R¹-R⁴, R⁵, R′, R⁷ and R^{E} are as defined under formula (I) herein.

Another object of the present invention is a compound of formula (VII)

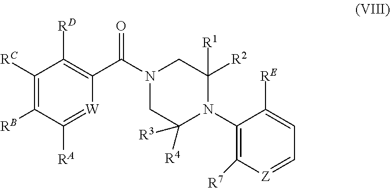

wherein W, Z, R^{A}-R^{D}, R¹-R⁴, R⁷ and R^{E} are as defined under formula (I) herein.

According to one preferred embodiment, in formula (VIII) hereinabove, Z does not represent N.

Another object of the present invention is a compound of formula (IX)

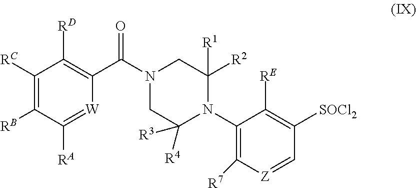

wherein W, Z, R^{A}-R^{D}, R¹-R⁴, R⁷ and R^{E} are as defined under formula (I) herein.

According to one preferred embodiment, in formula (IX) hereinabove, Z does not represent N.

### EXAMPLES

The present invention is further illustrated by the following examples.

### Example 1: Synthesis of the Compounds

### General Material and Methods

### Abbreviations

### List of Abbreviations:

Ac: acetyl
Ar: argon
BINAP: (2,2′-bis(diphenylphosphino)-1,1′-binaphthyl)
t-Bu: tert-butyl
cHex: cyclohexane
dba: dibenzylideneacetone
DCM: dichloromethane
DCE: dichloroethane
DMF: dimethylformamide
Et: ethyl
FC: flash chromatography
JohnPhos: 2-(di-tert-butylphosphino) biphenyl
MTBE: tert-butyl methyl ether
PTLC: preparative thin-layer chromatography
RT: room temperature
2-(1H-benzotriazole-1-yl)-1,1,3,3-tetramethylaminium TBTU:
tetrafluoroborate
TPTU: O-(2-oxo-1 (2H) pyridyl)-N,N,N′,N′-tetramethyluronium
tetrafluoroborate
TFA: trifluoroacetic acid
THF: tetrahydrofuran
XPhos: dicyclohexyl [2′,4′,6′-tris (propan-2-yl) [1,1′-biphenyl]-2-yl]phosphane
XantPhos: 4,5-bis(diphenylphosphino)-9,9-dimethylxanthene

### Analytical Methods

¹H NMR spectra (400 MHZ) and ¹⁹F NMR spectra (376 MHz) were recorded with a Bruker ULTRASHIELD 400 spectrometer. Processing and analyses of the spectra were performed with MestReNova. Data appear in the following order: chemical shifts in ppm which were referenced to the internal solvent signal, multiplicity, coupling constant J in Hertz and number of protons.

Reversed-phase HPLC/MS analyses were carried out with a Waters Alliance 2795 HPLC equipped with an autosampler, an inline membrane degasser, a column oven 10 (T°=45° C.), a UV detector, and a ZQ quadrupole mass detector working in ionization electrospray mode. Analyzed compounds (0.1 to 0.3 mg) were solubilized in a minimum amount of DMSO completed with acetonitrile (total volume: 1 mL). Standard analytical parameters: flow rate: 1 mL/min, Vinj.: 5 μL. Acidic conditions: Waters XSelect CSH C₁₈ column (3.5 μm, 2.1× 15 50 mm). Gradient: (H₂O+0.04% v/v HCO₂H (10 mM))/ACN from 95/5 to 0/100 in 2.5 min. Alkaline conditions: Waters Xbridge C₁₈ column (3.5 μm, 2.1×50 mm). Gradient: (H₂O+0.06% v/v NH₃ (aq.) (10 mM))/ACN from 95/5 to 0/100 in 2.5 min.

### General Synthetic Methods

### General Protocol 1 (GP-1): Peptide Coupling Using TBTU

To a solution of the required carboxylic acid in DMF at RT, was added TBTU. The mixture was stirred for 15 min at RT and a solution of the required piperazine and

Et₃N in THF was added dropwise. The mixture was stirred at RT for the required time. The volatiles were removed under reduced pressure and the residue was partitioned between 1N HCl and EtOAc. The layers were separated and the aqueous phase was extracted with EtOAc. The combined organic extracts were washed (IN HCl, aq. sat. NaHCO₃, brine), dried (Na₂SO₄), filtered and concentrated under reduced pressure. The residue was purified by FC (cHex/EtOAc gradient) or PTLC to afford the required product.

### General Protocol 2 (GP-2): Peptide Coupling Using TPTU (Small Scale)

To a solution of the required carboxylic acid in DCE/MeCN (1/1) at RT, were added iPr₂NEt and TPTU. After stirring for 5 min at RT, the required piperazine was added and the mixture was stirred at RT for the required time. 2 drops of ethylene diamine were added and the reaction mixture was concentrated under reduced pressure. The residue was purified by FC (cHex/EtOAc gradient) or PTLC to afford the required product.

### General Protocol 3 (GP-3): Chlorosulfonylation

To chlorosulfonic acid at 0° C., was added dropwise a solution of the required arene in DCM. The mixture was allowed to warm-up to the required temperature and allowed to stir at required temperature for the required time. The reaction mixture was poured dropwise on crushed ice while stirring (very exothermic quench). At the end of the addition, the residual ice was allowed to melt, DCM was added and the layers were separated. The aqueous phase was extracted with DCM and the combined organic extracts were dried (Na₂SO₄), filtered and concentrated under reduced pressure to afford the desired sulfonyl chloride.

### General Protocol 4 (GP-4): Sulfonamide Formation

To a solution of the required amine and Et₃N or iPr₂NEt in DCM, was added a solution of the required sulfonyl chloride in DCM at RT. The reaction mixture was stirred at RT for the required time. The reaction mixture was partitioned between EtOAc and aq. sat. NH₄Cl. The layers were separated and the aqueous phase was extracted with EtOAc. The combined organic extracts were washed (aq. sat. NH₄Cl, brine), dried (Na₂SO₄), filtered and concentrated under reduced pressure. The residue was purified by FC (cHex/EtOAc gradient) or PTLC to afford the required product.

### General Protocol 5 (GP-5): Buchwald Coupling Using Pd (OAc) 2 or Pd: (Dba) 3/Rac-BINAP

A microwave reaction vial was charged with the required arylbromide, the required piperazine, Cs₂cO₃, Pd (OAc) 2 or Pd2 (dba) 3 and rac-BINAP. The vial was flushed with argon and degassed toluene was added. The vial was sealed and the reaction was stirred at reflux in a preheated heating-block for the required time. After cooling down to RT, EtOAc was added and the suspension as filtered over Celite® (EtOAc rinses). The filtrate was concentrated under reduced pressure. The residue was purified by FC (cHex/EtOAc gradient) or PTLC to afford the required product.

### General Protocol 6 (GP-6): Buchwald Coupling Using XantPhos-Pd-G3

A microwave reaction vial was charged with the required arylbromide, the required piperazine, t-BuONa and XantPhos-Pd-G3. The vial was flushed with argon and degassed toluene was added. The vial was sealed and the reaction was stirred at reflux in a preheated heating-block for the required time. After cooling down to RT, EtOAc was added and the suspension as filtered over Celite® (EtOAc rinses). The filtrate was concentrated under reduced pressure. The residue was purified by FC (cHex/EtOAc gradient) or PTLC to afford the required product.

### General Protocol 7 (GP-7): Buchwald Coupling Using XPhos-Pd-G3

A microwave reaction vial was charged with the required arylbromide, the required piperazine, t-BuoNa and XPhos-Pd-G3. The vial was flushed with argon and degassed toluene was added. The vial was sealed and the reaction was stirred at reflux in a preheated heating-block for the required time. After cooling down to RT, EtOAc was added and the suspension as filtered over Celite® (EtOAc rinses). The filtrate was concentrated under reduced pressure. The residue was purified by FC (cHex/EtOAc gradient) or PTLC to afford the required product.

### Synthesis of Intermediate Compounds

### Synthesis of Common Intermediate Piperazines

### tert-butyl 3-(2-chloro-4-fluoro-benzoyl)-3,8-diazabicyclo[3.2.1]octane-8-carboxylate (Boc-1-001)

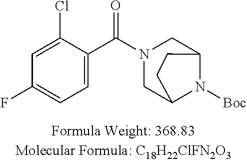 [SMILES: CC(C)(C)OC(=O)N1C2CCC1CN(C(=O)c1ccc(F)cc1Cl)C2]

To a solution of 2-chloro-4-fluorobenzoic acid (9.87 g, 56.5 mmol, 1.2 equiv.) in DMF (118 mL) at RT, was added TBTU (18.1 g, 56.5 mmol, 1.2 equiv.). The mixture was stirred 15 min at RT, and a solution of tert-butyl 3,8-diazabicyclo[3.2.1]octane-8-carboxylate (10.0 g, 47.1 mmol, 1 equiv.) and Et₃N (9.8 mL, 71 mmol, 1.5 equiv.) in THF (118 mL) was added dropwise. The mixture was stirred at RT for 16 h. The reaction mixture was concentrated under reduced pressure. The reaction mixture was partitioned between aq. sat. NH₄Cl and EtOAc. The layers were separated and the aqueous phase was extracted with EtOAc (2*). The combined organic extracts were washed (aq. sat. NH₄Cl, aq. sat. NaHCO₃, brine), dried (Na₂SO₄), filtered and concentrated under reduced pressure to afford 21.3 g (76% pure, 93%) of tert-butyl 3-(2-chloro-4-fluoro-benzoyl)-3,8-diazabicyclo[3.2.1]octane-8-carboxylate (Boc-I-001) as an orange oil. ¹H NMR (400 MHZ, Chloroform-d, 2 sets of rotamers) δ 7.33 (dd, J=8.5, 5.9 Hz, 0.5H), 7.23-7.09 (m, 1.5H), 7.09-6.97 (m, 1H), 4.54-4.40 (m, 1H), 4.33 (s, 1H), 4.23-4.01 (m, 1H), 3.56-3.17 (m, 1H), 3.17-2.93 (m, 2H), 2.11-1.70 (m, 4H), 1.47 (s, 9H). MS (ESI+): [M+H]⁺ 369.1/371.1.

### (2-chloro-4-fluoro-phenyl)-(3,8-diazabicyclo[3.2.1]octan-3-yl) methanone (1-001)

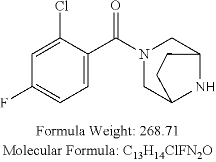 [SMILES: O=C(c1ccc(F)cc1Cl)N1CC2CCC(C1)N2]

To a solution of crude tert-butyl 3-(2-chloro-4-fluoro-benzoyl)-3,8-diazabicyclo[3.2.1]octane-8-carboxylate Boc-I-001 (21.3 g, 76% pure, 43.9 mmol, 1 equiv.) in dioxane (55 mL) at RT, was added HCl (4M solution in dioxane, 55 mL, 220 mmol, 5 equiv.). The mixture was stirred at RT for 60 h. The reaction mixture was concentrated under reduced pressure and the residue was partitioned between water and DCM. K₂CO₃ (s) was added portionwise until pH >11. The layers were separated and the aqueous phase was extracted with DCM (2*). The combined organic extracts were dried (Na₂SO₄), filtered and concentrated under reduced pressure. The residue was purified by FC (220 g column, dry load, DCM/MeOH (7N NH₃)=99/1 to 95/5) to afford 10.3 g (88%) of (2-chloro-4-fluoro-phenyl)-(3,8-diazabicyclo[3.2.1]octan-3-yl) methanone I-001 as a white solid. ¹H NMR (400 MHZ, Chloroform-d, 2 sets of rotamers) δ 7.32 (dd, J=8.5, 5.9 Hz, 0.5H), 7.20-7.15 (m, 0.5H), 7.15-7.10 (m, 1H), 7.09-6.98 (m, 1H), 4.47-4.37 (m, 1H), 3.69-3.56 (m, 1H), 3.44-3.34 (m, 1.5H), 3.22 (d, J=11.9 Hz, 0.5H), 3.10-2.98 (m, 2H), 1.95-1.48 (m, 4H). MS (ESI+): [M+H]⁺ 269.1/271.0.

### tert-butyl 3-[4-fluoro-2-(trifluoromethyl)benzoyl]-3,8-diazabicyclo[3.2.1]octane-8-carboxylate (Boc-1-002)

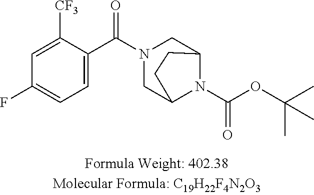 [SMILES: CC(C)(C)OC(=O)N1C2CCC1CN(C(=O)c1ccc(F)cc1C(F)(F)F)C2]

To a solution of commercial 4-fluoro-2-(trifluoromethyl)benzoic acid (784 mg, 3.77 mmol, 2 equiv. In DCE/ACN (1:1, 9 mL) at RT, was added iPr₂NEt (656 μL, 3.77 mmol, 2 equiv.) and TPTU (1.12 g, 3.77 mmol, 2 equiv.). The mixture was stirred for 15 min at RT and tert-butyl (1R,5S)-3,8-diazabicyclo[3.2.1]octane-8-carboxylate (400 mg, 1.88 mmol, 1 equiv.) was added. The mixture was stirred at RT for 16 h. The volatiles were removed under reduced pressure and the residue was partitioned between 1N HCl and EtOAc. The layers were separated and the aqueous phase was extracted with EtOAc. The combined organic extracts were washed (IN HCl, aq. sat. NaHCO₃, brine), dried (Na₂SO₄), filtered and concentrated under reduced pressure. The residue was purified by FC (cHex/EtOAc 95/5 to 40/60) affording 724 mg (95%) of tert-butyl 3-[4-fluoro-2-(trifluoromethyl)benzoyl]-3,8-diazabicyclo[3.2.1]octane-8-carboxylate (Boc-I-002) as a colorless oil. ¹H NMR (400 MHZ, DMSO-d₆, multiple sets of rotamers) δ 7.83-7.70 (m, 1H), 7.69-7.55 (m, 2H), 4.31-4.14 (m, 2H), 4.08-3.95 (m, 1H), 3.37-2.82 (m, 3H), 1.95-1.47 (m, 4H), 1.42 (d, J=7.8 Hz, 9H). MS (ESI+): [M+H]⁺ 403.2.

### 3,8-diazabicyclo[3.2.1]octan-3-yl-[4-fluoro-2-(trifluoromethyl)phenyl]methanone (1-002)

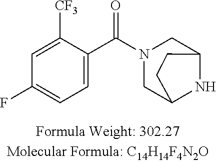 [SMILES: O=C(c1ccc(F)cc1C(F)(F)F)N1CC2CCC(C1)N2]

To a solution of tert-butyl 3-[4-fluoro-2-(trifluoromethyl)benzoyl]-3,8-diazabicyclo[3.2.1]octane-8-carboxylate Boc-I-002 (724 mg, 1.80 mmol, 1 equiv.) was treated with HCl (4M solution in dioxane, 4.5 mL, 18 mmol, 10 equiv.). The mixture was stirred at RT for 16 h. The reaction mixture was concentrated under reduced pressure and the residue was partitioned between water and DCM. K₂CO₃ (s) was added portionwise until pH >11. The layers were separated and the aqueous phase was extracted with DCM (2*). The combined organic extracts were dried (Na₂SO₄), filtered and concentrated under reduced pressure, affording 607 mg (quantitative yield) of I-002 as a colorless oil. 1H NMR (400 MHZ, DMSO-d₆. 2 sets of rotamers) δ 7.75 (ddd, J=9.3, 4.4, 2.6 Hz, 1H), 7.69-7.49 (m, 2H), 4.14 (ddd, J=19.8, 12.4, 2.3 Hz, 1H), 3.62-3.58 (m, 1H), 3.57 (s, 1H), 3.39-3.33 (m, 1H), 3.33-3.26 (m, 0.5H), 3.12-2.96 (m, 1H), 2.95-2.92 (m, 1.5H), 1.82-1.57 (m, 3H), 1.57-1.39 (m, 1H). MS (ESI+): [M+H]⁺ 303.0.

### tert-butyl 4-(2-chloro-4-fluoro-benzoyl)-2-methyl-piperazine-1-carboxylate (Boc-1-003)

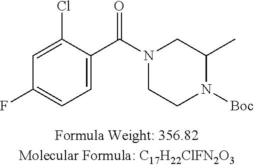 [SMILES: CC1CN(C(=O)c2ccc(F)cc2Cl)CCN1C(=O)OC(C)(C)C]

To a solution of 2-chloro-4-fluorobenzoic acid (10.5 g, 59.9 mmol, 1.2 equiv.) in THF (120 mL) at RT, was added TBTU (19.2 g, 59.9 mmol, 1 equiv.). The mixture was stirred 15 min at RT, and a solution of tert-butyl 2-methylpiperazine-1-carboxylate (10.0 g, 49.9 mmol, 1 equiv.) and Et₃N (6.5 mL, 37 mmol, 0.75 equiv.) in THF (120 mL) was added dropwise. The mixture was stirred at RT for 60 h. The reaction mixture was concentrated under reduced pressure. The reaction mixture was partitioned Between aq. sat. NH₄Cl and EtOAc. The layers were separated and the aqueous phase was extracted with EtOAc (2*). The combined organic extracts were Washed (aq. sat. NH₄Cl, aq. sat. NaHCO₃, brine), dried (Na₂SO₄), filtered and concentrated under reduced pressure. The residue was purified by FC (220 g column, dry load, cHex/EtOAc=90/10 to 50/50) to afford 16.8 g (94%) of tert-butyl 4-(2-chloro-4-fluoro-benzoyl)-2-methyl-piperazine-1-carboxylate (Boc-I-003) as a white solid. ¹H NMR (400 MHZ, Chloroform-d, multiple sets of rotamers) δ 7.36-7.21 (m, 1H), 7.20-7.09 (m, 1H), 7.08-6.97 (m, 1H), 4.67-4.13 (m, 2H), 3.99-3.74 (m, 1H), 3.41-3.23 (m, 1H), 3.23-2.83 (m, 3H), 1.44 (s, 9H), 1.29-0.97 (m, 3H). MS (ESI+): [M+H]⁺ 357.0/359.0.

### (2-chloro-4-fluoro-phenyl)-(3-methylpiperazin-1-yl) methanone (1-003)

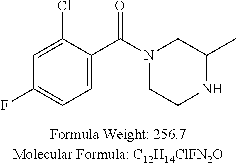 [SMILES: CC1CN(C(=O)c2ccc(F)cc2Cl)CCN1]

To a solution of crude tert-butyl 4-(2-chloro-4-fluoro-benzoyl)-2-methyl-piperazine-1-carboxylate Boc-I-003 (16.8 g, 47.1 mmol, 1 equiv.) in DCM (235 mL) at 0° C., was added TFA (72 mL, 941 mmol, 20 equiv.). The mixture was stirred at RT for 2 h. The reaction mixture was concentrated under reduced pressure and the residue was partitioned between water and DCM at 0° C. K₂CO₃ (s) was added portionwise until pH >11. The layers were separated and the aqueous phase was extracted with DCM (2*). The combined organic extracts were dried (Na₂SO₄), filtered and concentrated under reduced pressure. The residue was purified by FC (80 g column, dry load, DCM/MeOH (7N NH₃)=99/1 to 90/10) to afford 9.8 g (81%) of (2-chloro-4-fluoro-phenyl)-(3-methylpiperazin-1-yl) methanone I-003 as a white solid. ¹H NMR (400 MHZ, Chloroform-d, 2 sets of rotamers) δ 7.32 (ddd, J=8.6, 5.9, 1.3 Hz, 0.5H), 7.25 (ddd, J=8.5, 5.9, 1.4 Hz, 0.5H), 7.21-7.13 (m, 1H), 7.09-7.01 (m, 1H), 4.69-4.56 (m, 1H), 3.31-2.75 (m, 5H), 2.51 (m, 1H), 1.15 (dd, J=6.3, 2.4 Hz, 1.5H), 0.98 (dd, J=6.3, 4.5 Hz, 1.5H). MS (ESI+): [M+H]⁺ 257.1/259.1.

(2-chloro-4-fluoro-phenyl)-[(3S)-3-methylpiperazin-1-yl]methanone ((S)-1-003) & (2-chloro-4-fluoro-phenyl)-[(3R)-3-methylpiperazin-1-yl]methanone ((R)-1-003). (S)-I-003 and (R)-I-003 were obtained using the same synthetic sequence than the one used for I-003 starting respectively, from tert-butyl (2S)-2-methylpiperazine-1-carboxylate and tert-butyl (2R)-2-methylpiperazine-1-carboxylate. ¹H NMR (400 MHZ, Chloroform-d, 2 sets of rotamers) δ 7.32 (ddd, J=8.6, 5.9, 1.3 Hz, 0.5H), 7.25 (ddd, J=8.5, 5.9, 1.4 Hz, 0.5H), 7.21-7.13 (m, 1H), 7.09-7.01 (m, 1H), 4.69-4.56 (m, 1H), 3.31-2.75 (m, 5H), 2.51 (m, 1H), 1.15 (dd, J=6.3, 2.4 Hz, 1.5H), 0.98 (dd, J=6.3, 4.5 Hz, 1.5H). MS (ESI+): [M+H]⁺ 257.1/259.1.

### tert-butyl 3-(2-chloro-4,5-difluoro-benzoyl)-3,8-diazabicyclo[3.2.1]octane-8-carboxylate (Boc-1-033)

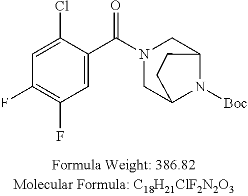 [SMILES: CC(C)(C)OC(=O)N1C2CCC1CN(C(=O)c1cc(F)c(F)cc1Cl)C2]

To a solution of 2-chloro-4,5-difluorobenzoic acid (2.72 g, 14.1 mmol, 1.2 equiv.) in DMF (29 mL) at RT, was added TBTU (4.54 g, 14.1 mmol, 1.2 equiv.). The mixture was stirred 15 min at RT, and a solution of tert-butyl 3,8-diazabicyclo[3.2.1]octane-8-carboxylate (2.50 g, 11.8 mmol, 1 equiv.) and Et₃N (2.5 mL, 17 mmol, 1.5 equiv.) in THF (29 mL) was added dropwise. The mixture was stirred at RT for 16 h. The reaction mixture was concentrated under reduced pressure. The reaction mixture was partitioned between aq. sat. NH₄Cl and EtOAc. The layers were separated and the aqueous phase was extracted with EtOAc (2*). The combined organic extracts were washed (aq. sat. NH₄Cl, aq. sat. NaHCO₃, brine), dried (Na₂SO₄), filtered and concentrated under reduced pressure to afford 6.76 g (88% pure) of tert-butyl 3-(2-chloro-4,5-difluoro-benzoyl)-3,8-diazabicyclo[3.2.1]octane-8-carboxylate (Boc-I-033) as an orange oil. ¹H NMR (400 MHZ, Chloroform-d, 2 sets of rotamers) δ 7.32-6.94 (m, 2H), 4.47-4.36 (m, 1H), 4.38-4.00 (m, 2H), 3.55-3.20 (m, 1H), 3.11-3.01 (m, 2H), 2.07-1.64 (m, 3.5H), 1.60-1.50 (m, 0.5H), 1.46 (s, 9H). MS (ESI+): [M+H]⁺ 387.1/389.1.

### (2-chloro-4,5-difluoro-phenyl)-(3,8-diazabicyclo[3.2.1]octan-3-yl) methanone (1-033)

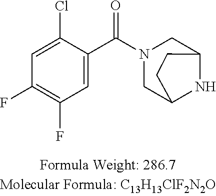 [SMILES: O=C(c1cc(F)c(F)cc1Cl)N1CC2CCC(C1)N2]

To a solution of crude tert-butyl 3-(2-chloro-4,5-difluoro-benzoyl)-3,8-diazabicyclo[3.2.1]octane-8-carboxylate Boc-I-033 (6.76 g, 88% pure, 15.3 mmol, 1 equiv.) in dioxane (77 mL) at RT, was added HCl (4M solution in dioxane, 19 mL, 77 mmol, 5 equiv.). The mixture was stirred at RT for 16 h. The reaction mixture was concentrated under reduced pressure and the residue was partitioned between water and DCM. K₂CO₃ (s) was added portionwise until pH >11. The layers were separated and the aqueous phase was extracted with DCM (2*). The combined organic extracts were dried (Na₂SO₄), filtered and concentrated under reduced pressure. The residue was purified by FC (24 g column, dry load, DCM/MeOH (7N NH₃)=99/1 to 95/5) to afford 3.15 g (70%) of (2-chloro-4,5-difluoro-phenyl)-(3,8-diazabicyclo[3.2.1]octan-3-yl) methanone I-033 as a white solid. ¹H NMR (400 MHz, Chloroform-d, 2 sets of rotamers) δ 7.32-7.22 (m, 1H), 7.21-6.96 (m, 1H), 4.45-4.30 (m, 1H), 3.62 (s, 1H), 3.44-3.20 (m, 2H), 3.10-2.98 (m, 2H), 1.94-1.71 (m, 3.5H), 1.62-1.53 (m, 0.5H). MS (ESI+): [M+H]⁺ 287.0/289.0.

### Synthesis of Required Amines

### tert-butyl 4-(3-pyrazol-1-ylphenyl) piperidine-1-carboxylate (Boc-1-034)

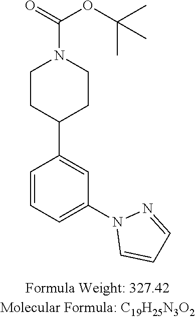 [SMILES: CC(C)(C)OC(=O)N1CCC(c2cccc(-n3cccn3)c2)CC1]

A MW vial was charged with tert-butyl 4-(3-bromophenyl) piperidine-1-carboxylate (100 mg, 294 μmol, 1 equiv.), pyrazole (40 mg, 0.59 mmol, 2 equiv.), Cs₂CO₃ (239 mg, 735 μmol, 2.5 equiv.), CuI (11 mg, 59 μmol, 0.2 equ′v.) and trans-N,N′-dimethylcyclohexane-1,2-diamine (0.012 mL, 0.074 mmol, 0.25 equiv.). The vial was purged and degassed NMP (1.5 mL) was added. The vial was sealed and the mixture was stirred at 150° C. for 18 h. After cooling down to RT, EtOAc was added and the suspension was filtered over Celite (EtOAc rinses). The filtrate was concentrated under reduced pressure. The residue was purified by FC (12 g column, dry load, cHex/EtOAc=95/5 to 50/50) to afford 49 mg (51%) of tert-butyl 4-(3-pyrazol-1-ylphenyl) piperidine-1-carboxylate Boc-I-034 as a colorless oil. ¹H NMR (400 MHZ, Chloroform-d) δ 7.85 (dd, J=2.5, 0.6 Hz, 1H), 7.65 (d, J=1.7 Hz, 1H), 7.53 (t, J=2.0 Hz, 1H), 7.42 (ddd, J=8.1, 2.2, 1.1 Hz, 1H), 7.31 (t, J=7.8 Hz, 1H), 7.12-7.00 (m, 1H), 6.39 (dd, J=2.5, 1.8 Hz, 1H), 4.19 (s, 2H), 2.83-2.48 (m, 3H), 1.80 (d, J=13.1 Hz, 2H), 1.61 (qd, J=12.6, 4.4 Hz, 2H), 1.42 (s, 9H). MS (ESI+): [M+H]⁺ 328.1.

### 4-(3-pyrazol-1-ylphenyl) piperidine; hydrochloride (1-034)

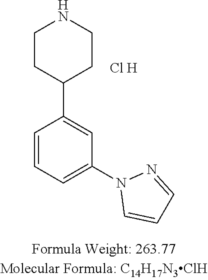

To a solution of crude tert-butyl 4-(3-pyrazol-1-ylphenyl) piperidine-1-carboxylate Boc-I-034 (49 mg, 0.15 mmol, 1 equiv.) in dioxane (71 mL) at RT, was added HCl (4M solution in dioxane, 0.37 mL, 1.5 mmol, 10 equiv.). The mixture was stirred at RT for 3 h. The reaction mixture was concentrated under reduced pressure to afford 344 mg (98%) 4-(3-pyrazol-1-ylphenyl) piperidine;hydrochloride I-034 as a white solid. ¹H NMR (400 MHZ, D₂₀) δ 8.14 (d, J=2.6 Hz, 1H), 7.78 (d, J=1.9 Hz, 1H), 7.61-7.41 (m, 3H), 7.29 (dt, J=7.2, 1.7 Hz, 1H), 6.56 (t, J=2.3 Hz, 1H), 3.59-3.44 (m, 2H), 3.23-3.07 (m, 2H), 2.98 (ddt, J=12.1, 7.3, 3.6 Hz, 1H), 2.21-2.03 (m, 2H), 2.01-1.80 (m, 2H).

### 2-(4-fluorophenyl)cyclopropanol (1-035)

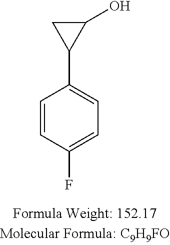 [SMILES: OC1CC1c1ccc(F)cc1]

A solution of 4-fluorophenylethyl magnesium bromide [prepared by slowly adding a solution of 1-(2-Bromoethyl)-4-fluorobenzene (3.0 g, 15 mmol, 3 equiv.) in dry THF (10 mL) to Mg (547 mg, 22.5 mmol, 4.5equiv.) in dry THF (5 mL) and refluxing the resulting suspension for 1 h only the supernatant was used] was added dropwise over 2 h on a solution of CITi (Oi-Pr) 3 (1.95 g, 7.49 mmol, 1.5 equiv.) and methyl formate (0.30 g, 5.0 mmol, 1 equiv.) in dry THF (35 mL) at RT. Ice-cold 10% H₂SO₄ was added dropwise, followed by Et₂O. The layers were separated and the aqueous phase was extracted with Et₂₀. The combined organic extracts were washed (sat. aq. NaHCO₃, brine), dried (Na₂SO₄), filtered and concentrated under reduced pressure. The residue was purified by FC (24 g column, dry load, cHex/EtOAc=96/4 to 60/40) to afford 231 mg (27%, 90% purity) of 2-(4-fluorophenyl)cyclopropanol I-035 as a colorless oil. ¹H NMR (400 MHZ, Chloroform-d) δ 7.03-6.92 (m, 4H), 3.63-3.56 (m, 1H), 2.29-2.08 (m, 2H), 1.28 (ddd, J=10.0, 6.1, 3.5 Hz, 1H), 1.02 (q, J=6.4 Hz, 1H).

### trans tert-butyl 4-[rac-(1R,2S)-2-(4-fluorophenyl)cyclopropyl]piperazine-1-carboxylate (Boc-1-036)

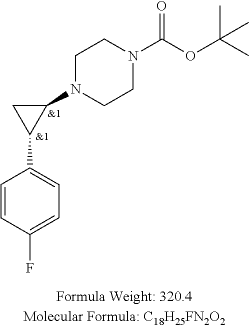 [SMILES: CC(C)(C)OC(=O)N1CCN([C@@H]2C[C@H]2c2ccc(F)cc2)CC1]

A MW vial was charged with ZnCl2 (372 mg, 2.73 mmol, 2 equiv.) Na₂CO₃ (290 mg, 2.73 mmol, 2 equiv.) and tert-butyl-1-piperazine carboxylate (254 mg, 1.37 mmol, 1 equiv.). The vial was purged with Ar and a solution of 2-(4-fluorophenyl)cyclopropanol I-035 (90% purity, 231 mg, 1.37 mmol, 1 equiv.). The vial was sealed and the mixture was stirred at 110° C. for 16 h. After cooling down to RT, the reaction mixture was partitioned between sat. aq. NaHCO₃ and EtOAc. The layers were separated and the aqueous phase was extracted with EtOAc. The combined organic extracts were washed (brine), dried (Na₂SO₄), filtered and concentrated under reduced pressure. The residue was purified by FC (24 g column, dry load, cHex/EtOAc=95/5 to 50/50) to afford 60 mg (14%) of trans tert-butyl 4-[rac-(1R,2S)-2-(4-fluorophenyl)cyclopropyl]piperazine-1-carboxylate Boc-I-036 as a colorless oil. ¹H NMR (400 MHZ, Chloroform-d) δ 7.02-6.98 (m, 2H), 6.96-6.91 (m, 2H), 3.49-3.31 (m, 4H), 2.58 (t, J=5.1 Hz, 4H), 1.98 (ddd, J=9.3, 5.8, 3.2 Hz, 1H), 1.80 (dt, J=7.3, 3.8 Hz, 1H), 1.46 (s, 9H), 1.10 (dt, J=9.5, 4.7 Hz, 1H), 0.91 (dt, J=6.9, 5.5 Hz, 1H). ¹⁹F NMR (376 MHz, Chloroform-d) 8-117.6. MS (ESI+): [M+H]⁺ 321.2.

### trans 1-[rac-(1R,2S)-2-(4-fluorophenyl)cyclopropyl]piperazine (1-036)

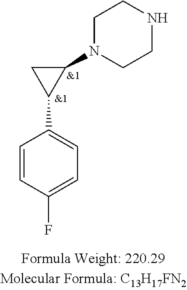 [SMILES: Fc1ccc([C@@H]2C[C@H]2N2CCNCC2)cc1]

To a solution of trans tert-butyl 4-[rac-(1R,2S)-2-(4-fluorophenyl)cyclopropyl]piperazine-1-carboxylate Boc-I-036 (60 mg, 0.19 mmol, 1 equiv.) in dioxane (0.9 mL) at RT, was added HCl (4M solution in dioxane, 0.94 mL, 3.7 mmol, 20 equiv.). The mixture was stirred at RT for 16 h. The reaction mixture was concentrated under reduced pressure and the residue was partitioned between water and DCM. K₂CO₃ (s) was added portionwise until pH >11. The layers were separated and the aqueous phase was extracted with DCM (2*). The combined organic extracts were dried (Na₂SO₄), filtered and concentrated under reduced pressure. The residue was purified by PTLC (DCM/MeOH (7N NH₃)=90/10) to afford 26 mg (63%) trans 1-[rac-(1R,2S)-2-(4-fluorophenyl)cyclopropyl]piperazine I-036 as a colorless oil. ¹H NMR (400 MHZ, Chloroform-d) δ 7.05-6.96 (m, 2H), 6.96-6.86 (m, 2H), 2.97-2.76 (m, 4H), 2.62 (s, 4H), 2.05 (s, 1H), 1.96 (ddd, J=9.3, 5.8, 3.2 Hz, 1H), 1.80 (dt, J=7.3, 3.8 Hz, 1H), 1.09 (dt, J=9.5, 4.7 Hz, 1H), 0.93-0.85 (m, 1H). ¹⁹F NMR (376 MHZ, Chloroform-d) 8-117.8. MS (ESI⁺): [M+H]⁺ 221.2.

Synthesis of intermediates required for final product 001

### 1-(2-methoxyphenyl)-2-methyl-piperazine (1-004)

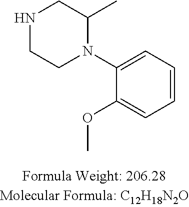

A microwave reaction vial was charged with 2-bromoanisole (471 μL, 3.74 mmol, 1.5 equiv.), tert-butyl 3-methylpiperazine-1-carboxylate (500 mg, 2.50 mmol, 1 equiv.), t-BuONa (360 mg, 3.74 mmol, 1.5 equiv.), Pd (OAc) 2 (56 mg, 0.25 mmol, 0.1 equiv.) and XPhos (143 mg, 300 μmol, 0.12 equiv.). The vial was flushed with argon and degassed toluene (12.5 mL) was added. The vial was sealed and the reaction was stirred at reflux in a preheated heating-block for 72 h. After cooling down to RT, EtOAc was added and the suspension was filtered over Celite® (EtOAc rinses). The filtrate was concentrated under reduced pressure. The residue was purified by FC (cHex/EtOAc gradient=95/5 to 50/50). The residue was directly treated with TFA (1.33 mL, 17.4 mmol, 20 equiv.). The mixture was stirred at RT for 5 h. The reaction mixture was concentrated under reduced pressure and the residue was partitioned between water and DCM. K₂CO₃ (s) was added dropwise until pH >11. The layers were separated and the aqueous phase was extracted with DCM (2*). The combined organic extracts were dried (Na₂SO₄), filtered and concentrated under reduced pressure. The residue was purified by FC (DCM/MeOH (7N NH₃)=99/1 to 95/5) affording 92 mg (18% over two steps) of I-004 as a yellow oil. ¹H NMR (400 MHZ, Chloroform-d, two sets of rotamers) δ 7.08-6.98 (m, 2H), 6.95-6.83 (m, 2H), 3.85 (s, 3H), 3.44 (pd, J=6.3, 3.0 Hz, 1H), 3.21 (ddd, J=11.5, 5.3, 3.1 Hz, 1H), 3.15-2.94 (m, 3H), 2.78-2.68 (m, 2H), 1.78 (s, 1H), 0.92 (s, 1.5H), 0.90 (s, 1.5H). MS (ESI⁺): [M+H]⁺ 207.4.

### (2-chloro-4-fluoro-phenyl)-[4-(2-methoxyphenyl)-3-methyl-piperazin-1-yl]methanone (1-005)

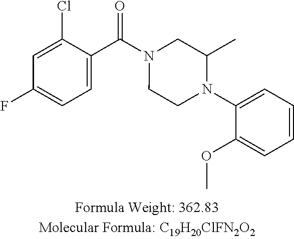 [SMILES: COc1ccccc1N1CCN(C(=O)c2ccc(F)cc2Cl)CC1C]

According to GP-1, 1-005 was obtained as a white solid in 68% yield using 2-chloro-4-fluorobenzoic acid (338 mg, 1.93 mmol, 1.5 equiv.) and TBTU (414 mg, 1.29 mmol, 1 equiv.) in DMF (3.3 mL), followed by piperazine I-004 (266 mg, 1.29 mmol, 1 equiv.) and Et₃N (270 μL, 1.93 mmol, 1.5 equiv.) in THF (3.3 mL) at RT for 16 h. Purification by FC (cHex/EtOAc=95/5 to 20/80). ¹H NMR (400 MHZ, Chloroform-d, multiple sets of rotamers) δ 7.36-7.29 (m, 1H), 7.21-7.14 (m, 1H), 7.11-7.01 (m, 2H), 7.01-6.84 (m, 3H), 3.85 (dd, J=5.7, 2.9 Hz, 3H), 3.76-3.20 (m, 5H), 3.19-3.04 (m, 1H), 3.01-2.71 (m, 1H), 1.04-0.96 (m, 1.5H), 0.82-0.79 (m, 1.5H). ¹⁹F NMR (376 MHz, Chloroform-d, multiple sets of rotamers) δ-109.59,-109.61,-109.63,-109.67. MS (ESI⁺): [M+H]⁺ 363.1.

### 3-[4-(2-chloro-4-fluoro-benzoyl)-2-methyl-piperazin-1-yl]-4-methoxy-benzenesulfonyl chloride (1-006)

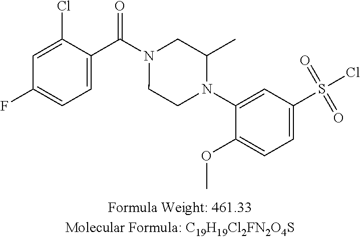 [SMILES: COc1ccc(S(=O)(=O)Cl)cc1N1CCN(C(=O)c2ccc(F)cc2Cl)CC1C]

According to GP-3, I-006 was obtained as an orange oil in 91% yield using I-005 (370 mg, 877 μmol, 1 equiv.) and HSO₃Cl (1.17 mL, 17.5 mmol, 20 equiv.) in DCM (4.4 mL) at RT for 1 h30. ¹H NMR (400 MHZ, Chloroform-d, multiple sets of rotamers) δ 7.83-7.72 (m, 1H), 7.51-7.45 (m, 1H), 7.37-7.28 (m, 1H), 7.24-7.15 (m, 1H), 7.13-7.04 (m, 1H), 7.04-6.96 (m, 1H), 4.27-2.81 (m, 10H), 1.10-0.78 (m, 3H). ¹⁹F NMR (376 MHz, Chloroform-d, multiple sets of rotamers) δ-109.13,-109.15,-109.21. MS (ESI⁺): [M+H]⁺ 461.0/463.0.

### Synthesis of Intermediates Required for Final Products 002 to 015 and 039 to 048

### (2-chloro-4-fluoro-phenyl)-[8-(2-methoxyphenyl)-3,8-diazabicyclo[3.2.1]octan-3-yl]methanone (1-007)

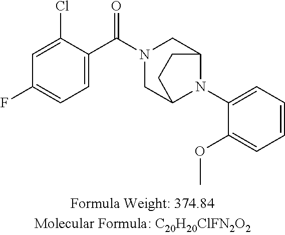 [SMILES: COc1ccccc1N1C2CCC1CN(C(=O)c1ccc(F)cc1Cl)C2]

According to GP-5, I-007 was obtained as an orange solid in 40% yield using 2-bromoanisole (1.28 mL, 10.3 mmol, 1.2 equiv.), piperazine I-001 (2.30 g, 8.56 mmol, 1 equiv.), Cs₂CO₃ (5.58 g, 17.2 mmol, 2 equiv.), Pd (OAc) 2 (192 mg, 856 μmol, 0.1 equiv.) and rac-BINAP (800 mg, 1.28 mmol, 0.15 equiv.) in toluene (43 mL) at reflux for 16 h. Purification by FC (DCM/MeOH 99:1 to 90:10) and FC (cHex/EtOAc=95/5 to 60/40). ¹H NMR (400 MHZ, Chloroform-d, 2 sets of rotamers) δ 7.36 (dd, J=8.5, 5.9 Hz, 0.5H), 7.20 (dd, J=8.5, 2.5 Hz, 0.5H), 7.18-7.10 (m, 1H), 7.07-7.00 (m, 1H), 6.97-6.90 (m, 1H), 6.90-6.82 (m, 2H), 6.82-6.75 (m, 1H), 4.55-4.42 (m, 1H), 4.23 (d, J=6.5 Hz, 1H), 4.07-4.00 (m, 1H), 3.86 (s, 1.5H), 3.85 (s, 1.5H), 3.69 (dd, J=12.9, 1.9 Hz, 0.5H), 3.52 (dd, J=12.2, 2.2 Hz, 0.5H), 3.35 (dt, J=12.9, 1.5 Hz, 1H), 3.22-3.04 (m, 1H), 2.12-1.80 (m, 4H). MS (ESI⁺): [M+H]⁺ 375.1/377.1.

### 3-[3-(2-chloro-4-fluoro-benzoyl)-3,8-diazabicyclo[3.2.1]octan-8-yl]-4-methoxy-benzenesulfonyl chloride (1-008)

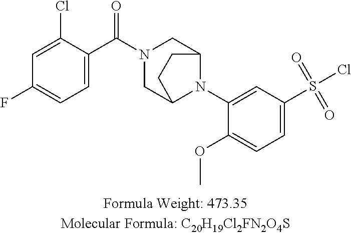 [SMILES: COc1ccc(S(=O)(=O)Cl)cc1N1C2CCC1CN(C(=O)c1ccc(F)cc1Cl)C2]

According to GP-3, 1-008 was obtained as a yellow solid in 85% yield using I-007 (1.25 g, 3.35 mmol, 1 equiv.) and HSO₃Cl (4.5 mL, 67 mmol, 20 equiv.) in DCM (17 mL) at RT for 2.5 h. ¹H NMR (400 MHZ, Chloroform-d, 2 sets of rotamers) δ 7.62 (dt, J=8.7, 2.1 Hz, 1H), 7.37 (dd, J=8.5, 5.9 Hz, 0.5H), 7.33 (t, J=2.6 Hz, 1H), 7.22 (dd, J=8.6, 2.5 Hz, 0.5H), 7.24-7.12 (m, 1H), 7.12-7.01 (m, 1H), 6.97 (d, J=8.7 Hz, 1H), 4.52 (td, J=10.1, 9.3, 4.6 Hz, 1H), 4.33 (dd, J=12.4, 6.3 Hz, 1H), 4.16-4.06 (m, 1H), 3.97 (s, 1.5H), 3.96 (s, 1.5H), 3.66 (d, J=12.5 Hz, 0.5H), 3.65 (d, J=12.5 Hz, 0.5H), 3.33-3.25 (m, 1H), 3.20-3.10 (m, 1H), 2.23-1.69 (m, 4H). MS (ESI⁺): [M+H]⁺ 473.0/474.9.

### Synthesis of Intermediates Required for Final Products 049 to 054

### (2-chloro-4,5-difluoro-phenyl)-[8-(2-methoxyphenyl)-3,8-diazabicyclo[3.2.1]octan-3-yl]methanone (1-038)

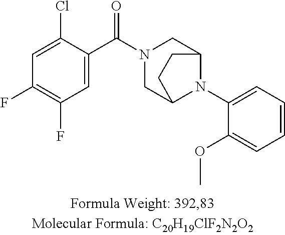 [SMILES: COc1ccccc1N1C2CCC1CN(C(=O)c1cc(F)c(F)cc1Cl)C2]

According to GP-5, I-038 was obtained as an orange solid in 90% yield using 2-bromoanisole (1.08 mL, 8.66 mmol, 1.2 equiv.), piperazine I-033 (2.07 g, 7.22 mmol, 1 equiv.), Cs₂CO₃ (2.00 g, 14.4 mmol, 2 equiv.), Pd (OAc) 2 (162 mg, 722 μmol, 0.1 equiv.) and rac-BINAP (670 mg, 1.08 mmol, 0.15 equiv.) in toluene (34 mL) at reflux for 16 h. Purification by FC (cHex/EtOAc=95/5 to 40/60) and FC (DCM/MeOH 99:1 to 95:5). ¹H NMR (400 MHZ, Chloroform-d, 2 sets of rotamers) δ 7.37-7.20 (m, 1.5H), 7.10-6.8 (m, 4.5H), 4.55-4.40 (m, 1H), 4.30-4.22 (m, 1H), 4.08 (s, 1H), 3.89 & 3.88 (s, 3H), 3.76-3.52 (m, 1H), 3.42-3.34 (m, 1H), 3.13 (t, J=12.2 Hz, 1H), 2.15-1.84 (m, 3.5H), 1.65-1.55 (m, 0.5H). MS (ESI⁺): [M+H]⁺ 393.1/395.1.

### 3-[3-(2-chloro-4,5-difluoro-benzoyl)-3,8-diazabicyclo[3.2.1]octan-8-yl]-4-methoxy-benzenesulfonyl chloride (1-039)

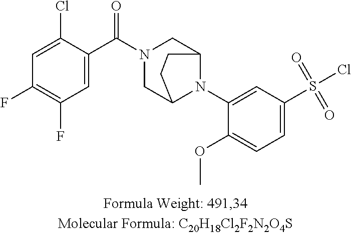 [SMILES: COc1ccc(S(=O)(=O)Cl)cc1N1C2CCC1CN(C(=O)c1cc(F)c(F)cc1Cl)C2]

According to GP-3, I-039 was obtained as a yellow solid in 91% yield using I-038 (845 mg, 2.15 mmol, 1 equiv.) and HSO₃Cl (2.9 mL, 43 mmol, 20 equiv.) in DCM (11 mL) at RT for 2.5 h. ¹H NMR (400 MHZ, Chloroform-d, 2 sets of rotamers) δ 7.64 & 7.62 (s, 1H), 7.40-7.15 (m, 2H), 7.10-6.95 (m, 2H), 4.49 (t, J=11.9 Hz, 1H), 4.32 (s, 1H), 4.11 (s, 1H), 3.97 & 3.96 (s, 3H), 3.72-3.48 (m, 1H), 3.35-3.25 (m, 1H), 3.20-3.08 (m, 1H), 2.15-1.90 (m, 3.5H), 1.70-1.60 (m, 0.5H).

### Synthesis of Intermediates Required for Final Products 055 to 060

### (2-chloro-4-fluoro-phenyl)-[8-(2,3-dihydrobenzofuran-7-yl)-3,8-diazabicyclo[3.2.1]octan-3-yl]methanone (1-040)

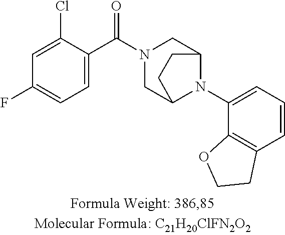 [SMILES: O=C(c1ccc(F)cc1Cl)N1CC2CCC(C1)N2c1cccc2c1OCC2]

According to GP-5, I-040 was obtained as a white foam in 36% yield using 7-bromo-2,3-dihydrobenzofuran (500 mg, 2.51 mmol, 1 equiv.), piperazine I-001 (877 mg, 3.27 mmol, 1.3 equiv.), Cs₂CO₃ (1.64 g, 5.02 mmol, 2 equiv.), Pd (OAc) 2 (56.4 mg, 251 μmol, 0.1 equiv.) and rac-BINAP (188 mg, 301 μmol, 0.12 equiv.) in toluene (13 mL) at reflux for 4 h. Purification by FC (cHex/EtOAc=96/4 to 60/40). ¹H NMR (400 MHZ, Chloroform-d, 2 sets of rotamers) δ 7.36-7.15 (m, 1H), 7.16-6.95 (m, 2H), 6.88-6.72 (m, 2H), 6.71-6.54 (m, 1H), 4.55 & 4.54 (t, J=8.8 Hz, 2H), 4.49-4.36 (m, 2H), 4.25 (s, 1H), 3.70-3.40 (m, 1H), 3.39-3.26 (m, 1H), 3.19 (t, J=8.8 Hz, 2H), 3.03 (t, J=11.9 Hz, 1H), 2.14-1.82 (m, 3.5H), 1.65-1.55 (m, 0.5H). ¹⁹F NMR (376 MHz, Chloroform-d, 2 sets of rotamers) δ-109.6,-109.7. MS (ESI⁺): [M+H]⁺ 387.1/389.1.

### 7-[3-(2-chloro-4-fluoro-benzoyl)-3,8-diazabicyclo[3.2.1]octan-8-yl]-2,3-dihydrobenzofuran-5-sulfonyl chloride (1-041)

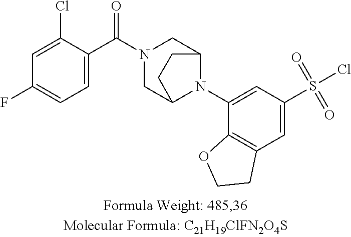 [SMILES: O=C(c1ccc(F)cc1Cl)N1CC2CCC(C1)N2c1cc(S(=O)(=O)Cl)cc2c1OCC2]

According to GP-3, I-041 was obtained as a yellow solid in 93% yield using I-040 (370 mg, 959 μmol, 1 equiv.) and HSO₃Cl (1.3 mL, 19 mmol, 20 equiv.) in DCM (5.6 mL) at RT for 2 h. ¹H NMR (400 MHZ, Chloroform-d, 2 sets of rotamers) δ 7.43 (s, 1H), 7.37 (dd, J=8.5, 5.8 Hz, 0.5H), 7.24-7.17 (m, 1.5H), 7.16-6.96 (m, 2H), 4.73 & 4.72 (t, J=9.0 Hz, 2H), 4.60-4.40 (m, 2H), 4.28 (s, 1H), 3.60 (d, J=12.7 Hz, 0.5H), 3.44 (d, J=12.7 Hz, 0.5H), 3.30 (t, J=8.9 Hz, 2H), 3.24 & 3.21 (s, 1H), 3.09 (t, J=11.3 Hz, 1H), 2.15-1.88 (m, 3.5H), 1.75-1.65 (m, 0.5H). ¹⁹F NMR (376 MHz, Chloroform-d, 2 sets of rotamers) δ-109.1,-109.2.

Synthesis of intermediates required for final product 061

(2-chloro-4,5-difluoro-phenyl)-[8-(2,3-dihydrobenzofuran-7-yl)-3,8-diazabicyclo[3.2.1]octan-3-yl]methanone (1-042)

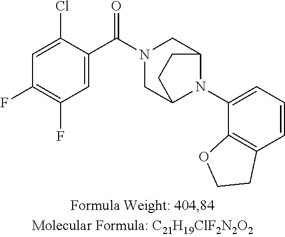 [SMILES: O=C(c1cc(F)c(F)cc1Cl)N1CC2CCC(C1)N2c1cccc2c1OCC2]

According to GP-5, I-042 was obtained as a white solid in 33% yield using 7-bromo-2,3-dihydrobenzofuran (162 mg, 816 μmol, 1.2 equiv.), piperazine I-033 (200 mg, 680 μmol, 1.2 equiv.), Cs₂CO₃ (0.44 g, 1.4 mmol, 2 equiv.), Pd (OAc) 2 (15 mg, 68 μmol, 0.1 equiv.) and rac-BINAP (51 mg, 82 μmol, 0.12 equiv.) in toluene (3.4 mL) at reflux for 16 h. Purification by FC (cHex/EtOAc=96/4 to 60/40). ¹H NMR (400 MHZ, Chloroform-d, 2 sets of rotamers) δ 7.35-7.17 (m, 1.5H), 7.01-6.93 (m, 0.5H), 6.82-6.75 (m, 2H), 6.60 (s, 1H), 4.55 & 4.54 (t, J=8.8 Hz, 2H), 4.44-4.31 (m, 2H), 4.27 (s, 1H), 3.70-3.45 (m, 1H), 3.36-3.27 (m, 1H), 3.19 (t, J=8.8 Hz, 2H), 3.02 (t, J=13.6 Hz, 1H), 2.12-1.82 (m, 3.5H), 1.65-1.55 (m, 0.5H). MS (ESI⁺): [M+H]⁺ 405.1/407.1.

### 7-[3-(2-chloro-4,5-difluoro-benzoyl)-3,8-diazabicyclo[3.2.1]octan-8-yl]-2,3-dihydrobenzofuran-5-sulfonyl chloride (1-043)

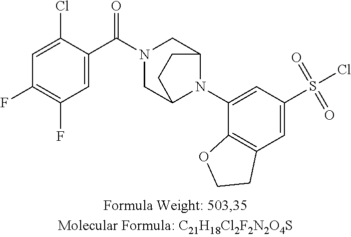 [SMILES: O=C(c1cc(F)c(F)cc1Cl)N1CC2CCC(C1)N2c1cc(S(=O)(=O)Cl)cc2c1OCC2]

According to GP-3, 1-043 was obtained as a yellow solid in 63% yield using I-042 (90.0 mg, 222 μmol, 1 equiv.) and HSO₃Cl (0.3 mL, 4 mmol, 20 equiv.) in DCM (1.1 mL) at RT for 2 h. ¹H NMR (400 MHZ, Chloroform-d, 2 sets of rotamers) δ 7.44 (s, 1H), 7.32 (dd, J=9.6, 6.8 Hz, 0.5H), 7.27-7.18 (m, 2H), 7.03-6.95 (m, 0.5H), 4.74 & 4.73 (t, J=9.0 Hz, 2H), 4.52 (s, 1H), 4.48-4.38 (m, 1H), 4.34-4.28 (m, 1H), 3.66-3.42 (m, 1H), 3.30 (t, J=8.9 Hz, 2H), 3.25 & 3.21 (s, 1H), 3.08 (t, J=11.6 Hz, 1H), 2.16-1.87 (m, 3.5H), 1.70-1.60 (m, 0.5H).

### Synthesis of Intermediates Required for Final Product 016

### 3-bromo-4-methoxy-N-methyl-N-propyl-benzenesulfonamide (1-009)

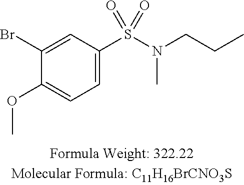 [SMILES: CCCN(C)S(=O)(=O)c1ccc(OC)c(Br)c1]

According to GP-4, I-009 was obtained in 97% yield using commercial 3-bromo-4-methoxy-benzenesulfonyl chloride (5.00 g, 17.5 mmol, 1 equiv.), methyl-N-propylamine (2.7 mL, 27 mmol, 1.5 equiv.) and Et₃N (3.7 mL, 27 mmol, 1.5 equiv.) in DCM (101 mL) at RT for 4 h. Purification by FC (cHex/EtOAc=95/5 to 50/50). ¹H NMR (400 MHZ, Chloroform-d) δ 7.99 (d, J=2.2 Hz, 1H), 7.74 (dd, J=8.7, 2.2 Hz, 1H), 7.00 (d, J=8.7 Hz, 1H), 3.99 (s, 3H), 2.99 (dd, J=8.0, 6.5 Hz, 2H), 2.75 (s, 3H), 1.71-1.51 (m, 2H), 0.96 (t, J=7.4 Hz, 3H). MS (ESI⁺): [M+H]⁺ 322.0/324.0.

### 4-methoxy-N-methyl-N-propyl-3-[2-(trifluoromethyl) piperazin-1-yl]benzenesulfonamide (1-010)

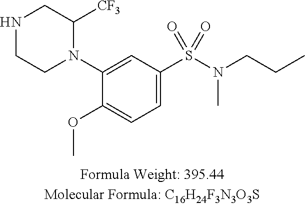 [SMILES: CCCN(C)S(=O)(=O)c1ccc(OC)c(N2CCNCC2C(F)(F)F)c1]

A microwave reaction vial was charged with the aryl bromide I-009 (100 mg, 310 μmol, 1 equiv.), commercial tert-butyl 3-(trifluoromethyl) piperazine-1-carboxylate (118 mg, 466 μmol, 1.5 equiv.), t-BuONa (89.5 mg, 931 μmol, 3 equiv.), Pd2 (dba) 3 (29 mg, 31 μmol, 0.1 equiv.) and JohnPhos (19 mg, 62 μmol, 0.2 equiv.). The vial was flushed with argon and degassed toluene (3.1 mL) was added. The vial was sealed and the reaction was stirred at reflux in a preheated heating-block for 16 h. After cooling down to RT, EtOAc was added and the suspension was filtered over Celite® (EtOAc rinses). The filtrate was concentrated under reduced pressure. The residue was purified by FC (cHex/EtOAc=95/5 to 50/50). The residue was directly treated with TFA (678 μL, 8.80 mmol, 10 equiv.). The mixture was stirred at RT for 1 h. The reaction mixture was concentrated under reduced pressure and the residue was partitioned between water and DCM. K₂CO₃ (s) was added portionwise until pH >11. The layers were separated and the aqueous phase was extracted with DCM (2*). The combined organic extracts were dried (Na₂SO₄), filtered and concentrated under reduced pressure. The residue was purified by FC (DCM/MeOH (7N NH₃)=99/1 to 90/10) to afford 61 mg (50% over two steps) of I-010 as an orange oil. MS (ESI⁺): [M+H]⁺ 396.1.

### Synthesis of Intermediates Required for Final Product 017

### 3-(2-ethylpiperazin-1-yl)-4-methoxy-N-methyl-N-propyl-benzenesulfonamide (1-011)

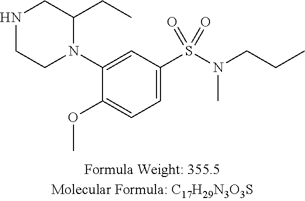 [SMILES: CCCN(C)S(=O)(=O)c1ccc(OC)c(N2CCNCC2CC)c1]

A microwave reaction vial was charged with the aryl bromide I-009 (600 mg, 1.86 mmol, 1 equiv.), commercial tert-butyl 3-ethylpiperazine-1-carboxylate (400 mg, 1.86 mmol, 1 equiv.), Cs₂CO₃ (1.21 g, 3.72 mmol, 2 equiv.), Pd (OAc) 2 (42 mg, 0.19 mmol, 0.1 equiv.) and rac-BINAP (139 mg, 223 μmol, 0.12 equiv.). The vial was flushed with argon and degassed toluene (9.5 mL) was added. The vial was sealed and the reaction was stirred at reflux in a preheated heating-block for 20 h. After cooling down to RT, EtOAc was added and the suspension was filtered over Celite® (EtOAc rinses). The filtrate was concentrated under reduced pressure and purified by FC (cHex/EtOAc=93/7 to 30/70). The residue was directly treated with HCl (4M solution in dioxane, 1.11 mL, 4.46 mmol, 20 equiv.). The mixture was stirred at RT for 2 h. The reaction mixture was concentrated under reduced pressure and the residue was partitioned between water and DCM. NaOH 2M was added dropwise until pH >11. The layers were separated and the aqueous phase was extracted with DCM (2*). The combined organic extracts were dried (Na₂SO₄), filtered and concentrated under reduced pressure. The residue was purified by FC (DCM/MeOH=99/1 to 80/20) to afford 74 mg (11% over two steps) of I-011 as an orange oil. ¹H NMR (400 MHZ, Chloroform-d) δ 7.51 (dd, J=8.5, 2.3 Hz, 1H), 7.36 (d, J=2.3 Hz, 1H), 6.96 (d, J=8.6 Hz, 1H), 3.94 (s, 3H), 3.58-3.42 (m, 1H), 3.42-3.22 (m, 2H), 3.15 (dt, J=6.7, 3.5 Hz, 2H), 3.08-2.88 (m, 4H), 2.72 (s, 3H), 1.73-1.46 (m, 3H), 1.44-1.34 (m, 1H), 0.94 (t, J=7.4 Hz, 3H), 0.80 (t, J=7.5 Hz, 3H). MS (ESI⁺): [M+H]⁺ 356.2.

### Synthesis of Intermediates Required for Final Products 018 and 019

### 4-benzyl-1-(3-bromo-4-methoxy-phenyl) sulfonyl-piperidine (1-012)

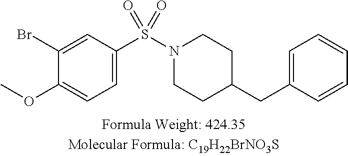 [SMILES: COc1ccc(S(=O)(=O)N2CCC(Cc3ccccc3)CC2)cc1Br]

According to GP-4, 1-012 was obtained in 95% yield using commercial 3-bromo-4-methoxy-benzenesulfonyl chloride (3.00 g, 10.5 mmol, 1 equiv.), 4-benzylpiperidine (2.40 g, 13.7 mmol, 1.3 equiv.) and Et₃N (2.2 mL, 16 mmol, 1.5 equiv.) in DCM (20 mL) at RT for 16 h. Purification by FC (cHex/EtOAc=97/3 to 70/30). ¹H NMR (400 MHZ, Chloroform-d) δ 7.94 (d, J=2.2 Hz, 1H), 7.69 (dd, J=8.7, 2.2 Hz, 1H), 7.39-7.24 (m, 2H), 7.24-7.15 (m, 1H), 7.14-7.06 (m, 2H), 6.98 (d, J=8.6 Hz, 1H), 3.98 (s, 3H), 3.84-3.67 (m, 2H), 2.54 (d, J=6.9 Hz, 2H), 2.23 (td, J=11.8, 2.5 Hz, 2H), 1.80-1.65 (m, 2H), 1.54-1.22 (m, 3H). MS (ESI⁺): [M+H]⁺ 424.0/426.0.

### 1-[5-[(4-benzyl-1-piperidyl) sulfonyl]-2-methoxy-phenyl]-2-methyl-piperazine (1-013)

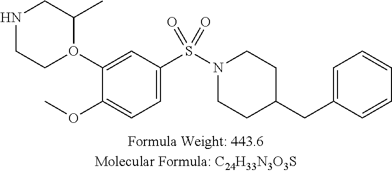

A microwave reaction vial was charged with the aryl bromide I-012 (1.24 g, 2.92 mmol, 1 equiv.), commercial tert-butyl 3-methylpiperazine-1-carboxylate (702 mg, 3.51 mmol, 1.2 equiv.), Cs₂CO₃ (1.90 g, 5.84 mmol, 2 equiv.), Pd (OAc) 2 (33 mg, 0.15 mmol, 0.05 equiv.) and rac-BINAP (110 mg, 175 μmol, 0.06 equiv.). The vial was flushed with argon and degassed toluene (15 mL) was added. The vial was sealed and the reaction was stirred at reflux in a preheated heating-block for 24 h. After cooling down to RT, EtOAc was added and the suspension was filtered over Celite® (EtOAc rinses). The filtrate was concentrated under reduced pressure and purified by FC (cHex/EtOAc=96/4 to 60/40). The residue was directly treated with HCl (4M solution in dioxane, 1.56 mL, 6.25 mmol, equiv.). The mixture was stirred at RT for 2 h. The reaction mixture was concentrated under reduced pressure and the residue was partitioned between water and DCM. NaOH 10 2M was added dropwise until pH >11. The layers were separated and the aqueous phase was extracted with DCM (2*). The combined organic extracts were dried (Na₂SO₄), filtered and concentrated under reduced pressure. The residue was purified by FC (DCM/MeOH=98/2 to 80/20) to afford 140 mg (10% over two steps) of I-013 as an orange oil. ¹H NMR (400 MHZ, Chloroform-d) δ 7.47 (dd, J=8.6, 2.2 Hz, 1H), 7.34 (d, J=2.3 Hz, 1H), 7.32-7.24 (m, 2H), 7.24-7.17 (m, 1H), 7.15-7.06 (m, 2H), 6.94 (d, J=8.6 Hz, 1H), 3.93 (s, 3H), 3.77 (d, J=11.5 Hz, 2H), 3.70-3.60 (m, 1H), 3.33-3.18 (m, 2H), 3.18-3.06 (m, 2H), 2.89-2.83 (m, 1H), 2.83-2.76 (m, 1H), 2.53 (d, J=6.6 Hz, 2H), 2.19 (td, J=11.8, 2.6 Hz, 2H), 1.70 (d, J=11.2 Hz, 2H), 1.55-1.31 (m, 3H), 0.96 (d, J=6.4 Hz, 3H). MS (ESI⁺): [M+H]⁺ 444.0.

Synthesis of Intermediates required for final product 020

### 3-bromo-N-tert-butyl-4-methoxy-benzenesulfonamide (1-014)

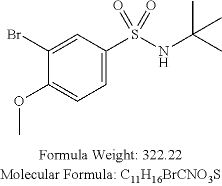 [SMILES: COc1ccc(S(=O)(=O)NC(C)(C)C)cc1Br]

According to GP-4, 1-014 was obtained in 90% yield using commercial 3-bromo-4-methoxybenzenesulfonyl chloride (2.02 g, 7.07 mmol, 1 equiv.), tert-butylamine (1.5 mL, 14 mmol, 2 equiv.) and Et₃N (2.0 mL, 14 mmol, 2 equiv.) in DCM (28 mL) at RT for 16 h. Purification by FC (cHex/EtOAc=100/0 to 20/80). ¹H NMR (400 MHZ, Chloroform-d) 8 8.06 (d, J=2.3 Hz, 1H), 7.82 (dd, J=8.7, 2.3 Hz, 1H), 6.94 (d, J=8.7 Hz, 1H), 4.48 (s, 1H), 3.96 (s, 3H), 1.24 (s, 9H). MS (ESI⁺): [M+H]⁺ 322.0/324.0.

### N-tert-butyl-4-methoxy-3-(2-methylpiperazin-1-yl)benzenesulfonamide (1-015)

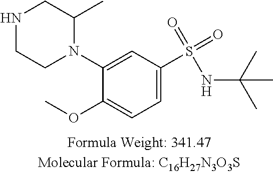 [SMILES: COc1ccc(S(=O)(=O)NC(C)(C)C)cc1N1CCNCC1C]

A microwave reaction vial was charged with the aryl bromide I-014 (844 mg, 2.54 mmol, 1 equiv.), commercial tert-butyl 3-methylpiperazine-1-carboxylate (763 mg, 3.81 mmol, 1.5 equiv.), Cs₂CO₃ (1.7 g, 5.2 mmol, 2 equiv.), Pd (OAc) 2 (57 mg, 0.25 mmol, 0.1 equiv.) and rac-BINAP (237 mg, 381 μmol, 0.15 equiv.). The vial was flushed with argon and degassed toluene (12 mL) was added. The vial was sealed and the reaction was stirred at reflux in a preheated heating-block for 24 h. After cooling down to RT, EtOAc was added and the suspension was filtered over Celite® (EtOAc rinses). The filtrate was concentrated under reduced pressure and purified by FC (cHex/EtOAc=100/0 to 50/50). The residue was directly treated with HCl (aq. 37%, 50 μL, 1.6 mmol, 10 equiv.). The mixture was stirred at RT for 2 h. The reaction mixture was concentrated under reduced pressure and the residue was partitioned between water and DCM. NaOH 2M was added dropwise until pH >11. The layers were separated and the aqueous phase was extracted with DCM (2*). The combined organic extracts were dried (Na₂SO₄), filtered and concentrated under reduced pressure. The residue was purified by PTLC (DCM/MeOH =85/15) to afford 45 mg (5% over two steps) of I-015 as a yellow oil. ¹H NMR (400 MHZ, Chloroform-d) δ 7.52 (dd, J=8.6, 2.3 Hz, 1H), 7.41 (d, J=2.3 Hz, 1H), 6.83 (d, J=8.6 Hz, 1H), 4.68 (s, 1H), 3.84 (s, 3H), 3.49 (td, J=6.4, 3.3 Hz, 1H), 3.16 (ddd, J=11.5, 6.2, 3.2 Hz, 1H), 3.07 (dd, J=12.2, 3.4 Hz, 1H), 2.97 (qdd, J=12.2, 6.6, 3.2 Hz, 2H), 2.77-2.59 (m, 2H), 1.14 (s, 9H), 0.85 (d, J=6.4 Hz, 3H). MS (ESI⁺): [M+H]⁺ 342.1.

### Synthesis of Intermediate Required for Final Product 021

### 4-methoxy-N-methyl-3-(2-methylpiperazin-1-yl)-N-propyl-benzenesulfonamide (1-016)

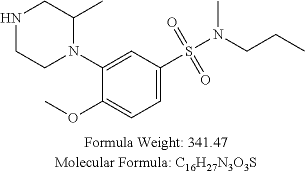 [SMILES: CCCN(C)S(=O)(=O)c1ccc(OC)c(N2CCNCC2C)c1]

A microwave reaction vial was charged with the aryl bromide I-009 (1.0 g, 3.1 mmol, 1 equiv.), commercial tert-butyl 3-methylpiperazine-1-carboxylate (932 mg, 4.66 mmol, 1.5 equiv.), t-BuONa (895 mg, 9.31 mmol, 3 equiv.), Pd (OAc) 2 (70 mg, 0.31 mmol, 0.1 equiv.) and rac-BINAP (290 mg, 466 μmol, 0.15 equiv.). The vial was flushed with argon and degassed toluene (16 mL) was added. The vial was sealed and the reaction was stirred at reflux in a preheated heating-block for 16 h. After cooling down to RT, EtOAc was added and the suspension was filtered over Celite® (EtOAc rinses). The filtrate was concentrated under reduced pressure and purified by FC (cHex/EtOAc=95/5 to 40/60). The residue was directly treated with HCl (aq. 37%, 800 μL, 793 μmol, 10 equiv.). The mixture was stirred at RT for 16 h. The reaction mixture was concentrated under reduced pressure and the residue was partitioned between water and DCM. NaOH 2M was added dropwise until pH >11. The layers were separated and the aqueous phase was extracted with DCM (2*). The combined organic extracts were dried (Na₂SO₄), filtered and concentrated under Reduced pressure. residue was purified by FC (DCM/MeOH (7N NH₃)=99/1 to 90/10) to afford 96 mg (9% over two steps) of I-016 as an orange oil. 1H NMR (400 MHZ, DMSO-d₆) δ 7.41 (dd, J=8.5, 2.3 Hz, 1H), 7.23-7.07 (m, 2H), 3.87 (s, 3H), 3.51 (dt, J=6.4, 3.2 Hz, 1H), 3.11 (td, J=7.1, 3.3 Hz, 1H), 2.99 (dd, J=12.0, 3.3 Hz, 1H), 2.92-2.76 (m, 4H), 2.65-2.55 (m, 5H), 1.46 (q, J=7.2 Hz, 2H), 0.85-0.82 (m, 6H). MS (ESI⁺): [M+H]⁺ 342.2.

### Synthesis of Intermediate Required for Final Product 022

### 4-methoxy-3-[2-(methoxymethyl) piperazin-1-yl]-N-methyl-N-propyl-benzenesulfonamide (1-017)

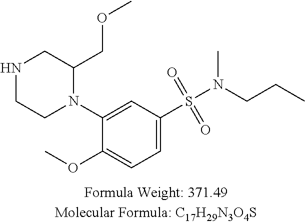 [SMILES: CCCN(C)S(=O)(=O)c1ccc(OC)c(N2CCNCC2COC)c1]

A microwave reaction vial was charged with the aryl bromide I-009 (600 mg, 1.86 mmol, 1 equiv.), tert-butyl 3-(methoxymethyl) piperazine-1-carboxylate (429 mg, 1.86 mmol, 1 equiv.), Cs₂CO₃ (1.82 g, 5.59 mmol, 3 equiv.), Pd (OAc) 2 (42 mg, 0.19 mmol, 0.1 equiv.) and rac-BINAP (174 mg, 279 μmol, 0.15 equiv.). The vial was flushed with argon and degassed toluene (9.5 mL) was added. The vial was sealed and the reaction was stirred at reflux in a preheated heating-block for 3 h. After cooling down to RT, EtOAc was added and the suspension was filtered over Celite® (EtOAc rinses). The filtrate was concentrated under reduced pressure and purified by FC (DCM/EtOAc=95/5 to 40/60). The residue was directly treated with HCl (4M solution in dioxane, 1.03 mL, 4.14 mmol, 10 equiv.). The mixture was stirred at RT for 16 h. The reaction mixture was concentrated under reduced pressure and the residue was partitioned between water and DCM. K₂CO₃ (s) was added portionwise until pH >11. The layers were separated and the aqueous phase was extracted with DCM (2*). The combined organic extracts were dried (Na₂SO₄), filtered and concentrated under Reduced pressure. residue was purified by FC (DCM/MeOH (7N NH₃)=99/1 to 90/10) to afford 78 mg (8% over two steps) of I-017 as an orange oil. ¹H NMR (400 MHZ, Chloroform-d) δ 7.46 (dd, J=8.6, 2.0 Hz, 1H), 7.36 (d, J=2.1 Hz, 1H), 6.94 (dd, J=8.5, 1.6 Hz, 1H), 3.93 (d, J=1.7 Hz, 3H), 3.80 (dt, J=7.6, 3.7 Hz, 1H), 3.65-3.58 (m, 1H), 3.40-3.26 (m, 2H), 3.22 (d, J=1.7 Hz, 3H), 3.19-2.99 (m, 4H), 2.99-2.87 (m, 3H), 2.72 (d, J=1.6 Hz, 3H), 1.56 (q, J=7.4 Hz, 2H), 0.94 (td, J=7.4, 1.6 Hz, 3H). MS (ESI⁺): [M+H]⁺ 372.2.

### Synthesis of Intermediate Required for Final Product 023

### 4-methoxy-N-methyl-3-(3-oxa-7,9-diazabicyclo[3.3.1]nonan-9-yl)-N-propyl-benzenesulfonamide (1-018)

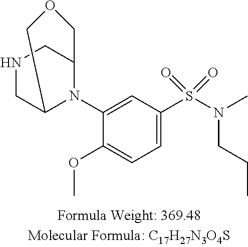 [SMILES: CCCN(C)S(=O)(=O)c1ccc(OC)c(N2C3CNCC2COC3)c1]

A microwave reaction vial was charged with the aryl bromide I-009 (100 mg, 310 μmol, 1 equiv.), tert-butyl 3-oxa-7,9-diazabicyclo[3.3.1]nonane-7-carboxylate (71 mg, 0.31 mmol, 1 equiv.), Cs₂CO₃ (304 g, 931 μmol, 3 equiv.), Pd (OAc) 2 (7.0 mg, 31 μmol, 0.1 equiv.) and rac-BINAP (29 mg, 47 μmol, 0.15 equiv.). The vial was flushed with argon and degassed toluene (1.6 mL) was added. The vial was sealed and the reaction was stirred at reflux in a preheated heating-block for 16 h. After cooling down to RT, EtOAc was added and the suspension was filtered over Celite® (EtOAc rinses). The filtrate was concentrated under reduced pressure and purified by FC (cHex/EtOAc=93/7 to 30/70). The residue was directly treated with HCl (4M solution in dioxane, 650 μL, 2.60 mmol, 10 equiv.). The mixture was stirred at RT for 16 h. The reaction mixture was concentrated under reduced pressure and the residue was partitioned between water and DCM. K₂CO₃ (s) was added portionwise until pH >11. The layers were separated and the aqueous phase was extracted with DCM (2*). The combined organic extracts were dried (Na₂SO₄), filtered and concentrated under reduced pressure. The residue was purified by FC (DCM/MeOH (7N NH₃)=99/1 to 90/10) to afford 90 mg (79% over two steps) of I-018 as a colorless oil. ¹H NMR (400 MHZ, Chloroform-d, 2 sets of rotamers) δ 7.39-7.25 (m, 2H), 6.93 (d, J=8.8 Hz, 1H), 4.24-4.04 (m, 4H), 3.92 (s, 3H), 3.90-3.78 (m, 1H), 3.63 (s, 1H), 3.53-3.41 (m, 2H), 3.37-3.35 (m, 2H), 3.14-2.93 (m, 3H), 2.72 (d, J=6.4 Hz, 3H), 1.58 (q, J=7.3 Hz, 2H), 0.95 (t, J=7.4 Hz, 3H). MS (ESI⁺): [M+H]⁺ 370.2.

### Synthesis of Intermediate Required for Final Product 026

### 3-bromo-4-fluoro-N-methyl-N-propyl-benzenesulfonamide (1-019)

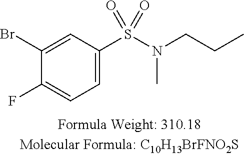 [SMILES: CCCN(C)S(=O)(=O)c1ccc(F)c(Br)c1]

According to GP-4, 1-019 was obtained as a white solid in 95% yield using commercial 3-bromo-4-fluorobenzenesulfonyl chloride (388 mg, 1.35 mmol, 1 equiv.), methyl-N-propylamine (145 μL, 1.41 mmol, 1.05 equiv.) and Et₃N (281 μL, 2.02 mmol, 1.5 equiv.) in DCM (7 mL) at RT for 1 h. ¹H NMR (400 MHZ, Chloroform-d) δ 8.01 (dd, J=6.3, 2.3 Hz, 1H), 7.73 (ddd, J=8.6, 4.5, 2.2 Hz, 1H), 7.26 (dd, J=8.6, 8.0 Hz, 1H), 3.08-2.90 (m, 2H), 2.75 (s, 3H), 1.58 (q, J=7.3 Hz, 2H), 0.94 (t, J=7.4 Hz, 3H). ¹⁹F NMR (376 MHz, Chloroform-d) 8-99.95. MS (ESI⁺): [M+H]⁺ 310.0/312.0.

### Synthesis of Intermediate Required for Final Product 027

### 3-bromo-N-methyl-N-propyl-4-(trifluoromethoxy)benzenesulfonamide (1-020)

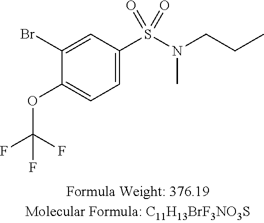 [SMILES: CCCN(C)S(=O)(=O)c1ccc(OC(F)(F)F)c(Br)c1]

According to GP-4, 1-020 was obtained as a white solid in 77% yield using commercial 3-bromo-4-(trifluoromethoxy)benzene-1-sulfonyl chloride (500 mg, 1.47 mmol, 1 equiv.), methyl-N-propylamine (159 μL, 1.55 mmol, 1.05 equiv.) and Et₃N (308 μL, 2.21 mmol, 1.5 equiv.) in DCM (7.5 mL) at RT for 1 h. ¹H NMR (400 MHZ, Chloroform-d) δ 8.07 (d, J=2.2 Hz, 1H), 7.76 (dd, J=8.6, 2.2 Hz, 1H), 7.48-7.39 (m, 1H), 3.07-2.98 (m, 2H), 2.78 (s, 3H), 1.59 (q, J=7.3 Hz, 2H), 0.94 (t, J=7.4 Hz, 3H). ¹⁹F NMR (376 MHZ, Chloroform-d) 8-57.36. MS (ESI⁺): [M+H]⁺ 376.5/378.3.

### Synthesis of Intermediate Required for Final Product 028

### 3-bromo-N-methyl-N-propyl-benzenesulfonamide (1-021)

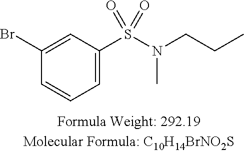

According to GP-4, I-021 was obtained as a white solid in 100% yield using commercial 3-bromobenzenesulfonyl chloride (500 mg, 1.96 mmol, 1 equiv.), methyl-N-propylamine (301 μL, 2.94 mmol, 1.5 equiv.) and Et₃N (408 μL, 2.94 mmol, 1.5 equiv.) in DCM (4 mL) at RT for 2 h. ¹H NMR (400 MHZ, Chloroform-d) δ 7.95 (t, J=1.8 Hz, 1H), 7.73 (dddd, J=6.4, 4.9, 2.4, 1.1 Hz, 2H), 7.42 (t, J=7.9 Hz, 1H), 3.06-2.94 (m, 2H), 2.77 (s, 3H), 1.69-1.48 (m, 2H), 0.95 (t, J=7.4 Hz, 3H). MS (ESI⁺): [M+H]⁺ 292.2/294.2.

Synthesis of Intermediates required for final product 029

### 3-bromo-4-hydroxy-N-methyl-N-propyl-benzenesulfonamide (1-022)

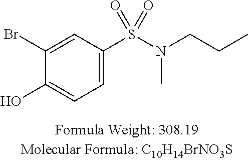 [SMILES: CCCN(C)S(=O)(=O)c1ccc(O)c(Br)c1]

To a solution of I-009 (2.37 g, 7.34 mmol, 1.00 eq) in DCM (90 mL) at 0° C. under argon atmosphere, was added boron tribromide (1M in DCM, 11 mL, 11 mmol, 1.5 equiv.). The reaction mixture was stirred at RT for 48 h. More boron tribromide (1M in DCM, 11 mL, 11 mmol, 1.50 eq.) was added at 0° C. and the reaction was stirred at RT for 72 h. Water was added at 0° C. and the layers were separated. The aqueous phase was extracted with DCM. The combined organic extracts were dried (Na₂SO₄), filtered and concentrated under reduced pressure. The residue was purified by FC (cHex/EtOAc=99/1 to 70/30) affording 2.1 g (86%) of I-022 as a grey oil. ¹H NMR (400 MHZ, Chloroform-d) δ 7.93 (d, J=2.1 Hz, 1H), 7.64 (dd, J=8.6, 2.2 Hz, 1H), 7.12 (d, J=8.6 Hz, 1H), 6.08 (s, 1H), 3.00-2.93 (m, 2H), 2.72 (s, 3H), 1.57 (h, J=7.4 Hz, 2H), 0.93 (t, J=7.4 Hz, 3H). MS (ESI⁺): [M+H]⁺ 308.0/310.0.

### 3-bromo-4-ethoxy-N-methyl-N-propyl-benzenesulfonamide (1-023)

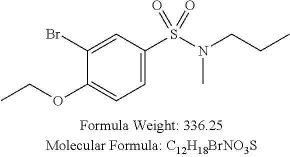 [SMILES: CCCN(C)S(=O)(=O)c1ccc(OCC)c(Br)c1]

To a solution of I-022 (100 mg, 325 μmol, 1 equiv.) in dry DMF (1.7 mL) under argon atmosphere were added K₂CO₃ (90 mg, 0.65 mmol, 2 equiv.) and bromoethane (53 mg, 0.49 mmol, 1.5 equiv.). The solution was stirred at RT for 16 h. Water was added and the product was extracted with EtOAc. The combined organic extracts were washed (brine), dried (MgSO₄), filtered and concentrated under reduced pressure. The residue was purified by FC (cHex/EtOAc=95/5 to 70/30) to afford 97 mg (88%) of I-023 as a colorless oil. ¹H NMR (400 MHZ, Chloroform-d) δ 7.95 (d, J=2.3 Hz, 1H), 7.68 (dd, J=8.6, 2.3 Hz, 1H), 6.94 (d, J=8.7 Hz, 1H), 4.17 (q, J=7.0 Hz, 2H), 3.02-2.88 (m, 2H), 2.71 (s, 3H), 1.60-1.45 (m, 5H), 0.93 (t, J=7.4 Hz, 3H). MS (ESI⁺): [M+H]⁺ 336.0/338.0.

### Synthesis of Intermediate Required for Final Product 030

### 3-bromo-4-(cyclobutoxy)-N-methyl-N-propyl-benzenesulfonamide (1-024)

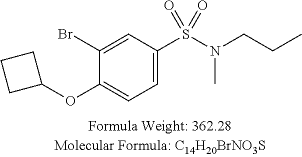 [SMILES: CCCN(C)S(=O)(=O)c1ccc(OC2CCC2)c(Br)c1]

To a solution of I-022 (100 mg, 325 μmol, 1 equiv.) in dry DMF (1.6 mL) under argon atmosphere were added K₂CO₃ (90 mg, 0.65 mmol, 2 equiv.) and bromocyclobutane (46 μL, 0.49 mmol, 1.5 equiv.). The solution was stirred at RT for 16 h. Water was added and the product was extracted with EtOAc. The combined organic extracts were washed (brine), dried (MgSO₄), filtered and concentrated under reduced pressure. The residue was purified by FC (cHex/EtOAc=100/0 to 50/50) to afford 53 mg (43%) of I-024 as a colorless oil. ¹H NMR (400 MHZ, Chloroform-d) δ 7.95 (d, J=1.9 Hz, 1H), 7.65 (dd, J=8.6, 2.2 Hz, 1H), 6.79 (d, J=8.6 Hz, 1H), 4.81-4.68 (m, 1H), 2.96 (t, J=7.3 Hz, 2H), 2.72 (s, 3H), 2.56-2.45 (m, 2H), 2.28 (ddd, J=12.7, 6.3, 2.4 Hz, 2H), 1.93 (q, J=10.6 Hz, 1H), 1.81-1.66 (m, 1H), 1.56 (dd, J=14.4, 6.9 Hz, 2H), 0.93 (t, J=7.4 Hz, 3H). MS (ESI⁺): [M+H]⁺ 362.0/364.0.

### Synthesis of Intermediate Required for Final Product 031

### 3-bromo-4-(cyclobutoxy)-N-methyl-N-propyl-benzenesulfonamid3-bromo-4-(methoxymethoxy)-N-methyl-N-propyl-benzenesulfonamide (1-025)

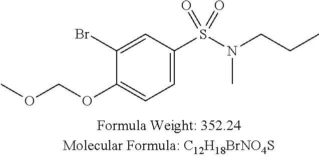 [SMILES: CCCN(C)S(=O)(=O)c1ccc(OCOC)c(Br)c1]

To a solution of I-022 (265 mg, 860 μmol, 1 equiv.) in dry DMF (3.5 mL) under argon atmosphere were added iPr₂NEt (450 μL, 2.58 mmol, 3 equiv.) and chloromethyl methyl ether (131 μL, 1.72 mmol, 2 equiv.). The solution was stirred at RT for 1 h. The reaction mixture was partitioned between MTBE and HCl. The layers were separated and the aqueous phase was extracted with MTBE. the combined organic extracts were washed (HCl, brine), dried (Na₂SO₄), filtered and concentrated under reduced pressure affording 300 mg (96%) of I-025 as a colorless oil. ¹H NMR (400 MHZ, Chloroform-d) δ 8.00 (d, J=2.2 Hz, 1H), 7.70 (dd, J=8.7, 2.3 Hz, 1H), 7.26 (d, J=8.7 Hz, 1H), 5.34 (s, 2H), 3.55 (s, 3H), 3.00 (dd, J=8.0, 6.5 Hz, 2H), 2.75 (s, 3H), 1.67-1.45 (m, 2H), 0.96 (t, J=7.4 Hz, 3H). MS (ESI⁺): [M+H]⁺ 352.0/354.0.

### Synthesis of Intermediate Required for Final Product 032

### 5-bromo-6-chloro-N,N-diethyl-pyridine-3-sulfonamide (1-026)

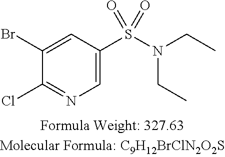 [SMILES: CCN(CC)S(=O)(=O)c1cnc(Cl)c(Br)c1]

According to GP-4, I-026 was obtained as a white solid in 100% yield using commercial 5-bromo-6-chloro-pyridine-3-sulfonyl chloride (438 mg, 1.50 mmol, 1 equiv.), diethylamine (100 mg, 1.37 mmol, 1 equiv.) and Et₃N (229 μL, 1.64 mmol, 1.2 equiv.) in DCM (6.9 mL) at RT for 1 h. ¹H NMR (400 MHZ, Chloroform-d) δ 8.74 (d, J=2.2 Hz, 1H), 8.32 (d, J=2.2 Hz, 1H), 3.31 (q, J=7.2 Hz, 4H), 1.21 (t, J=7.1 Hz, 6H). MS (ESI⁺): [M+H]⁺ 326.8/328.9.

### Synthesis of Intermediate Required for Final Product 033

### 5-bromo-N,N-diethyl-6-methoxy-pyridine-3-sulfonamide (1-027)

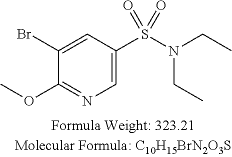 [SMILES: CCN(CC)S(=O)(=O)c1cnc(OC)c(Br)c1]

To a solution of I-026 (150 mg, 458 μmol, 1.00 equiv.) in dry MeOH (1.83 mL) at RT, was added MeONa (124 mg, 2.29 mmol, 5 equiv.). The mixture was stirred at RT overnight. The RM was partitioned between aq. sat. NH₄Cl and EtOAc. The layers were separated and the aqueous phase was extracted with EtOAc. The combined organic extracts were washed (brine), dried (Na₂SO₄), filtered and concentrated under reduced pressure to afford 138 mg (95%) of I-027 as a light-yellow solid. ¹H NMR (400 MHZ, Chloroform-d) δ 8.52 (d, J=2.2 Hz, 1H), 8.16 (d, J=2.2 Hz, 1H), 4.08 (s, 3H), 3.25 (q, J=7.2 Hz, 4H), 1.17 (t, J=7.1 Hz, 6H). MS (ESI⁺): [M+H]⁺ 323.0/324.9.

### Synthesis of Intermediate Required for Final Product 034

### 6-benzyloxy-5-bromo-N,N-diethyl-pyridine-3-sulfonamide (1-028)

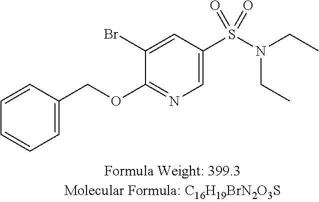 [SMILES: CCN(CC)S(=O)(=O)c1cnc(OCc2ccccc2)c(Br)c1]

To a solution of commercial benzyl alcohol (77 μL, 0.73 mmol, 1.2 equiv.) in dry toluene (1.22 mL) at 0° C., was added NaH (60% in mineral oil, 29 mg, 0.73 mmol, 1.2 equiv.) and the mixture was stirred at 0° C. for 40 min before the addition of I-026 (200 mg, 610 μmol, 1.00 equiv.). The solution was stirred at RT for 16 h. The mixture was partitioned between water and EtOAc. The layers were separated and the aqueous phase was extracted with EtOAc. The combined organic extracts were washed (brine), dried (Na₂SO₄), filtered and concentrated under reduced pressure to afford 239 mg (98%) of I-028 as a yellow oil. 1H NMR (400 MHZ, Chloroform-d) δ 8.52 (d, J=2.2 Hz, 1H), 8.18 (d, J=2.2 Hz, 1H), 7.54-7.43 (m, 2H), 7.42-7.31 (m, 3H), 5.52 (s, 2H), 3.25 (q, J=7.1 Hz, 4H), 1.18 (t, J=7.1 Hz, 6H). MS (ESI⁺): [M+H]⁺ 398.9/401.0.

### Synthesis of Intermediate Required for Final Product 036

### 4-benzyloxy-3-bromo-N-methyl-N-propyl-benzenesulfonamide (1-029)

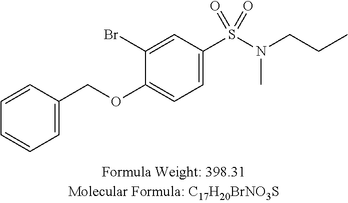 [SMILES: CCCN(C)S(=O)(=O)c1ccc(OCc2ccccc2)c(Br)c1]

To a solution of I-022 (2.09 g, 6.30 mmol, 1 equiv.) in dry DMF (68 mL) under argon atmosphere were added K₂CO₃ (1.74 g, 12.6 mmol, 2 equiv.) and benzyl bromide (750 μL, 6.30 mmol, 1 equiv.). The solution was stirred at RT for 16 h. Water was added and the product was extracted with EtOAc. The combined organic extracts were washed (brine), dried (MgSO₄), filtered and concentrated under reduced pressure. The residue was purified by FC (cHex/EtOAc=95/5 to 70/30) to afford 2.37 g (91%) of I-029 as a colorless oil. ¹H NMR (400 MHZ, Chloroform-d) δ 7.99 (d, J=2.2 Hz, 1H), 7.67 (dd, J=8.6, 2.3 Hz, 1H), 7.50-7.44 (m, 2H), 7.44-7.38 (m, 2H), 7.38-7.31 (m, 1H), 7.01 (d, J=8.7 Hz, 1H), 5.23 (s, 2H), 2.97 (dd, J=7.9, 6.6 Hz, 2H), 2.72 (s, 3H), 1.66-1.50 (m, 2H), 0.93 (t, J=7.4 Hz, 3H). MS (ESI⁺): [M+H]⁺ 399.0/401.0.

### 4-benzyloxy-3-[3-(2-chloro-4-fluoro-benzoyl)-3,8-diazabicyclo[3.2.1]octan-8-yl]-N-methyl-N-propyl-benzenesulfonamide (1-030)

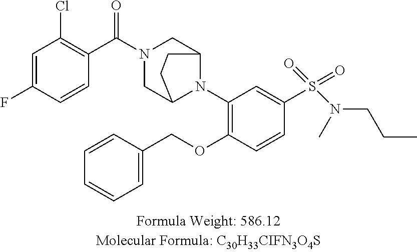 [SMILES: CCCN(C)S(=O)(=O)c1ccc(OCc2ccccc2)c(N2C3CCC2CN(C(=O)c2ccc(F)cc2Cl)C3)c1]

According to GP-5, I-030 was obtained as a white foam in 72% yield using aryl bromide I-029 (1.13 g, 2.84 mmol, 1 equiv.), piperazine I-001 (915 mg, 3.40 mmol, 1.2 equiv.), Cs₂CO₃ (2.31 g, 7.09 mmol, 2.5 equiv.), Pd (OAc) 2 (64 mg, 0.28 mmol, 0.1 equiv.) and rac-BINAP (212 mg, 340 μmol, 0.12 equiv.) in toluene (12 mL) at reflux for 16 h. Purification by FC (cHex/EtOAc=100/0 to 50/50) and PTLC (DCM/MeOH=99/1). 1H NMR (400 MHZ, Chloroform-d, 2 sets of rotamers) δ 7.52-7.31 (m, 6H), 7.24-6.95 (m, 4H), 5.21-5.10 (m, 2H), 4.47 (d, J=13.1 Hz, 1H), 4.35 (d, J=13.5 Hz, 1H), 4.09 (d, J=10.5 Hz, 1H), 3.70-3.50 (m, 1H), 3.40-3.23 (m, 1H), 3.14-3.06 (m, 1H), 3.00-2.88 (m, 2H), 2.69 (s, 3H), 2.09-1.84 (m, 4H), 1.67-1.43 (m, 3H), 0.91 (t, J=7.3 Hz, 3H). 19F NMR (376 MHz, Chloroform-d, 2 sets of rotamers) δ-109.21,-109.23. MS (ESI⁺): [M+H]⁺ 586.6/588.6.

Synthesis of Intermediates required for final product 037

### (2-chloro-4-fluoro-phenyl)-[8-[5-(3-hydroxypyrrolidin-1-yl) sulfonyl-2-methoxy-phenyl]-3,8-diazabicyclo[3.2.1]octan-3-yl]methanone (1-031)

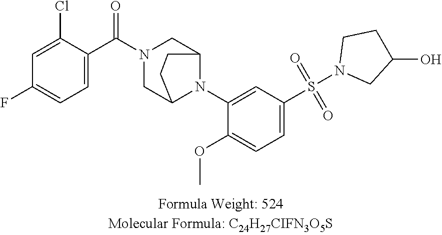 [SMILES: COc1ccc(S(=O)(=O)N2CCC(O)C2)cc1N1C2CCC1CN(C(=O)c1ccc(F)cc1Cl)C2]

According to GP-4, I-031 was obtained as a white solid in 83% yield using I-008 (400 mg, 845 μmol, 1 equiv.), 3-Pyrrolidinol (103 μL, 1.27 mmol, 1.5 equiv.) and iPr₂NEt (295 μL, 1.69 mmol, 2 equiv.) in DCM (5 mL) at RT for 4 h. Purification by FC (cHex/EtOAc=70/30 to 0/100). ¹H NMR (400 MHZ, Chloroform-d, 2 sets of rotamers) δ 7.42 (dd, J=8.5, 2.1 Hz, 1H), 7.36 (dd, J=8.5, 5.8 Hz, 0.5H), 7.24-7.19 (m, 1.5H), 7.15 (dd, J=8.4, 2.5 Hz, 1H), 7.11-6.98 (m, 1H), 6.93 (d, J=8.4 Hz, 1H), 4.57-4.45 (m, 1H), 4.37 (s, 1H), 4.34-4.26 (m, 1H), 4.07 (s, 1H), 3.92 & 3.91 (s, 3H), 3.70-3.45 (m, 1H), 3.44-3.25 (m, 4H), 3.20 (d, J=11.3 Hz, 1H), 3.12 (t, J=11.0 Hz, 1H), 2.06-1.78 (m, 5.5H), 1.65-1.55 (m, 0.5H). MS (ESI⁺): [M+H]⁺ 524.1/526.1.

Synthesis of Intermediates required for final product 038

### 3-[4-(2-chloro-4-fluoro-benzoyl)-2-methyl-piperazin-1-yl]-N—(3-fluoropropyl)-4-methoxy-benzenesulfonamide (1-032)

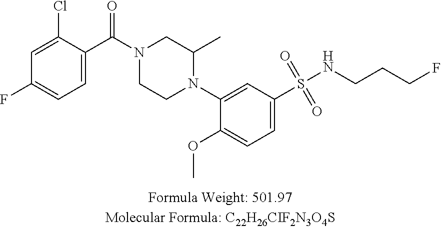 [SMILES: COc1ccc(S(=O)(=O)NCCCF)cc1N1CCN(C(=O)c2ccc(F)cc2Cl)CC1C]

According to GP-4, 1-032 was obtained as a white solid in 32% yield using I-006 (175 mg, 379 μmol, 1 equiv.), 3-Fluoropropan-1-amine hydrochloride (87 mg, 0.80 mmol, 2 equiv.) and Et₃N (212 μL, 1.52 mmol, 4 equiv.) in DCM (1.9 mL) at RT for 16 h. Purification by FC (DCM/MeOH=99/1 to 90/10). ¹H NMR (400 MHZ, Chloroform-d, multiple sets of rotamers) δ 7.65-7.55 (m, 1H), 7.45-7.28 (m, 2H), 7.23-7.15 (m, 1H), 7.11-7.03 (m, 1H), 6.96-6.90 (m, 1H), 4.59-4.37 (m, 3H), 3.98-3.70 (m, 5H), 3.60-3.27 (m, 3H), 3.20-2.73 (m, 4H), 1.91 (p, J=5.9 Hz, 1H), 1.82 (p, J=6.0 Hz, 1H), 1.06-0.78 (m, 3H). ¹⁹F NMR (376 MHz, Chloroform-d, multiple sets of rotamers) δ-109.28,-109.33,-109.35,-221.18,-221.20. MS (ESI⁺): [M+H]⁺ 502.1/504.1.

Synthesis of Intermediates required for final products 046 to 048

### tert-butyl 4-[3-[3-(2-chloro-4-fluoro-benzoyl)-3,8-diazabicyclo[3.2.1]octan-8-yl]-4-methoxy-phenyl]sulfonylpiperazine-1-carboxylate (Boc-1-037)

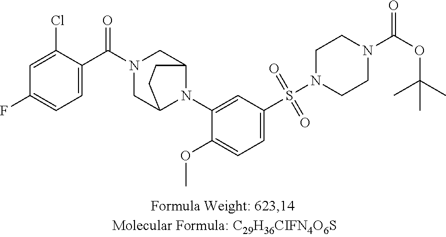 [SMILES: COc1ccc(S(=O)(=O)N2CCN(C(=O)OC(C)(C)C)CC2)cc1N1C2CCC1CN(C(=O)c1ccc(F)cc1Cl)C2]

According to GP-4, Boc-I-037 was obtained as a white solid in 74% yield using I-008 (2.14 g, 4.24 mmol, 1 equiv.), tert-butyl piperazine-1-carboxylate (1.2 g, 6.4 mmol, 1.5 equiv.) and iPr₂NEt (2.22 mL, 12.7 mmol, 3 equiv.) in DCM (21 mL) at RT for 16 h. Purification by FC (cHex/EtOAc=95/5 to 0/100). ¹H NMR (400 MHZ, Chloroform-d, 2 sets of rotamers) δ 7.40-7.28 (m, 1.5H), 7.24-7.00 (m, 3.5H), 6.94 (dd, J=8.5, 1.3 Hz, 1H), 4.51 (dd, J=13.0, 4.8 Hz, 1H), 4.28 (s, 1H), 4.05 (s, 1H), 3.93 & 3.92 (s, 3H), 3.70 -3.45 (m, 5H), 3.33-3.09 (m, 2H), 2.93 (t, J=5.1 Hz, 4H), 2.04-1.85 (m, 3.5H), 1.70 -1.60 (m, 0.5H), 1.41 (s, 9H). MS (ESI⁺): [M+H]⁺ 623.3/625.3.

### (2-chloro-4-fluoro-phenyl)-[8-(2-methoxy-5-piperazin-1-ylsulfonyl-phenyl)-3,8-diazabicyclo[3.2.1]octan-3-yl]methanone (1-037)

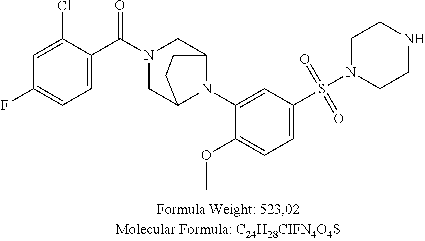 [SMILES: COc1ccc(S(=O)(=O)N2CCNCC2)cc1N1C2CCC1CN(C(=O)c1ccc(F)cc1Cl)C2]

To a solution of tert-butyl 4-[3-[3-(2-chloro-4-fluoro-benzoyl)-3,8-diazabicyclo[3.2.1]octan-8-yl]-4-methoxy-phenyl]sulfonylpiperazine-1-carboxylate Boc-I-037 (2.02 g, 3.14 mmol, 1 equiv.) in DCM (4 mL) at RT, was added HCl (4M solution in dioxane, 3.9 mL, 16 mmol, 5 equiv.). The mixture was stirred at RT for 16 h. The reaction mixture was concentrated under reduced pressure and the residue was partitioned between water and DCM. 2N NaOH was added dropwise until pH >11. The layers were separated and the aqueous phase was extracted with DCM (2*). The combined organic extracts were dried (Na₂SO₄), filtered and concentrated under reduced pressure to afford 1.54 g (94%) of (2-chloro-4-fluoro-phenyl)-[8-(2-methoxy-5-piperazin-1-ylsulfonyl-phenyl)-3,8-diazabicyclo[3.2.1]octan-3-yl]methanone I-037 as a white solid. ¹H NMR (400 MHZ, Chloroform-d, 2 sets of rotamers) δ 7.41-7.29 (m, 1.5H), 7.23-7.18 (m, 0.5H), 7.19-7.12 (m, 1H), 7.12-7.00 (m, 2H), 6.93 (dd, J=8.5, 1.3 Hz, 1H), 4.56-4.46 (m, 1H), 4.28 & 4.27 (s, 1H), 4.05 (s, 1H), 3.92 & 3.91 (s, 3H), 3.70 -3.46 (m, 1H), 3.37-3.24 (m, 1H), 3.13 (t, J=10.6 Hz, 1H), 2.99-2.88 (m, 8H), 2.09 -1.85 (m, 3.5H), 1.70-1.60 (m, 0.5H). ¹⁹F NMR (376 MHz, Chloroform-d, 2 sets of rotamers) δ-109.3,-109.4. MS (ESI⁺): [M+H]⁺ 523.1/525.1.

Synthesis of Intermediates required for final product 062

### 1-(3-bromo-4-methoxy-phenyl) sulfonyl-4-(2-phenylethyl) piperidine (1-044)

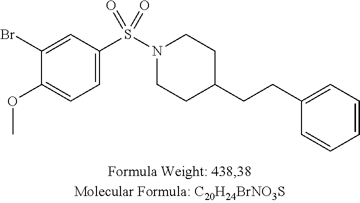 [SMILES: COc1ccc(S(=O)(=O)N2CCC(CCc3ccccc3)CC2)cc1Br]

According to GP-4, I-044 was obtained as a white solid in 93% yield using 3-bromo-4-methoxybenzenesulfonyl chloride (1.50 g, 5.25 mmol, 1 equiv.), 4-phenethyl-piperidine (1.12 g, 6.30 mmol, 1.2 equiv.) and Et₃N (1.1 mL, 7.9 mmol, 1.5 equiv.) in DCM (10 mL) at RT for 2 h. ¹H NMR (400 MHZ, Chloroform-d) δ 7.95 (d, J=2.2 Hz, 1H), 7.71 (dd, J=8.6, 2.2 Hz, 1H), 7.34-7.24 (m, 2H), 7.24-7.06 (m, 3H), 7.00 (d, J=8.6 Hz, 1H), 3.99 (s, 3H), 3.78 (dt, J=11.2, 2.7 Hz, 2H), 2.66-2.55 (m, 2H), 2.26 (td, J=11.8, 2.5 Hz, 2H), 1.89-1.71 (m, 2H), 1.64-1.50 (m, 2H), 1.45-1.15 (m, 3H). MS (ESI⁺): [M+H]⁺ 438.0/440.0

### 1-[3-bromo-4-(methoxymethoxy)phenyl]sulfonyl-4-(2-phenylethyl) piperidine (I-045)

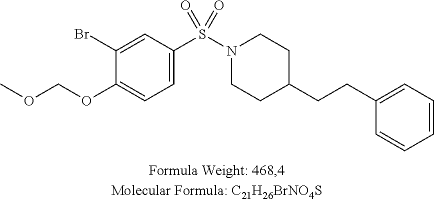 [SMILES: COCOc1ccc(S(=O)(=O)N2CCC(CCc3ccccc3)CC2)cc1Br]

To a solution of I-044 (2.15 g, 4.90 mmol, 1 equiv.) in DCM (10 mL) at 0° C., was added BBr3 (1M solution in DCM, 9.8 mL, 9.8 mmol, 2 equiv.). The mixture was stirred at RT for 60 h. Water was carefully added and the layers were separated. The aqueous phase was extracted with DCM and the combined organic extracts were dried (Na₂SO₄), filtered and concentrated under reduced pressure. The residue was dissolved in DCM (20 ml) at RT and iPr₂NEt (3.4 mL, 20mmoL, 4 equiv) and chloromethyl methyl ether (0.74 mL, 9.8 mmol, 2 equiv.) were added dropwise. The mixture was stirred at RT for 1 h. It was partitioned between EtOAc and 1N HCl. The layers were separated and the aqueous phase was extracted with EtOAc. The combined organic extracts were washed (1N HCl, brine), dried (Na₂SO₄), filtered and concentrated under reduced pressure. The residue was purified by FC (cHex/EtOAc=97/3 to 70/30) to afford 1.38 g of I-045 (61% over two steps) as a white solid. ¹H NMR (400 MHZ, Chloroform-d) δ 7.96 (d, J=2.2 Hz, 1H), 7.66 (dd, J=8.7, 2.3 Hz, 1H), 7.32-7.23 (m, 3H), 7.25-7.12 (m, 3H), 5.34 (s, 2H), 3.89-3.66 (m, 2H), 3.55 (s, 3H), 2.65-2.60 (m, 2H), 2.30-2.20 (m, 2H), 1.86-1.72 (m, 2H), 1.66-1.53 (m, 2H), 1.45-1.15 (m, 3H). MS (ESI⁺): [M+H]⁺ 468.0/470.0

### (2-chloro-4-fluoro-phenyl)-[8-[2-(methoxymethoxy)-5-[[4-(2-phenylethyl)-1-piperidyl]sulfonyl]phenyl]-3,8-diazabicyclo[3.2.1]octan-3-yl]methanone (1-046)

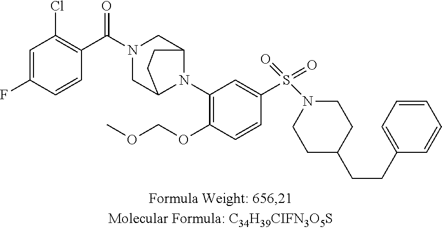 [SMILES: COCOc1ccc(S(=O)(=O)N2CCC(CCc3ccccc3)CC2)cc1N1C2CCC1CN(C(=O)c1ccc(F)cc1Cl)C2]

According to GP-5, I-046 was obtained as a white foam in 21% yield using aryl bromide I-045 (1.38 g, 2.96 mmol, 1 equiv.), piperazine I-001 (1.12 g, 4.15 mmol, 1.4 equiv.), Cs₂CO₃ (1.93 g, 5.93 mmol, 2 equiv.), Pd (OAc) 2 (67 mg, 0.30 mmol, 0.1 equiv.) and rac-BINAP (222 mg, 356 μmol, 0.12 equiv.) in toluene (15 mL) at reflux for 2 h. Purification by FC (cHex/EtOAc=95/5 to 0/100). ¹H NMR (400 MHZ, Chloroform-d, 2 sets of rotamers) δ 7.40-7.33 (m, 1H), 7.30-7.20 (m, 2H), 7.20-7.10 (m, 6H), 7.10-6.98 (m, 2H), 5.30-5.20 (m, 2H), 4.53 & 4.50 (s, 1H), 4.31 & 4.29 (s, 1H), 4.06 (s, 1H), 3.80-3.65 (m, 2.5H), 3.55-3.45 (m 3.5H), 3.30 & 3.27 (s, 1H), 3.30-3.10 (m, 1H), 2.65-2.60 (m, 2H), 2.30-2.15 (m, 2H), 3.10-1.85 (m, 3.5H), 1.85-1.60 (m, 2.5H), 1.60-1.45 (m, 3H), 1.40-1.25 (m, 2H). ¹⁹F NMR (376 MHz, Chloroform-d, 2 sets of rotamers) δ-109.2,-109.3. MS (ESI⁺): [M+H]⁺ 656.2/658.2.

Synthesis of Intermediates required for final product 063

### 1-(3-bromo-4-methoxy-phenyl) sulfonyl-4-phenyl-piperidine (1-047)

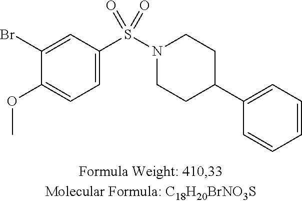 [SMILES: COc1ccc(S(=O)(=O)N2CCC(c3ccccc3)CC2)cc1Br]

According to GP-4, 1-047 was obtained as a white solid in 99% yield using 3-bromo-4-methoxybenzenesulfonyl chloride (1.50 g, 5.25 mmol, 1 equiv.), 4-phenylpiperidine (1.02 g, 6.30 mmol, 1.2 equiv.) and Et₃N (1.1 mL, 7.9 mmol, 1.5 equiv.) in DCM (10 mL) at RT for 2 h. ¹H NMR (400 MHZ, Chloroform-d) δ 8.00 (d, J=2.3 Hz, 1H), 7.76 (dd, J=8.6, 2.2 Hz, 1H), 7.40-7.27 (m, 2H), 7.27-7.19 (m, 1H), 7.19-7.14 (m, 2H), 7.03 (d, J=8.7 Hz, 1H), 4.01 (s, 3H), 3.98-3.89 (m, 2H), 2.62-2.32 (m, 3H), 2.01-1.73 (m, 4H). MS (ESI⁺): [M+H]⁺ 410.0/412.0.

### 1-[3-bromo-4-(methoxymethoxy)phenyl]sulfonyl-4-phenyl-piperidine (1-048)

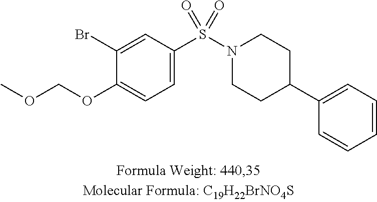 [SMILES: COCOc1ccc(S(=O)(=O)N2CCC(c3ccccc3)CC2)cc1Br]

To a solution of I-047 (2.25 g, 5.21 mmol, 1 equiv.) in DCM (10 mL) at 0° C., was added BBr3 (1M solution in DCM, 10.4 mL, 10.4 mmol, 2 equiv.). The mixture was stirred at RT for 60 h. Water was carefully added and the layers were separated. The aqueous phase was extracted with DCM and the combined organic extracts were dried (Na₂SO₄), filtered and concentrated under reduced pressure. The residue was dissolved in DCM (20 ml) at RT and iPr₂NEt (3.6 mL, 21 mmol, 4 equiv) and chloromethyl methyl ether (0.79 mL, 10.4 mmol, 2 equiv.) were added dropwise. The mixture was stirred at RT for 1 h. It was partitioned between EtOAc and 1N HCl. The layers were separated and the aqueous phase was extracted with EtOAc. The combined organic extracts were washed (1N HCl, brine), dried (Na₂SO₄), filtered and concentrated under reduced pressure. The residue was purified by FC (cHex/EtOAc=97/3 to 70/30) to afford 1.74 g of I-048 (74% over two steps) as a white solid. ¹H NMR (400 MHZ, Chloroform-d) δ 8.01 (d, J=2.2 Hz, 1H), 7.71 (dd, J=8.7, 2.2 Hz, 1H), 7.36-7.28 (m, 3H), 7.27-7.20 (m, 1H), 7.20-7.13 (m, 2H), 5.36 (s, 2H), 3.96 (d, J=11.7 Hz, 2H), 3.57 (s, 3H), 2.57-2.32 (m, 3H), 1.99-1.77 (m, 4H). MS (ESI⁺): [M+H]⁺ 440.0/442.0

### (2-chloro-4-fluoro-phenyl)-[8-[2-(methoxymethoxy)-5-[(4-phenyl-1-piperidyl) sulfonyl]phenyl]-3,8-diazabicyclo[3.2.1]octan-3-yl]methanone (1-049)

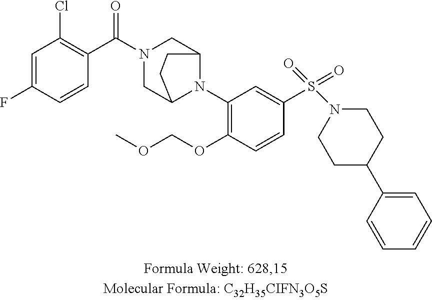 [SMILES: COCOc1ccc(S(=O)(=O)N2CCC(c3ccccc3)CC2)cc1N1C2CCC1CN(C(=O)c1ccc(F)cc1Cl)C2]

According to GP-5, 1-049 was obtained as a white foam in 87% yield using aryl bromide I-048 (1.20 g, 2.72 mmol, 1 equiv.), piperazine I-001 (0.88 g, 3.3 mmol, 1.2 equiv.), Cs₂CO₃ (1.77 g, 5.45 mmol, 2 equiv.), Pd (OAc) 2 (61 mg, 0.27 mmol, 0.1 equiv.) and rac-BINAP (204 mg, 327 μmol, 0.12 equiv.) in toluene (14 mL) at reflux for 3.5 h. Purification by FC (cHex/EtOAc=94/6 to 40/60). ¹H NMR (400 MHZ, Chloroform-d, 2 sets of rotamers) δ 7.43-7.27 (m, 3.5H), 7.26-7.14 (m, 6.5H), 7.16-7.05 (m, 1H), 5.39-5.19 (m, 2H), 4.57 & 4.53 (s, 1H), 4.36 & 4.35 (s, 1H), 4.10 (s, 1H), 3.95-3.87 (m, 2H), 3.75-3.50 (m, 4H), 3.33 & 3.30 (s, 1H), 3.24-3.13 (m, 1H), 2.51-2.25 (m, 3H), 2.17-1.76 (m, 7.5H), 1.72-1.62 (m, 0.5H). ¹⁹F NMR (376 MHz, Chloroform-d, 2 sets of rotamers) δ-109.1,-109.3. MS (ESI⁺): [M+H]⁺ 628.2/630.2.

Synthesis of Intermediates required for final product 064

### 2-bromo-4-[4-[2-(4-chlorophenyl)ethyl]piperazin-1-yl]sulfonyl-phenol (1-050)

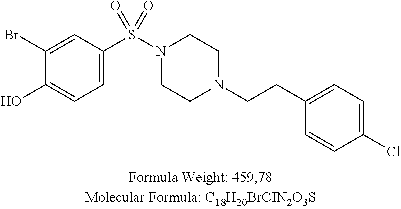 [SMILES: O=S(=O)(c1ccc(O)c(Br)c1)N1CCN(CCc2ccc(Cl)cc2)CC1]

To a solution of 1-[2-(4-chlorophenyl)ethyl]piperazine dihydrochloride (1.08 g, 3.64 mmol, 1.3 equiv.) and Et₃N (3.9 mL, 28 mmol, 10 equiv.) in DCM (14 mL) at RT, was added dropwise a solution of 3-bromo-4-hydroxy-benzenesulfonyl chloride (800 mg, 2.80 mmol, 1 equiv.) in DCM (10 mL). The mixture was stirred at RT for 4 h. Sat. aq. NH₄Cl and EtOAc were added. The layers were separated and the aqueous phase was extracted with EtOAc. The combined organic extracts were dried (Na₂SO₄), filtered and concentrated under reduced pressure. The residue was purified by FC (DCM/MeOH=99/1 to 90/10) to afford 623 mg of I-050 (44%) as a beige solid. ¹H NMR (400 MHZ, DMSO-d₆) δ 11.48 (s, 1H), 7.78 (d, J=2.2 Hz, 1H), 7.58 (dd, J=8.6, 2.3 Hz, 1H), 7.36 -7.25 (m, 2H), 7.25-7.19 (m, 2H), 7.15 (d, J=8.6 Hz, 1H), 2.88 (s, 4H), 2.75-2.50 (m, 8H). MS (ESI⁺): [M+H]⁺ 459.1/461.1/463.0.

### 1-[3-bromo-4-(methoxymethoxy)phenyl]sulfonyl-4-[2-(4-chlorophenyl)ethyl]piperazine (1-051)

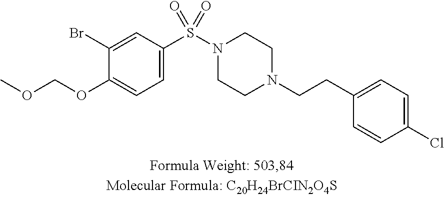 [SMILES: COCOc1ccc(S(=O)(=O)N2CCN(CCc3ccc(Cl)cc3)CC2)cc1Br]

To a solution of I-050 (623 mg, 1.22 mmol, 1 equiv.) and iPr₂NEt (0.64 mL, 3.7 mmol, 3 equiv.) in DCM (6 mL) at RT, was added chloromethyl methyl ether (0.19 mL, 2.4 mmol, 2 equiv.). The mixture was stirred at RT for 16 h. It was partitioned between EtOAc and sat. aq. NaHCO₃. The layers were separated and the aqueous phase was extracted with EtOAc. The combined organic extracts were dried (Na₂SO₄), filtered and concentrated under reduced pressure. The residue was purified by FC (cHex/EtOAc=95/5 to 50/50) to afford 0.51 g of I-051 (83%) as a white solid. ¹H NMR (400 MHZ, Chloroform-d) 8 7.94 (d, J=2.3 Hz, 1H), 7.64 (dd, J=8.7, 2.2 Hz, 1H), 7.25-7.21 (m, 3H), 7.10-7.05 (m, 2H), 5.32 (s, 2H), 3.53 (s, 3H), 3.11-2.99 (m, 4H), 2.74-2.66 (m, 2H), 2.62-2.52 (m, 6H).MS (ESI⁺): [M+H]⁺ 503.1/505.1/507.1.

### (2-chloro-4-fluoro-phenyl)-[8-[5-[4-[2-(4-chlorophenyl)ethyl]piperazin-1-yl]sulfonyl-2-(methoxymethoxy)phenyl]-3,8-diazabicyclo[3.2.1]octan-3-yl]methanone (1-052)

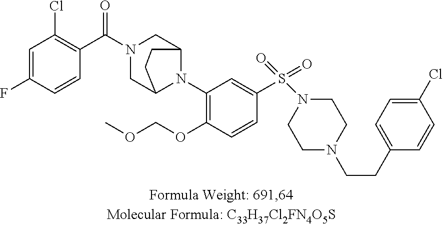 [SMILES: COCOc1ccc(S(=O)(=O)N2CCN(CCc3ccc(Cl)cc3)CC2)cc1N1C2CCC1CN(C(=O)c1ccc(F)cc1Cl)C2]

According to GP-5, I-052 was obtained as a white foam in 51% yield using aryl bromide I-051 (0.51 g, 1.0 mmol, 1 equiv.), piperazine I-001 (0.33 g, 1.2 mmol, 1.2 equiv.), K₂CO₃ (0.28 g, 2.0 mmol, 2 equiv.), Pd (OAc) 2 (23 mg, 0.10 mmol, 0.1 equiv.) and rac-BINAP (75.6 mg, 121 μmol, 0.12 equiv.) in toluene (5 mL) at reflux for 16 h. Purification by FC (cHex/EtOAc=90/10 to 10/90). ¹H NMR (400 MHZ, Chloroform-d, 2 sets of rotamers) 8 7.40-7.00 (m, 10H), 5.26 & 5.25 (s, 2H), 4.57-4.47 (m, 1H), 4.30 & 4.28 (s, 1H), 4.08 (s, 1H), 3.70-3.45 (m, 4H), 3.34-3.11 (m, 2H), 3.10-2.90 (m, 4H), 2.75-2.65 (m, 2H), 2.65-2.45 (m, 6H), 2.05-1.80 (m, 3.5H), 1.70-1.60 (m, 0.5H). ¹⁹F NMR (376 MHz, Chloroform-d, 2 sets of rotamers) δ-109.2,-109.3. MS (ESI⁺): [M+H]⁺ 691.4/693.4/695.4.

Synthesis of Intermediates required for final product 065

### 1-(3-bromo-4-methoxy-phenyl) sulfonyl-4-(4-fluorophenyl) piperidine (1-053)

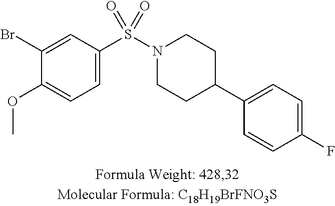 [SMILES: COc1ccc(S(=O)(=O)N2CCC(c3ccc(F)cc3)CC2)cc1Br]

According to GP-4, I-053 was obtained as a white solid in 90% yield using 3-bromo-4-methoxybenzenesulfonyl chloride (637 mg, 2.23 mmol, 1 equiv.), 4-(4-fluorophenyl) piperidine (400 g, 2.23 mmol, 1 equiv.) and iPr₂NEt (0.78 mL, 4.5 mmol, 2 equiv.) in DCM (11 mL) at RT for 16 h. ¹H NMR (400 MHZ, Chloroform-d) δ 7.97 (d, J=2.2 Hz, 1H), 7.73 (dd, J=8.7, 2.2 Hz, 1H), 7.15-7.07 (m, 2H), 7.04-6.93 (m, 3H), 3.99 (s, 3H), 3.94-3.84 (m, 2H), 2.47-2.26 (m, 3H), 2.00-1.71 (m, 4H). MS (ESI⁺): [M+H]⁺ 428.0/430.0.

### 2-bromo-4-[[4-(4-fluorophenyl)-1-piperidyl]sulfonyl]phenol (1-054)

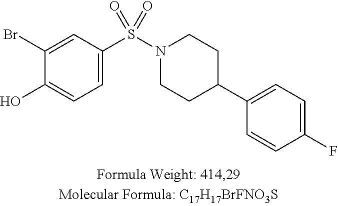 [SMILES: O=S(=O)(c1ccc(O)c(Br)c1)N1CCC(c2ccc(F)cc2)CC1]

To a solution of I-053 (0.86 g, 2.0 mmol, 1 equiv.) in DCM (4 mL) at 0° C., was added BBr3 (1M solution in DCM, 4.0 mL, 4.0 mmol, 2 equiv.). The mixture was stirred at RT for 60 h. Water was carefully added and the layers were separated. The aqueous phase was extracted with DCM and the combined organic extracts were dried (Na₂SO₄), filtered and concentrated under reduced pressure. The residue was purified by FC (DCM 100%) to afford 285 mg of I-054 (34%) as a white solid. ¹H NMR (400 MHZ, Chloroform-d) δ 7.94 (d, J=2.2 Hz, 1H), 7.66 (dd, J=8.6, 2.1 Hz, 1H), 7.19-7.06 (m, 3H), 7.03-6.94 (m, 2H), 5.99 (s, 1H), 3.98-3.82 (m, 2H), 2.48-2.31 (m, 3H), 1.97-1.71 (m, 4H). MS (ESI⁺): [M+H]⁺ 414.0/416.0.

### 1-[3-bromo-4-(methoxymethoxy)phenyl]sulfonyl-4-(4-fluorophenyl) piperidine (1-055)

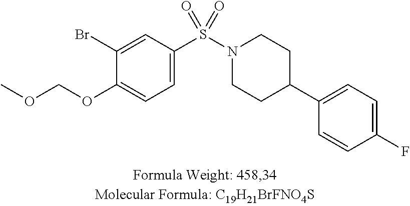 [SMILES: COCOc1ccc(S(=O)(=O)N2CCC(c3ccc(F)cc3)CC2)cc1Br]

To a solution of I-054 (285 mg, 674 μmol, 1 equiv.) and iPr₂NEt (0.35 mL, 2.0 mmol, 3 equiv.) in DCM (3 mL) at RT, was added chloromethyl methyl ether (0.10 mL, 1.3 mmol, 2 equiv.). The mixture was stirred at RT for 16 h. It was partitioned between MTBE and 1N HCl. The layers were separated and the aqueous phase was extracted with MTBE. The combined organic extracts were washed (1N HCl, brine), dried (Na₂SO₄), filtered and concentrated under reduced pressure to afford 0.31 g of I-055 (81%) as a yellow oil.

¹H NMR (400 MHz, Chloroform-d) δ 7.96 (d, J=2.2 Hz, 1H), 7.66 (dd, J=8.7, 2.3 Hz, 1H), 7.27-7.20 (m, 1H), 7.12-7.04 (m, 2H), 6.99-6.92 (m, 2H), 5.31 (s, 2H), 3.94-3.86 (m, 2H), 3.52 (s, 3H), 2.49-2.31 (m, 3H), 1.90-1.72 (m, 4H). MS (ESI⁺): [M+H]⁺ 458.0/460.0.

### (2-chloro-4-fluoro-phenyl)-[8-[5-[[4-(4-fluorophenyl)-1-piperidyl]sulfonyl]-2-(methoxymethoxy)phenyl]-3,8-diazabicyclo[3.2.1]octan-3-yl]methanone (1-056)

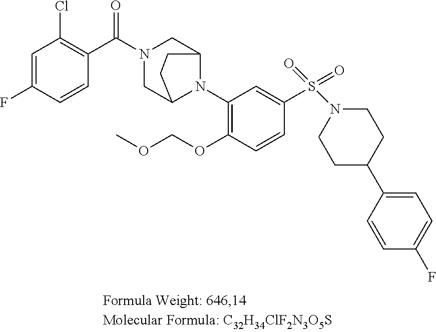 [SMILES: COCOc1ccc(S(=O)(=O)N2CCC(c3ccc(F)cc3)CC2)cc1N1C2CCC1CN(C(=O)c1ccc(F)cc1Cl)C2]

According to GP-5, I-056 was obtained as a white foam in 66% yield using aryl bromide I-055 (309 mg, 613 μmol, 1 equiv.), piperazine I-001 (0.20 g, 0.73 mmol, 1.2 equiv.), Cs₂CO₃ (600 mg, 1.84 mmol, 3 equiv.), Pd (OAc) 2 (14 mg, 61 μmol, 0.1 equiv.) and rac-BINAP (46 mg, 74 μmol, 0.12 equiv.) in toluene (3.1 mL) at reflux for 16 h. Purification by FC (cHex/EtOAc=95/5 to 50/50). ¹H NMR (400 MHZ, Chloroform-d, 2 sets of rotamers) δ 7.43-7.27 (m, 1.5H), 7.25-7.01 (m, 6.5H), 7.01-6.91 (m, 2H), 5.35-5.23 (m, 2H), 4.54 & 4.51 (s, 1H), 4.33 & 4.32 (s, 1H), 4.08 (s, 1H), 3.91 & 3.88 (s, 2H), 3.75-3.47 (m, 4H), 3.31 & 3.27 (s, 1H), 3.17 & 3.14 (d, J=6.7 Hz, 1H), 2.50-2.28 (m, 3H), 2.03-1.60 (m, 8H). MS (ESI⁺): [M+H]⁺ 646.2/648.2.

### Synthesis of Intermediate Required for Final Product 066

### 1-(3-bromo-4-chloro-phenyl) sulfonyl-4-phenyl-piperidine (1-057)

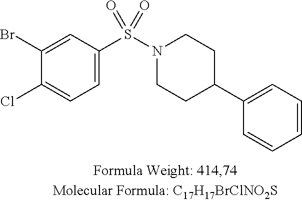 [SMILES: O=S(=O)(c1ccc(Cl)c(Br)c1)N1CCC(c2ccccc2)CC1]

According to GP-4, 1-057 was obtained as a white solid in 58% yield using commercial 3-bromo-4-chlorobenzenesulfonyl chloride (200 mg, 690 μmol, 1 equiv.), 4-phenylpiperidine (122 mg, 759 μmol, 1.1 equiv.) and Et₃N (144 μL, 1.03 mmol, 1.5 equiv.) in DCM (3.4 mL) at RT for 16 h. Purification by FC (cHex/EtOAc=97/3 to 70/30). 1H NMR (400 MHZ, Chloroform-d) δ 8.07 (d, J=2.0 Hz, 1H), 7.73-7.61 (m, 2H), 7.38-7.30 (m, 2H), 7.27-7.21 (m, 1H), 7.21-7.14 (m, 2H), 4.06-3.89 (m, 2H), 2.59-2.34 (m, 3H), 2.03-1.77 (m, 4H). MS (ESI⁺): [M+H]⁺ 414.0/416.0/418.0.

### Synthesis of Intermediate Required for Final Product 067

### 1-(3-bromo-4-methyl-phenyl) sulfonyl-4-phenyl-piperidine (1-058)

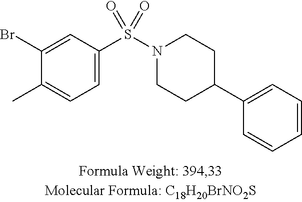 [SMILES: Cc1ccc(S(=O)(=O)N2CCC(c3ccccc3)CC2)cc1Br]

According to GP-4, 1-058 was obtained as a white solid in 72% yield using commercial 3-bromo-4-methylbenzenesulfonyl chloride (200 mg, 741 μmol, 1 equiv.), 4-phenylpiperidine (132 mg, 816 μmol, 1.1 equiv.) and Et₃N (144 μL, 1.03 mmol, 1.5 equiv.) in DCM (3.4 mL) at RT for 16 h. ¹H NMR (400 MHZ, Chloroform-d) δ 7.89 (d, J=1.8 Hz, 1H), 7.55 (dd, J=7.9, 1.9 Hz, 1H), 7.37-7.30 (m, 1H), 7.27-7.20 (m, 2H), 7.17-7.11 (m, 1H), 7.11-7.03 (m, 2H), 3.92-3.81 (m, 2H), 2.42 (s, 3H), 2.40-2.24 (m, 3H), 1.89-1.70 (m, 4H). MS (ESI⁺): [M+H]⁺ 394.0/396.0.

Synthesis of Intermediate Required for Final Product 068 3-bromo-N,4-dimethyl-N-propyl-benzenesulfonamide (1-059)

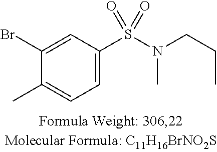 [SMILES: CCCN(C)S(=O)(=O)c1ccc(C)c(Br)c1]

According to GP-4, 1-059 was obtained as a white solid in 88% yield using commercial 3-bromo-4-methylbenzenesulfonyl chloride (60.0 mg, 223 μmol, 1 equiv.), methyl-N-propylamine (30 μL, 0.29 mmol, 1.3 equiv.) and Et₃N (93 μL, 0.67 mmol, 3 equiv.) in DCM (1.1 mL) at RT for 1 h. ¹H NMR (400 MHZ, Chloroform-d) δ 7.94 (d, J=1.9 Hz, 1H), 7.61 (dd, J=7.9, 1.9 Hz, 1H), 7.37 (d, J=8.0 Hz, 1H), 3.00-2.94 (m, 2H), 2.73 (s, 3H), 2.47 (s, 3H), 1.63-1.56 (m, 2H), 0.93 (t, J=7.4 Hz, 3H). MS (ESI⁺): [M+H]⁺ 306.0/308.0.

### Synthesis of Intermediate Required for Final Product 069

### methyl 2-bromo-4-[(4-phenyl-1-piperidyl) sulfonyl]benzoate (1-060)

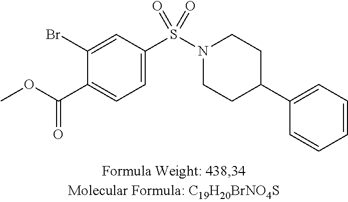 [SMILES: COC(=O)c1ccc(S(=O)(=O)N2CCC(c3ccccc3)CC2)cc1Br]

According to GP-4, 1-060 was obtained as a white solid in 48% yield using 3-bromo-4-methyl 2-bromo-4-chlorosulfonyl benzoate (1.36 g, 4.34 mmol, 1 equiv.), 4-phenylpiperidine (769 mg, 4.77 mmol, 1.1 equiv.) and iPr₂NEt (1.14 mL, 6.50 mmol, 1.5 equiv.) in DCM (43 mL) at RT for 16 h. Purification by FC (cHex/EtOAc=93/7 to 30/70). ¹H NMR (400 MHZ, Chloroform-d) δ 8.10 (d, J=1.7 Hz, 1H), 7.93 (d, J=8.1 Hz, 1H), 7.79 (dd, J=8.1, 1.7 Hz, 1H), 7.37-7.30 (m, 2H), 7.27-7.22 (m, 1H), 7.20-7.15 (m, 2H), 4.01 (s, 3H), 4.01-3.96 (m, 2H), 2.54-2.36 (m, 3H), 2.01-1.79 (m, 4H). MS (ESI⁺): [M+H]⁺ 438.0/440.0.

### Synthesis of Intermediate Required for Final Product 074

### 3-bromo-5-chloro-4-methyl-aniline (1-061)

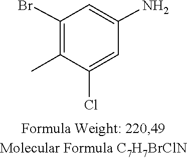

To a solution of 1-bromo-3-chloro-2-methyl-5-nitro-benzene (1.39 g, 5.53 mmol, 1 equiv.) in AcOH (31 mL) at RT, was added Zn (3.62 g, 55.3 mmol, 10 equiv.). The mixture was stirred at RT for 4 h. The suspension was filtered over Celite (EtOAc rinses) and the combined filtrates were concentrated under reduced pressure. The residue was purified by FC (cHex/EtOAc=98/2 to 80/20) to afford 967 mg (79%) of I-061 as a brown solid. ¹H NMR (400 MHZ, Chloroform-d) δ 6.83 (d, J=2.4 Hz, 1H), 6.68 (d, J=2.3 Hz, 1H), 3.52 (s, 2H), 2.38 (s, 3H). MS (ESI⁺): [M+H]⁺ 220.0/222.0/224.0.

### 3-bromo-5-chloro-4-methyl-benzenesulfonyl chloride (1-062)

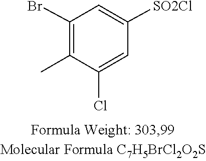

To a solution of CuCl (27 mg, 0.27 mmol, 0.12 equiv.) in water (7 mL) at 0° C., was added dropwise thionyl chloride (1.3 mL, 18 mmol, 8 equiv.). The mixture was stirred at RT for 1.5 h. In the meantime, to a solution of aniline I-061 (500 mg, 2.27 mmol, 1 equiv.) in water (30 mL) at 0° C., were added 37% HCl (16 mL) and a solution of NaNO2 (172 mg, 2.49 mmol, 1.1 equiv.) in water (7 mL). The mixture was stirred at 0° C. for 30 min. The first solution was then added dropwise and the resulting mixture was vigorously stirred at RT for 2 h. It was filtered over fritted glass and the filter cake was washed with water. The filter cake was dissolved using DCM and the resulting wet organic solution was dried (Na₂SO₄), filtered and concentrated under reduced pressure to afford 157 mg (23%) of I-062 as a brown solid. ¹H NMR (400 MHZ, Chloroform-d) δ 8.12 (d, J=2.0 Hz, 1H), 8.00 (d, J=2.0 Hz, 1H), 2.65 (s, 3H).

### 3-bromo-N-tert-butyl-5-chloro-4-methyl-benzenesulfonamide (1-063)

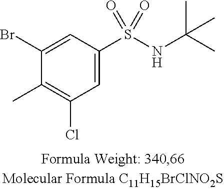 [SMILES: Cc1c(Cl)cc(S(=O)(=O)NC(C)(C)C)cc1Br]

According to GP-4, 1-063 was obtained as a beige solid in 68% yield using I-062 (157 mg, 0.516 mmol, 1 equiv.) and t-butylamine (0.54 mL, 5.17 mmol, 10 equiv.) in DCM (2 mL) at RT for 16 h. Purification by FC (cHex/EtOAc=98/2 to 80/20). ¹H NMR (400 MHZ, Chloroform-d) δ 7.97 (d, J=1.9 Hz, 1H), 7.83 (d, J=1.9 Hz, 1H), 4.51 (s, 1H), 2.58 (s, 3H), 1.28 (s, 9H). MS (ESI⁺): [M+H]⁺ 338.1/340.1/342.1.

### Synthesis of Intermediate Required for Final Product 075

### 3-bromo-4,5-dichloro-benzenesulfonyl chloride (1-064)

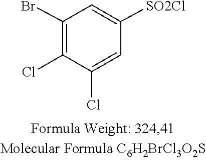 [SMILES: O=S(=O)(Cl)c1cc(Cl)c(Cl)c(Br)c1]

To a solution of CuCl (25 mg, 0.25 mmol, 0.12 equiv.) in water (30 mL) at 0° C., was added dropwise thionyl chloride (1.2 mL, 17 mmol, 8 equiv.). The mixture was stirred at RT for 1.5 h. In the meantime, to a solution of 3-bromo-4,5-dichloroaniline (500 mg, 2.08 mmol, 1 equiv.) in water (30 mL) at 0° C., were added 37% HCl (16 mL) and a solution of NaNO2 (158 mg, 2.28 mmol, 1.1 equiv.) in water (7 mL). The mixture was stirred at 0° C. for 30 min. The first solution was then added dropwise and the resulting mixture was vigorously stirred at RT for 2 h. It was filtered over fritted glass and the filter cake was washed with water. The filter cake was dissolved using DCM and the resulting wet organic solution was dried (Na₂SO₄), filtered and concentrated under reduced pressure to afford 673 mg (100%) of I-064 as a brown solid. ¹H NMR (400 MHZ, Chloroform-d) δ 8.20 (d, J=2.2 Hz, 1H), 8.09 (d, J=2.2 Hz, 1H).

### 3-bromo-N-tert-butyl-4,5-dichloro-benzenesulfonamide (1-065)

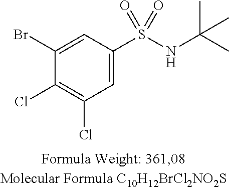 [SMILES: CC(C)(C)NS(=O)(=O)c1cc(Cl)c(Cl)c(Br)c1]

According to GP-4, I-065 was obtained as an orange solid in 14% yield using I-064 (673 mg, 2.07 mmol, 1 equiv.), t-butylamine (0.26 mL, 2.49 mmol, 1.2 equiv.) and Et₃N (0.43 mL, 3.1 mmol, 1.5 equiv.) in DCM (4 mL) at RT for 16 h. Purification by FC (cHex/EtOAc=98/2 to 80/20). ¹H NMR (400 MHZ, Chloroform-d) δ 8.04 (d, J=2.1 Hz, 1H), 7.93 (d, J=2.1 Hz, 1H), 4.54 (s, 1H), 1.30 (s, 9H). MS (ESI⁺): [M+H]⁺ 358.0/360.0/362.0.

### Synthesis of Intermediate Required for Final Product 079

### [2-[3-(2-chloro-4-fluoro-benzoyl)-3,8-diazabicyclo[3.2.1]octan-8-yl]-4-[methyl (propyl) sulfamoyl]phenyl]trifluoromethanesulfonate (1-066)

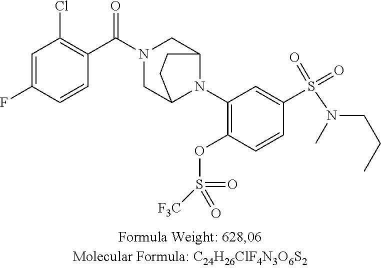 [SMILES: CCCN(C)S(=O)(=O)c1ccc(OS(=O)(=O)C(F)(F)F)c(N2C3CCC2CN(C(=O)c2ccc(F)cc2Cl)C3)c1]

To a solution of 036 (70% pure, 410 mg, 579 μmol, 1 equiv.) dry DCM (5.8 mL) at 0° C., were added Et₃N (0.16 mL, 1.2 mmol, 2 equiv.) and Tf2O (0.19 mL, 1.2 mmol, 2 equiv.). The mixture was stirred at RT for 2 h. Sat. aq. NaHCO₃ and EtOAc were added. The layers were separated and the aqueous phase was extracted with EtOAc. The combined organic extracts were washed (water, brine), dried (Na₂SO₄), filtered and concentrated under reduced pressure. The residue was purified by FC (cHex/EtOAc=95/5 to 50/50) to afford 359 mg (99%) of I-066 as a white foam. ¹H NMR (400 MHZ, Chloroform-d, 2 sets of rotamers) δ 7.46-7.30 (m, 3.5H), 7.25-6.97 (m, 2.5H), 4.61 & 4.58 (s, 1H), 4.16 (s, 1H), 4.00-3.83 (m, 1H), 3.81-3.57 (m, 1H), 3.38 & 3.34 (s, 1H), 3.25-3.15 (m, 1H), 3.05-2.97 (m, 2H), 2.78 (s, 3H), 2.15-1.85 (m, 3.5H), 1.75-1.65 (m, 0.5H), 1.62-1.51 (m, 2H), 0.95 (t, J=7.4 Hz, 3H). ¹⁹F NMR (376 MHz, Chloroform-d, 2 sets of rotamers) δ-73.82,-73.84,-109.0,-109.1. MS (ESI⁺): [M+H]⁺ 628.1/630.1.

### Synthesis of Intermediate Required for Final Products 080 & 81

### [2-[3-(2-chloro-4-fluoro-benzoyl)-3,8-diazabicyclo[3.2.1]octan-8-yl]-4-[(4-phenyl-1-piperidyl) sulfonyl]phenyl]trifluoromethanesulfonate (1-067)

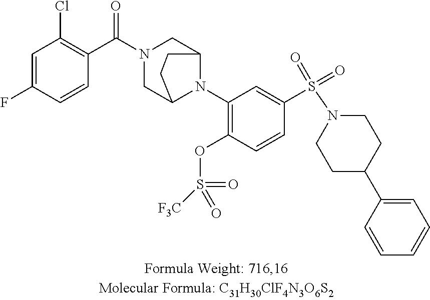 [SMILES: O=C(c1ccc(F)cc1Cl)N1CC2CCC(C1)N2c1cc(S(=O)(=O)N2CCC(c3ccccc3)CC2)ccc1OS(=O)(=O)C(F)(F)F]

To a solution of 063 (100 mg, 171 μmol, 1 equiv.) dry DCM (1.7 mL) at 0° C., were added Et₃N (0.12 mL, 0.86 mmol, 5 equiv.) and Tf2O (0.060 mL, 0.34 mmol, 2 equiv.). The mixture was stirred at RT for 2 h. Sat. aq. NH₄Cl and EtOAc were added. The layers were separated and the aqueous phase was extracted with EtOAc. The combined organic extracts were washed (water, brine), dried (Na₂SO₄), filtereda and concentrated under reduced pressure. The residue was purified by FC (cHex/EtOAc=96/4 to 60/40) to afford 115 mg (94%) of I-067 as a yellow foam. ¹H NMR (400 MHZ, Chloroform-d, 2 sets of rotamers) δ 7.46-7.33 (m, 3.5H), 7.33-7.27 (m, 2H), 7.24-7.18 (m, 2H), 7.18-7.11 (m, 2.5H), 7.12-7.02 (m, 1H), 4.60 & 4.56 (s, 1H), 4.23-4.12 (m, 1H), 3.98-3.85 (m, 3H), 3.77 & 3.61 (d, J=12.5 Hz, 1H), 3.36 & 3.33 (s, 1H), 3.18 (d, J=12.5 Hz, 1H), 2.52 -2.37 (m, 3H), 2.10-1.74 (m, 7.5H), 1.73-1.62 (m, 0.5H). ¹⁹F NMR (376 MHZ, Chloroform-d, 2 sets of rotamers) δ-73.78,-73.81,-108.9,-109.1. MS (ESI⁺): [M+H]⁺ 716.2/718.2.

Synthesis of Intermediates required for final product 083

### 3-bromo-5-fluoro-4-hydroxy-benzenesulfonyl chloride (1-068)

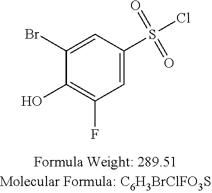 [SMILES: O=S(=O)(Cl)c1cc(F)c(O)c(Br)c1]

According to GP-3, 1-068 was obtained as a yellow solid in 82% yield using 2-bromo-6-fluoro-phenol (1.02 g, 5.35 mmol, 1 equiv.) in HSO₃Cl (8.36 mL, 107 mmol, 20 equiv.) at RT for 2 h. ¹H NMR (400 MHZ, Chloroform-d) 8.02 (t, J=2.0 Hz, 1H), 7.77 (dd, J=8.8, 2.3 Hz, 1H), 5.75 (s, 1H). ¹⁹F NMR (376 MHz, Chloroform-d) 8-128.4.

### 2-bromo-6-fluoro-4-[(4-phenyl-1-piperidyl) sulfonyl]phenol (1-069)

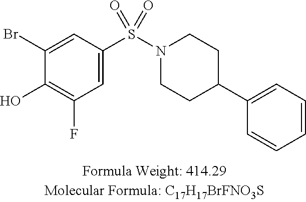 [SMILES: O=S(=O)(c1cc(F)c(O)c(Br)c1)N1CCC(c2ccccc2)CC1]

According to GP-4, I-069 was obtained as a white solid in 88% yield using I-068 (1.27 g, 4.39 mmol, 1 equiv.) 4-phenylpiperidine (1.06 g, 6.58 mmol, 1.5 equiv.) and iPr₂NEt (2.29 mL, 13.1 mmol, 3 equiv.) in DCM (22 mL) at RT for 1 h. Purification by FC (cHex/EtOAc=95/5 to 50/50). ¹H NMR (400 MHz, DMSO-d₆) δ 7.58 (s, 1H), 7.43 (dd, J=10.3, 2.2 Hz, 1H), 7.32-7.24 (m, 2H), 7.23-7.15 (m, 3H), 3.79-3.67 (m, 2H), 3.62 (p, J=6.6 Hz, 1H), 3.40 (br s, 1H), 2.34 (td, J=12.0, 2.5 Hz, 2H), 1.87-1.77 (m, 2H), 1.66 (qd, J=12.6, 4.0 Hz, 2H). ¹⁹F NMR (376 MHz, DMSO-d₆) δ -130.4. MS (ESI⁺): [M+H]⁺ 414.0/416.0/418.0.

### 1-[3-bromo-5-fluoro-4-(methoxymethoxy)phenyl]sulfonyl-4-phenyl-piperidine (1-070)

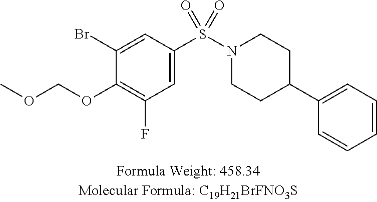 [SMILES: COCOc1c(F)cc(S(=O)(=O)N2CCC(c3ccccc3)CC2)cc1Br]

To a solution of I-069 (1.60 g, 3.86 mmol, 1 equiv.) and iPr₂NEt (2.02 mL, 11.6 mmol, 3 equiv.) in DCM (3 mL) at RT, was added chloromethyl methyl ether (0.59 mL, 7.7 mmol, 2 equiv.). The mixture was stirred at RT for 1 h. Sat. aq. NaHCO₃ was added. The layers were separated and the aqueous phase was extracted with DCM. The combined organic extracts were dried (Na₂SO₄), filtered and concentrated under reduced pressure. The residue was purified by FC (cHex/EtOAc=95/5 to 50/50) to afford 1.85 g of I-070 (100%) as a colorless oil. ¹H NMR (400 MHZ, Chloroform-d) δ 7.80 (s, 1H), 7.51 (dd, J=10.0, 2.0 Hz, 1H), 7.31 (t, J=7.4 Hz, 2H), 7.25-7.20 (m, 1H), 7.19-7.13 (m, 2H), 5.32 (s, 2H), 3.94 (d, J=11.6 Hz, 2H), 3.65 (s, 3H), 2.55-2.40 (m, 3H), 1.99-1.77 (m, 4H). ¹⁹F NMR (376 MHZ, Chloroform-d) 8-122.5. MS (ESI⁺): [M+H]⁺ 458.0/460.0.

### (2-chloro-4-fluoro-phenyl)-[8-[3-fluoro-2-(methoxymethoxy)-5-[(4-phenyl-1-piperidyl) sulfonyl]phenyl]-3,8-diazabicyclo[3.2.1]octan-3-yl]methanone (1-071)

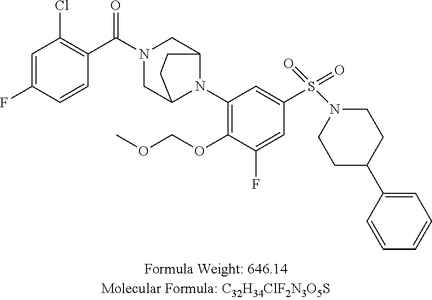 [SMILES: COCOc1c(F)cc(S(=O)(=O)N2CCC(c3ccccc3)CC2)cc1N1C2CCC1CN(C(=O)c1ccc(F)cc1Cl)C2]

According to GP-5, 1-071 was obtained as a white foam in 14% yield using aryl bromide I-070 (300 mg, 622 μmol, 1 equiv.), piperazine I-001 (0.20 g, 0.75 mmol, 1.2 equiv.), Cs₂CO₃ (608 mg, 1.86 mmol, 3 equiv.), Pd (OAc) 2 (14 mg, 62 μmol, 0.1 equiv.) and rac-BINAP (58 mg, 93 μmol, 0.15 equiv.) in toluene (3.1 mL) at reflux for 16 h. Purification by FC (cHex/EtOAc=95/5 to 50/50) and PTLC (DCM/EtOAc=95/5). ¹H NMR (400 MHz, Chloroform-d, 2 sets of rotamers) δ 7.39-7.28 (m, 2.5H), 7.24-6.98 (m, 7.5H), 5.22 & 5.21 (s, 2H), 4.55 & 4.52 (s, 1H), 4.39 (s, 1H), 4.16 (s, 1H), 3.91 (d, J=11.5 Hz, 2H), 3.64-3.40 (m, 4H), 3.27-3.10 (m, 2H), 2.55-2.40 (m, 3H), 2.02-1.77 (m, 7.5H), −1.70-1.60 (m, 0.5H). ¹⁹F NMR (376 MHz, Chloroform-d, 2 sets of rotamers) δ-108.9,-109.1,-125.2,-125.3. MS (ESI⁺): [M+H]⁺ 646.2/648.1.

Synthesis of Intermediates required for final products 084 to 86

### 1-bromo-3-fluoro-2-(methoxymethoxy)benzene (1-072)

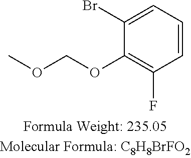

To a solution of 2-bromo-6-fluoro-phenol (1.00 g, 5.24 mmol, 1 equiv.) and iPr₂NEt (2.74 mL, 15.7 mmol, 3 equiv.) in DCM (26 mL) at RT, was added chloromethyl methyl ether (0.60 mL, 7.8 mmol, 2 equiv.). The mixture was stirred at RT for 1 h. 1N HCl was added. The layers were separated and the aqueous phase was extracted with DCM. The combined organic extracts were dried (Na₂SO₄), filtered and concentrated under reduced pressure. The residue was purified by FC (cHex/EtOAc=95/5 to 50/50) to afford 920 mg of I-072 (75%) as a colorless oil. ¹H NMR (400 MHz, Chloroform-d) δ 7.34 (dt, J=8.1, 1.6 Hz, 1H), 7.06 (ddd, J=10.6, 8.3, 1.5 Hz, 1H), 6.94 (td, J=8.2, 5.3 Hz, 1H), 5.20 (s, 2H), 3.65 (s, 3H). ¹⁹F NMR (376 MHz, Chloroform-d) 8-126.0.

### (2-chloro-4-fluoro-phenyl)-[8-[3-fluoro-2-(methoxymethoxy)phenyl]-3,8-diazabicyclo[3.2.1]octan-3-yl]methanone (1-073)

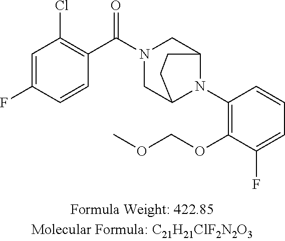 [SMILES: COCOc1c(F)cccc1N1C2CCC1CN(C(=O)c1ccc(F)cc1Cl)C2]

According to GP-5, I-073 was obtained as a white foam in 73% yield using aryl bromide I-072 (920 mg, 3.91 mmol, 1 equiv.), piperazine I-001 (1.16 g, 4.31 mmol, 1.1 equiv.), Cs₂CO₃ (3.82 g, 11.7 mmol, 3 equiv.), Pd (OAc) 2 (87.9 mg, 391 μmol, 0.1 equiv.) and rac-BINAP (292 mg, 470 μmol, 0.12 equiv.) in toluene (20 mL) at reflux for 16 h. Purification by FC (cHex/EtOAc=92/8 to 20/80). ¹H NMR (400 MHZ, Chloroform-d, 2 sets of rotamers) δ 7.36 (dd, J=8.5, 5.9 Hz, 0.5H), 7.24-6.99 (m, 2.5H), 6.97-6.88 (m, 1H), 6.74-6.66 (m, 1H), 6.58 (dd, J=8.2, 4.0 Hz, 1H), 5.17-5.10 (m, 2H), 4.55-4.45 (m, 1H), 4.28 (d, J=6.4 Hz, 1H), 4.15-4.08 (m, 1H), 3.65-3.40 (m, 4H), 3.27 & 3.24 (m, 1H), 3.16-3.06 (m, 1H), 2.06-1.82 (m, 3.5H), 1.65-1.55 (m, 0.5H).¹⁹F NMR (376 MHz, Chloroform-d, 2 sets of rotamers) δ-109.3,-109.4,-129.2,-129.3. MS (ESI⁺): [M+H]⁺ 423.1/425.1.

### (2-chloro-4-fluoro-phenyl)-[8-(3-fluoro-2-hydroxy-phenyl)-3,8-diazabicyclo[3.2.1]octan-3-yl]methanone (1-074)

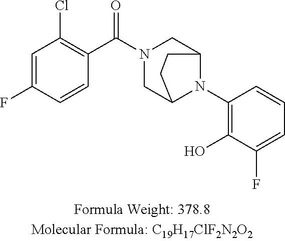 [SMILES: O=C(c1ccc(F)cc1Cl)N1CC2CCC(C1)N2c1cccc(F)c1O]

To a solution of I-073 (1.20 g, 2.84 mmol, 1 equiv.) in DCM (14 mL) at RT, was added CF₃CO₂H (4.4 mL, 57 mmol, 20 equiv.). The mixture was stirred at RT for 2 h. It was concentrated under reduced pressure and the residue was azeotroped with toluene. The residue was purified by FC (cHex/EtOAc=94/6 to 40/60) to afford 833 mg of I-074 (78%) as a white foam. ¹H NMR (400 MHZ, Chloroform-d, 2 sets of rotamers) δ 7.37 (dd, J=8.5, 5.8 Hz, 0.5H), 7.23-7.13 (m, 1.5H), 7.12-7.00 (m, 1H), 6.84-6.70 (m, 2H), 6.63 (t, J=8.9 Hz, 1H), 4.87 (s, 1H), 4.57 & 4.55 (s, 1H), 4.06 (s, 1H), 3.84 (s, 1H), 3.71-3.50 (m, 1H), 3.35 &3.31 (d, J=5.9 Hz, 1H), 3.18 (d, J=12.5 Hz, 1H), 2.04-1.88 (m, 3.5H), 1.70-1.60 (m, 0.5H). ¹⁹F NMR (376 MHz, Chloroform-d, 2 sets of rotamers) δ-109.13,-109.14,-137.5,-137.7. MS (ESI⁺): [M+H]⁺ 379.1/381.1.

### 3-[3-(2-chloro-4-fluoro-benzoyl)-3,8-diazabicyclo[3.2.1]octan-8-yl]-5-fluoro-4-hydroxy-benzenesulfonyl chloride (1-075)

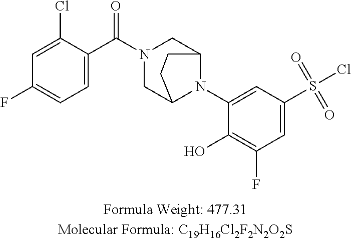 [SMILES: O=C(c1ccc(F)cc1Cl)N1CC2CCC(C1)N2c1cc(S(=O)(=O)Cl)cc(F)c1O]

According to GP-3, I-075 was obtained as a white foam in 83% yield using I-074 (510 mg, 1.34 mmol, 1 equiv.) and HSO₃Cl (1.8 mL, 27 mmol, 20 equiv.) in DCM (7 mL) at reflux for 5 h. ¹H NMR (400 MHZ, Chloroform-d, 2 sets of rotamers) & 7.47 (dt, J=8.4, 1.9 Hz, 1H), 7.38 (dd, J=8.5, 5.8 Hz, 0.5H), 7.25-7.01 (m, 3.5H), 4.65-4.55 (m, 1H), 4.35 (br s, 1H), 4.35-4.20 (m, 1H), 4.03 (s, 1H), 3.70-3.50 (m, 1H), 3.36-3.27 (m, 1H), 3.21 (d, J=12.6 Hz, 1H), 2.17-1.94 (m, 3.5H), 1.75-1.65 (m, 0.5H). ¹⁹F NMR (376 MHZ, Chloroform-d, 2 sets of rotamers) δ-108.65,-108.72,-133.2,-133.4.

Synthesis of Intermediates required for final products 087 to 89

### 1-bromo-2-(methoxymethoxy)-3-(trifluoromethyl)benzene (1-076)

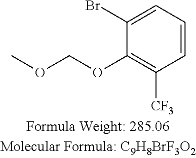

To a solution of 2-bromo-6-(trifluoromethyl) phenol (675 mg, 2.80 mmol, 1 equiv.) and iPr₂NEt (0.73 mL, 4.2 mmol, 1.5 equiv.) in DCM (6 mL) at RT, was added chloromethyl methyl ether (0.26 mL, 3.4 mmol, 1.2 equiv.). The mixture was stirred at RT for 1 h. IN HCl was added. The layers were separated and the aqueous phase was extracted with DCM. The combined organic extracts were dried (Na₂SO₄), filtered and concentrated under reduced pressure. The residue was purified by FC (cHex/EtOAc=98/2 to 90/10) to afford 700 mg of I-076 (88%) as a colorless oil. ¹H NMR (400 MHZ, Chloroform-d) 8 7.79 (dd, J=8.0, 1.5 Hz, 1H), 7.61 (dd, J=7.9, 1.5 Hz, 1H), 7.19-7.04 (m, 1H), 5.22 (s, 2H), 3.70 (s, 3H).¹⁹F NMR (376 MHz, Chloroform-d) 8-60.6.

### (2-chloro-4-fluoro-phenyl)-[8-[2-(methoxymethoxy)-3-(trifluoromethyl)phenyl]-3,8-diazabicyclo[3.2.1]octan-3-yl]methanone (1-077)

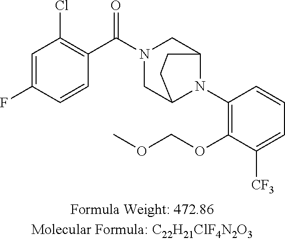 [SMILES: COCOc1c(N2C3CCC2CN(C(=O)c2ccc(F)cc2Cl)C3)cccc1C(F)(F)F]

According to GP-5, I-077 was obtained as a white foam in 71% yield using aryl bromide I-076 (690 mg, 2.42 mmol, 1 equiv.), piperazine I-001 (780 mg, 2.90 mmol, 1.2 equiv.), Cs₂CO₃ (2.37 g, 7.26 mmol, 3 equiv.), Pd (OAc) 2 (54.3 mg, 242 μmol, 0.1 equiv.) and rac-BINAP (181 mg, 291 μmol, 0.12 equiv.) in toluene (12 mL) at reflux for 5 h. Purification by FC (cHex/EtOAc=95/5 to 50/50). ¹H NMR (400 MHZ, Chloroform-d, 2 sets of rotamers) δ 7.39 (dd, J=8.5, 5.8 Hz, 0.5H), 7.25-7.15 (m, 2.5H), 7.14-6.99 (m, 3H), 5.30-5.10 (m, 2H), 4.58-4.50 (m, 1H), 4.35-4.25 (m, 1H), 4.20-4.12 (m, 1H), 3.67-3.45 (m, 4H), 3.35-3.26 (m, 1H), 3.20-3.10 (m, 1H), 2.10-1.85 (m, 3.5H), 1.70-1.60 (m, 0.5H). ¹⁹F NMR (376 MHz, Chloroform-d, 2 sets of rotamers) δ-60.36, -60.40,-109.3,-109.4. MS (ESI⁺): [M+H]⁺ 473.1/475.0.

### (2-chloro-4-fluoro-phenyl)-[8-[2-hydroxy-3-(trifluoromethyl)phenyl]-3,8-diazabicyclo[3.2.1]octan-3-yl]methanone (1-078)

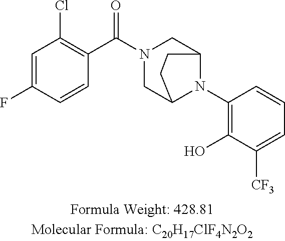 [SMILES: O=C(c1ccc(F)cc1Cl)N1CC2CCC(C1)N2c1cccc(C(F)(F)F)c1O]

To a solution of I-077 (810 mg, 1.71 mmol, 1 equiv.) in DCM (8.6 mL) at RT, was added CF₃CO₂H (2.64 mL, 34 mmol, 20 equiv.). The mixture was stirred at RT for 1 h. It was concentrated under reduced pressure and the residue was azeotroped with toluene. The residue was purified by FC (cHex/EtOAc=95/5 to 50/50) to afford 713 mg of 1-078 (97%) as a white foam. ¹H NMR (400 MHZ, Chloroform-d, 2 sets of rotamers) δ 7.40-7.15 (m, 4H), 7.15-7.03 (m, 2H), 6.95-6.81 (m, 1H), 4.78-4.58 (m, 1H), 3.79-3.39 (m, 3H), 3.39-3.14 (m, 2H), 2.28-1.90 (m, 3.5H), 1.82-1.69 (m, 0.5H). ¹⁹F NMR (376 MHz, Chloroform-d, 2 sets of rotamers) δ-62.31,-62.33,-108.9,-109.0. MS (ESI⁺): [M+H]⁺ 429.0/431.0.

### 3-[3-(2-chloro-4-fluoro-benzoyl)-3,8-diazabicyclo[3.2.1]octan-8-yl]-4-hydroxy-5-(trifluoromethyl)benzenesulfonyl chloride (1-079)

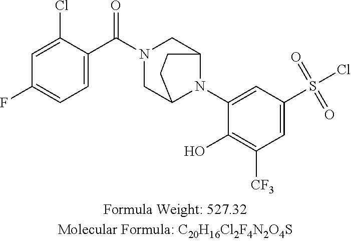 [SMILES: O=C(c1ccc(F)cc1Cl)N1CC2CCC(C1)N2c1cc(S(=O)(=O)Cl)cc(C(F)(F)F)c1O]

According to GP-3, 1-079 was obtained as a white foam in 100% yield using I-078 (710 mg, 1.66 mmol, 1 equiv.) and HSO₃Cl (2.2 mL, 33 mmol, 20 equiv.) in DCM (2 mL) at reflux for 16 h. ¹H NMR (400 MHZ, Chloroform-d, 2 sets of rotamers) δ 8.05 (d, J=2.1 Hz, 1H), 7.67 & 7.64 (d, J=2.2 Hz, 1H), 7.39 (dd, J=8.5, 5.8 Hz, 0.5H), 7.25-7.04 (m, 2.5H), 4.80 (br s, 1H), 4.70 & 4.67 (s, 1H), 3.82 (s, 1H), 3.71-3.48 (m, 2H), 3.36-3.26 (m, 2H), 2.26-2.03 (m, 3.5H), 1.80-1.70 (m, 0.5H). ¹⁹F NMR (376 MHZ, Chloroform-d, 2 sets of rotamers) δ-62.94,-62.96,-108.3,-108.4.

Synthesis of Intermediates required for final product 090

### 3-bromo-4-hydroxy-5-methyl-benzenesulfonyl chloride (1-080)

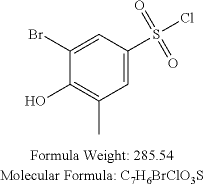

According to GP-3, I-080 was obtained as a brown solid in 68% yield using 2-bromo-6-methyl-phenol (1.00 g, 5.35 mmol, 1 equiv.) in HSO₃Cl (8.36 mL, 107 mmol, 20 equiv.) at RT for 2 h. ¹H NMR (400 MHZ, Chloroform-d) δ 8.03 (d, J=2.4 Hz, 1H), 7.78 (dd, J=2.4, 0.9 Hz, 1H), 6.28 (s, 1H), 2.40 (d, J=0.7 Hz, 3H).

### 2-bromo-6-methyl-4-[(4-phenyl-1-piperidyl) sulfonyl]phenol (1-081)

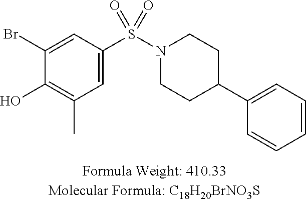 [SMILES: Cc1cc(S(=O)(=O)N2CCC(c3ccccc3)CC2)cc(Br)c1O]

According to GP-4, I-081 was obtained as a white solid in 60% yield using I-080 (1.04 g, 3.64 mmol, 1 equiv.) 4-phenylpiperidine (881 mg, 5.46 mmol, 1.5 equiv.) and iPr₂NEt (1.90 mL, 10.9 mmol, 3 equiv.) in DCM (18 mL) at RT for 1 h. Purification by FC (cHex/EtOAc=95/5 to 50/50). ¹H NMR (400 MHZ, DMSO-d₆) δ 10.23 (s, 1H), 7.68 (d, J=2.3 Hz, 1H), 7.53 (d, J=2.2 Hz, 1H), 7.33-7.24 (m, 2H), 7.23-7.16 (m, 3H), 3.81 -3.67 (m, 2H), 2.58-2.46 (m, 1H), 2.37-2.26 (m, 5H), 1.89-1.76 (m, 2H), 1.75-1.55 (m, 2H). MS (ESI⁺): [M+H]⁺ 410.0/412.0.

### 1-[3-bromo-4-(methoxymethoxy)-5-methyl-phenyl]sulfonyl-4-phenyl-piperidine (1-082)

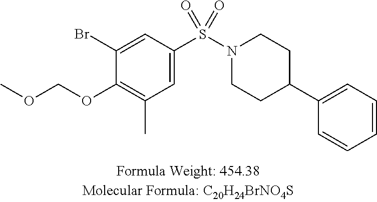 [SMILES: COCOc1c(C)cc(S(=O)(=O)N2CCC(c3ccccc3)CC2)cc1Br]

To a solution of I-081 (900 mg, 2.19 mmol, 1 equiv.) and iPr₂NEt (1.15 mL, 6.58 mmol, 3 equiv.) in DCM (11 mL) at RT, was added chloromethyl methyl ether (0.33 mL, 4.4 mmol, 2 equiv.). The mixture was stirred at RT for 1 h. Sat. aq. NaHCO₃ was added. The layers were separated and the aqueous phase was extracted with DCM. The combined organic extracts were dried (Na₂SO₄), filtered and concentrated under reduced pressure. The residue was purified by FC (cHex/EtOAc=95/5 to 50/50) to afford 880 mg of I-082 (89%) as a colorless oil. ¹H NMR (400 MHZ, Chloroform-d) δ 7.84 (d, J=2.2 Hz, 1H), 7.58-7.54 (m, 1H), 7.34-7.27 (m, 2H), 7.25-7.18 (m, 1H), 7.18-7.13 (m, 2H), 5.16 (s, 2H), 3.97-3.88 (m, 2H), 3.67 (s, 3H), 2.53-2.38 (m, 6H), 1.97-1.79 (m, 4H). MS (ESI⁺): [M+H]⁺ 454.0/456.0.

### (2-chloro-4-fluoro-phenyl)-[8-[2-(methoxymethoxy)-3-methyl-5-[(4-phenyl-1-piperidyl) sulfonyl]phenyl]-3,8-diazabicyclo[3.2.1]octan-3-yl]methanone (1-083)

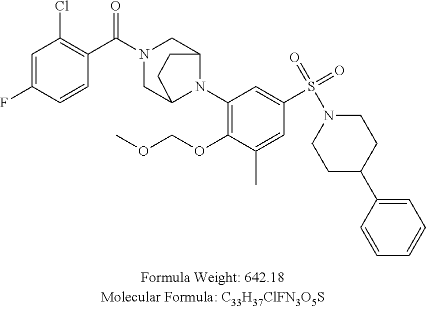 [SMILES: COCOc1c(C)cc(S(=O)(=O)N2CCC(c3ccccc3)CC2)cc1N1C2CCC1CN(C(=O)c1ccc(F)cc1Cl)C2]

According to GP-5, 1-083 was obtained as a white foam in 44% yield using aryl bromide I-082 (230 mg, 460 μmol, 1 equiv.), piperazine I-001 (0.15 g, 0.55 mmol, 1.2 equiv.), Cs₂CO₃ (450 mg, 1.38 mmol, 3 equiv.), Pd (OAc) 2 (10 mg, 46 μmol, 0.1 equiv.) and rac-BINAP (43 mg, 69 μmol, 0.15 equiv.) in toluene (2.3 mL) at reflux for 16 h. Purification by FC (cHex/EtOAc=90/10 to 10/90). ¹H NMR (400 MHZ, Chloroform-d, 2 sets of rotamers) δ 7.40-7.27 (m, 2.5H), 7.24-7.00 (m, 7.5H), 5.21-5.14 (m, 2H), 4.55 & 4.52 (m, 1H), 4.29 (s, 1H), 4.06 (s, 1H), 3.95-3.85 (m, 2H), 3.65-3.40 (m, 4H), 3.36-3.12 (m, 2H), 2.50-2.32 (m, 6H), 2.03-1.76 (m, 7.5H), 1.70-1.60 (m, 0.5H). ¹⁹F NMR (376 MHz, Chloroform-d, 2 sets of rotamers) δ-109.1,-109.2. MS (ESI⁺): [M+H]⁺ 642.2/644.1.

Synthesis of Intermediates required for final products 091 to 93

### 1-bromo-2-(methoxymethoxy)-3-methyl-benzene (1-084)

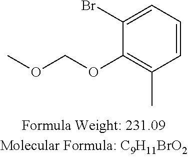

To a solution of 2-bromo-6-methyl-phenol (1.00 g, 5.35 mmol, 1 equiv.) and iPr₂NEt (1.4 mL, 8.1 mmol, 1.5 equiv.) in DCM (21 mL) at RT, was added chloromethyl methyl ether (0.49 mL, 6.4 mmol, 1.2 equiv.). The mixture was stirred at RT for 60 h. 1N HCl was added. The layers were separated and the aqueous phase was extracted with DCM. The combined organic extracts were dried (Na₂SO₄), filtered and concentrated under reduced pressure to afford 1.22 g of I-084 (99%) as a colorless oil. ¹H NMR (400 MHZ, Chloroform-d) δ 7.42 (ddd, J=8.0, 1.6, 0.7 Hz, 1H), 7.15 (ddd, J=7.6, 1.6, 0.8 Hz, 1H), 6.92 (t, J=7.8 Hz, 1H), 5.11 (s, 2H), 3.68 (s, 3H), 2.39 (s, 3H).

### (2-chloro-4-fluoro-phenyl)-[8-[2-(methoxymethoxy)-3-methyl-phenyl]-3,8-diazabicyclo[3.2.1]octan-3-yl]methanone (1-085)

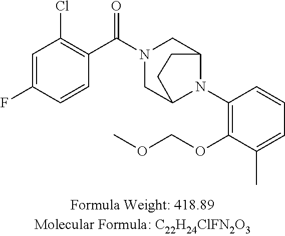 [SMILES: COCOc1c(C)cccc1N1C2CCC1CN(C(=O)c1ccc(F)cc1Cl)C2]

According to GP-7, 1-085 was obtained as a white foam in 24% yield using aryl bromide I-084 (811 mg, 3.51 mmol, 1 equiv.), piperazine I-001 (1.22 g, 4.56 mmol, 1.3 equiv.), t-BuONa (1.01 g, 10.5 mmol, 3 equiv.) and XPhos-Pd-G3 (148 mg, 175 μmol, 0.05 equiv.) in toluene (14 mL) at reflux for 4 h. Purification by FC (cHex/EtOAc=95/5 to 50/50). 1H NMR (400 MHZ, Chloroform-d, 2 sets of rotamers) δ 7.42-7.35 (m, 0.5H), 7.26-7.14 (m, 1.5H), 7.13-7.00 (m, 1H), 6.97-6.89 (m, 1H), 6.87-6.76 (m, 1H), 6.68 (t, J=6.7 Hz, 1H), 5.27-4.98 (m, 2H), 4.54 & 4.50 (d, J=6.1 Hz, 1H), 4.24 (s, 1H), 4.06 (s, 1H), 3.64-3.42 (m, 4H), 3.31-3.24 (m, 1H), 3.14 (t, J=11.1 Hz, 1H), 2.33 (s, 3H), 2.05-1.83 (m, 3.5H), 1.64-1.56 (m, 0.5H). ¹⁹F NMR (376 MHZ, Chloroform-d, 2 sets of rotamers) δ-109.5,-109.6. MS (ESI⁺): [M+H]⁺ 419.1/421.0.

### (2-chloro-4-fluoro-phenyl)-[8-(2-hydroxy-3-methyl-phenyl)-3,8-diazabicyclo[3.2.1]octan-3-yl]methanone (1-086)

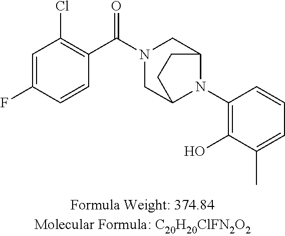 [SMILES: Cc1cccc(N2C3CCC2CN(C(=O)c2ccc(F)cc2Cl)C3)c1O]

To a solution of I-085 (350 mg, 0.835 mmol, 1 equiv.) in DCM (4.2 mL) at RT, was added CF₃CO₂H (1.3 mL, 17 mmol, 20 equiv.). The mixture was stirred at RT for 24 h. It was concentrated under reduced pressure and the residue was azeotroped with toluene to afford 330 mg of I-086 (100%) as a white foam. ¹H NMR (400 MHZ, Chloroform-d, 2 sets of rotamers) δ 7.40 (dd, J=8.5, 5.8 Hz, 0.5H), 7.26-7.15 (m, 1.5H), 7.15-7.05 (m, 1H), 7.01-6.88 (m, 1H), 6.88-6.60 (m, 2H), 4.65 & 4.62 (dd, J=2.7, 1.4 Hz, 1H), 3.84 -3.61 (m, 2H), 3.56-3.48 (m, 2H), 3.40-3.17 (m, 2H), 2.29 (s, 3H), 2.24-1.90 (m, 3.5H), 1.73-1.65 (m, 0.5H). ¹⁹F NMR (376 MHz, Chloroform-d, 2 sets of rotamers) δ-109.07,-109.11. MS (ESI⁺): [M+H]⁺ 375.1/377.1.

### 3-[3-(2-chloro-4-fluoro-benzoyl)-3,8-diazabicyclo[3.2.1]octan-8-yl]-4-hydroxy-5-methyl-benzenesulfonyl chloride (1-087)

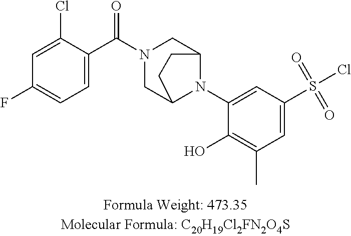 [SMILES: Cc1cc(S(=O)(=O)Cl)cc(N2C3CCC2CN(C(=O)c2ccc(F)cc2Cl)C3)c1O]

According to GP-3, 1-087 was obtained as a beige foam in 100% yield using I-086 (330 mg, 0.863 mmol, 1 equiv.) and HSO₃Cl (1.4 mL, 22 mmol, 20 equiv.) in DCM (3.5 mL) at RT for 1 h. ¹H NMR (400 MHZ, Chloroform-d, 2 sets of rotamers) δ 7.70-7.68 (m, 1H), 7.44-7.38 (m, 1.5H), 7.27-7.16 (m, 1.5H), 7.17-7.05 (m, 1H), 4.69 & 4.66 (s, 1H), 3.83-3.73 (m, 1H), 3.72-3.45 (m, 2H), 3.35-3.25 (m, 2H), 2.37 (s, 3H), 2.26-2.00 (m, 3.5H), 1.82-1.72 (m, 0.5H).¹⁹F NMR (376 MHz, Chloroform-d, 2 sets of rotamers) δ-108.6,-108.8.

Synthesis of Intermediates required for final product 094

### 1-bromo-3-ethyl-2-(methoxymethoxy)benzene (1-088)

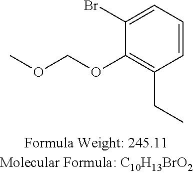

To a solution of 2-bromo-6-ethyl-phenol (500 mg, 2.49 mmol, 1 equiv.) and iPr₂NEt (0.65 mL, 3.7 mmol, 1.5 equiv.) in DCM (12 mL) at RT, was added chloromethyl methyl ether (0.23 mL, 3.0 mmol, 1.2 equiv.). The mixture was stirred at RT for 16 h. Sat. aq. NH₄Cl and MTBE were added. The layers were separated and the aqueous phase was extracted with MTBE. The combined organic extracts were washed (brine), dried (Na₂SO₄), filtered and concentrated under reduced pressure to afford 647 mg of I-088 (100%) as a colorless oil. ¹H NMR (400 MHZ, Chloroform-d) δ 7.32 (dd, J=7.9, 1.6 Hz, 1H), 7.13-6.99 (m, 1H), 6.87 (t, J=7.8 Hz, 1H), 5.01 (s, 2H), 3.57 (s, 3H), 2.68 (q, J=7.5 Hz, 2H), 1.16 (t, J=7.6 Hz, 3H).

### (2-chloro-4-fluoro-phenyl)-[8-[3-ethyl-2-(methoxymethoxy)phenyl]-3,8-diazabicyclo[3.2.1]octan-3-yl]methanone (1-089)

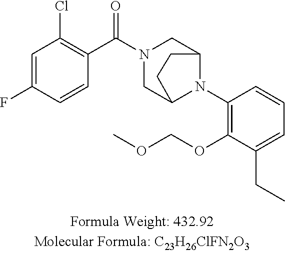 [SMILES: CCc1cccc(N2C3CCC2CN(C(=O)c2ccc(F)cc2Cl)C3)c1OCOC]

According to GP-5, I-089 was obtained as a white foam in 68% yield using aryl bromide I-088 (640 mg, 2.61 mmol, 1 equiv.), piperazine I-001 (842 mg, 3.13 mmol, 1.2 equiv.), Cs₂CO₃ (2.55 g, 7.83 mmol, 3 equiv.), Pd (OAc) 2 (58.6 mg, 261 μmol, 0.1 equiv.) and rac-BINAP (195 mg, 313 μmol, 0.12 equiv.) in toluene (13 mL) at reflux for 3 h. Purification by FC (cHex/EtOAc=90/10 to 10/90). ¹H NMR (400 MHZ, Chloroform-d, 2 sets of rotamers) ¹H NMR (400 MHZ, Chloroform-d, 2 sets of rotamers) δ 7.38 (dd, J=8.5, 5.8 Hz, 0.5H), 7.25-7.14 (m, 1.5H), 7.14-7.01 (m, 1H), 7.01-6.93 (m, 1H), 6.91 -6.80 (m, 1H), 6.76-6.61 (m, 1H), 5.23-5.03 (m, 2H), 4.62-4.45 (m, 1H), 4.30-4.19 (m, 1H), 4.09-4.00 (m, 1H), 3.67-3.40 (m, 4H), 3.36-3.21 (m, 1H), 3.21-3.03 (m, 1H), 2.79-2.64 (m, 2H), 2.04-1.82 (m, 3.5H), 1.65-1.55 (m, 0.5H), 1.25 (t, J=7.4 Hz, 3H). ¹⁹F NMR (376 MHZ, Chloroform-d, 2 sets of rotamers) δ-109.5,-109.6. MS (ESI⁺): [M+H]⁺ 433.1/435.0.

### (2-chloro-4-fluoro-phenyl)-[8-(3-ethyl-2-hydroxy-phenyl)-3,8-diazabicyclo[3.2.1]octan-3-yl]methanone (1-090)

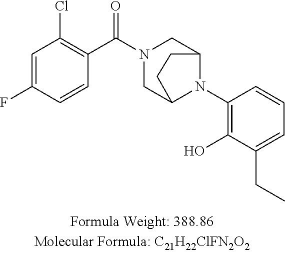 [SMILES: CCc1cccc(N2C3CCC2CN(C(=O)c2ccc(F)cc2Cl)C3)c1O]

To a solution of I-089 (750 mg, 1.73 mmol, 1 equiv.) in DCM (8.7 mL) at RT, was added CF₃CO₂H (2.7 mL, 34 mmol, 20 equiv.). The mixture was stirred at RT for 24 h. It was concentrated under reduced pressure and the residue was azeotroped with toluene. The residue was purified by FC (cHex/EtOAc=95/5 to 50/50) to afford 618 mg of I-090 (92%) as a white foam. ¹H NMR (400 MHZ, Chloroform-d, 2 sets of rotamers) δ 7.40 (dd, J=8.5, 5.8 Hz, 0.5H), 7.26-7.15 (m, 1.5H), 7.15-7.02 (m, 1H), 7.02-6.90 (m, 1H), 6.90 -6.66 (m, 3H), 4.68-4.54 (m, 1H), 3.78-3.41 (m, 3H), 3.36-3.14 (m, 2H), 2.70 (q, J=7.5 Hz, 2H), 2.32-1.86 (m, 3.5H), 1.77-1.65 (m, 0.5H), 1.25 (t, J=7.6 Hz, 3H).19F NMR (376 MHz, Chloroform-d, 2 sets of rotamers) δ-109.2,-109.3. MS (ESI⁺): [M+H]⁺ 389.0/391.0.

### 3-[3-(2-chloro-4-fluoro-benzoyl)-3,8-diazabicyclo[3.2.1]octan-8-yl]-5-ethyl-4-hydroxy-benzenesulfonyl chloride (1-091)

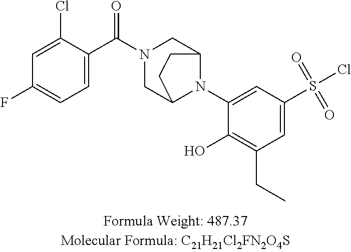 [SMILES: CCc1cc(S(=O)(=O)Cl)cc(N2C3CCC2CN(C(=O)c2ccc(F)cc2Cl)C3)c1O]

According to GP-3, 1-091 was obtained as a beige foam in 80% yield using I-090 (150 mg, 0.386 mmol, 1 equiv.) and HSO₃Cl (0.51 mL, 7.7 mmol, 20 equiv.) in DCM (1 mL) at RT for 1 h. ¹H NMR (400 MHZ, Chloroform-d, 2 sets of rotamers) δ 7.74-7.67 (m, 1H), 7.48 -7.36 (m, 1.5H), 7.24-7.03 (m, 2.5H), 4.72-4.63 (m, 1H), 3.92-3.54 (m, 3H), 3.43-3.26 (m, 2H), 2.75 (q, J=7.5 Hz, 2H), 2.22-1.99 (m, 3.5H), 1.80-1.70 (m, 0.5H), 1.28 (t, J=7.5 Hz, 3H). ¹⁹F NMR (376 MHz, Chloroform-d) 8-108.5.

Synthesis of Intermediates required for final products 095 to 109

### 1-bromo-3-chloro-2-(methoxymethoxy)benzene (1-092)

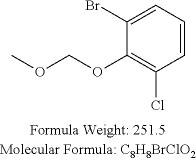

To a solution of 2-bromo-6-chlorophenol (3.17 g, 10.5 mmol, 1 equiv.) and iPr₂NEt (3.73 mL, 15.7 mmol, 1.5 equiv.) in DCM (21 mL) at RT, was added chloromethyl methyl ether (0.95 mL, 13 mmol, 1.2 equiv.). The mixture was stirred at RT for 16 h. The volatiles were removed under reduced pressure. EtOAc was added and the organic solution was washed washed (1N HCl, brine), dried (Na₂SO₄), filtered and concentrated under reduced pressure. The residue was purified by FC (cHex/EtOAc=95/5) to afford 2.59 g of I-092 (98%) as a colorless oil. ¹H NMR (400 MHZ, Chloroform-d) δ 7.50 (dd, J=8.0, 1.6 Hz, 1H), 7.37 (dd, J=8.1, 1.5 Hz, 1H), 6.97 (t, J=8.0 Hz, 1H), 5.21 (s, 2H), 3.74 (s, 3H).

### (2-chloro-4-fluoro-phenyl)-[8-[3-chloro-2-(methoxymethoxy)phenyl]-3,8-diazabicyclo[3.2.1]octan-3-yl]methanone (1-093)

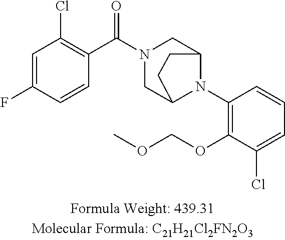 [SMILES: COCOc1c(Cl)cccc1N1C2CCC1CN(C(=O)c1ccc(F)cc1Cl)C2]

According to GP-5, 1-093 was obtained as a white foam in 79% yield using aryl bromide I-092 (1.20 g, 4.77 mmol, 1 equiv.), piperazine I-001 (1.54 g, 5.73 mmol, 1.2 equiv.), Cs₂CO₃ (4.66 g, 14.3 mmol, 3 equiv.), Pd (OAc) 2 (107 mg, 477 μmol, 0.1 equiv.) and rac-BINAP (357 mg, 573 μmol, 0.12 equiv.) in toluene (14 mL) at reflux for 5 h. Purification by FC (cHex/EtOAc=95/5 to 50/50). ¹H NMR (400 MHZ, Chloroform-d, 2 sets of rotamers) δ 7.38 (dd, J=8.5, 5.8 Hz, 0.5H), 7.26-7.14 (m, 1.5H), 7.14-7.02 (m, 1H), 7.02-6.92 (m, 2H), 6.80-6.69 (m, 1H), 5.24-5.11 (m, 2H), 4.54 & 4.50 (dd, J=5.4, 2.3 Hz, 1H), 4.32-4.21 (m, 1H), 4.14-4.10 (m, 1H), 3.70-3.40 (m, 4H), 3.29 & 3.26 (d, J=2.0 Hz, 1H), 3.20-3.08 (m, 1H), 2.10-1.80 (m, 3.5H), 1.67-1.57 (m, 0.5H).¹⁹F NMR (376 MHZ, Chloroform-d, 2 sets of rotamers) δ-109.3,-109.4. MS (ESI⁺): [M+H]⁺ 439.1/441.1/443.1.

### (2-chloro-4-fluoro-phenyl)-[8-(3-chloro-2-hydroxy-phenyl)-3,8-diazabicyclo[3.2.1]octan-3-yl]methanone (1-094)

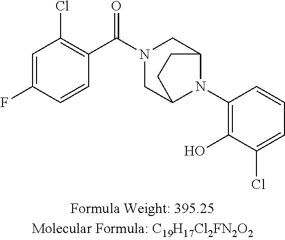 [SMILES: O=C(c1ccc(F)cc1Cl)N1CC2CCC(C1)N2c1cccc(Cl)c1O]

To a solution of I-093 (1.60 g, 3.64 mmol, 1 equiv.) in DCM (18 mL) at RT, was added CF₃CO₂H (5.6 mL, 73 mmol, 20 equiv.). The mixture was stirred at RT for 3 h. It was concentrated under reduced pressure and the residue was azeotroped with toluene. The residue was purified by FC (cHex/EtOAc=95/5 to 50/50) to afford 1.47 g of I-094 (99%) as a white foam. ¹H NMR (400 MHZ, Chloroform-d, 2 sets of rotamers) δ 7.39 (dd, J=8.5, 5.8 Hz, 0.5H), 7.27-7.14 (m, 1.5H), 7.14-7.00 (m, 2H), 6.85-6.75 (m, 2H), 5.63 (br s, 1H), 4.66-4.54 (m, 1H), 4.07-4.00 (m, 1H), 3.88-3.74 (m, 1H), 3.73-3.48 (m, 1H), 3.35 & 3.32 (s, 1H), 3.26-3.15 (m, 1H), 2.20-1.90 (m, 3.5H), 1.75-1.65 (m, 0.5H). ¹⁹F NMR (376 MHZ, Chloroform-d, 2 sets of rotamers) δ-109.1,-109.2. MS (ESI⁺): [M+H]⁺ 395.0/397.0/399.0.

### 3-chloro-5-[3-(2-chloro-4-fluoro-benzoyl)-3,8-diazabicyclo[3.2.1]octan-8-yl]-4-hydroxy-benzenesulfonyl chloride (1-095)

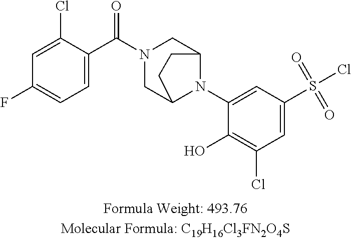 [SMILES: O=C(c1ccc(F)cc1Cl)N1CC2CCC(C1)N2c1cc(S(=O)(=O)Cl)cc(Cl)c1O]

According to GP-3, I-095 was obtained as a beige solid in 97% yield using I-094 (735 mg, 1.77 mmol, 1 equiv.) and HSO₃Cl (2.9 mL, 44 mmol, 20 equiv.) in DCM (7 mL) at reflux for 4 h. ¹H NMR (400 MHZ, Chloroform-d, 2 sets of rotamers) δ 7.75 (d, J=2.1 Hz, 1H), 7.40 (dd, J=8.5, 5.8 Hz, 0.5H), 7.38-7.32 (m, 1H), 7.27-7.16 (m, 1.5H), 7.16-7.03 (m, 1H), 4.70-4.53 (m, 1H), 4.33-4.15 (m, 1H), 3.97 (s, 1H), 3.71-3.48 (m, 1H), 3.41 -3.17 (m, 2H), 2.30-1.89 (m, 3.5H), 1.80-1.70 (m, 0.5H). ¹⁹F NMR (376 MHz, Chloroform-d, 2 sets of rotamers) δ-108.7,-108.8.

Synthesis of Intermediates required for final product 110

### (2-chloro-4-fluoro-phenyl)-[(3S)-4-[3-chloro-2-(methoxymethoxy)phenyl]-3-methyl-piperazin-1-yl]methanone ((S)-1-096)

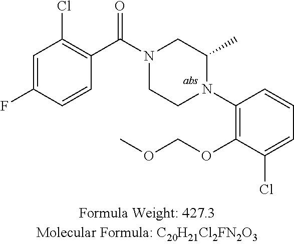 [SMILES: COCOc1c(Cl)cccc1N1CCN(C(=O)c2ccc(F)cc2Cl)C[C@@H]1C]

According to GP-5, (S)-I-096 was obtained as a white foam in 4% yield using aryl bromide I-092 (0.41 g, 1.63 mmol, 1 equiv.), piperazine(S)-I-003 (502 mg, 1.96 mmol, 1.2 equiv.), Cs₂CO₃ (4.66 g, 4.89 mmol, 3 equiv.), Pd (OAc) 2 (36.6 mg, 163 μmol, 0.1 equiv.) and rac-BINAP (122 mg, 196 μmol, 0.12 equiv.) in toluene (8.2 mL) at reflux for 6 h. Purification by FC (cHex/EtOAc=94/6 to 40/60) and PTLC (cHex/EtOAc=70/30). 1H NMR (400 MHZ, Chloroform-d, multiple sets of rotamers) δ 7.27-7.20 (m, 1H), 7.16-6.88 (m, 4H), 6.88-6.73 (m, 1H), 5.26-5.18 (m, 2H), 5.17-5.10 (m, 1H), 4.17-3.81 (m, 1H), 3.80-3.50 (m, 4H), 3.48-2.95 (m, 3H), 2.87-2.50 (m, 1H), 1.02-0.70 (m, 3H). ¹⁹F NMR (376 MHz, Chloroform-d, multiple sets of rotamers) δ-109.17,-109.20,-109.24,-109.27. MS (ESI⁺): [M+H]⁺ 427.0/429.0/431.0.

### (2-chloro-4-fluoro-phenyl)-[(3S)-4-(3-chloro-2-hydroxy-phenyl)-3-methyl-piperazin-1-yl]methanone ((S)-1-097)

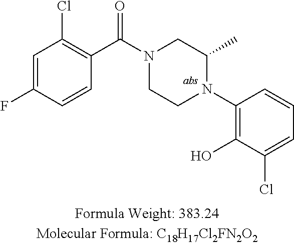 [SMILES: C[C@H]1CN(C(=O)c2ccc(F)cc2Cl)CCN1c1cccc(Cl)c1O]

To a solution of(S)-1-096 (26 mg, 61 μmol, 1 equiv.) in DCM (0.3 mL) at RT, was added CF₃CO₂H (90 μL, 1.2 mol, 20 equiv.). The mixture was stirred at RT for 2 h. It was concentrated under reduced pressure and the residue was azeotroped with toluene. The residue was purified by FC (cHex/EtOAc=95/5 to 50/50) to afford 22 mg of(S)-I-097 (94%) as a white foam. ¹H NMR (400 MHZ, Chloroform-d, multiple sets of rotamers) 8 7.56-7.19 (m, 2H), 7.15-7.07 (m, 2H), 7.05-6.90 (m, 2H), 6.80-6.72 (m, 1H), 4.83 -4.54 (m, 1H), 3.45-2.50 (m, 6H), 0.90-0.60 (m, 3H). ¹⁹F NMR (376 MHZ, Chloroform-d, multiple sets of rotamers) δ-108.87,-108.88,-108.93,-108.96. MS (ESI⁺): [M+H]⁺ 383.0/385.0/387.0.

### 3-chloro-5-[(2S)-4-(2-chloro-4-fluoro-benzoyl)-2-methyl-piperazin-1-yl]-4-hydroxy-benzenesulfonyl chloride ((S)-1-098)

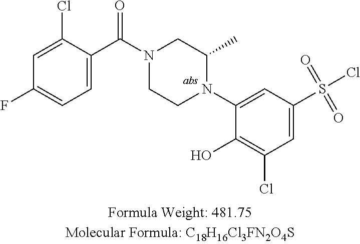 [SMILES: C[C@H]1CN(C(=O)c2ccc(F)cc2Cl)CCN1c1cc(S(=O)(=O)Cl)cc(Cl)c1O]

According to GP-3, (S)-I-098 was obtained as a beige solid in 90% yield using(S)-I-097 (22 mg, 57 μmol, 1 equiv.) and HSO₃Cl (0.10 mL, 1.4 mmol, 20 equiv.) in DCM (0.3 mL) at reflux for 4 h. ¹H NMR (400 MHZ, Chloroform-d, multiple sets of rotamers) δ 7.90-7.86 (m, 1H), 7.66-7.62 (m, 1H), 7.36-7.08 (m, 3H), 7.08-6.98 (m, 1H), 4.82-4.54 (m, 1H), 3.50-2.72 (m, 6H), 0.95-0.65 (m, 3H). ¹⁹F NMR (376 MHZ, Chloroform-d, multiple sets of rotamers) δ-108.40,-108.46,-108.49,-108.53.

Synthesis of Intermediates required for final product 111

### 3-chloro-5-[(2R)-4-(2-chloro-4-fluoro-benzoyl)-2-methyl-piperazin-1-yl]-4-hydroxy-benzenesulfonyl chloride ((R)-1-098)

(R)-I-098 was obtained using the same synthetic sequence than the one used for(S)-I-098 starting from (R)-I-003. ¹H NMR (400 MHZ, Chloroform-d, multiple sets of rotamers) δ 7.90-7.86 (m, 1H), 7.66-7.62 (m, 1H), 7.36-7.08 (m, 3H), 7.08-6.98 (m, 1H), 4.82-4.54 (m, 1H), 3.50-2.72 (m, 6H), 0.95-0.65 (m, 3H). ¹⁹F NMR (376 MHz, Chloroform-d, multiple sets of rotamers) δ-108.40,-108.46,-108.49,-108.53.

### Synthesis of Intermediate Required for Final Product 115

3-bromo-4-chloro-N-methyl-N-propyl-benzenesulfonamide (1-099)

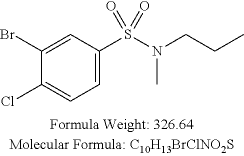 [SMILES: CCCN(C)S(=O)(=O)c1ccc(Cl)c(Br)c1]

According to GP-4, 1-099 was obtained as a white solid in 89% yield using commercial 3-bromo-4-chlorobenzenesulfonyl chloride (300 mg, 1.03 mmol, 1 equiv.), methyl-N-propylamine (120 μL, 1.14 mmol, 1.1 equiv.) and Et₃N (216 μL, 2.55 mmol, 1.5 equiv.) in DCM (5 mL) at RT for 16 h. ¹H NMR (400 MHZ, Chloroform-d) § 8.04 (d, J=2.0 Hz, 1H), 7.66 (dd, J=8.4, 2.0 Hz, 1H), 7.61 (d, J=8.4 Hz, 1H), 3.00 (dd, J=7.9, 6.6 Hz, 2H), 2.76 (s, 3H), 1.59 (h, J=7.3 Hz, 2H), 0.94 (t, J=7.4 Hz, 3H). MS (ESI⁺): [M+H]⁺ 326.0/328.0/329.9.

Synthesis of Intermediate Required for Final Product 116 1-(3-bromo-4-methyl-phenyl) sulfonyl-4-[2-(4-fluorophenyl)ethyl]piperazine (I-100)

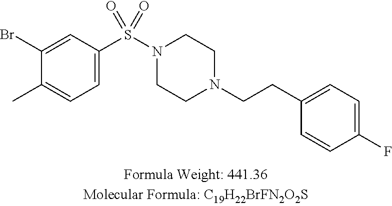 [SMILES: Cc1ccc(S(=O)(=O)N2CCN(CCc3ccc(F)cc3)CC2)cc1Br]

According to GP-4, I-100 was obtained as a white solid in 88% yield using commercial 3-bromo-4-methylbenzenesulfonyl chloride (200 mg, 742 μmol, 1 equiv.), 1-[2-(4-fluorophenyl)ethyl]piperazine dihydrochloride (229 mg, 0.816 mmol, 1.1 equiv.) and Et₃N (0.52 mL, 3.7 mmol, 5 equiv.) in DCM (4 mL) at RT for 16 h. Purification by FC (cHex/EtOAc=97/3 to 70/30). ¹H NMR (400 MHZ, Chloroform-d) δ 7.92 (d, J=1.8 Hz, 1H), 7.59 (dd, J=8.0, 1.9 Hz, 1H), 7.40 (d, J=8.0 Hz, 1H), 7.16-7.06 (m, 2H), 7.02-6.90 (m, 2H), 3.06 (t, J=4.8 Hz, 4H), 2.75-2.68 (m, 2H), 2.66-2.52 (m, 6H), 2.48 (s, 3H). ¹⁹F NMR (376 MHz, Chloroform-d) 8-117.0. MS (ESI⁺): [M+H]⁺ 440.9/442.9.

Synthesis of Intermediates required for final products 117

### [8-(1,3-benzodioxol-4-yl)-3,8-diazabicyclo[3.2.1]octan-3-yl]-(2-chloro-4-fluoro-phenyl) methanone (1-101)

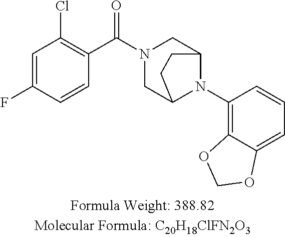 [SMILES: O=C(c1ccc(F)cc1Cl)N1CC2CCC(C1)N2c1cccc2c1OCO2]

According to GP-7, I-101 was obtained as a white solid in 90% yield using 4-bromo-1,3-benzodioxole (400 mg, 1.99 mmol, 1 equiv.), piperazine I-001 (642 mg, 2.39 mmol, 1.2 equiv.), t-BuONa (574 mg, 5.97 mmol, 3 equiv.) and XPhos-Pd-G3 (168 mg, 199 μmol, 0.1 equiv.) in toluene (10 mL) at reflux for 2 h. Purification by FC (cHex/EtOAc=95/5 to 0/100). ¹H NMR (400 MHZ, Chloroform-d, 2 sets of rotamers) δ 7.36 (dd, J=8.5, 5.8 Hz, 0.5H), 7.23-6.95 (m, 2.5H), 6.77 (t, J=8.1 Hz, 1H), 6.46-6.33 (m, 2H), 5.90-5.84 (m, 2H), 4.46-4.35 (m, 2H), 4.26-4.20 (m, 1H), 3.66-3.43 (m, 1H), 3.33-3.25 (m, 1H), 3.06-2.95 (m, 1H), 2.12-1.87 (m, 3.5H), 1.66-1.58 (m, 0.5H). ¹⁹F NMR (376 MHz, Chloroform-d, 2 sets of rotamers) δ-109.4,-109.5. MS (ESI⁺): [M+H]⁺ 389.1/391.1.

### 7-[3-(2-chloro-4-fluoro-benzoyl)-3,8-diazabicyclo[3.2.1]octan-8-yl]-1,3-benzodioxole-5-sulfonyl chloride (1-102)

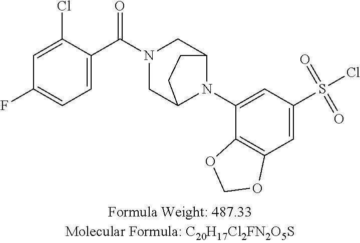 [SMILES: O=C(c1ccc(F)cc1Cl)N1CC2CCC(C1)N2c1cc(S(=O)(=O)Cl)cc2c1OCO2]

According to GP-3, I-102 was obtained as a beige solid in 50% yield using I-101 (100 mg, 0.257 mmol, 1 equiv.) and HSO₃Cl (0.34 mL, 5.1 mmol, 20 equiv.) in DCM (1.3 mL) at RT for 1 h. ¹H NMR (400 MHZ, Chloroform-d, 2 sets of rotamers) δ 7.40-7.33 (m, 0.5H), 7.27-7.08 (m, 2.5H), 7.06-7.00 (m, 2H), 6.11-6.05 (m, 2H), 4.55-4.41 (m, 2H), 4.26 (s, 1H), 3.63-3.38 (m, 1H), 3.26-3.03 (m, 2H), 2.15-1.90 (m, 3.5H), 1.80-1.60 (m, 0.5H). ¹⁹F NMR (376 MHz, Chloroform-d, 2 sets of rotamers) δ-108.9,-109.0.

### Synthesis of Intermediate Required for Final Product 118

### 1-[(5-bromo-2,3-dihydro-1,4-benzodioxin-7-yl) sulfonyl]-4-phenyl-piperidine (I-103)

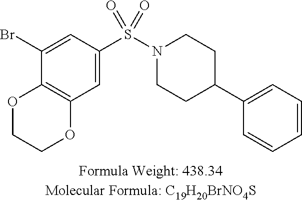 [SMILES: O=S(=O)(c1cc(Br)c2c(c1)OCCO2)N1CCC(c2ccccc2)CC1]

According to GP-4, I-103 was obtained as a white solid in 93% yield using 8-bromo-2,3-dihydro-1,4-benzodioxine-6-sulfonyl chloride (75 mg, 0.27 mmol, 1 equiv.), 4-phenylpiperidine (46 mg, 0.29 mmol, 1.2 equiv.) and Et₃N (100 μL, 0.718 mmol, 3 equiv.) in DCM (1.2 mL) at RT for 1 h. ¹H NMR (400 MHZ, Chloroform-d) δ 7.56 (d, J=2.1 Hz, 1H), 7.33-7.27 (m, 3H), 7.24-7.19 (m, 1H), 7.17-7.13 (m, 2H), 4.48-4.30 (m, 4H), 3.91 (dp, J=11.3, 1.8 Hz, 2H), 2.51-2.32 (m, 3H), 1.96-1.79 (m, 4H). MS (ESI⁺): [M+H]⁺ 438.0/439.9.

Synthesis of Intermediates required for final product 119 & 120

### 1-[5-[(4-phenyl-1-piperidyl) sulfonyl Jindolin-1-yl Jethanone (1-104)

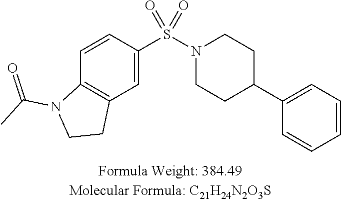 [SMILES: CC(=O)N1CCc2cc(S(=O)(=O)N3CCC(c4ccccc4)CC3)ccc21]

According to GP-4, I-104 was obtained as a white solid in 90% yield using 1-Acetyl-2,3-dihydro-1H-indole-5-sulfonyl chloride (300 mg, 1.15 mmol, 1 equiv.), 4-phenylpiperidine (223 mg, 1.39 mmol, 1.2 equiv.) and Et₃N (480 μL, 3.46 mmol, 3 equiv.) in DCM (5.8 mL) at RT for 16 h. Purification by FC (DCM/EtOAc=100/0 to 40/60). ¹H NMR (400 MHZ, Chloroform-d) δ 8.34 (d, J=8.5 Hz, 1H), 7.64 (dd, J=8.6, 1.9 Hz, 1H), 7.59 (d, J=1.9 Hz, 1H), 7.29 (dd, J=8.2, 6.7 Hz, 2H), 7.24-7.17 (m, 1H), 7.17-7.11 (m, 2H), 4.16 (t, J=8.6 Hz, 2H), 3.95-3.85 (m, 2H), 3.28 (t, J=8.6 Hz, 2H), 2.46-2.30 (m, 3H), 2.27 (s, 3H), 1.94-1.73 (m, 4H). MS (ESI⁺): [M+H]⁺ 385.0.

### 5-[(4-phenyl-1-piperidyl) sulfonyl]indoline (1-105)

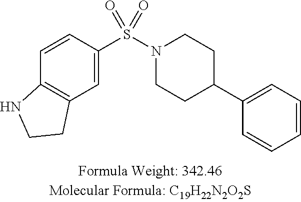 [SMILES: O=S(=O)(c1ccc2c(c1)CCN2)N1CCC(c2ccccc2)CC1]

To a solution of I-104 (400 mg, 1.04 mmol, 1 equiv.) in THF/MeOH/H₂O (4/2/1, 5 mL) at RT, was added NaOH 32% (0.35 mL, 5.2 mmol, 5 equiv.). The mixture was stirred at reflux for 16 h. The volatiles were removed under reduced pressure. The residue was partitioned between water and DCM. The layers were separated and the aqueous phase was extracted with DCM. The combined organic extracts were dried (Na₂SO₄), filtered and concentrated under reduced pressure to afford 315 mg of I-105 (88%) as a light brown solid. ¹H NMR (400 MHZ, Chloroform-d) δ 7.49-7.43 (m, 2H), 7.29 (dd, J=8.1, 6.7 Hz, 2H), 7.24-7.11 (m, 3H), 6.66-6.60 (m, 1H), 3.93-3.85 (m, 2H), 3.71 (t, J=8.6 Hz, 2H), 3.11 (t, J=8.6 Hz, 2H), 2.48-2.30 (m, 3H), 1.98-1.75 (m, 4H). MS (ESI⁺): [M+H]⁺ 343.1.

### 1-methyl-5-[(4-phenyl-1-piperidyl) sulfonyl]indoline (1-106)

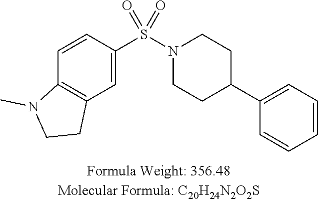 [SMILES: CN1CCc2cc(S(=O)(=O)N3CCC(c4ccccc4)CC3)ccc21]

To a solution of I-105 (530 mg, 1.39 mmol, 1 equiv.) in DMF (5.6 mL) at 0° C., was added NaH (60% in mineral oil, 84 mg, 2.1 mmol, 1.5 equiv.). The mixture was stirred at RT for 30 min and iodomethane (0.13 mL, 2.09 mmol, 1.5 equiv.) was added. The resulting mixture was stirred at RT for 16 h. Sat. aq. NH₄Cl and EtOAc were added. The layers were added and the aqueous phase was extracted with EtOAc. The combined organic extracts were washed (water, brine), dried (Na₂SO₄), filtered and concentrated under reduced pressure. The residue was purified by FC (cHex/DCM (70/30)/EtOAc=98/2 to 80/20) to afford 333 mg of I-106 (67%) as a white solid. ¹H NMR (400 MHZ, Chloroform-d) δ 7.54 (dd, J=8.3, 1.9 Hz, 1H), 7.44-7.40 (m, 1H), 7.37-7.28 (m, 2H), 7.26-7.11 (m, 3H), 6.43 (d, J=8.3 Hz, 1H), 3.95-3.86 (m, 2H), 3.54 (t, J=8.5 Hz, 2H), 3.06 (t, J=8.4 Hz, 2H), 2.88 (s, 3H), 2.50-2.30 (m, 3H), 1.99-1.76 (m, 4H). MS (ESI⁺): [M+H]⁺ 357.1.

### 7-bromo-1-methyl-5-[(4-phenyl-1-piperidyl) sulfonyl]indoline (1-107)

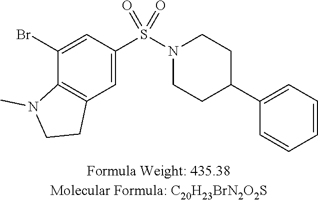 [SMILES: CN1CCc2cc(S(=O)(=O)N3CCC(c4ccccc4)CC3)cc(Br)c21]

To a solution of I-106 (333 mg, 0.831 mmol, 1 equiv.) in DMF (8.3 mL) at 0° C., was added N-bromosuccinimide (237 mg, 1.33 mmol, 1.6 equiv.). The mixture was stirred at RT for 2 h. Water and EtOAc were added. The layers were separated and the aqueous phase was extracted with EtOAc. The combined organic extracts were washed (water, brine), dried (Na₂SO₄), filtered and concentrated under reduced pressure. The residue was purified by FC (cHex/EtOAc=97/3 to 70/30) to afford 341 mg of I-107 (90%) as a white solid. 1H NMR (400 MHZ, Benzene-d₆) δ 7.95 (d, J=1.8 Hz, 1H), 7.33-7.28 (m, 1H), 7.14-7.08 (m, 2H), 7.08-7.00 (m, 1H), 6.91-6.86 (m, 2H), 3.80 (dq, J=11.4, 2.1 Hz, 2H), 2.77 -2.70 (m, 5H), 2.32 (t, J=8.8 Hz, 2H), 1.99 (td, J=11.9, 2.6 Hz, 2H), 1.90-1.80 (m, 1H), 1.62-1.43 (m, 2H), 1.42-1.31 (m, 2H). MS (ESI⁺): [M+H]⁺ 433.0/435.0.

### Synthesis of Intermediate Required for Final Product 121

### 1-(3-bromo-4-fluoro-phenyl) sulfonyl-4-phenyl-piperidine (1-108)

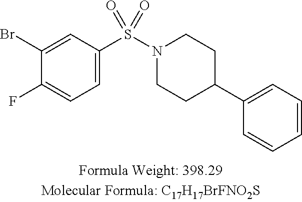 [SMILES: O=S(=O)(c1ccc(F)c(Br)c1)N1CCC(c2ccccc2)CC1]

According to GP-4, I-108 was obtained as a white solid in 100% yield using commercial 3-bromo-4-fluorobenzenesulfonyl chloride (500 mg, 1.83 mmol, 1 equiv.), 4-phenylpiperidine (442 mg, 2.74 mmol, 1.5 equiv.) and Et₃N (510 μL, 3.66 mmol, 2 equiv.) in DCM (7.3 mL) at RT for 2 h. ¹H NMR (400 MHZ, Chloroform-d) δ 8.05 (dd, J=6.3, 2.2 Hz, 1H), 7.77 (ddd, J=8.6, 4.5, 2.2 Hz, 1H), 7.37-7.29 (m, 3H), 7.27-7.21 (m, 1H), 7.21-7.13 (m, 2H), 4.06-3.89 (m, 2H), 2.58-2.35 (m, 3H), 2.01-1.81 (m, 4H). ¹⁹F NMR (376 MHZ, Chloroform-d) 8-99.4. MS (ESI⁺): [M+H]⁺ 398.0/399.9.

### 2-bromo-N,N-dimethyl-4-[(4-phenyl-1-piperidyl) sulfonyl]aniline (1-109)

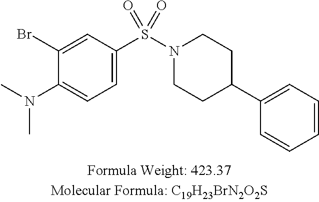 [SMILES: CN(C)c1ccc(S(=O)(=O)N2CCC(c3ccccc3)CC2)cc1Br]

A mixture of I-108 (725 mg, 1.82 mmol, 1 equiv) and dimethylamine (2M solution in THF, 4.55 mL, 9.10 mmol, 5 equiv.) was stirred at 50° C. for 18 h. IN NaOH and EtOAc were added. The layers were separated and the aqueous phase was extracted with EtOAc. The combined organic extracts were washed (water, brine), dried (Na₂SO₄), filtered and concentrated under reduced pressure to afford 685 mg of I-109 (89%) as a beige solid. 1H NMR (400 MHZ, Chloroform-d) δ 7.97 (d, J=2.2 Hz, 1H), 7.68 (dd, J=8.5, 2.2 Hz, 1H), 7.36-7.29 (m, 2H), 7.27-7.20 (m, 1H), 7.20-7.08 (m, 3H), 4.00-3.90 (m, 2H), 2.95 (s, 6H), 2.54-2.33 (m, 3H), 1.98-1.78 (m, 4H). MS (ESI⁺): [M+H]⁺ 423.1/425.0.

Synthesis of Intermediates required for final product 122

### benzyl 8-[2-fluoro-5-[(4-phenyl-1-piperidyl) sulfonyl]phenyl]-3,8-diazabicyclo[3.2.1]octane-3-carboxylate (1-110)

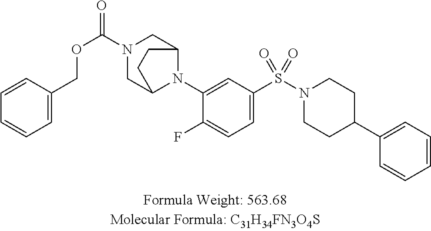 [SMILES: O=C(OCc1ccccc1)N1CC2CCC(C1)N2c1cc(S(=O)(=O)N2CCC(c3ccccc3)CC2)ccc1F]

According to GP-5, I-110 was obtained as a white foam in 64% yield using aryl bromide I-108 (0.40 g, 1.0 mmol, 1.2 equiv.), benzyl 3,8-diazabicyclo[3.2.1]octane-3-carboxylate (205 mg, 0.832 mmol, 1 equiv.), Cs₂CO₃ (542 mg, 1.66 mmol, 2 equiv.), Pd (OAc) 2 (19 mg, 83 μmol, 0.1 equiv.) and rac-BINAP (77.7 mg, 125 μmol, 0.15 equiv.) in toluene (4.2 mL) at reflux for 16 h. Purification by FC (cHex/EtOAc=90/10 to 50/50). ¹H NMR (400 MHZ, Chloroform-d, multiple sets of rotamers) δ 7.39-7.27 (m, 7H), 7.25-7.12 (m, 6H), 5.15 (s, 2H), 4.30-4.17 (m, 2H), 3.96-3.87 (m, 3H), 3.86-3.80 (m, 1H), 3.38-3.23 (m, 2H), 2.50-2.33 (m, 3H), 2.06-1.76 (m, 8H). ¹⁹F NMR (376 MHz, Chloroform-d, 2 sets of rotamers) δ-115.9. MS (ESI⁺): [M+H]⁺ 564.2.

### benzyl 8-[2-(dimethylamino)-5-[(4-phenyl-1-piperidyl) sulfonyl]phenyl]-3,8-diazabicyclo[3.2.1]octane-3-carboxylate (1-111)

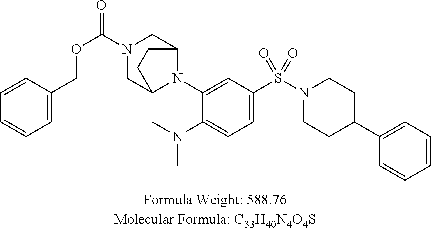 [SMILES: CN(C)c1ccc(S(=O)(=O)N2CCC(c3ccccc3)CC2)cc1N1C2CCC1CN(C(=O)OCc1ccccc1)C2]

To a solution of I-110 (136 mg, 0.241 mmol, 1 equiv.) in NMP (4.8 mL) at RT, were added dimethylamine hydrochloride (393 mg, 4.82 mmol, 20 equiv.) and iPr₂NEt (840 μL, 1.82 mmol, 20 equiv.). The mixture was stirred at 150° C. under MW irradiation for 40 h. The reaction mixture was partitioned between water and EtOAc. The layers were separated and the aqueous phase was extracted with EtOAc. The combined organic extracts were washed (water, brine), dried (Na₂SO₄), filtered and concentrated under reduced pressure. The residue was dissolved in DCM (1 mL) and Et₃N (67 μL, 0.48 mmol, 2 equiv.) and CbzCl (34 μL, 0.24 mmol, 1 equiv.) were added. The mixture was stirred at RT for 16 h. The mixture was concentrated under reduced pressure and the residue was purified by FC (cHex/EtOAc=95/5 to 50/50) to afford 127 mg of I-111 (89%) as a white solid. ¹H NMR (400 MHZ, Chloroform-d) δ 7.42-7.27 (m, 8H), 7.25-7.11 (m, 4H), 6.98 (d, J=8.4 Hz, 1H), 5.15 (s, 2H), 4.36-4.22 (m, 2H), 3.98-3.80 (m, 4H), 3.38-3.22 (m, 2H), 2.85 (s, 6H), 2.49-2.33 (m, 3H), 1.99-1.76 (m, 8H). MS (ESI⁺): [M+H]⁺ 589.4.

Synthesis of Intermediates required for final product 123

### tert-butyl N—[1-[2-bromo-4-[(4-phenyl-1-piperidyl) sulfonyl]phenyl]azetidin-3-yl]-N-methyl-carbamate (1-112)

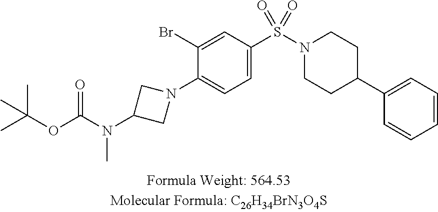 [SMILES: CN(C(=O)OC(C)(C)C)C1CN(c2ccc(S(=O)(=O)N3CCC(c4ccccc4)CC3)cc2Br)C1]

A mixture of I-108 (200 mg, 0.502 mmol, 1 equiv), tert-butyl azetidin-3-yl(methyl) carbamate hydrochloride (335 mg, 1.50 mmol, 3 equiv.) and K₂CO₃ (386 mg, 3.12 mmol, 6 equiv.) in dry DMF (2 mL) was stirred at 80° C. for 48 h. The mixture was partitioned between water and EtOAc. The layers were separated and the aqueous phase was extracted with EtOAc. The combined organic extracts were washed (water, brine), dried (Na₂SO₄), filtered and concentrated under reduced pressure. The residue was purified by FC (cHex/EtOAc=100/0 to 0/100 to afford 166 mg of I-112 (59%) as a yellow solid. ¹H NMR (400 MHZ, Chloroform-d) δ 7.81 (d, J=2.1 Hz, 1H), 7.56 (dd, J=8.6, 2.1 Hz, 1H), 7.33-7.27 (m, 2H), 7.24-7.13 (m, 3H), 6.50 (d, J=8.6 Hz, 1H), 5.08-4.77 (br s, 1H), 4.48 (t, J=8.3 Hz, 2H), 4.17 (dd, J=8.7, 6.0 Hz, 2H), 3.98-3.86 (m, 2H), 2.96 (s, 3H), 2.48-2.32 (m, 3H), 1.96-1.78 (m, 4H), 1.48 (s, 9H). MS (ESI⁺): [M+H]⁺ 564.2/566.0.

### tert-butyl N—[1-[2-[3-(2-chloro-4-fluoro-benzoyl)-3,8-diazabicyclo[3.2.1]octan-8-yl]-4-[(4-phenyl-1-piperidyl) sulfonyl]phenyl]azetidin-3-yl]-N-methyl-carbamate (1-113)

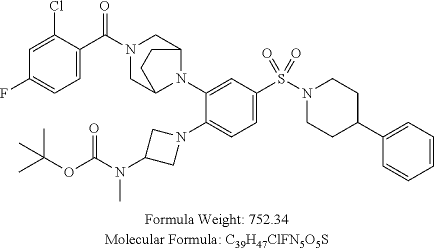 [SMILES: CN(C(=O)OC(C)(C)C)C1CN(c2ccc(S(=O)(=O)N3CCC(c4ccccc4)CC3)cc2N2C3CCC2CN(C(=O)c2ccc(F)cc2Cl)C3)C1]

According to GP-5, I-113 was obtained as a white foam in 50% yield using aryl bromide I-112 (73.0 mg, 129 μmol, 1 equiv.), piperazine I-001 (52.1 mg, 194 μmol, 1.5 equiv.), Cs₂CO₃ (126 mg, 0.388 mmol, 3 equiv.), Pd (OAc) 2 (2.9 mg, 13 μmol, 0.1 equiv.) and rac-BINAP (9.7 mg, 15 μmol, 0.12 equiv.) in toluene (0.7 mL) at reflux for 16 h. Purification by FC (cHex/EtOAc=95/5 to 50/50). ¹H NMR (400 MHZ, Chloroform-d, multiple sets of rotamers) δ 7.40-7.27 (m, 3.5H), 7.23-7.02 (m, 6.5H), 6.61 (d, J=8.4 Hz, 1H), 4.90 (br s, 1H), 4.60-4.40 (m, 1H), 4.26-4.18 (m, 2H), 3.95-3.80 (m, 5H), 3.70-3.45 (m, 2H), 3.36-3.10 (m, 2H), 3.00-2.89 (m, 3H), 2.50-2.25 (m, 3H), 2.01 -1.75 (m, 7.5H), 1.65-1.58 (m, 0.5H), 1.48 (s, 9H). MS (ESI⁺): [M+H]⁺ 752.0/754.0.

### Synthesis of Intermediates Required for Final Product 124

### 3,5-dibromo-4-methyl-benzenesulfonyl chloride (1-114)

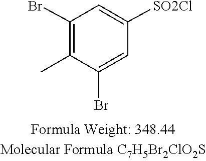

To a solution of CuCl (45 mg, 0.45 mmol, 0.12 equiv.) in water (13 mL) at 0° C., was added dropwise thionyl chloride (2.2 mL, 30 mmol, 8 equiv.). The mixture was stirred at RT for 16 h. In the meantime, to a solution of 3,5-dibromo-4-methylaniline (1.00 g, 3.77 mmol, 1 equiv.) in water (50 mL) at 0° C., were added 37% HCl (14 mL) and a solution of NaNO₂ (286 mg, 4.15 mmol, 1.1 equiv.) in water (13 mL). The mixture was stirred at 0° C. for 30 min. The first solution was then added dropwise and the resulting mixture was vigorously stirred at RT for 2 h. It was filtered over fritted glass and the filter cake was washed with water. The filter cake was dissolved using DCM and the resulting wet organic solution was dried (Na₂SO₄), filtered and concentrated under reduced pressure to afford 1.09 g (82%) of I-114 as an orange solid. ¹H NMR (400 MHZ, Chloroform-d) 8 8.16 (s, 2H), 2.69 (s, 3H).

### 1-(3,5-dibromo-4-methyl-phenyl) sulfonyl-4-phenyl-piperidine (1-115)

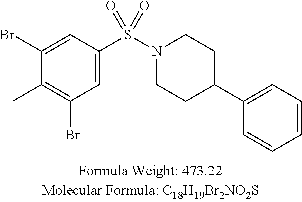 [SMILES: Cc1c(Br)cc(S(=O)(=O)N2CCC(c3ccccc3)CC2)cc1Br]

According to GP-4, I-115 was obtained as a beige solid in 62% yield using I-114 (1.08 g, 3.10 mmol, 1 equiv.), 4-phenylpiperidine (600 mg, 3.72 mmol, 1.2 equiv.) and iPr₂NEt (0.81 mL, 4.7 mmol, 1.5 equiv.) in DCM (15 mL) at RT for 1 h. Purification by FC (cHex/EtOAc=95/5 to 50/50). ¹H NMR (400 MHZ, Chloroform-d) δ 7.94 (s, 2H), 7.37 -7.30 (m, 2H), 7.27-7.21 (m, 1H), 7.20-7.14 (m, 2H), 4.05-3.89 (m, 2H), 2.68 (s, 3H), 2.57-2.41 (m, 3H), 2.00-1.79 (m, 4H). MS (ESI⁺): [M+H]⁺ 471.9/473.8/475.8.

### 3-bromo-2-methyl-5-[(4-phenyl-1-piperidyl) sulfonyl]phenol (1-116)

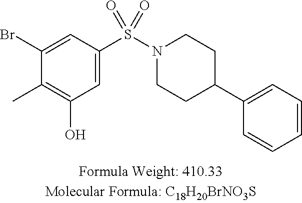 [SMILES: Cc1c(O)cc(S(=O)(=O)N2CCC(c3ccccc3)CC2)cc1Br]

A MW was charged with I-115 (573 mg, 1.21 mmol, 1 equiv.), KOAc (356 mg, 3.63 mmol, 3 equiv.), PdCl2 (dppf).CH₂Cl2 complex (99 mg, 0.12 mmol, 0.1 equiv.) and B2pin2 (277 mg, 1.09 mmol, 0.9 equiv.). The vial was purged with Ar and degassed 1,4-dioxane (6.1 mL) was added. The mixture was stirred at 80° C. for 16 h. The mixture was partitioned between EtOAc and water. The organic phase was washed (brine), dried (Na₂SO₄), filtered and concentrated under reduced pressure. The residue was dissolved in acetone (6 mL) at RT and a solution of oxone (968 mg, 1.57 mmol, 1.3 equiv.) in water (4 mL) was added. The mixture was stirred at RT for 20 min. Aq. Na₂S205 and EtOAc were added. The layers were separated and the aqueous phase was extracted with EtOAc. The combined organic extracts were dried (Na₂SO₄), filtered and concentrated under reduced pressure. The residue was purified by FC (cHex/EtOAc=97/3 to 70/30) to afford 100 mg of I-116 (20% over two steps) as an orange solid. ¹H NMR (400 MHZ, Chloroform-d) 8 7.55 (d, J=1.7 Hz, 1H), 7.34-7.27 (m, 2H), 7.24-7.12 (m, 4H), 5.77 (s, 1H), 3.90 (d, J=11.6 Hz, 2H), 2.53-2.34 (m, 3H), 1.96-1.76 (m, 4H), 1.43 (s, 3H). MS (ESI⁺): [M+H]⁺ 410.0/412.0.

### 1-[3-bromo-5-(methoxymethoxy)-4-methyl-phenyl]sulfonyl-4-phenyl-piperidine (1-117)

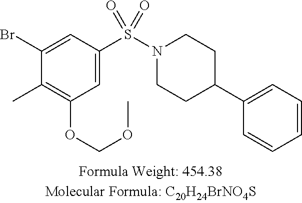 [SMILES: COCOc1cc(S(=O)(=O)N2CCC(c3ccccc3)CC2)cc(Br)c1C]

To a solution of phenol I-116 (100 mg, 0.243 mmol, 1 equiv.) and iPr₂NEt (64 μL, 0.37 mmol, 1.5 equiv.) in DCM (1.2 mL) at RT, was added chloromethyl methyl ether (22 μL, 0.29 mmol, 1.2 equiv.). The mixture was concentrated under reduced pressure. The residue was purified by FC (cHex/EtOAc=97/3 to 70/30) to afford 72 mg of I-117 (65%) as a white solid. ¹H NMR (400 MHz, Chloroform-d) δ 7.64 (d, J=1.6 Hz, 1H), 7.40 (d, J=1.6 Hz, 1H), 7.33-7.27 (m, 2H), 7.24-7.18 (m, 1H), 7.17-7.11 (m, 2H), 5.26 (s, 2H), 3.99-3.89 (m, 2H), 3.49 (s, 3H), 2.51-2.38 (m, 6H), 1.96-1.77 (m, 4H). MS (ESI⁺): [M+H]⁺ 454.0.0/455.9.

### (2-chloro-4-fluoro-phenyl)-[8-[3-(methoxymethoxy)-2-methyl-5-[(4-phenyl-1-piperidyl) sulfonyl]phenyl]-3,8-diazabicyclo[3.2.1]octan-3-yl]methanone (1-118)

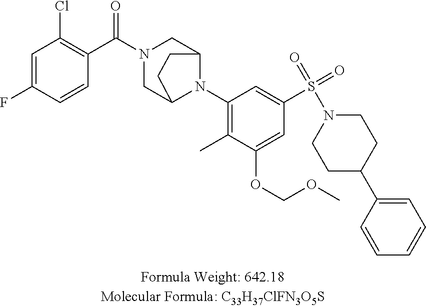 [SMILES: COCOc1cc(S(=O)(=O)N2CCC(c3ccccc3)CC2)cc(N2C3CCC2CN(C(=O)c2ccc(F)cc2Cl)C3)c1C]

According to GP-7, I-118 was obtained as a white solid in 59% yield using aryl bromide I-117 (72 mg, 0.16 mmol, 1 equiv.), piperazine I-001 (47 mg, 0.17 mmol, 1.1 equiv.), t-BuONa (46 mg, 0.48 mmol, 3 equiv.), XPhos-Pd-G3 (13 mg, 16 μmol, 0.1 equiv.) in toluene (0.8 mL) at reflux for 4 h. Purification by PTLC (cHex/EtOAc=70/30). ¹H NMR (400 MHZ, Chloroform-d, 2 sets of rotamers) δ 7.45-7.27 (m, 2.5H), 7.27-7.03 (m, 6.5H), 6.96 (d, J=7.2 Hz, 1H), 5.27 (s, 2H), 4.65-4.55 (m, 1H), 3.98-3.88 (m, 3H), 3.75-3.48 (m, 5H), 3.40-3.29 (m, 1H), 3.30-3.20 (m, 1H), 2.51-2.28 (m, 6H), 2.04 -1.77 (m, 7.5H), 1.70-1.60 (m, 0.5H).¹⁹F NMR (376 MHz, Chloroform-d, 2 sets of rotamers) δ-109.2,-109.3. MS (ESI⁺): [M+H]⁺ 642.2/644.1.

Synthesis of Intermediates required for final product 125 3-bromo-4-cyano-benzenesulfonyl chloride (1-119)

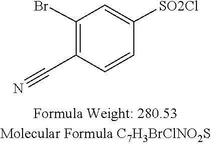 [SMILES: N#Cc1ccc(S(=O)(=O)Cl)cc1Br]

To a solution of CuCl (121 mg, 1.22 mmol, 0.12 equiv.) in water (34 mL) at 0° C., was added dropwise thionyl chloride (5.9 mL, 81 mmol, 8 equiv.). The mixture was stirred at RT for 14 h. In the meantime, to a solution of 4-amino-2-bromobenzonitrile (2.00 g, 10.1 mmol, 1 equiv.) in water (130 mL) at 0° C., were added 37% HCl (37 mL) and a solution of NaNO₂ (770 mg, 11.2 mmol, 1.1 equiv.) in water (34 mL). The mixture was stirred at 0° C. for 30 min. The first solution was then added dropwise and the resulting mixture was vigorously stirred at RT for 1 h. It was filtered over fritted glass and the filter cake was washed with water. The filter cake was dissolved using DCM and the resulting wet organic solution was dried (Na₂SO₄), filtered and concentrated under reduced pressure to afford 2.5 g (88%) of I-119 as an orange solid. ¹H NMR (400 MHz, Chloroform-d) δ 8.16 8 7.67 (d, J=1.8 Hz, 1H), 7.42 (dd, J=8.3, 1.9 Hz, 1H), 7.25 (d, J=8.3 Hz, 1H).

### 2-bromo-4-[(4-phenyl-1-piperidyl) sulfonyl]benzonitrile (1-120)

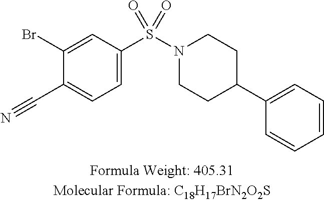 [SMILES: N#Cc1ccc(S(=O)(=O)N2CCC(c3ccccc3)CC2)cc1Br]

According to GP-4, I-120 was obtained as a beige solid in 47% yield using I-119 (2.5 g, 8.91 mmol, 1 equiv.), 4-phenylpiperidine (2.16 g, 13.4 mmol, 1.5 equiv.) and Et₃N (1.86 mL, 13.4 mmol, 1.5 equiv.) in DCM (45 mL) at RT for 1 h. Purification by FC (DCM/EtOAc=96/4 to 60/40). ¹H NMR (400 MHz, Chloroform-d) δ 8.11 (dd, J=1.4, 0.7 Hz, 1H), 7.86-7.80 (m, 2H), 7.35-7.28 (m, 2H), 7.25-7.21 (m, 1H), 7.18-7.12 (m, 2H), 4.03-3.94 (m, 2H), 2.54-2.42 (m, 3H), 1.98-1.91 (m, 2H), 1.91-1.79 (m, 2H). MS (ESI⁺): [M+H]⁺ 404.9/406.9.

Synthesis of Intermediates required for final product 139 3-bromo-1-chloro-4-fluoro-2-(methoxymethoxy)benzene (1-121)

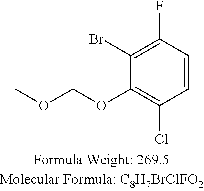

To a solution of 2-bromo-6-chloro-3-fluorophenol (1.11 g, 4.44 mmol, 1 equiv.) and iPr₂NEt (0.933 mL, 5.33 mmol, 1.2 equiv.) in DCM (22 mL) at RT, was added chloromethyl methyl ether (0.37 mL, 4.9 mmol, 1.1 equiv.). The mixture was concentrated under reduced pressure. The residue was purified by FC (cHex/EtOAc=95/5 to 50/50) to afford 756 mg of I-121 (55%) as a colorless oil. ¹H NMR (400 MHZ, Chloroform-d) 8 7.26 (dd, J=9.0, 5.7 Hz, 1H), 6.83 (dd, J=9.0, 7.5 Hz, 1H), 5.14 (s, 2H), 3.64 (s, 3H). ¹⁹F NMR (376 MHz, Chloroform-d) 8-104.5.

### [8-[3-chloro-6-fluoro-2-(methoxymethoxy)phenyl]-3,8-diazabicyclo[3.2.1]octan-3-yl]-(2-chloro-4-fluoro-phenyl) methanone (1-122)

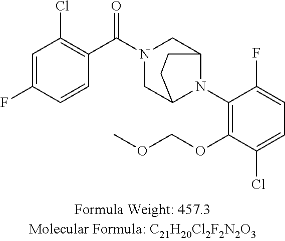 [SMILES: COCOc1c(Cl)ccc(F)c1N1C2CCC1CN(C(=O)c1ccc(F)cc1Cl)C2]

According to GP-5, I-122 was obtained as a yello sticky oil in 26% yield using aryl bromide I-121 (756 mg, 2.81 mmol, 1 equiv.), piperazine I-001 (905 mg, 3.37 mmol, 1.2 equiv.), Cs₂CO₃ (2.74 g, 8.41 mmol, 3 equiv.), Pd (OAc) 2 (63 mg, 0.28 mmol, 0.1 equiv.) and rac-BINAP (210 mg, 337 μmol, 0.12 equiv.) in toluene (14 mL) at reflux for 16 h. Purification by FC (cHex/EtOAc=97/3 to 70/30). ¹H NMR (400 MHZ, Chloroform-d, 2 sets of rotamers) ¹H NMR (400 MHZ, Chloroform-d) δ 7.29 (dd, J=8.5, 5.8 Hz, 0.5H), 7.16-7.06 (m, 1.5H), 7.03-6.93 (m, 1H), 6.85-6.80 (m, 1H), 6.72-6.63 (m, 1H), 5.13-5.02 (m, 2H), 4.47-4.36 (m, 1H), 4.23 (s, 1H), 4.05-3.95 (m, 1H), 3.59 (s, 3H), 3.56-3.34 (m, 1H), 3.21-3.12 (m, 1H), 3.10-3.00 (m, 1H), 2.05-1.75 (m, 3.5H), 1.57 (s, 0.5H). ¹⁹F NMR (376 MHz, Chloroform-d, 2 sets of rotamers) δ-109.4,-109.5,-126.8, 126.9. MS (ESI⁺): [M+H]⁺ 457.0/459.0.

### [8-(3-chloro-6-fluoro-2-hydroxy-phenyl)-3,8-diazabicyclo[3.2.1]octan-3-yl]-(2-chloro-4-fluoro-phenyl) methanone (1-123)

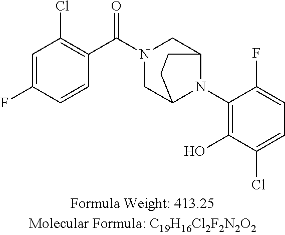 [SMILES: O=C(c1ccc(F)cc1Cl)N1CC2CCC(C1)N2c1c(F)ccc(Cl)c1O]

To a solution of I-122 (336 mg, 0.735 mmol, 1 equiv.) in DCM (3.7 mL) at RT, was added HCl (4M solution in dioxane, 3.67 mL, 14.7 mmol, 20 equiv.). The mixture was stirred at RT for 16 h. It was concentrated under reduced pressure. The residue was purified by FC (cHex/EtOAc=95/5 to 50/50) to afford 222 mg of I-123 (73%) as a white foam. ¹H NMR (400 MHz, Chloroform-d, 2 sets of rotamers) δ 7.30 (dd, J=8.5, 5.8 Hz, 0.5H), 7.16-7.06 (m, 1.5H), 7.05-6.95 (m, 1H), 6.95-6.87 (m, 1H), 6.68 (br s, 1H), 6.53-6.43 (m, 1H), 4.56-4.44 (m, 1H), 3.92-3.83 (m, 1H), 3.66-3.35 (m, 2H), 3.25-3.09 (m, 2H), 2.24-1.80 (m, 3.5H), 1.65-1.55 (m, 0.5H). ¹⁹F NMR (376 MHz, Chloroform-d, 2 sets of rotamers) δ-109.2,-109.3,-124.3,-124.5. MS (ESI⁺): [M+H]⁺ 413.1/415.1.

### 5-chloro-3-[3-(2-chloro-4-fluoro-benzoyl)-3,8-diazabicyclo[3.2.1]octan-8-yl]-2-fluoro-4-hydroxy-benzenesulfonyl chloride (1-124)

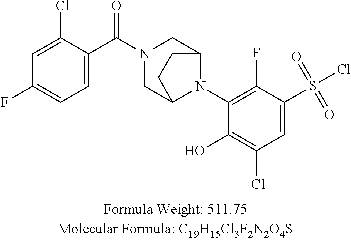 [SMILES: O=C(c1ccc(F)cc1Cl)N1CC2CCC(C1)N2c1c(O)c(Cl)cc(S(=O)(=O)Cl)c1F]

According to GP-3, I-124 was obtained as a beige solid in 89% yield using I-123 (110 mg, 0.215 mmol, 1 equiv.) in HSO₃Cl (0.32 mL, 4.8 mmol, 20 equiv.) at 50° C. for 16 h and then at 80° C. for 4 h. ¹H NMR (400 MHZ, Chloroform-d, 2 sets of rotamers) δ 7.62 & 7.60 (s, 1H), 7.35-7.38 (m, 0.5H), 7.18-7.07 (m, 1.5H), 7.06-6.95 (m, 1H), 4.63-4.45 (m, 1H), 4.07-3.95 (m, 1H), 3.77 (s, 1H), 3.65-3.40 (m, 1H), 3.27-3.13 (m, 2H), 2.20-1.85 (m, 3.5H), 1.70-1.60 (m, 0.5H). ¹⁹F NMR (376 MHz, Chloroform-d, 2 sets of rotamers) δ-108.6,-108.7,-120.3,-120.5.

### Synthesis of Final Compounds

### Synthetic Methods for Final Compounds

The synthetic protocols for the final compounds are presented on the following Table 2.

TABLE 2

| Cpd | Synthetic protocols | Purity |
|---|---|---|
| 001 | According to GP-4, 001 was obtained in 85% yield using | >95% |
| | sulfonyl chloride I-006 (175 mg, 379 μmol, 1 equiv.), N-methyl- | |
| | N-propylamine (76 μL, 0.76 mmol, 2 equiv.) and Et₃N (106 μL, | |
| | 759 μmol, 2 equiv.) in DCM (1.9 mL) at RT for 16 h. Purification | |
| | by FC (cHex/EtOAc = 95/5 to 0/100). | |
| 002 | According to GP-4, 002 was obtained in 81% yield using | >95% |
| | sulfonyl chloride I-008 (80% purity, 60 mg, 0.10 mmol, 1 equiv.), | |
| | tert-butylamine (16 μL, 0.15 mmol, 1.5 equiv.) and iPr₂NEt | |
| | (35 μL, 0.20 mmol, 2 equiv.) in DCM (0.6 mL) at RT for 16 h. | |
| | Purification by FC (cHex/EtOAc = 70/30 to 0/100). | |
| 003 | According to GP-4, 003 was obtained in 50% yield using | >95% |
| | sulfonyl chloride I-008 (80% purity, 60 mg, 0.10 mmol, 1 equiv.), | |
| | N-methyl-N-propylamine (16 μL, 0.15 mmol, 1.5 equiv.) and | |
| | iPr₂NEt (35 μL, 0.20 mmol, 2 equiv.) in DCM (0.6 mL) at RT for | |
| | 16 h. Purification by FC (cHex/EtOAc = 70/30 to 0/100). | |
| 004 | According to GP-4, 004 was obtained in 64% yield using | >95% |
| | sulfonyl chloride I-008 (80% purity, 60 mg, 0.10 mmol, 1 equiv.), | |
| | pyrrolidine (13 μL, 0.15 mmol, 1.5 equiv.) and iPr₂NEt (35 μL, | |
| | 0.20 mmol, 2 equiv.) in DCM (0.6 mL) at RT for 16 h. Purification | |
| | by FC (cHex/EtOAc = 70/30 to 0/100). | |
| 005 | According to GP-4, 005 was obtained in 60% yield using | >95% |
| | sulfonyl chloride I-008 (80% purity, 60 mg, 0.10 mmol, 1 equiv.), | |
| | N-(cyclopropylmethyl)-N-methylamine (14 μL, 0.15 mmol, 1.5 | |
| | equiv.) and iPr₂NEt (35 μL, 0.20 mmol, 2 equiv.) in DCM | |
| | (0.6 mL) at RT for 16 h. Purification by FC (cHex/EtOAc = 70/30 | |
| | to 0/100) and then PTLC (cHex/EtOAc = 50/50). | |
| 006 | According to GP-4, 006 was obtained in 84% yield using | >95% |
| | sulfonyl chloride I-008 (80% purity, 60 mg, 0.10 mmol, 1 equiv.), | |
| | 4-fluoropiperidine hydrochloride (21 mg, 0.15 mmol, 1.5 equiv.) | |
| | and iPr₂NEt (53 μL, 0.30 mmol, 3 equiv.) in DCM (0.6 mL) at RT | |
| | for 16 h. Purification by FC (cHex/EtOAc = 70/30 to 0/100). | |
| 007 | According to GP-4, 007 was obtained in 60% yield using | >95% |
| | sulfonyl chloride I-008 (80% purity, 60 mg, 0.10 mmol, 1 equiv.), | |
| | 4-(trifluoromethyl)piperidine hydrochloride (29 mg, 0.15 mmol, | |
| | 1.5 equiv.) and iPr₂NEt (53 μL, 0.30 mmol, 3 equiv.) in DCM | |
| | (0.6 mL) at RT for 16 h. Purification by FC (cHex/EtOAc = 70/30 | |
| | to 0/100) and then PTLC (cHex/EtOAc = 50/50). | |
| 008 | According to GP-4, 008 was obtained in 43% yield using | >95% |
| | sulfonyl chloride I-008 (80% purity, 30 mg, 61 μmol, 1 equiv.), | |
| | 4-benzylidenepiperidine hydrochloride (19 mg, 91 μmol, 1.5 | |
| | equiv.) and iPr₂NEt (32 μL, 0.18 mmol, 3 equiv.) in DCM | |
| | (0.6 mL) at RT for 16 h. Purification by FC (cHex/EtOAc = 70/30 | |
| | to 0/100) and then PTLC (DCM/Et₂O = 95/5). | |
| 009 | According to GP-4, 009 was obtained in 65% yield using | >95% |
| | sulfonyl chloride I-008 (50 mg, 0.11 mmol, 1 equiv.), | |
| | 4-phenylpiperidine (26 mg, 0.16 mmol, 1.5 equiv.) and iPr₂NEt | |
| | (37 μL, 0.21 mmol, 2 equiv.) in DCM (0.6 mL) at RT for 16 h. | |
| | Purification by FC (cHex/EtOAc = 70/30 to 0/100). | |
| 010 | According to GP-4, 010 was obtained in 68% yield using | >95% |
| | sulfonyl chloride I-008 (50 mg, 0.11 mmol, 1 equiv.), | |
| | 4-phenethyl-piperidine (30 mg, 0.16 mmol, 1.5 equiv.) and | |
| | iPr₂NEt (37 μL, 0.21 mmol, 2 equiv.) in DCM (0.6 mL) at RT for | |
| | 16 h. Purification by FC (cHex/EtOAc = 70/30 to 0/100) and | |
| | PTLC (DCM/MeOH = 99/1). | |
| 011 | According to GP-4, 011 was obtained in 65% yield using | >95% |
| | sulfonyl chloride I-008 (50 mg, 0.11 mmol, 1 equiv.), | |
| | 3-benzylpiperidine (28 mg, 0.16 mmol, 1.5 equiv.) and iPr₂NEt | |
| | (37 μL, 0.21 mmol, 2 equiv.) in DCM (0.6 mL) at RT for 16 h. | |
| | Purification by FC (cHex/EtOAc = 95/5 to 0/100). | |
| 012 | According to GP-4, 012 was obtained in 76% yield using | >95% |
| | sulfonyl chloride I-008 (50 mg, 0.11 mmol, 1 equiv.), | |
| | 1-phenylpiperazine (26 mg, 0.16 mmol, 1.5 equiv.) and iPr₂NEt | |
| | (37 μL, 0.21 mmol, 2 equiv.) in DCM (0.6 mL) at RT for 16 h. | |
| | Purification by FC (cHex/EtOAc = 95/5 to 0/100). | |
| 013 | According to GP-4, 013 was obtained in 77% yield using | >95% |
| | sulfonyl chloride I-008 (50 mg, 0.11 mmol, 1 equiv.), | |
| | 1-(2-pyrimidyl)-piperazine (23 μL, 0.16 mmol, 1.5 equiv.) and | |
| | iPr₂NEt (37 μL, 0.21 mmol, 2 equiv.) in DCM (0.6 mL) at RT for | |
| | 72 h. Purification by FC (cHex/EtOAc = 70/30 to 0/100). | |
| 014 | According to GP-4, 014 was obtained in 73% yield using | >95% |
| | sulfonyl chloride I-008 (50 mg, 0.11 mmol, 1 equiv.), N-benzyl | |
| | piperazine (27 μL, 0.16 mmol, 1.5 equiv.) and iPr₂NEt (37 μL, | |
| | 0.21 mmol, 2 equiv.) in DCM (0.6 mL) at RT for 16 h. Purification | |
| | by FC (cHex/EtOAc = 70/30 to 0/100). | |
| 015 | According to GP-4, 015 was obtained in 82% yield using | >95% |
| | sulfonyl chloride I-008 (60 mg, 0.11 mmol, 1 equiv.), cis- | |
| | (3aR,7aS)-octahydro-1H-isoindole hydrochloride (25 mg, | |
| | 0.15 mmol, 1.5 equiv.) and iPr₂NEt (53 μL, 0.30 mmol, 3 equiv.) | |
| | in DCM (0.6 mL) at RT for 72 h. Purification by FC (cHex/EtOAc = | |
| | 70/30 to 0/100). | |
| 016 | According to GP-1, 016 was obtained in 19% yield using | >95% |
| | 4-fluoro-2-(trifluoromethyl)benzoic acid (47 mg, 228 μmol, 1.5 | |
| | equiv.) and TBTU (73 mg, 228 μmol, 1.5 equiv.) in DMF | |
| | (0.4 mL), followed by piperazine I-010 (60 mg, 152 μmol, | |
| | 1 equiv.) and Et₃N (42 μL, 0.30 mmol, 2 equiv.) in THF (0.4 mL) | |
| | at RT for 16 h. Purification by FC (cHex/EtOAc = 93/7 to 30/70) | |
| | and PTLC (cHex/EtOAc = 70/30). | |
| 017 | According to GP-1, 017 was obtained in 73% yield using | >95% |
| | 4-fluoro-2-(trifluoromethyl)benzoic acid (62 mg, 295 μmol, | |
| | 1.5 equiv.) and TBTU (95 mg, 295 μmol, 1.5 equiv.) in DMF | |
| | (1 mL), followed by piperazine I-011 (70 mg, 197 μmol, 1 equiv.) | |
| | and Et₃N (55 μL, 0.40 mmol, 2 equiv.) in THF (1 mL) at RT for | |
| | 16 h. Purification by FC (cHex/EtOAc = 93/7 to 30/70). | |
| 018 | According to GP-1, 018 was obtained in 71% yield using | >95% |
| | 4-fluoro-2-(trifluoromethyl)benzoic acid (48 mg, 230 μmol, | |
| | 1.5 equiv.) and TBTU (74 mg, 230 μmol, 1.5 equiv.) in DMF | |
| | (1.5 mL), followed by piperazine I-013 (68 mg, 153 μmol, 1 | |
| | equiv.) and Et₃N (43 μL, 0.31 mmol, 2 equiv.) in THF (1.5 mL) at | |
| | RT for 16 h. Purification by FC (cHex/EtOAc = 94/6 to 40/60). | |
| 019 | According to GP-1, 019 was obtained in 98% yield using | >95% |
| | 2-chloro-4-fluorobenzoic acid (40 mg, 230 μmol, 1.5 equiv.) and | |
| | TBTU (74 mg, 230 μmol, 1.5 equiv.) in DMF (1.5 mL), followed | |
| | by piperazine I-013 (68 mg, 153 μmol, 1 equiv.) and Et₃N (43 μL, | |
| | 0.31 mmol, 2 equiv.) in THF (1.5 mL) at RT for 16 h. Purification | |
| | by FC (cHex/EtOAc = 94/6 to 60/40). | |
| 020 | According to GP-1, 020 was obtained in 58% yield using | >95% |
| | 2-chloro-4-fluorobenzoic acid (37 mg, 212 μmol, 1.5 equiv.) and | |
| | TBTU (71 mg, 221 μmol, 1.6 equiv.) in DMF (1.5 mL), followed | |
| | by piperazine I-015 (47 mg, 138 μmol, 1 equiv.) and Et₃N (50 μL, | |
| | 0.36 mmol, 2.6 equiv.) in THF (1.5 mL) at RT for 16 h. | |
| | Purification by FC (cHex/EtOAc = 100/0 to 20/80) and PTLC | |
| | (cHex/EtOAc = 70/30). | |
| 021 | According to GP-2, 021 was obtained in 60% yield using | >95% |
| | 4-fluoro-2-(trifluoromethyl)benzoic acid (70.2 mg, 337 μmol, 1.2 | |
| | equiv.), iPr₂NEt (98 μL, 0.56 mmol, 2 equiv.), TPTU (100 mg, | |
| | 337 μmol, 1.2 equiv.) and piperazine I-016 (96.0 mg, 281 mmol, | |
| | 1 equiv.) in DCE/MeCN (3 mL) at RT for 16 h. Purification by | |
| | PTLC (cHex/EtOAc = 50/50). | |
| 022 | According to GP-2, 022 was obtained in 38% yield using | >95% |
| | 4-fluoro-2-(trifluoromethyl)benzoic acid (52 mg, 0.25 mmol, | |
| | 1.2 equiv.), iPr₂NEt (73 μL, 0.42 mmol, 2 equiv.), TPTU (75 mg, | |
| | 0.25 mmol, 1.2 equiv.) and piperazine I-017 (78 mg, 0.21 mmol, 1 | |
| | equiv.) in DCE/MeCN (3 mL) at RT for 16 h. Purification by FC | |
| | (cHex/EtOAc = 91/9 to 10/90). | |
| 023 | According to GP-2, 023 was obtained in 68% yield using | >95% |
| | 4-fluoro-2-(trifluoromethyl)benzoic acid (61 mg, 0.29 mmol, | |
| | 1.2 equiv.), iPr₂NEt (85 μL, 0.49 mmol, 2 equiv.), TPTU (87 mg, | |
| | 0.29 mmol, 1.2 equiv.) and piperazine I-018 (90 mg, 024 mmol, | |
| | 1 equiv.) in DCE/MeCN (1.2 mL) at RT for 72 h. Purification by | |
| | FC (cHex/EtOAc = 95/5 to 0/100). | |
| 024 | According to GP-5, 024 was obtained in 96% yield using aryl | >95% |
| | bromide I-009 (128 mg, 397 μmol, 1.2 equiv.), piperazine I-002 | |
| | (100 mg, 331 μmol, 1 equiv.), Cs₂CO₃ (334 mg, 992 μmol, 3 | |
| | equiv.), Pd(OAc)₂ (7.4 mg, 33 μmol, 0.1 equiv.) and rac-BINAP | |
| | (31 mg, 50 μmol, 0.15 equiv.) in toluene (1.7 mL) at reflux for | |
| | 16 h. Purification by FC (cHex/EtOAc = 95/5 to 40/60). | |
| 025 | According to GP-5, 025 was obtained in 51% yield using aryl | >95% |
| | bromide I-012 (100 mg, 236 μmol, 1 equiv.), piperazine I-001 | |
| | (82 mg, 31 μmol, 1.3 equiv.), Cs₂CO₃ (116 mg, 354 μmol, 1.5 | |
| | equiv.), Pd(OAc)₂ (2.7 mg, 12 μmol, 0.05 equiv.) and rac-BINAP | |
| | (8.8 mg, 14 μmol, 0.15 equiv.) in toluene (1.2 mL) at reflux for | |
| | 24 h. Purification by FC (cHex/EtOAc = 90/10 to 0/100). | |
| 026 | According to GP-5, 026 was obtained in 75% yield using aryl | 95% |
| | bromide I-019 (100 mg, 322 μmol, 1 equiv.), piperazine I-001 | |
| | (120 mg, 447 μmol, 1.4 equiv.), Cs₂CO₃ (316 mg, 970 μmol, | |
| | 3 equiv.), Pd(OAc)₂ (10 mg, 43 μmol, 0.1 equiv.) and rac-BINAP | |
| | (60 mg, 96 μmol, 0.3 equiv.) in toluene (1.6 mL) at reflux for 3 h. | |
| | Purification by FC (cHex/EtOAc = 100/0 to 70/30). | |
| 027 | According to GP-5, 027 was obtained in 60% yield using aryl | 95% |
| | bromide I-020 (100 mg, 266 μmol, 1 equiv.), piperazine I-001 | |
| | (86.0 mg, 319 μmol, 1.2 equiv.), Cs₂CO₃ (217 mg, 665 μmol, | |
| | 2.5 equiv.), Pd(OAc)₂ (6.0 mg, 27 μmol, 0.1 equiv.) and | |
| | rac-BINAP (20 mg, 32 μmol, 0.12 equiv.) in toluene (1.4 mL) at | |
| | reflux for 16 h. Purification by FC (cHex/EtOAc = 100/0 to | |
| | 60/40). | |
| 028 | According to GP-5, 028 was obtained in 43% yield using aryl | >95% |
| | bromide I-021 (100 mg, 342 μmol, 1 equiv.), piperazine I-001 | |
| | (138 mg, 513 μmol, 1.5 equiv.), Cs₂CO₃ (223 mg, 685 μmol, 2 | |
| | equiv.), Pd(OAc)₂ (8.0 mg, 34 μmol, 0.1 equiv.) and rac-BINAP | |
| | (26 mg, 41 μmol, 0.12 equiv.) in toluene (1.7 mL) at reflux for 3 h. | |
| | Purification by FC (cHex/EtOAc = 95/5 to 50/50) and a second | |
| | FC (DCM/EtOAc = 98/2 to 80/20). | |
| 029 | According to GP-5, 029 was obtained in 26% yield using aryl | 95% |
| | bromide I-023 (97 mg, 287 μmol, 1 equiv.), piperazine I-001 | |
| | (93.0 mg, 344 μmol, 1.2 equiv.), Cs₂CO₃ (234 mg, 718 μmol, 2.5 | |
| | equiv.), Pd(OAc)₂ (6.5 mg, 29 μmol, 0.1 equiv.) and rac-BINAP | |
| | (21 mg, 34 μmol, 0.12 equiv.) in toluene (1.2 mL) at reflux for 3 h. | |
| | Purification by FC (cHex/EtOAc = 100/0 to 50/50) and PTLC | |
| | (DCM/EtOAc = 90/10). | |
| 030 | According to GP-5, 030 was obtained in 51% yield using aryl | 95% |
| | bromide I-024 (53.0 mg, 146 μmol, 1 equiv.), piperazine I-001 | |
| | (47.0 mg, 176 μmol, 1.2 equiv.), Cs₂CO₃ (120 mg, 366 μmol, 2.5 | |
| | equiv.), Pd(OAc)₂ (4.0 mg, 15 μmol, 0.1 equiv.) and rac-BINAP | |
| | (11 mg, 18 μmol, 0.12 equiv.) in toluene (0.8 mL) at reflux for | |
| | 2.5 h. Purification by FC (DCM/EtOAc = 100/0 to 90/10). | |
| 031 | According to GP-5, 031 was obtained in 51% yield using aryl | 95% |
| | bromide I-025 (293 mg, 832 μmol, 1 equiv.), piperazine I-001 | |
| | (291 mg, 1.08 mmol, 1.3 equiv.), Cs₂CO₃ (542 mg, 1.66 mmol, | |
| | 2 equiv.), Pd(OAc)₂ (19 mg, 83 μmol, 0.1 equiv.) and rac-BINAP | |
| | (62.1 mg, 100 μmol, 0.12 equiv.) in toluene (4.2 mL) at reflux for | |
| | 2 h. Purification by FC (cHex/EtOAc = 93/7 to 30/70). | |
| 032 | According to GP-6, 032 was obtained in 14% yield using aryl | >95% |
| | bromide I-026 (205 mg, 625 μmol, 1.2 equiv.), piperazine I-001 | |
| | (140 mg, 521 μmol, 1 equiv.), t-BuONa (75 mg, 0.78 mmol, 1.5 | |
| | equiv.), XantPhos-Pd-G3 (25 mg, 26 μmol, 0.05 equiv.) in | |
| | toluene (2.6 mL) at reflux for 16 h. Purification by FC | |
| | (cHex/EtOAc = 100/0 to 50/50). | |
| 033 | According to GP-6, 033 was obtained in 17% yield using aryl | >95% |
| | bromide I-027 (138 mg, 426 μmol, 1.2 equiv.), piperazine I-001 | |
| | (95 mg, 0.35 mmol, 1 equiv.), t-BuONa (51 mg, 0.53 mmol, 1.5 | |
| | equiv.), XantPhos-Pd-G3 (17 mg, 18 μmol, 0.05 equiv.) in | |
| | toluene (1.8 mL) at reflux for 16 h. Purification by FC | |
| | (cHex/EtOAc = 100/0 to 50/50) and PTLC (cHex/EtOAc = | |
| | 50/50). | |
| 034 | According to GP-6, 034 was obtained in 20% yield using aryl | >95% |
| | bromide I-028 (232 mg, 581 μmol, 1.2 equiv.), piperazine I-001 | |
| | (130 mg, 484 μmol, 1 equiv.), t-BuONa (70 mg, 0.73 mmol, 1.5 | |
| | equiv.), XantPhos-Pd-G3 (23 mg, 24 μmol, 0.05 equiv.) in | |
| | toluene (2.4 mL) at reflux for 16 h. Purification by FC | |
| | (cHex/EtOAc = 100/0 to 50/50) and PTLC (cHex/EtOAc = 50/50 | |
| | then DCM 100:0). | |
| 035 | A suspension of 034 (54 mg, 92 μmol, 1 equiv.) and Pd(OH)₂ | 95% |
| | (20% on activated charcoal, 50% wet, 6 mg, 5 μmol, 0.05 equiv.) | |
| | in EtOH (2 mL) was stirred under and H₂ atmosphere at RT for | |
| | 5 h. The suspension was filtered over celite (EtOH rinses), | |
| | concentrated and the residue was purified by PTLC | |
| | (cHex/EtOAc = 20/80) affording 035 in 75% yield. | |
| 036 | A suspension of I-030 (1.10 g, 1.88 mmol, 1 equiv.) and Pd(OH)₂ | 93% |
| | (20% on activated charcoal, 50% wet, 110 mg, 392 μmol, 0.2 | |
| | equiv.) in EtOH (9.5 mL) was stirred under and H₂ atmosphere at | |
| | RT for 16. The suspension was filtered over celite (EtOH rinses), | |
| | concentrated and the residue was purified by FC (DCM/MeOH = | |
| | 100/0 to 95/5) affording 036 in 87% yield. | |
| 037 | To a solution of I-031 (64 mg, 0.12 mmol, 1 equiv.) in THF | >95% |
| | (0.6 mL) at RT, was added sodium hydride (60% in mineral oil, | |
| | 7.4 mg, 0.18 mmol, 1.5 equiv.) and the reaction was stirred for | |
| | 15 min. Benzyl bromide (29 μL, 0.25 mmol, 2 equiv.) was added | |
| | and the mixture was stirred at RT for 3 h. The reaction mixture | |
| | was partitioned between EtOAc and aq. sat. NH₄Cl. The layers | |
| | were separated and the aqueous phase was extracted with EtOAc. | |
| | The combined organic extracts were washed (aq. sat. NaHCO₃, | |
| | brine), dried (Na₂SO4), filtered and concentrated under reduced | |
| | pressure. The residue was purified by FC (cHex/EtOAc = 100/0 | |
| | to 50/50) affording 037 in 60% yield. | |
| 038 | To a solution of I-032 (60.0 mg, 120 mmol, 1 equiv.) in DMF | >95% |
| | (0.6 mL) at RT, were added K₂CO₃ (50 mg, 0.36 mmol, 3 equiv.) | |
| | and iodomethane (23 μL, 0.36 mmol, 3 equiv.) and the suspension | |
| | was stirred at 50° C. for 2 h 30. More iodomethane (23 μL, | |
| | 0.36 mmol, 3 equiv.) was added and the resulting suspension was | |
| | stirred a further 1 h. The reaction mixture was partitioned | |
| | between EtOAc and H₂O. The layers were separated and the | |
| | aqueous phase was extracted with EtOAc. The combined organic | |
| | extracts were washed with brine, dried (Na₂SO₄), filtered and | |
| | concentrated under reduced pressure. The residue was purified | |
| | by FC (cHex/EtOAc = 100/0 to 50/50) affording 038 in 80% | |
| | yield. | |
| 039 | According to GP-4, 039 was obtained in 46% yield using | >95% |
| | sulfonyl chloride I-008 (80% purity, 80 mg, 0.14 mmol, 1 equiv.), | |
| | 4-(4-fluorophenyl)piperidine (29 mg, 0.16 mmol, 1.2 equiv.) and | |
| | Et₃N (40 μL, 0.27 mmol, 2 equiv.) in DCM (0.7 mL) at RT for 2 h. | |
| | Purification by PTLC (DCM/MeOH = 95/5). | |
| 040 | According to GP-4, 040 was obtained in 28% yield using | >95% |
| | sulfonyl chloride I-008 (80 mg, 0.17 mmol, 1 equiv.), 3- | |
| | (piperidin-4-yl)benzonitrile (63 mg, 0.34 mmol, 2 equiv.) and | |
| | Et₃N (50 μL, 0.34 mmol, 2 equiv.) in DCM (0.8 mL) at RT for | |
| | 16 h. Purification by PTLC (cHex/EtOAc = 50/50). | |
| 041 | According to GP-4, 041 was obtained in 49% yield using | >95% |
| | sulfonyl chloride I-008 (105 mg, 222 μmol, 1 equiv.), methyl 3- | |
| | (4-piperidyl)benzoate (85 mg, 0.33 mmol, 1.5 equiv.) and iPr₂NEt | |
| | (155 μL, 0.887 mmol, 4 equiv.) in DCM (1.1 mL) at RT for 5 h. | |
| | Purification by PTLC (cHex/EtOAc = 40/60). | |
| 042 | According to GP-4, 042 was obtained in 85% yield using | >95% |
| | sulfonyl chloride I-008 (65 mg, 137 μmol, 1 equiv.), methyl 4-(4- | |
| | piperidyl)benzoate (45 mg, 0.21 mmol, 1.5 equiv.) and iPr₂NEt | |
| | (96 μL, 0.54 mmol, 4 equiv.) in DCM (0.7 mL) at RT for 16 h. | |
| | Purification by FC (cHex/EtOAc = 80/20 to 0/100). | |
| 043 | According to GP-4, 043 was obtained in 46% yield using | >95% |
| | sulfonyl chloride I-008 (105 mg, 176 μmol, 1.2 equiv.), amine | |
| | hydrochloride I-034 (44 mg, 0.15 mmol, 1 equiv.) and Et₃N | |
| | (100 μL, 0.733 mmol, 5 equiv.) in DCM (1.5 mL) at RT for 16 h. | |
| | Purification by PTLC (cHex/EtOAc = 50/50). | |
| 044 | According to GP-4, 044 was obtained in 35% yield using | >95% |
| | sulfonyl chloride I-008 (67 mg, 0.14 mmol, 1.2 equiv.), amine I- | |
| | 036 (26 mg, 0.12 mmol, 1 equiv.) and iPr₂NEt (40 μL, 0.24 mmol, | |
| | 2 equiv.) in DCM (0.6 mL) at RT for 16 h. Purification by PTLC | |
| | (cHex/EtOAc = 50/50). | |
| 045 | According to GP-4, 045 was obtained in 29% yield using | >95% |
| | sulfonyl chloride I-008 (50 mg, 0.11 mmol, 1 equiv.), 1-[rac- | |
| | (2R)-2-(4-fluorophenyl)propyl]piperazine (26 mg, 0.12 mmol, | |
| | 1.1 equiv.) and iPr₂NEt (40 μL, 0.21 mmol, 2 equiv.) in DCM | |
| | (1.1 mL) at RT for 16 h. Purification by PTLC (cHex/EtOAc = | |
| | 40/60). | |
| 046 | To a solution of piperazine I-037 (80 mg, 0.15 mmol, 1 equiv.) in | >95% |
| | DCM (2 mL) at RT, were added AcOH (17 μL, 0.30 mmol, 2 | |
| | equiv.), 2-(4-fluorophenyl)acetaldehyde (106 mg, 0.765 mmol, 5 | |
| | equiv.) and NaBH(OAc)₃ (65 mg, 0.31 mmol, 2 equiv.). The | |
| | mixture was stirred at RT for 4 h. Sat. aq. NaHCO₃ was added. | |
| | The layers were separated and the aqueous phase was extracted | |
| | with DCM. The combined organic extracts were washed with | |
| | brine, dried (Na₂SO₄), filtered and concentrated under reduced | |
| | pressure. The residue was purified by FC (cHex/EtOAc = 70/30 | |
| | to 0/100) affording 046 in 78% yield. | |
| 047 | To a solution of piperazine I-037 (80 mg, 0.15 mmol,1 equiv.) in | >95% |
| | DCM (2 mL) at RT, were added AcOH (17 μL, 0.30 mmol, 2 | |
| | equiv.), 2-Phenylpropionaldehyde (103 mg, 0.765 mmol, 5 | |
| | equiv.) and NaBH(OAc)₃ (65 mg, 0.31 mmol, 2 equiv.). The | |
| | mixture was stirred at RT for 4 h. Sat. aq. NaHCO₃ was added. | |
| | The layers were separated and the aqueous phase was extracted | |
| | with DCM. The combined organic extracts were washed with | |
| | brine, dried (Na₂SO₄), filtered and concentrated under reduced | |
| | pressure. The residue was purified by FC (cHex/EtOAc = 70/30 | |
| | to 0/100) affording 047 in 79% yield. | |
| 048 | To a solution of piperazine I-037 (80 mg, 0.15 mmol, 1 equiv.) in | >95% |
| | DCM (2 mL) at RT, were added AcOH (17 μL, 0.30 mmol, 2 | |
| | equiv.), 2-(4-chlorophenyl)acetaldehyde (47 mg, 0.31 mmol, 2 | |
| | equiv.) and NaBH(OAc)3 (65 mg, 0.31 mmol, 2 equiv.). The | |
| | mixture was stirred at RT for 16 h. Sat. aq. NaHCO₃ was added. | |
| | The layers were separated and the aqueous phase was extracted | |
| | with DCM. The combined organic extracts were washed with | |
| | brine, dried (Na₂SO₄), filtered and concentrated under reduced | |
| | pressure. The residue was purified by FC (DCM/MeOH = 99/1 | |
| | to 95/5) affording 048 in 89% yield. | |
| 049 | According to GP-4, 049 was obtained in 51% yield using | >95% |
| | sulfonyl chloride I-039 (70 mg, 0.14 mmol, 1 equiv.), 4- | |
| | phenylpiperidine (44 mg, 0.27 mmol, 2 equiv.) and Et₃N (40 μL, | |
| | 0.27 mmol, 2 equiv.) in DCM (0.7 mL) at RT for 16 h. Purification | |
| | by PTLC (cHex/EtOAc = 50/50). | |
| 050 | According to GP-4, 050 was obtained in 48% yield using | >95% |
| | sulfonyl chloride I-039 (70 mg, 0.14 mmol, 1 equiv.), 4- | |
| | phenethylpiperidine (52 mg, 0.27 mmol, 2 equiv.) and Et₃N | |
| | (40 μL, 0.27 mmol, 2 equiv.) in DCM (0.7 mL) at RT for 16 h. | |
| | Purification by PTLC (cHex/EtOAc = 50/50). | |
| 051 | According to GP-4, 051 was obtained in 50% yield using | >95% |
| | sulfonyl chloride I-039 (50 mg, 0.10 mmol, 1 equiv.), 1- | |
| | phenylpiperazine (32 mg, 0.20 mmol, 2 equiv.) and Et₃N (30 μL, | |
| | 0.19 mmol, 2 equiv.) in DCM (0.5 mL) at RT for 16 h. Purification | |
| | by PTLC (cHex/EtOAc = 50/50). | |
| 052 | According to GP-4, 052 was obtained in 31% yield using | >95% |
| | sulfonyl chloride I-039 (70 mg, 0.14 mmol, 1 equiv.), 4- | |
| | benzylpiperidine (48 mg, 0.27 mmol, 2 equiv.) and Et₃N (40 μL, | |
| | 0.27 mmol, 2 equiv.) in DCM (0.7 mL) at RT for 16 h. Purification | |
| | by PTLC (cHex/EtOAc = 50/50). | |
| 053 | According to GP-4, 053 was obtained in 35% yield using | >95% |
| | sulfonyl chloride I-039 (70 mg, 0.14 mmol, 1 equiv.), 4- | |
| | benzylidenepiperidine (47 mg, 0.27 mmol, 2 equiv.) and Et₃N | |
| | (40 μL, 0.27 mmol, 2 equiv.) in DCM (0.7 mL) at RT for 16 h. | |
| | Purification by PTLC (cHex/EtOAc = 50/50). | |
| 054 | According to GP-4, 054 was obtained in 49% yield using | >95% |
| | sulfonyl chloride I-039 (70 mg, 0.14 mmol, 1 equiv.), N-methyl- | |
| | tert-butylamine (24 mg, 0.27 mmol, 2 equiv.) and Et₃N (40 μL, | |
| | 0.27 mmol, 2 equiv.) in DCM (0.7 mL) at RT for 16 h. Purification | |
| | by PTLC (cHex/EtOAc = 50/50). | |
| 055 | According to GP-4, 055 was obtained in 81% yield using | >95% |
| | sulfonyl chloride I-041 (121 mg, 0.249 mmol, 1 equiv.), 4- | |
| | phenylpiperidine (60 mg, 0.37 mmol, 1.5 equiv.) and Et₃N (70 μL, | |
| | 0.50 mmol, 2 equiv.) in DCM (1.2 mL) at RT for 16 h. Purification | |
| | by FC (cHex/EtOAc = 96/4 to 40/60). | |
| 056 | According to GP-4, 056 was obtained in 79% yield using | >95% |
| | sulfonyl chloride I-041 (50 mg, 0.10 mmol, 1 equiv.), 4-(4- | |
| | fluorophenyl)piperidine (22 mg, 0.12 mmol, 1.2 equiv.) and Et₃N | |
| | (30 μL, 0.21 mmol, 2 equiv.) in DCM (0.5 mL) at RT for 16 h. | |
| | Purification by FC (cHex/EtOAc = 95/5 to 50/50). | |
| 057 | According to GP-4, 057 was obtained in 50% yield using | >95% |
| | sulfonyl chloride I-041 (75 mg, 0.15 mmol, 1 equiv.), tert- | |
| | butylamine (14 mg, 0.19 mmol, 1.2 equiv.) and Et₃N (32 μL, | |
| | 0.23 mmol, 1.5 equiv.) in DCM (0.8 mL) at RT for 16 h. | |
| | Purification by PTLC (DCM/EtOAc = 90/10). | |
| 058 | According to GP-4, 058 was obtained in 55% yield using | >95% |
| | sulfonyl chloride I-041 (75 mg, 0.15 mmol, 1 equiv.), N- | |
| | (cyclopropylmethyl)-N-methylamine (16 mg, 0.19 mmol, 1.2 | |
| | equiv.) and Et₃N (32 μL, 0.23 mmol, 1.5 equiv.) in DCM (0.8 mL) | |
| | at RT for 2 h. Purification by PTLC (cHex/EtOAc = 50/50). | |
| 059 | According to GP-4, 059 was obtained in 86% yield using | >95% |
| | sulfonyl chloride I-041 (60 mg, 0.12 mmol, 1 equiv.), 1-[2-(4- | |
| | fluorophenyl)ethyl]piperazine (33 mg, 0.16 mmol, 1.3 equiv.) and | |
| | Et₃N (33 μL, 0.25 mmol, 2 equiv.) in DCM (0.6 mL) at RT for 2 h. | |
| | Purification by FC (cHex/EtOAc = 90/10 to 0/100). | |
| 060 | According to GP-4, 060 was obtained in 85% yield using | >95% |
| | sulfonyl chloride I-041 (60 mg, 0.12 mmol, 1 equiv.), 1-[2-(4- | |
| | chlorophenyl)ethyl]piperazine (35 mg, 0.16 mmol, 1.3 equiv.) | |
| | and Et₃N (33 μL, 0.25 mmol, 2 equiv.) in DCM (0.6 mL) at RT for | |
| | 2 h. Purification by FC (cHex/EtOAc = 90/10 to 0/100). | |
| 061 | According to GP-4, 061 was obtained in 11% yield using | >95% |
| | sulfonyl chloride I-043 (70 mg, 0.14 mmol, 1 equiv.), 4- | |
| | phenylpiperidine (34 mg, 0.21 mmol, 1.5 equiv.) and Et₃N (40 μL, | |
| | 0.28 mmol, 2 equiv.) in DCM (0.7 mL) at RT for 2 h. Purification | |
| | by PTLC (cHex/EtOAc = 50/50). | |
| 062 | To a solution of I-046 (400 mg, 610 μmol, 1 equiv.) in DCM | >95% |
| | (3 mL) at RT, was added CF₃CO₂H (0.93 mL, 12 mmol, 20 | |
| | equiv.). The mixture was stirred at 40° C. for 2 h. It was | |
| | concentrated under reduced pressure and the residue was | |
| | azeotroped with toluene. The residue was purified by FC | |
| | (cHex/EtOAc = 95/5 to 50/50) to afford 320 mg of 062 (86%) as | |
| | a white foam. | |
| 063 | To a solution of I-049 (1.49g, 2.37 mmol, 1 equiv.) in DCM | >95% |
| | (10 mL) at RT, was added CF₃CO₂H (3.63 mL, 47 mmol, 20 | |
| | equiv.). The mixture was stirred at RT for 20 h. It was | |
| | concentrated under reduced pressure and the residue was | |
| | azeotroped with toluene. The residue was purified by FC | |
| | (cHex/EtOAc = 98/2 to 80/20) to afford 871 mg of 063 (63%) as | |
| | a white foam. | |
| 064 | To a solution of I-052 (400 mg, 0.578 mmol, 1 equiv.) in DCM | >95% |
| | (3 mL) at RT, was added CF₃CO₂H (0.89 mL, 12 mmol, 20 | |
| | equiv.). The mixture was stirred at 40° C. for 16 h. It was | |
| | concentrated under reduced pressure. The residue was | |
| | partitioned between DCM and water and the pH was adjusted to | |
| | 8 using 1N NaOH. The phases were separated and the aqueous | |
| | phase was extracted with DCM. The combined organic extracts | |
| | were dried (Na₂SO₄), filtered and concentrated under reduced | |
| | pressure. The residue was purified by FC (cHex/(EtOAc/EtOH | |
| | 3/1) = 95/5 to 50/50) to afford 310 mg of 064 (83%) as a white | |
| | foam. | |
| 065 | To a solution of I-056 (280 mg, 403 μmol, 1 equiv.) in DCM | >95% |
| | (2 mL) at RT, was added CF₃CO₂H (0.62 mL, 8.1 mmol, 20 | |
| | equiv.). The mixture was stirred at RT for 20 h. It was | |
| | concentrated under reduced pressure and the residue was | |
| | azeotroped with toluene. The residue was purified by FC | |
| | (cHex/EtOAc = 98/2 to 80/20) to afford 120 mg of 065 (49%) as | |
| | a white foam. | |
| 066 | According to GP-7, 066 was obtained in 70% yield using aryl | >95% |
| | bromide I-057 (80 mg, 193 μmol, 1 equiv.), piperazine I-001 | |
| | (62.2 mg, 231 μmol, 1.2 equiv.), t-BuONa (56 mg, 0.58 mmol, 3 | |
| | equiv.), XPhos-Pd-G3 (16 mg, 19 μmol, 0.1 equiv.) in toluene | |
| | (1 mL) at reflux for 2 h. Purification by FC (cHex/EtOAc = 95/5 | |
| | to 0/100) and PTLC (cHex/EtOAc = 50/50). | |
| 067 | According to GP-7, 067 was obtained in 49% yield using aryl | >95% |
| | bromide I-058 (80.0 mg, 203 μmol, 1 equiv.), piperazine I-001 | |
| | (65.4 mg, 242 μmol, 1.2 equiv.), t-BuONa (58 mg, 0.61 mmol, 3 | |
| | equiv.), XPhos-Pd-G3 (17 mg, 20 μmol, 0.1 equiv.) in toluene | |
| | (1 mL) at reflux for 2 h. Purification by FC (cHex/EtOAc = 94/6 | |
| | to 40/60). | |
| 068 | According to GP-7, 068 was obtained in 52% yield using aryl | >95% |
| | bromide I-059 (60.0 mg, 196 μmol, 1 equiv.), piperazine I-001 | |
| | (63.2 mg, 235 μmol, 1.2 equiv.), t-BuONa (56 mg, 0.59 mmol, 3 | |
| | equiv.), XPhos-Pd-G3 (17 mg, 20 μmol, 0.1 equiv.) in toluene | |
| | (1 mL) at reflux for 2 h. Purification by FC (cHex/EtOAc = 94/6 | |
| | to 40/60) and PTLC (DCM/MeOH = 95/5). | |
| 069 | A MW vial was charged with piperazine I-001 (221 mg, | >95% |
| | 0.821 mmol, 1.2 equiv.), aryl bromide I-060 (300 mg, 0.684 mmol, | |
| | 1 equiv.), RuPhos-Pd-G2 (51 mg, 68 μmol, 0.1 equiv.), Cs₂CO₃ | |
| | (669 mg, 2.05 mmol, 3 equiv.) and degassed 1,4-dioxane (3.4 mL) | |
| | and the reaction was stirred at 120° C. for 16 h. The reaction | |
| | mixture was filtered over celite (EtOAc rinses) and the filtrate | |
| | concentrated under reduced pressure. The residue was purified | |
| | by FC (cHex/EtOAc = 90/10 to 0/100) to afford 185 mg of 069 | |
| | (43%). | |
| 070 | To a solution of 069 (40 mg, 64 μmol, 1 equiv.) in dry THF | >95% |
| | (0.3 mL) at RT, was added LiBH₄ (2M in THF, 60 μL, 0.13 mmol, | |
| | 2 equiv.). The mixture was stirred at RT for 5 h. The reaction | |
| | mixture was partitioned between water and EtOAc. The layers | |
| | were separated and the aqueous phase was extracted with EtOAc. | |
| | The combined organic extracts were washed (brine), dried | |
| | (Na₂SO₄), filtered and concentrated under reduced pressure. The | |
| | residue was purified by PTLC (cHex/EtOAc = 50/50) to afford | |
| | 12 mg of 070 (31%). | |
| 071 | To a solution of 069 (40 mg, 64 μmol, 1 equiv.) in | >95% |
| | THF/MeOH/H₂O (3/1/1, 1 mL) at RT, was added LiOH (2.3 mg, | |
| | 96 μmol, 1.5 equiv.). The mixture was stirred at RT for 1 h. HCl | |
| | 1N was added until pH = 2 and EtOAc was added. The layers | |
| | were separated and the aqueous phase was extracted with EtOAc. | |
| | The combined organic extracts were washed (brine), dried | |
| | (Na₂SO₄), filtered and concentrated under reduced pressure. The | |
| | residue was purified by PTLC (cHex/EtOAc = 50/50) to afford | |
| | 17 mg of 071 (43%). | |
| 072 | To a solution of 069 (35 mg, 56 μmol, 1 equiv.) in dry THF | >95% |
| | (0.2 mL) at 0° C., was added MeMgBr (3M in Et₂O, 93 μL, | |
| | 0.28 mmol, 5 equiv.). The mixture was stirred at RT for 2 h. The | |
| | reaction mixture was partitioned between water and DCM. The | |
| | layers were separated and the aqueous phase was extracted with | |
| | DCM. The combined organic extracts were dried (Na₂SO₄), | |
| | filtered and concentrated under reduced pressure. The residue | |
| | was purified by PTLC (cHex/EtOAc = 50/50) to afford 4 mg of | |
| | 072 (10%). | |
| 073 | To a solution of alcohol 070 (70 mg, 0.12 mmol, 1 equiv) in DCM | >92% |
| | (0.6 mL) at 0° C., were added Et₃N (20 μL, 0.18 mmol, 1.5 equiv.) | |
| | and MsCl (10 μL, 0.14 mmol, 1.2 equiv). The mixture was stirred | |
| | at RT for 16 h. water was wadded and the layers were separated. | |
| | The organic extract was dried (Na₂SO₄), filtered and | |
| | concentrated under reduced pressure. The residue was dissolved | |
| | in THF (0.6 mL) at RT and M₂NH (2M solution in THF, 1.17 mL, | |
| | 2.34 mmol, 20 equiv.) was added. The mixture was stirred at RT | |
| | for 3 h before being concentrated under reduced pressure. The | |
| | residue was purified by PTLC (EtOAc/MeOH = 97/3) to afford | |
| | 24 mg of 073 (33%). | |
| 074 | According to GP-5, 074 was obtained in 49% yield using aryl | >95% |
| | bromide I-063 (126 mg, 351 μmol, 1 equiv.), piperazine I-001 | |
| | (113 mg, 422 μmol, 1.2 equiv.), Cs₂CO₃ (343 mg, 1.05 mmol, 3 | |
| | equiv.), Pd(OAc)₂ (7.9 mg, 35 μmol, 0.1 equiv.) and rac-BINAP | |
| | (26 mg, 42 μmol, 0.12 equiv.) in toluene (1.8 mL) at reflux for 2 h. | |
| | Purification by FC (cHex/EtOAc = 94/6 to 40/60) and PTLC | |
| | (cHex/EtOAc = 60/40). | |
| 075 | According to GP-5, 075 was obtained in 43% yield using aryl | >95% |
| | bromide I-065 (114 mg, 300 μmol, 1 equiv.), piperazine I-001 | |
| | (96.7 mg, 360 μmol, 1.2 equiv.), Cs₂CO₃ (293 mg, 0.900 mmol, 3 | |
| | equiv.), Pd(OAc)₂ (6.7 mg, 30 μmol, 0.1 equiv.) and rac-BINAP | |
| | (22 mg, 36 μmol, 0.12 equiv.) in toluene (1.5 mL) at reflux for 1 h. | |
| | Purification by FC (cHex/EtOAc = 94/6 to 40/60) and PTLC | |
| | (cHex/EtOAc = 60/40). | |
| 076 | A suspension of 063 (122 mg, 182 μmol, 1 equiv), 2- | >95% |
| | bromoethanol (38 μL, 0.57 mmol, 3 equiv.) and K₂CO₃ (100 mg, | |
| | 0.726 mmol, 4 equiv.) in DMF (0.9 mL) was stirred at 50° C. for | |
| | 6 h. The reaction mixture was partitioned between water and | |
| | EtOAc. The layers were separated and the aqueous phase was | |
| | extracted with EtOAc. The combined organic extracts were | |
| | washed (brine), dried (Na₂SO₄), filtered and concentrated under | |
| | reduced pressure. The residue was purified by FC (cHex/EtOAc = | |
| | 90/10 to 0/100) to afford 74 mg of 076 (65%). | |
| 077 | A suspension of 062 (90.0 mg, 147 μmol, 1 equiv), 2- | >95% |
| | bromoethanol (21 μL, 0.29 mmol, 2 equiv.) and K₂CO₃ (41 mg mg, | |
| | 0.29 mmol, 2 equiv.) in DMF (0.7 mL) was stirred at 50° C. for | |
| | 60 h. The reaction mixture was partitioned between water and | |
| | EtOAc. The layers were separated and the aqueous phase was | |
| | extracted with EtOAc. The combined organic extracts were | |
| | washed (brine), dried (Na₂SO₄), filtered and concentrated under | |
| | reduced pressure. The residue was purified by FC (cHex/EtOAc = | |
| | 90/10 to 0/100) to afford 26 mg of 077 (27%). | |
| 078 | A suspension of 064 (50 mg, 77 μmol, 1 equiv), 2-bromoethanol | >95% |
| | (6.6 μL, 93 μmol, 1.2 equiv.) and K₂CO₃ (21 mg, 0.15 mmol, 2 | |
| | equiv.) in DMF (0.4 mL) was stirred at 50° C. for 2 h. The reaction | |
| | mixture was partitioned between water and EtOAc. The layers | |
| | were separated and the aqueous phase was extracted with EtOAc. | |
| | The combined organic extracts were washed (brine), dried | |
| | (Na₂SO₄), filtered and concentrated under reduced pressure. The | |
| | residue was purified by PTLC (DCM/MeOH = 90/10) to afford | |
| | 18 mg of 078 (34%). | |
| 079 | A MW vial was charged with triflate I-066 (80 mg, 0.13 mmol, 1 | >90% |
| | equiv.), cyclopropylboronic acid (15 μL, 0.19 mmol, 1.5 equiv.), | |
| | K₂CO₃ (53 mg, 0.38 mmol, 3 equiv.) and Pd(PPh₃)₄ (15 mg, | |
| | 13 μmol, 0.1 equiv.). The vial was flushed with Ar and degassed | |
| | 1,4-dioxane (1.3 mL) was added. The vial was sealed and the | |
| | mixture was stirred at 110° C. for 2 h. The reaction mixture was | |
| | partitioned between water and EtOAc. The layers were separated | |
| | and the aqueous phase was extracted with EtOAc. The combined | |
| | organic extracts were washed (brine), dried (Na₂SO₄), filtered | |
| | and concentrated under reduced pressure. The residue was | |
| | purified by FC (cHex/EtOAc = 95/5 to 0/100) and PTLC | |
| | (DCM/MeOH = 95/5) to afford 30 mg of 079 (45%). | |
| 080 | A MW vial was charged with triflate I-067 (115 mg, 0.161 mmol, | >95% |
| | 1 equiv.), cyclopropylboronic acid (21 mg, 0.24 mmol, 1.5 | |
| | equiv.), K₂CO₃ (67 mg, 0.48 mmol, 3 equiv.) and Pd(PPh₃)₄ | |
| | (19 mg, 16 μmol, 0.1 equiv.). The vial was flushed with Ar and | |
| | degassed 1,4-dioxane (1.6 mL) was added. The vial was sealed | |
| | and the mixture was stirred at 110° C. for 1 h. The reaction mixture | |
| | was partitioned between water and EtOAc. The layers were | |
| | separated and the aqueous phase was extracted with EtOAc. The | |
| | combined organic extracts were washed (brine), dried (Na₂SO₄), | |
| | filtered and concentrated under reduced pressure. The residue | |
| | was purified by FC (cHex/EtOAc = 98/2 to 60/40) to afford | |
| | 72 mg of 080 (74%). | |
| 081 | A MW vial was charged with triflate I-067 (363 mg, 0.507 mmol, | >95% |
| | 1 equiv.), trimethyl-[2-[[4-(4,4,5,5-tetramethyl-1,3,2- | |
| | dioxaborolan-2-yl)imidazol-1-yl]methoxy]ethyl]silane (233 mg, | |
| | 0.718 mmol, 1.5 equiv.), Na₂CO₃ (228 mg, 2.15 mmol, 4.3 equiv.) | |
| | and Pd(PPh₃)₄ (83 mg, 72 μmol, 0.14 equiv.). The vial was | |
| | flushed with Ar and degassed 1,4-dioxane (3.4 mL) and water | |
| | (1 mL) were added. The vial was sealed and the mixture was | |
| | stirred at 100° C. for 2 h. The reaction mixture was partitioned | |
| | between water and EtOAc. The layers were separated and the | |
| | aqueous phase was extracted with EtOAc. The combined organic | |
| | extracts were washed (brine), dried (Na₂SO₄), filtered and | |
| | concentrated under reduced pressure. | |
| | The residue was dissolved in DCM (4 mL) at RT and CF₃CO₂H | |
| | (0.78 mL, 10 mmol, 20 equiv.) was added. The mixture was | |
| | stirred at reflux for 5 h before being concentrated under reduced | |
| | pressure. The residue was partitioned between sat. aq. NaHCO₃ | |
| | and DCM. The layers were separated and the aqueous phase was | |
| | extracted with DCM. The combined organic extracts were dried | |
| | (Na₂SO₄), filtered and concentrated under reduced pressure. The | |
| | residue was purified by FC (DCM/MeOH = 99/1 to 90/10) to | |
| | afford 28 mg of 081 (6%). | |
| 082 | To a solution of triflate I-067 (75 mg, 0.11 mmol, 1 equiv) in | >95% |
| | degassed 1,4-dioxane (0.5 mL) at RT, were added KOAc (31 mg, | |
| | 0.31 mmol, 3 equiv.), B₂pin₂ (40 mg, 0.15 mmol, 1.5 equiv) and | |
| | PdCl₂(dppf). CH₂Cl₂ complex (4.3 mg, 5.5 μmol, 0.1 equiv). the | |
| | mixture was stirred at 80° C. for 20 h. The mixture was partitioned | |
| | between water and DCM. The layers were separated and the | |
| | aqueous phase was extracted with DCM. The combined organic | |
| | extracts were dried (Na₂SO₄), filtered and concentrated under | |
| | reduced pressure. The residue was dissolved in degassed 1,4- | |
| | dioxane/water (5/1, 1 mL) at RT and Na₂CO₃ (32 mg, 0.30 mmol, | |
| | 3 equiv.), Pd(PPh₃)₄ (3.5 mg, 3.3 μmol, 0.03 equiv.) and 4-bromo- | |
| | 1-methyl-1H-imidazole (20 mg, 0.12 mmol, 1.2 equiv.) were | |
| | added. The mixture was stirred at 100° C. for 20 h. The reaction | |
| | mixture was filtered over Celite (EtOAc rinses) and the filtrate | |
| | was concentrated under reduced pressure. The residue was | |
| | purified by PTLC (EtOAc) to afford 9 mg of 082 (13%). | |
| 083 | To a solution of I-071 (55 mg, 85 μmol, 1 equiv.) in DCM | >95% |
| | (0.4 mL) at RT, was added CF₃CO₂H (0.13 mL, 1.7 mmol, 20 | |
| | equiv.). The mixture was stirred at RT for 0.5 h. It was | |
| | concentrated under reduced pressure and the residue was | |
| | azeotroped with toluene. The residue was purified by PTLC | |
| | (cHex/EtOAc = 30/70) to afford 15 mg of 083 (29%) as a white | |
| | foam. | |
| 084 | According to GP-4, 084 was obtained in 51% yield using | >95% |
| | sulfonyl chloride I-075 (107 mg, 224 μmol, 1 equiv.), 4-(4- | |
| | fluorophenyl)piperidine (61 mg, 0.34 mmol, 1.5 equiv.) and Et₃N | |
| | (47 μL, 0.34 mmol, 1.5 equiv.) in DCM (2.2 mL) at RT for 16 h. | |
| | Purification by FC (cHex/EtOAc = 90/10 to 10/90). | |
| 085 | According to GP-4, 085 was obtained in 30% yield using | >95% |
| | sulfonyl chloride I-075 (107 mg, 224 μmol, 1 equiv.), tert- | |
| | butylamine (35 μL, 0.34 mmol, 1.5 equiv.) and Et₃N (47 μL, | |
| | 0.34 mmol, 1.5 equiv.) in DCM (2.2 mL) at RT for 16 h. | |
| | Purification by FC (cHex/EtOAc = 90/10 to 10/90) and PTLC | |
| | (cHex/EtOAc/EtOH = 70/22/8). | |
| 086 | According to GP-4, 086 was obtained in 40% yield using | >95% |
| | sulfonyl chloride I-075 (107 mg, 224 μmol, 1 equiv.), N- | |
| | (cyclopropylmethyl)-N-methylamine (30 μL, 0.34 mmol, 1.5 | |
| | equiv.) and Et₃N (47 μL, 0.34 mmol, 1.5 equiv.) in DCM (2.2 mL) | |
| | at RT for 16 h. Purification by FC (cHex/EtOAc = 90/10 to | |
| | 10/90). | |
| 087 | According to GP-4, 087 was obtained in 32% yield using | >95% |
| | sulfonyl chloride I-079 (150 mg, 284 μmol, 1 equiv.), 4- | |
| | phenylpiperidine (69 mg, 0.43 mmol, 1.5 equiv.) and Et₃N (60 μL, | |
| | 0.43 mmol, 1.5 equiv.) in DCM (2 mL) at RT for 16 h. Purification | |
| | by FC (cHex/EtOAc = 95/5 to 50/50). | |
| 088 | According to GP-4, 088 was obtained in 46% yield using | >95% |
| | sulfonyl chloride I-079 (150 mg, 284 μmol, 1 equiv.), 4-(4- | |
| | fluorophenyl)piperidine (76 mg, 0.43 mmol, 1.5 equiv.) and Et₃N | |
| | (60 μL, 0.43 mmol, 1.5 equiv.) in DCM (2 mL) at RT for 16 h. | |
| | Purification by FC (cHex/EtOAc = 95/5 to 50/50). | |
| 089 | According to GP-4, 089 was obtained in 39% yield using | >95% |
| | sulfonyl chloride I-079 (150 mg, 284 μmol, 1 equiv.), tert- | |
| | butylamine (45 μL, 0.43 mmol, 1.5 equiv.) and Et₃N (60 μL, | |
| | 0.43 mol, 1.5 equiv.) in DCM (2 mL) at RT for 16 h. Purification | |
| | by FC (cHex/EtOAc = 94/6 to 40/60). | |
| 090 | To a solution of I-083 (130 mg, 202 μmol, 1 equiv.) in DCM | >90% |
| | (1 mL) at RT, was added CF₃CO₂H (0.31 mL, 4.0 mmol, 20 | |
| | equiv.). The mixture was stirred at RT for 0.5 h. It was | |
| | concentrated under reduced pressure and the residue was | |
| | azeotroped with toluene. The residue was purified by PTLC | |
| | (DCM/EtOAc = 90/10) to afford 75 mg of 090 (62%) as a white | |
| | foam. | |
| 091 | According to GP-4, 091 was obtained in 40% yield using | >95% |
| | sulfonyl chloride I-087 (81.9 mg, 173 μmol, 1 equiv.), 4-(4- | |
| | fluorophenyl)piperidine (47 mg, 0.26 mmol, 1.5 equiv.) and Et₃N | |
| | (36 μL, 0.26 mmol, 1.5 equiv.) in DCM (1.7 mL) at RT for 4 h. | |
| | Purification by FC (cHex/EtOAc = 95/5 to 50/50). | |
| 092 | According to GP-4, 092 was obtained in 38% yield using | >95% |
| | sulfonyl chloride I-087 (81.9 mg, 173 μmol, 1 equiv.), N- | |
| | (cyclopropylmethyl)-N-methylamine (22 mg, 0.26 mmol, 1.5 | |
| | equiv.) and Et₃N (36 μL, 0.26 mmol, 1.5 equiv.) in DCM (1.7 mL) | |
| | at RT for 4 h. Purification by FC (cHex/EtOAc = 95/5 to 50/50). | |
| 093 | According to GP-4, 093 was obtained in 48% yield using | >95% |
| | sulfonyl chloride I-087 (81.9 mg, 173 μmol, 1 equiv.), tert- | |
| | butylamine (19 mg, 0.26 mmol, 1.5 equiv.) and Et₃N (36 μL, | |
| | 0.26 mmol, 1.5 equiv.) in DCM (1.7 mL) at RT for 4 h. | |
| | Purification by FC (cHex/EtOAc = 95/5 to 50/50). | |
| 094 | According to GP-4, 094 was obtained in 34% yield using | >95% |
| | sulfonyl chloride I-091 (150 mg, 308 μmol, 1 equiv.), 4- | |
| | phenylpiperidine (74 mg, 0.46 mmol, 1.5 equiv.) and Et₃N (90 μL, | |
| | 0.62 mmol, 2 equiv.) in DCM (1.5 mL) at RT for 16 h. Purification | |
| | by FC (cHex/EtOAc = 95/5 to 50/50). | |
| 095 | According to GP-4, 095 was obtained in 33% yield using | >95% |
| | sulfonyl chloride I-095 (143 mg, 0.29 mmol, 1 equiv.), tert- | |
| | butylamine (32 mg, 0.44 mmol, 1.5 equiv.) and Et₃N (61 μL, | |
| | 0.44 mmol, 1.5 equiv.) in DCM (2.9 mL) at RT for 16 h. | |
| | Purification by FC (cHex/EtOAc = 95/5 to 50/50). | |
| 096 | According to GP-4, 096 was obtained in 46% yield using | >95% |
| | sulfonyl chloride I-095 (143 mg, 0.29 mmol, 1 equiv.), N- | |
| | (cyclopropylmethyl)-N-methylamine (37 mg, 0.44 mmol, 1.5 | |
| | equiv.) and Et₃N (61 μL, 0.44 mmol, 1.5 equiv.) in DCM (2.9 mL) | |
| | at RT for 16 h. Purification by FC (cHex/EtOAc = 95/5 to 50/50). | |
| 097 | According to GP-4, 097 was obtained in 45% yield using | >95% |
| | sulfonyl chloride I-095 (143 mg, 0.29 mmol, 1 equiv.), 4- | |
| | fluoropiperidine hydrochloride (61 mg, 0.44 mmol, 1.5 equiv.) | |
| | and Et₃N (121 μL, 0.870 mmol, 3 equiv.) in DCM (2.9 mL) at RT | |
| | for 16 h. Purification by FC (cHex/EtOAc = 95/5 to 50/50). | |
| 098 | According to GP-4, 098 was obtained in 43% yield using | >95% |
| | sulfonyl chloride I-095 (143 mg, 0.29 mmol, 1 equiv.), 4- | |
| | phenylpiperidine (70 mg, 0.44 mmol, 1.5 equiv.) and Et₃N (61 μL, | |
| | 0.44 mmol, 1.5 equiv.) in DCM (2.9 mL) at RT for 16 h. | |
| | Purification by FC (cHex/EtOAc = 95/5 to 50/50). | |
| 099 | According to GP-4, 099 was obtained in 49% yield using | >95% |
| | sulfonyl chloride I-095 (143 mg, 0.29 mmol, 1 equiv.), 4-(4- | |
| | fluorophenyl)piperidine (78 mg, 0.44 mmol, 1.5 equiv.) and Et₃N | |
| | (61 μL, 0.44 mmol, 1.5 equiv.) in DCM (2.9 mL) at RT for 16 h. | |
| | Purification by FC (cHex/EtOAc = 95/5 to 50/50). | |
| 100 | According to GP-4, 100 was obtained in 26% yield using | >95% |
| | sulfonyl chloride I-095 (171 mg, 0.346 mmol, 1 equiv.), | |
| | isopropylamine (41 mg, 0.69 mmol, 2 equiv.) and Et₃N (96 μL, | |
| | 0.69 mmol, 2 equiv.) in DCM (3.5 mL) at RT for 16 h. Purification | |
| | by FC (cHex/EtOAc = 95/5 to 50/50). | |
| 101 | According to GP-4, 101 was obtained in 27% yield using | >95% |
| | sulfonyl chloride I-095 (171 mg, 0.346 mmol, 1 equiv.), 1- | |
| | methylcyclopropanamine hydrochloride (75 mg, 0.69 mmol, 2 | |
| | equiv.) and Et₃N (193 μL, 1.39 mmol, 4 equiv.) in DCM (3.5 mL) | |
| | at RT for 16 h. Purification by FC (cHex/EtOAc = 95/5 to 50/50). | |
| 102 | According to GP-4, 102 was obtained in 31% yield using | >95% |
| | sulfonyl chloride I-095 (171 mg, 0.346 mmol, 1 equiv.), 1- | |
| | methylcyclobutan-1-amine hydrochloride (84 mg, 0.69 mmol, 2 | |
| | equiv.) and Et₃N (193 μL, 1.39 mmol, 4 equiv.) in DCM (3.5 mL) | |
| | at RT for 16 h. Purification by FC (cHex/EtOAc = 95/5 to 50/50). | |
| 103 | According to GP-4, 103 was obtained in 7% yield using sulfonyl | >95% |
| | chloride I-095 (171 mg, 0.346 mmol, 1 equiv.), 2-amino-2- | |
| | methyl-propanenitrile hydrochloride (84 mg, 0.69 mmol, 2 | |
| | equiv.) and Et₃N (193 μL, 1.39 mmol, 4 equiv.) in DCM (3.5 mL) | |
| | at RT for 16 h. Purification by FC (cHex/EtOAc = 95/5 to 50/50) | |
| | and PTLC (cHex/EtOAc = 40/60). | |
| 104 | According to GP-4, 104 was obtained in 7% yield using sulfonyl | >95% |
| | chloride I-095 (160 mg, 0.324 mmol, 1 equiv.), rac-1,1,1- | |
| | trifluoropropan-2-amine hydrochloride (97 mg, 0.65 mmol, 2 | |
| | equiv.) and Et₃N (181 μL, 1.30 mmol, 4 equiv.) in DCM (3.2 mL) | |
| | at RT for 16 h. Purification by FC (cHex/EtOAc = 95/5 to 50/50) | |
| | and PTLC (DCM/Acetone = 90/10). | |
| 105 | According to GP-4, 105 was obtained in 5% yield using sulfonyl | >95% |
| | chloride I-095 (160 mg, 0.324 mmol, 1 equiv.), 2,2,2-trifluoro- | |
| | 1,1-dimethyl-ethylamine hydrochloride (106 mg, 0.648 mmol, 2 | |
| | equiv.) and Et₃N (181 μL, 1.30 mmol, 4 equiv.) in DCM (3.2 mL) | |
| | at RT for 16 h. Purification by FC (cHex/EtOAc = 95/5 to 50/50) | |
| | and PTLC (DCM/Acetone = 90/10). | |
| 106 | According to GP-4, 106 was obtained in 46% yield using | >95% |
| | sulfonyl chloride I-095 (160 mg, 0.324 mmol, 1 equiv.), N,3- | |
| | dimethyl-3-oxetanamine hydrochloride (89 mg, 0.65 mmol, 2 | |
| | equiv.) and Et₃N (181 μL, 1.30 mmol, 4 equiv.) in DCM (3.2 mL) | |
| | at RT for 16 h. Purification by FC (cHex/EtOAc = 95/5 to 20/80). | |
| 107 | According to GP-4, 107 was obtained in 22% yield using | >95% |
| | sulfonyl chloride I-095 (92 mg, 0.19 mmol, 1 equiv.), N-methyl- | |
| | tert-butylamine (32 mg, 0.37 mmol, 2 equiv.) and Et₃N (52 μL, | |
| | 0.37 mmol, 2 equiv.) in DCM (1.9 mL) at RT for 16 h. Purification | |
| | by FC (cHex/EtOAc = 95/5 to 50/50). | |
| 108 | According to GP-4, 108 was obtained in 13% yield using | >95% |
| | sulfonyl chloride I-095 (120 mg, 0.24 mmol, 1 equiv.), 3- | |
| | methyloxetan-3-amine (42 mg, 0.49 mmol, 2 equiv.) and Et₃N | |
| | (70 μL, 0.49 mmol, 2 equiv.) in DCM (2.4 mL) at RT for 16 h. | |
| | Purification by PTLC (cHex/(EtOAc/EtOH = 3/1) = 80/20). | |
| 109 | According to GP-4, 109 was obtained in 23% yield using | >95% |
| | sulfonyl chloride I-095 (100 mg, 0.20 mmol, 1 equiv.), N-methyl- | |
| | 3-oxetaneamine (35 mg, 0.41 mmol, 2 equiv.) and Et₃N (60 μL, | |
| | 0.41 mmol, 2 equiv.) in DCM (1 mL) at RT for 16 h. Purification | |
| | by PTLC (cHex/(EtOAc/EtOH = 3/1) = 80/20). | |
| 110 | According to GP-4, 110 was obtained in 71% yield using | >95% |
| | sulfonyl chloride (S)-I-098 (25 mg, 52 μmol, 1 equiv.) and tert- | |
| | butylamine (0.11 mL, 1.0 mmol, 20 equiv.) in DCM (1 mL) at RT | |
| | for 2 h. Purification by FC (cHex/EtOAc = 95/5 to 50/50). | |
| 111 | According to GP-4, 111 was obtained in 11% yield using | >90% |
| | sulfonyl chloride (R)-I-098 (23 mg, 48 μmol, 1 equiv.) and tert- | |
| | butylamine (0.10 mL, 0.95 mmol, 20 equiv.) in DCM (1 mL) at | |
| | RT for 2 h. Purification by FC (cHex/EtOAc = 95/5 to 50/50). | |
| 112 | A suspension of 095 (100 mg, 0.189 mmol, 1 equiv), 2- | >95% |
| | bromoethanol (20 μL, 0.28 mmol, 1.5 equiv.) and K₂CO₃ (29 mg, | |
| | 0.21 mmol, 1.1 equiv.) in DMF (0.9 mL) was stirred at RT for | |
| | 60 h. The reaction mixture was partitioned between water and | |
| | EtOAc. The layers were separated and the aqueous phase was | |
| | extracted with EtOAc. The combined organic extracts were | |
| | washed (brine), dried (Na₂SO₄), filtered and concentrated under | |
| | reduced pressure. The residue was purified by FC (cHex/EtOAc = | |
| | 100/0 to 40/60) to afford 40 mg of 112 (37%). | |
| 113 | A suspension of 095 (100 mg, 0.189 mmol, 1 equiv), 2- | >95% |
| | bromoethyl methyl ether (27 μL, 0.28 mmol, 1.5 equiv.) and | |
| | K₂CO₃ (29 mg, 0.21 mmol, 1.1 equiv.) in DMF (0.9 mL) was | |
| | stirred at RT for 60 h. The reaction mixture was partitioned | |
| | between water and EtOAc. The layers were separated and the | |
| | aqueous phase was extracted with EtOAc. The combined organic | |
| | extracts were washed (brine), dried (Na₂SO₄), filtered and | |
| | concentrated under reduced pressure. The residue was purified | |
| | by FC (cHex/EtOAc = 100/0 to 0/100) to afford 22 mg of 113 | |
| | (20%). | |
| 114 | A suspension of 095 (100 mg, 0.189 mmol, 1 equiv), iodomethane | >95% |
| | (18 μL, 0.28 mmol, 1.5 equiv.) and K₂CO₃ (29 mg, 0.21 mmol, 1.1 | |
| | equiv.) in DMF (0.9 mL) was stirred at RT for 16 h. The reaction | |
| | mixture was partitioned between water and EtOAc. The layers | |
| | were separated and the aqueous phase was extracted with EtOAc. | |
| | The combined organic extracts were washed (brine), dried | |
| | (Na₂SO₄), filtered and concentrated under reduced pressure. The | |
| | residue was purified by FC (cHex/EtOAc = 100/0 to 40/60) to | |
| | afford 61 mg of 114 (60%). | |
| 115 | According to GP-7, 115 was obtained in 32% yield using aryl | >95% |
| | bromide I-099 (100 mg, 306 μmol, 1 equiv.), piperazine I-001 | |
| | (107 mg, 398 μmol, 1.2 equiv.), t-BuONa (88 mg, 0.92 mmol, 3 | |
| | equiv.), XPhos-Pd-G3 (26 mg, 31 μmol, 0.1 equiv.) in toluene | |
| | (1.5 mL) at reflux for 4 h. Purification by FC (cHex/EtOAc = 95/5 | |
| | to 0/100). | |
| 116 | According to GP-7, 116 was obtained in 62% yield using aryl | >95% |
| | bromide I-100 (80.0 mg, 181 μmol, 1 equiv.), piperazine I-001 | |
| | (58.5 mg, 217 μmol, 1.2 equiv.), t-BuONa (52 mg, 0.54 mmol, 3 | |
| | equiv.), XPhos-Pd-G3 (15 mg, 18 μmol, 0.1 equiv.) in toluene | |
| | (0.9 mL) at reflux for 4 h. Purification by FC (cHex/EtOAc = 94/6 | |
| | to 40/60). | |
| 117 | According to GP-4, 117 was obtained in 59% yield using | >95% |
| | sulfonyl chloride I-102 (50 mg, 0.10 mmol, 1 equiv.), 4- | |
| | phenylpiperidine (20 mg, 0.12 mmol, 1.2 equiv.) and Et₃N (40 μL, | |
| | 0.31 mmol, 3 equiv.) in DCM (0.5 mL) at RT for 16 h. Purification | |
| | by PTLC (cHex/EtOAc/EtOH = 70/23/7). | |
| 118 | According to GP-7, 118 was obtained in 41% yield using aryl | >95% |
| | bromide I-103 (97.0 mg, 221 μmol, 1 equiv.), piperazine I-001 | |
| | (71.3 mg, 266 μmol, 1.2 equiv.), t-BuONa (64 mg, 0.66 mmol, 3 | |
| | equiv.), XPhos-Pd-G3 (9.4 mg, 11 μmol, 0.05 equiv.) in toluene | |
| | (1.1 mL) at reflux for 2 h. Purification by FC (cHex/EtOAc = 95/5 | |
| | to 50/50). | |
| 119 | According to GP-7, 119 was obtained in 17% yield using aryl | >95% |
| | bromide I-107 (60.0 mg, 131 μmol, 1 equiv.), piperazine I-001 | |
| | (42.2 mg, 157 μmol, 1.2 equiv.), t-BuONa (38 mg, 0.39 mmol, 3 | |
| | equiv.), XPhos-Pd-G3 (11 mg, 13 μmol, 0.1 equiv.) in toluene | |
| | (0.7 mL) at reflux for 2 h. Purification by FC (cHex/EtOAc = | |
| | 90/10 to 0/100) and PTLC (DCM/MeOh = 98/2). | |
| 120 | According to GP-7, 120 was obtained in 31% yield using aryl | >95% |
| | bromide I-107 (80.0 mg, 184 μmol, 1 equiv.), piperazine I-033 | |
| | (63.2 mg, 221 μmol, 1.2 equiv.), t-BuONa (53 mg, 0.55 mmol, 3 | |
| | equiv.), XPhos-Pd-G3 (16 mg, 18 μmol, 0.1 equiv.) in toluene | |
| | (0.9 mL) at reflux for 2 h. Purification by FC (cHex/EtOAc = | |
| | 90/10 to 0/10). | |
| 121 | According to GP-5, 121 was obtained in 17% yield using aryl | >95% |
| | bromide I-109 (100 mg, 236 μmol, 1 equiv.), piperazine I-001 | |
| | (82.5 mg, 307 μmol, 1.3 equiv.), t-BuONa (45.4 mg, 0.472 mmol, | |
| | 2 equiv.), Pd₂(dba)₃ (10.8 mg, 12 μmol, 0.05 equiv.) and rac- | |
| | BINAP (22 mg, 35 μmol, 0.15 equiv.) in toluene (1.2 mL) at reflux | |
| | for 24 h. Purification by FC (cHex/EtOAc = 90/10 to 0/100) and | |
| | PTLC (cHex/EtOAc/EtOH = 80/15/5). | |
| 122 | To a solution of I-111 (127 mg, 0.216 mmol, 1 equiv.) in EtOH | >95% |
| | (4.3 mL) at RT, was added Pd/C (10% on C, 23 mg, 11 μmol, 0.05 | |
| | equiv.). The mixture was stirred under a H₂ atmosphere for 4 h. | |
| | The suspension was filtered over Celite (EtOH rinses) and the | |
| | filtrate was concentrated under reduced pressure to afford 50 mg | |
| | of impure residue. It was dissolved in DCM (0.5 mL) and a | |
| | solution of TPTU (27 mg, 92 μmol, 0.45 equiv.), 2-chloro-4,5- | |
| | difluorobenzoic acid (18 mg, 92 μmol, 0.45 equiv.) and iPr2Net | |
| | (27 μL, 15 μmol, 0.75equiv.) in DCM (0.5 mL). The mixture was | |
| | stirred at RT for 16 h. Sat. aq. NH₄Cl and EtOAc were added. The | |
| | layers were separated and the organic extract was washed | |
| | (brine), dried (Na₂SO₄), filtered and concentrated under reduced | |
| | pressure. The residue was purified by PTLC (cHex/EtOAc/EtOH = | |
| | 50/38/12) to afford 14 mg of 122 (10%). | |
| 123 | A solution of I-113 (32 mg, 34 μmol, 1 equiv.) in 1,1,1,3,3,3- | >85% |
| | hexafluoropropan-2-ol (1.8 mL) was stirred at 135° C. under MW | |
| | irradiation for 3 h. The solution was concentrated under reduced | |
| | pressure. The residue was purified by PTLC (DCM/MeOH = | |
| | 95/5) to afford 9 mg of 123 (85% pure, 36% yield). | |
| 124 | To a solution of I-118 (60 mg, 93 μmol, 1 equiv.) in DCM | >95% |
| | (0.5 mL) at RT, was added HCl (4M in dioxane, 0.23 mL, | |
| | 0.93 mmol, 10 equiv.). The mixture was stirred at RT for 16 h. It | |
| | was concentrated under reduced pressure. The residue was | |
| | purified by PTLC (cHex/EtOAc = 50/50) to afford 52 mg of 124 | |
| | (93%) as a white foam. | |
| 125 | According to GP-7, 125 was obtained in 23% yield using aryl | >95% |
| | bromide I-120 (1.64 g, 4.05 mmol, 1 equiv.), piperazine I-001 | |
| | (1.30 g, 4.85 mol, 1.2 equiv.), t-BuONa (1.17 g, 12.1 mmol, 3 | |
| | equiv.), XPhos-Pd-G3 (171 g, 202 μmol, 0.05 equiv.) in toluene | |
| | (20 mL) at reflux for 4 h. Purification by FC (DCM/EtOAc = 95/5 | |
| | to 50/50). | |
| 126 | According to GP-4, 126 was obtained in 97% yield using | >95% |
| | sulfonyl chloride I-008 (50.0 mg, 106 μmol, 1 equiv.), 3- | |
| | phenoxypyrrolidine (26 mg, 0.16 mmol, 1.5 equiv.) and iPr₂NEt | |
| | (37 μL, 0.21 mmol, 2 equiv.) in DCM (0.5 mL) at RT for 60 h. | |
| | Purification by FC (cHex/EtOAc = 70/30 to 0/100) and then | |
| | PTLC (DCM/MeOH = 95/5). | |
| 127 | According to GP-4, 127 was obtained in 60% yield using | >95% |
| | sulfonyl chloride I-008 (50.0 mg, 127 μmol, 1 equiv.), N-methyl- | |
| | N-phenyl-pyrrolidin-3-amine dihydrochloride (47 mg, | |
| | 0.19 mmol, 1.5 equiv.) and iPr₂NEt (0.15 mL, 0.89 mmol, 7 | |
| | equiv.) in DCM (0.6 mL) at RT for 16 h. Purification by FC | |
| | (cHex/EtOAc = 98/2 to 20/80). | |
| 128 | To a solution of piperazine I-037 (80 mg, 0.15 mmol, 1 equiv.) in | >95% |
| | isopropanol (1 mL) at RT, was added 2-(4-Fluorophenyl)oxirane | |
| | (21 mg, 0.15 mmol, 2 equiv.). The mixture was stirred at 80° C. for | |
| | 8 h. The recation mixture was concentrated under reduced | |
| | pressure. The residue was purified by FC (cHex/EtOAc = 90/10 | |
| | to 20/80) affording 128 in 48% yield. | |
| 129 | According to GP-4, 129 was obtained in 61% yield using | >95% |
| | sulfonyl chloride I-039 (70 mg, 0.14 mmol, 1 equiv.), tert- | |
| | butylmaine (20 mg, 0.27 mmol, 2 equiv.) and Et₃N (40 μL, | |
| | 0.27 mmol, 2 equiv.) in DCM (0.7 mL) at RT for 16 h. Purification | |
| | by PTLC (DCM/MeOH = 95/5). | |
| 130 | According to GP-4, 130 was obtained in 52% yield using | >95% |
| | sulfonyl chloride I-041 (75 mg, 0.15 mmol, 1 equiv.), 4- | |
| | fluoropiperidine hydrochloride (26 mg, 0.18 mmol, 1.2 equiv.) | |
| | and Et₃N (64 μL, 0.46 mmol, 3 equiv.) in DCM (0.8 mL) at RT for | |
| | 2 h. Purification by PTLC (cHex/EtOAc = 50/50). | |
| 131 | According to GP-4, 131 was obtained in 16% yield using | >95% |
| | sulfonyl chloride I-095 (100 mg, 0.202 mmol, 1 equiv.), 3,3- | |
| | dimethylmorpholine (30 μL, 0.24 mmol, 1.2 equiv.) and Et₃N | |
| | (56 μL, 0.40 mmol, 2 equiv.) in DCM (1 mL) at RT for 16 h. | |
| | Purification by PTLC (DCM/MeOH = 95/5). | |
| 132 | A suspension of 074 (50 mg, 95 μmol, 1 equiv.), iodomethane | >95% |
| | (20 μL, 0.28 mmol, 3 equiv.) and K₂CO₃ (39 mg, 0.28 mmol, 3 | |
| | equiv.) in DMF (0.5 mL) was stirred at 50° C. for 16 h. The | |
| | reaction mixture was partitioned between water and EtOAc. The | |
| | layers were separated and the aqueous phase was extracted with | |
| | EtOAc. The combined organic extracts were washed (brine), | |
| | dried (Na₂SO₄), filtered and concentrated under reduced | |
| | pressure. The residue was purified by PTLC (cHex/EtOAc = | |
| | 60/40) to afford 34 mg of 132 (67%). | |
| 133 | A suspension of 075 (30 mg, 55 μmol, 1 equiv.), iodomethane | >95% |
| | (12 μL, 0.16 mmol, 3 equiv.) and K₂CO₃ (23 mg, 0.16 mmol, 3 | |
| | equiv.) in DMF (0.5 mL) was stirred at 50° C. for 16 h. The | |
| | reaction mixture was partitioned between water and EtOAc. The | |
| | layers were separated and the aqueous phase was extracted with | |
| | EtOAc. The combined organic extracts were washed (brine), | |
| | dried (Na₂SO₄), filtered and concentrated under reduced | |
| | pressure. The residue was purified by PTLC (cHex/EtOAc = | |
| | 60/40) to afford 20 mg of 133 (64%). | |
| 134 | A suspension of 107 (75 mg, 0.14 mmol, 1 equiv.), 2- | >95% |
| | bromoethanol (15 μL, 0.21 mmol, 1.5 equiv.) and K₂CO₃ (38 mg, | |
| | 0.27 mmol, 2 equiv.) in DMF (0.7 mL) was stirred at RT for 6 | |
| | days. The reaction mixture was partitioned between water and | |
| | EtOAc. The layers were separated and the aqueous phase was | |
| | extracted with EtOAc. The combined organic extracts were | |
| | washed (brine), dried (Na₂SO₄), filtered and concentrated under | |
| | reduced pressure. The residue was purified by FC (cHex/EtOAc = | |
| | 98/2 to 50/50) to afford 66 mg of 134 (82%). | |
| 135 | A suspension of 107 (75 mg, 0.14 mmol, 1 equiv.), 2-bromoethyl | >95% |
| | methyl ether (19 μL, 0.21 mmol, 1.5 equiv.) and K₂CO₃ (38 mg, | |
| | 0.27 mmol, 2 equiv.) in DMF (0.7 mL) was stirred at RT for 6 | |
| | days. The reaction mixture was partitioned between water and | |
| | EtOAc. The layers were separated and the aqueous phase was | |
| | extracted with EtOAc. The combined organic extracts were | |
| | washed (brine), dried (Na₂SO₄), filtered and concentrated under | |
| | reduced pressure. The residue was purified by FC (cHex/EtOAc = | |
| | 98/2 to 50/50) to afford 83 mg of 135 (100%). | |
| 136 | A suspension of 107 (70 mg, 0.13 mmol, 1 equiv.), benzyl | >95% |
| | bromide (23 μL, 0.19 mmol, 1.5 equiv.) and K₂CO₃ (36 mg, | |
| | 0.26 mmol, 2 equiv.) in DMF (0.6 mL) was stirred at RT for 4 h. | |
| | The reaction mixture was partitioned between water and EtOAc. | |
| | The layers were separated and the aqueous phase was extracted | |
| | with EtOAc. The combined organic extracts were washed | |
| | (brine), dried (Na₂SO₄), filtered and concentrated under reduced | |
| | pressure. The residue was purified by FC (cHex/EtOAc = 90/10 | |
| | to 50/50) to afford 64 mg of 136 (78%). | |
| 137 | A suspension of 107 (70 mg, 0.13 mmol, 1 equiv.), 3- | >95% |
| | (bromomethyl)pyridine hydrobromide (49 mg, 0.19 mmol, 1.5 | |
| | equiv.) and K₂CO₃ (71 mg, 0.51 mmol, 4 equiv.) in DMF (0.6 mL) | |
| | was stirred at RT for 16 h. The reaction mixture was partitioned | |
| | between water and EtOAc. The layers were separated and the | |
| | aqueous phase was extracted with EtOAc. The combined organic | |
| | extracts were washed (brine), dried (Na₂SO₄), filtered and | |
| | concentrated under reduced pressure. The residue was purified | |
| | by FC (cHex/EtOAc = 90/10 to 50/50) to afford 60 mg of 137 | |
| | (73%). | |
| 138 | A suspension of 107 (70 mg, 0.13 mmol, 1 equiv.), 4- | >95% |
| | (bromomethyl)pyridine hydrobromide (49 mg, 0.19 mmol, 1.5 | |
| | equiv.) and K₂CO₃ (71 mg, 0.51 mmol, 4 equiv.) in DMF (0.6 mL) | |
| | was stirred at RT for 16 h. The reaction mixture was partitioned | |
| | between water and EtOAc. The layers were separated and the | |
| | aqueous phase was extracted with EtOAc. The combined organic | |
| | extracts were washed (brine), dried (Na₂SO₄), filtered and | |
| | concentrated under reduced pressure. The residue was purified | |
| | by FC (cHex/EtOAc = 90/10 to 50/50) to afford 67 mg of 138 | |
| | (82%). | |
| 139 | According to GP-4, 139 was obtained in 34% yield using | >95% |
| | sulfonyl chloride I-124 (110 mg, 215 μmol, 1 equiv.) and tert- | |
| | butylamine (0.23 mL, 2.1 mmol, 10 equiv.) in DCM (1.1 mL) at | |
| | RT for 2 h. Purification by FC (cHex/EtOAc = 95/5 to 50/50). | |

Analytical data for the final compounds

The analytical data for the final compounds are presented on the following Table 3.

TABLE 3

| Cpd | Description |
|---|---|
| 001 | White solid. ¹H NMR (400 MHz, Chloroform-d, multiple sets of rotamers) |
| | δ 7.55-7.45 (m, 1H), 7.39-7.28 (m, 2H), 7.23-7.14 (m, 1H), 7.12-7.02 (m, |
| | 1H), 6.98-6.90 (m, 1H), 4.24-3.65 (m, 6H), 3.60-2.75 (m, 6H), 2.72-2.65 |
| | (m, 3H), 1.62-1.48 (m, 2H), 1.06-0.76 (m, 6H). ¹⁹F NMR (376 MHz, |
| | Chloroform-d, multiple sets of rotamers) δ-109.28, -109.29 -109.34, -109.36. |
| | MS (ESI⁺): [M + H]⁺ 498.1/500.1. |
| 002 | White solid. ¹H NMR (400 MHz, Chloroform-d, 2 sets of rotamers) δ 7.47 & |
| | 7.46 (d, J = 8.5, 1H), 7.38-7.12 (m, 2H), 7.18-7.13 (m, 1H), 7.10-7.00 (m, |
| | 1H), 6.90 (d, J = 8.5 Hz, 1H), 4.50 & 4.49 (d, J = 5.6 Hz, 1H), 4.38 (s, 1H), |
| | 4.29-4.26 (m, 1H), 4.06-4.02 (m, 1H), 3.92 & 3.91 (s, 3H), 3.74-3.62 (m, |
| | 1H), 3.69-3.48 (m, 1H), 3.31 & 3.28 (d, J = 5.1 Hz, 1H), 3.13 (t, J = 10.5 |
| | Hz, 1H), 2.07-1.87 (m, 3H), 1.21 (s, 9H). ¹⁹F NMR (376 MHz, Chloroform- |
| | d, 2 sets of rotamers) δ-109.3, -109.4. MS (ESI⁺): [M + H]⁺ 510.1/512.1. |
| 003 | White solid. ¹H NMR (400 MHz, Chloroform-d, 2 sets of rotamers) δ 7.38- |
| | 7.34 (m, 1.5H), 7.22-7.13 (m, 2.5H), 7.10-7.00 (m, 1H), 6.93 (d, J = 8.5 Hz, |
| | 1H), 4.53-4.48 (m, 1H), 4.31-4.26 (m, 1H), 4.06-4.03 (m, 1H), 3.92 & 3.92 |
| | (s, 3H), 3.69-3.49 (m, 1H), 3.31-3.27 (m, 1H), 3.17-3.10 (m, 1H), 2.92 (t, |
| | J = 7.1 Hz, 2H), 2.68 (s, 3H), 2.05-1.88 (m, 3H), 1.66-1.49 (m, 3H), 0.91 (t, |
| | J = 7.4 Hz, 3H). ¹⁹F NMR (376 MHz, Chloroform-d, 2 sets of rotamers) |
| | δ-109.3, -109.4. MS (ESI⁺): [M + H]⁺ 510.1/512.1. |
| 004 | White solid. ¹H NMR (400 MHz, Chloroform-d, 2 sets of rotamers) δ 7.42- |
| | 7.35 (m, 1.5H), 7.23-7.20 (m, 1.5H), 7.18-7.13 (m, 1H), 7.10-7.01 (m, 1H), |
| | 6.94 (d, J = 8.5 Hz, 1H), 4.53-4.48 (m, 1H), 4.31-4.28 (m, 1H), 4.08-4.04 |
| | (m, 1H), 3.93 & 3.92 (s, 3H), 3.69-3.49 (m, 1H), 3.31-3.26 (m, 1H), 3.22- |
| | 3.11 (m, 5H), 2.06-1.88 (m, 4H), 1.76-1.73 (m, 4H). ¹⁹F NMR (376 MHz, |
| | Chloroform-d, 2 sets of rotamers) δ-109.3, -109.4. MS (ESI⁺): [M + H]⁺ |
| | 508.1/510.1. |
| 005 | White solid. ¹H NMR (400 MHz, Chloroform-d, 2 sets of rotamers) δ 7.39- |
| | 7.32 (m, 1.5H), 7.23-7.13 (m, 2.5H), 7.10-7.01 (m, 1H), 6.92 (d, J = 8.5 Hz, |
| | 1H), 4.53-4.47 (m, 1H), 4.31-4.25 (m, 1H), 4.07-4.03 (m, 1H), 3.92 & 3.91 |
| | (s, 3H), 3.69-3.47 (m, 1H), 3.32-3.25 (m, 1H), 3.17-3.10 (m, 1H), 2.86 (d, |
| | J = 6.8 Hz, 2H), 2.79 (s, 3H), 2.07-1.60 (m, 4H), 0.91-0.80 (m, 1H), 0.56-0.48 |
| | (m, 2H), 0.18-0.12 (m, 2H). ¹⁹F NMR (376 MHz, Chloroform-d, 2 sets of |
| | rotamers) δ-109.3, -109.4. MS (ESI⁺): [M + H]⁺ 522.1/524.1. |
| 006 | White solid. ¹H NMR (400 MHz, Chloroform-d, 2 sets of rotamers) δ 7.38- |
| | 7.32 (m, 1H), 7.23-7.13 (m, 2H), 7.12-7.11 (m, 1H), 7.10-7.01 (m, 1H), 6.94 |
| | (d, J = 8.5, 1H), 4.83-4.66 (m, 1H), 4.54-4.48 (m, 1H), 4.30-4.25 (m, 1H), |
| | 4.08-4.04 (m, 1H), 3.93 & 3.92 (s, 3H), 3.70-3.48 (m, 1H), 3.37-3.26 (m, |
| | 3H), 3.17-3.10 (m, 1H), 2.86-2.78 (m, 2H), 2.08-1.82 (m, 8H). ¹⁹F NMR |
| | (376 MHz, Chloroform-d, 2 sets of rotamers) δ-109.3, -109.4, -185.8. MS |
| | (ESI⁺): [M + H]⁺ 540.1/542.1. |
| 007 | White solid. ¹H NMR (400 MHz, Chloroform-d, 2 sets of rotamers) δ 7.38- |
| | 7.32 (m, 1.5H), 7.23-7.13 (m, 1.5H), 7.12-7.01 (m, 2H), 6.94 (d, J = 8.5 Hz, |
| | 1H), 4.54-4.48 (m, 1H), 4.30-4.25 (m, 1H), 4.08-4.03 (m, 1H), 3.93 & 3.92 |
| | (s, 3H), 3.88-3.82 (m, 2H), 3.69-3.47 (m, 1H), 3.31-3.25 (m, 1H), 3.17- |
| | 3.10 (m, 1H), 2.30-2.21 (m, 2H), 2.07-1.61 (m, 9H). ¹⁹F NMR (376 MHz, |
| | Chloroform-d, 2 sets of rotamers) δ-73.7, -73.7, -109.2, -109.3. |
| | MS (ESI⁺): [M + H]⁺ 590.1/592.1. |
| 008 | White solid. ¹H NMR (400 MHz, Chloroform-d, 2 sets of rotamers) δ 7.38- |
| | 7.28 (m, 4H), 7.22-6.99 (m, 6H), 6.91 (d, J = 8.5 Hz, 1H), 6.31 (s, 1H), 4.52- |
| | 4.46 (m, 1H), 4.29-4.27 (m, 1H), 4.06-4.01 (m, 1H), 3.91 & 3.90 (s, 3H), |
| | 3.69-3.47 (m, 1H), 3.30-3.24 (m, 1H), 3.16-3.06 (m, 3H), 2.99-2.94 (m, |
| | 2H), 2.56 (t, J = 5.8 Hz, 2H), 2.44 (t, J = 5.9 Hz, 2H), 2.06-1.86 (m, 4H). |
| | ¹⁹F NMR (376 MHz, Chloroform-d, 2 sets of rotamers) δ-109.3, -109.4. MS |
| | (ESI⁺): [M + H]⁺ 610.1/612.1. |
| 009 | White solid. ¹H NMR (400 MHz, Chloroform-d, 2 sets of rotamers) δ 7.39- |
| | 7.12 (m, 9H), 7.11-7.00 (m, 1H), 6.96 (d, J = 8.5 Hz, 1H), 4.55-4.47 (m, |
| | 1H), 4.34-4.28 (m, 1H), 4.10-4.00 (m, 1H), 3.94 & 3.94 (s, 3H), 3.92-3.85 |
| | (m, 2H), 3.72-3.48 (m, 1H), 3.30 (d, J = 13.0 Hz, 1H), 3.18-3.11 (m, 1H), |
| | 2.44-2.27 (m, 3H), 2.10-1.60 (m, 8H). ¹⁹F NMR (376 MHz, Chloroform-d, |
| | 2 sets of rotamers) δ-109.3, -109.4. MS (ESI⁺): [M + H]⁺ 598.1/600.2. |
| 010 | White solid. ¹H NMR (400 MHz, Chloroform-d, 2 sets of rotamers) δ 7.39- |
| | 7.01 (m, 10H), 6.92 (dd, J = 8.6, 1.2 Hz, 1H), 4.54-4.47 (m, 1H), 4.30-4.25 |
| | (m, 1H), 4.07-4.02 (m, 1H), 3.92 & 3.92 (s, 3H), 3.76-3.26 (m, 4H), 3.17- |
| | 3.10 (m, 1H), 2.61-2.55 (m, 2H), 2.18 (t, J = 11.6 Hz, 2H), 2.06-1.86 (m, |
| | 4H), 1.80-1.58 (m, 3H), 1.38-1.18 (m, 4H). ¹⁹F NMR (376 MHz, |
| | Chloroform-d, 2 sets of rotamers) δ-109.3, -109.4. MS (ESI⁺): [M + H]⁺ |
| | 626.2/628.2. |
| 011 | White solid. ¹H NMR (400 MHz, Chloroform-d, 2 sets of rotamers) δ 7.40- |
| | 6.99 (m, 10H), 6.93 (d, J = 8.5 Hz, 1H), 4.55-4.48 (m, 1H), 4.30-4.23 (m, |
| | 1H), 4.07-3.99 (m, 1H), 3.93 & 3.92 (s, 3H), 3.70-3.46 (m, 3H), 3.33-3.10 |
| | (m, 2H), 2.62-2.53 (m, 1H), 2.47-2.29 (m, 2H), 2.13-1.84 (m, 6H), 1.75- |
| | 1.59 (m, 3H), 0.99-0.84 (m, 1H). ¹⁹F NMR (376 MHz, Chloroform-d, 2 sets |
| | of rotamers) δ-109.3, -109.4. MS (ESI⁺): [M + H]⁺ 612.1/614.2. |
| 012 | White solid. ¹H NMR (400 MHz, Chloroform-d, 2 sets of rotamers) δ 7.39- |
| | 7.27 (m, 2H), 7.26-6.99 (m, 5H), 6.97-6.93 (m, 1H), 6.92-6.85 (m, 3H), |
| | 4.54-4.48 (m, 1H), 4.31-4.26 (m, 1H), 4.09-4.05 (m, 1H), 3.92 & 3.93 (s, |
| | 3H), 3.71-3.47 (m, 1H), 3.33-3.06 (m, 10H), 2.08-1.60 (m, 4H). ¹⁹F NMR |
| | (376 MHz, Chloroform-d, 2 sets of rotamers) δ-109.3, -109.4. |
| | MS (ESI⁺): [M + H]⁺ 599.2/602.2. |
| 013 | White solid. ¹H NMR (400 MHz, Chloroform-d, 2 sets of rotamers) δ 8.26 |
| | (d, J = 4.7 Hz, 2H), 7.39-7.31 (m, 1.5H), 7.23-7.00 (m, 3.5H), 6.92 (d, J = |
| | 8.5 Hz, 1H), 6.50 (t, J = 4.7 Hz, 1H), 4.52-4.45 (m, 1H), 4.30-4.24 (m, 1H), |
| | 4.05-4.00 (m, 1H), 3.93-3.88 (m, 7H), 3.68-3.46 (m, 1H), 3.29-3.23 (m, 1H), |
| | 3.16-3.08 (m, 1H), 3.02 (t, J = 5.1 Hz, 4H), 2.07-1.64 (m, 4H). ¹⁹F NMR |
| | (376 MHz, Chloroform-d, 2 sets of rotamers) δ-109.3, -109.4. |
| | MS (ESI⁺): [M + H]⁺ 601.2/604.1. |
| 014 | White solid. ¹H NMR (400 MHz, Chloroform-d, 2 sets of rotamers) δ 7.38- |
| | 6.99 (m, 10H), 6.92 (dd, J = 8.5, 1.5 Hz, 1H), 4.53-4.46 (m, 1H), 4.29-4.25 |
| | (m, 1H), 4.07-4.03 (m, 1H), 3.92 & 3.91 (s, 3H), 3.69-3.49 (m, 1H), 3.48 (s, |
| | 2H), 3.31-3.25 (m, 1H), 3.16-3.09 (m, 1H), 2.98 (s, 4H), 2.57-2.49 (m, 4H), |
| | 2.06-1.58 (m, 4H). ¹⁹F NMR (376 MHz, Chloroform-d, 2 sets of rotamers) |
| | δ-109.3, -109.4. MS (ESI⁺): [M + H]⁺ 613.2/615.2. |
| 015 | White solid. ¹H NMR (400 MHz, Chloroform-d, 2 sets of rotamers) δ 7.44- |
| | 7.34 (m, 1.5H), 7.23-7.20 (m, 1.5H), 7.18-7.13 (m, 1H), 7.10-7.01 (m, 1H), |
| | 6.94 (d, J = 8.4 Hz, 1H), 4.54-4.48 (m, 1H), 4.32-4.27 (m, 1H), 4.09-4.03 |
| | (m, 1H), 3.93 & 3.92 (s, 3H), 3.70-3.49 (m, 1H), 3.33-3.24 (m, 3H), 3.17- |
| | 3.08 (m, 3H), 2.11-1.87 (m, 5H), 1.48-1.13 (m, 9H). ¹⁹F NMR (376 MHz, |
| | Chloroform-d, 2 sets of rotamers) δ-109.3, -109.4. MS |
| | (ESI⁺): [M + H]⁺562.2/564.2. |
| 016 | White solid. ¹H NMR (400 MHz, DMSO-d₆, multiple sets of rotamers) δ |
| | 7.93-7.09 (m, 6H), 5.06-4.33 (m, 2H), 3.98-3.85 (m, 3H), 3.65-2.95 (m, 5H), |
| | 2.90-2.75 (m, 2H), 2.64-2.55 (m, δ3H), 1.44 (h, J = 7.2 Hz, 2H), 0.92-0.75 |
| | (m, 3H). ¹⁹F NMR (376 MHz, DMSO-d6, multiple sets of rotamers) δ-58.95, |
| | -58.97, -59.24, -59.37, -65.14, -65.84, -66.30, -66.32, -66.33, -109.35, |
| | -109.45, -109.77, -109.87.MS (ESI⁺): [M + H]⁺ 586.1. |
| 017 | White solid. ¹H NMR (400 MHz, DMSO-d₆, multiple sets of rotamers) δ |
| | 7.83-7.55 (m, 3H), 7.48-7.35 (m, 1H), 7.25-7.08 (m, 2H), 4.35-3.97 (m, 1H), |
| | 3.95-3.83 (m, 3H), 3.72-3.55 (m, 1H), 3.51-2.70 (m, 7H), 2.64-2.57 (m, 3H), |
| | 1.62-1.35 (m, 3H), 1.30-1.12 (m, 1H), 0.90-0.35 (m, 6H). ¹⁹F NMR (376 |
| | MHz, DMSO-d₆, multiple sets of rotamers) δ-58.2, -58.82, -58.83, -59.12, |
| | -59.13, -109.8, -110.0, -110.14, -110.16, -110.24. MS (ESI⁺): [M + H]⁺ |
| | 546.2. |
| 018 | White solid. ¹H NMR (400 MHz, DMSO-d₆, multiple sets of rotamers) δ |
| | 7.82-7.75 (m, 1H), 7.72-7.52 (m, 2H), 7.44-7.31 (m, 1H), 7.31-7.05 (m, 7H), |
| | 3.95-2.63 (m, 12H), 2.49-2.39 (m, 2H), 2.18-2.03 (m, 2H), 1.71-1.34 (m, |
| | 3H), 1.26-1.04 (m, 2H), 0.95-0.59 (m, 3H). ¹⁹F NMR (376 MHz, DMSO-d₆, |
| | multiple sets of rotamers) δ-58.84, -58.85, -59.02, -59.03, -110.10, |
| | -110.15, -110.17, -110.19. MS (ESI⁺): [M + H]⁺ 634.1. |
| 019 | White solid. ¹H NMR (400 MHz, DMSO-d₆, multiple sets of rotamers) δ |
| | 7.65-7.44 (m, 2H), 7.44-7.29 (m, 2H), 7.29-7.20 (m, 2H), 7.21-7.05 (m, 5H), |
| | 4.00-2.65 (m, 12H), 2.50-2.40 (m, 2H), 2.18-2.01 (m, 2H), 1.66-1.37 (m, |
| | 3H), 1.25-1.10 (m, 2H), 0.97-0.68 (m, 3H). ¹⁹F NMR (376 MHz, DMSO-d₆, |
| | multiple sets of rotamers) δ-110.06, -110.07, -110.14. MS |
| | (ESI⁺): [M + H]⁺ 600.1/602.1. |
| 020 | White solid. ¹H NMR (400 MHz, Chloroform-d, multiple sets of rotamers) |
| | δ 7.67-7.60 (m, 1H), 7.55-7.31 (m, 2H), 7.24-7.18 (m, 1H), 7.13-7.06 (m, |
| | 1H), 6.95-6.91 (m, 1H), 4.37 (d, J = 3.9 Hz, 1H), 4.29-3.97 (m, 1H), 3.98- |
| | 3.89 (m, 3H), 3.91-3.69 (m, 2H), 3.64-3.06 (m, 3H), 3.05-2.76 (m, 1H), |
| | 1.31-1.19 (m, 9H), 1.12-0.77 (m, 3H). ¹⁹F NMR (376 MHz, Chloroform-d) |
| | δ-109.33, -109.37, -109.39. MS (ESI⁺): [M + H]⁺ 498.0/500.0. |
| 021 | White solid. ¹H NMR (400 MHz, DMSO-d₆, multiple sets of rotamers) δ |
| | 7.87-7.52 (m, 3H), 7.50-7.40 (m, 1H), 7.30-7.10 (m, 2H), 4.10-3.45 (m, 6H), |
| | 3.45-3.10 (m, 2H), 3.10-2.65 (m, 4H), 2.63-2.57 (m, 3H), 1.53-1.37 (m, 2H), |
| | 0.97-0.62 (m, 6H). ¹⁹F NMR (376 MHz, DMSO-d₆, multiple sets of |
| | rotamers) δ-58.83, -58.86, -58.95, -58.98, -110.07, -110.09, -110.13, |
| | -110.15, -110.18. MS (ESI⁺): [M + H]⁺ 532.2. |
| 022 | White solid. ¹H NMR (400 MHz, DMSO-d₆) δ 7.83-7.10 (m, 6H), 4.36-4.02 |
| | (m, 1H), 3.98-3.67 (m, 4H), 3.57-3.35 (m, 2H), 3.30-3.15 (m, 3H), 3.15- |
| | 3.01 (m, 4H), 2.95-2.79 (m, 3H), 2.65-2.55 (m, 3H), 1.55-1.38 (m, 2H), |
| | 0.89-0.74 (m, 3H). ¹⁹F NMR (376 MHz, DMSO-d₆, multiple sets of |
| | rotamers) δ-58.7, -58.8, -59.1, -59.2, 109.9, -110.07, -110.10, -110.12. MS |
| | (ESI⁺): [M + H]⁺ 562.2. |
| 023 | White solid. ¹H NMR (400 MHz, Chloroform-d, 2 sets of rotamers) δ 7.62 |
| | (dd, J = 8.5, 5.5 Hz, 1H), 7.50-7.30 (m, 3H), 7.27-7.18 (m, 1H), 7.00-6.87 |
| | (m, 1H), 4.98-4.59 (m, 1H), 4.17-3.98 (m, 3H), 3.92 (d, J = 1.7 Hz, 3H), |
| | 3.88-3.82 (m, 1H), 3.78-3.67 (m, 2H), 3.62 (s, 1H), 3.53-3.39 (m, 2H), 2.97 |
| | (t, J = 7.2 Hz, 2H), 2.77-2.66 (m, 3H), 1.61-1.52 (m, 2H), 1.00-0.88 (m, |
| | 3H). ¹⁹F NMR (376 MHz, Chloroform-d, 2 sets of rotamers) δ-60.2, -60.9, |
| | -109.36, -109.74. MS (ESI⁺): [M + H]⁺ 560.0. |
| 024 | White solid. ¹H NMR (400 MHz, DMSO-d₆) 8 7.82-7.74 (m, 1H), 7.67 (d, |
| | J = 6.3 Hz, 1H), 7.62 (d, J = 6.5 Hz, 1H), 7.28 (dd, J = 8.4, 2.0 Hz, 1H), |
| | 7.14 (dd, J = 8.5, 2.4 Hz, 1H), 7.05 (s, 1H), 4.39-4.30 (m, 1H), 4.30-4.17 |
| | (m, 1H), 4.11 (d, J = 6.1 Hz, 1H), 3.87 (d, J = 3.5 Hz, 3H), 3.26-3.13 (m, |
| | 1H), 3.08-2.97 (m, 2H), 2.90-2.80 (m, 2H), 2.60 (s, 3H), 2.00-1.70 (m, 3H), |
| | 1.69-1.52 (m, 1H), 1.52-1.40 (m, 2H), 0.90-0.80 (m, 3H). ¹⁹F NMR (376 |
| | MHz, DMSO-d₆) δ-59.1, -110.2. MS (ESI⁺): [M + H]⁺ 544.1. |
| 025 | White solid. ¹H NMR (400 MHz, DMSO-d₆, 2 sets of rotamers) δ 7.62-7.10 |
| | (m, 10H), 6.98 (d, J = 2.1 Hz, 1H), 4.42-4.16 (m, 2H), 4.15-4.03 (m, 1H), |
| | 3.86 (s, 3H), 3.58 (d, J = 11.5 Hz, 2H), 3.41 (dd, J = 11.9, 7.8 Hz, 1H), 3.15- |
| | 2.90 (m, 2H), 2.46 (d, J = 7.1 Hz, 2H), 2.10 (t, J = 11.9 Hz, 2H), 1.98-1.84 |
| | (m, 1H), 1.83-1.54 (m, 5H), 1.54-1.38 (m, 1H), 1.28-1.03 (m, 2H). ¹⁹F NMR |
| | (376 MHz, DMSO-d₆, 2 sets of rotamers) δ-110.1, -110.3. MS |
| | (ESI⁺): [M + H]⁺ 612.2/614.2. |
| 026 | White solid. ¹H NMR (400 MHz, Chloroform-d, 2 sets of rotamers) δ 7.30- |
| | 7.13 (m, 3H), 7.12-6.89 (m, 3H), 4.51-4.35 (m, 1H), 4.38-4.21 (m, 1H), |
| | 4.07-4.02 (m, 1H), 3.57 & 3.41 (d, J = 12.9 Hz, 1H), 3.18 (d, J = 13.1 Hz, |
| | 1H), 3.11-3.00 (m, 1H), 2.93-2.86 (m, 2H), 2.65 (s, 3H), 2.10-1.55 (m, 4H), |
| | 1.55-1.34 (m, 2H), 0.85 (t, J = 7.4 Hz, 3H). ¹⁹F NMR (376 MHz, |
| | Chloroform-d, 2 sets of rotamers) δ-108.99, -109.12, -116.30, -116.35. |
| | (ESI⁺): [M + H]⁺ 498.1/500.1. |
| 027 | White solid. ¹H NMR (400 MHz, Chloroform-d, 2 sets of rotamers) δ 7.40- |
| | 7.13 (m, 5H), 7.13-7.01 (m, 1H), 4.55 (d, J = 13.0 Hz, 1H), 4.29-4.20 (m, |
| | 1H), 4.07-3.97 (m, 1H), 3.53 (dd, J = 63.7, 12.6 Hz, 1H), 3.29-3.21 (m, 1H), |
| | 3.21-3.13 (m, 1H), 2.99 (t, J = 7.2 Hz, 2H), 2.74 (s, 3H), 2.12-1.87 (m, 4H), |
| | 1.59-1.49 (m, 2H), 0.93 (t, J = 7.4 Hz, 3H). ¹⁹F NMR (376 MHz, |
| | Chloroform-d) δ-56.3, -109.0. MS (ESI⁺): [M + H]⁺ 564.2/566.1. |
| 028 | White solid. ¹H NMR (400 MHz, Chloroform-d, 2 sets of rotamers) δ 7.45- |
| | 7.25 (m, 2H), 7.21-7.00 (m, 4H), 7.00-6.94 (m, 1H), 4.47 (t, J = 13.2 Hz, |
| | 1H), 4.40 (d, J = 6.2 Hz, 1H), 4.22-4.14 (m, 1H), 3.65 & 3.48 (d, J = 12.6 |
| | Hz, 1H), 3.27 (dd, J = 13.2, 4.9 Hz, 1H), 3.08 (t, J = 12.3 Hz, 1H), 3.01 (td, |
| | J = 6.9, 1.3 Hz, 2H), 2.76 (s, 3H), 2.07-1.70 (m, 4H), 1.66-1.53 (m, 2H), |
| | 0.95 (t, J = 7.4 Hz, 3H). ¹⁹F NMR (376 MHz, Chloroform-d, 2 sets of |
| | rotamers) δ-108.97, -109.11. (ESI⁺): [M + H]⁺ 480.1/482.1. |
| 029 | White solid. ¹H NMR (400 MHz, Chloroform-d, 2 set of rotamers) δ 7.40- |
| | 7.31 (m, 1.5H), 7.24-7.12 (m, 2.5H), 7.12-7.00 (m, 1H), 6.91 (d, J = 8.5 Hz, |
| | 1H), 4.51 (d, J = 12.9 Hz, 1H), 4.31 (t, 1H), 4.15-4.02 (m, 3H), 3.67 (d, J = |
| | 12.4 Hz, 0.5H), 3.53 (d, J = 12.4 Hz, 0.5H), 3.33 (d, J = 12.8 Hz, 1H), 3.15 |
| | (q, 1H), 2.97-2.86 (m, 2H), 2.68 (s, 3H), 2.07-1.84 (m, 4H), 1.67-1.45 (m, |
| | 5H), 0.91 (t, J = 7.4 Hz, 3H). ¹⁹F NMR (376 MHz, Chloroform-d, 2 set of |
| | rotamers) δ-109.26, -109.34. MS (ESI⁺): [M + H]⁺ 524.1/526.1 |
| 030 | White solid. ¹H NMR (400 MHz, Chloroform-d, 2 sets of rotamers) δ 7.41- |
| | 7.27 (m, 2H), 7.24-7.12 (m, 2H), 7.12-7.00 (m, 1H), 6.78-6.72 (m, 1H), |
| | 4.78-4.65 (m, 1H), 4.51 (d, J = 12.9 Hz, 1H), 4.32 (s, 1H), 4.08 (s, 1H), 3.68 |
| | (d, J = 12.6 Hz, 0.5H), 3.53 (d, J = 12.2 Hz, 0.5H), 3.33 (d, J = 13.0 Hz, |
| | 1H), 3.16 (d, J = 10.7 Hz, 1H), 2.98-2.85 (m, 2H), 2.68 (s, 3H), 2.51 (s, 2H), |
| | 2.20 (s, 2H), 2.09-1.83 (m, 4H), 1.82-1.69 (m, 2H), 1.57-1.45 (m, 2H), 0.91 |
| | (q, J = 7.1 Hz, 3H). ¹⁹F NMR (376 MHz, Chloroform-d, 2 set of rotamers) |
| | δ-109.28, -109.37. MS (ESI⁺): [M + H]⁺ 550.2/552.2 |
| 031 | White solid. ¹H NMR (400 MHz, Chloroform-d, 2 sets of rotamers) δ 7.39 |
| | (dd, J = 8.5, 5.8 Hz, 0.5H), 7.32 (dt, J = 8.3, 1.4 Hz, 1H), 7.26-7.15 (m, |
| | 3.5H), 7.15-7.00 (m, 1H), 5.33-5.22 (m, 2H), 4.54 (dd, J = 13.0, 6.5 Hz, |
| | 1H), 4.33 (s, 1H), 4.09 (s, 1H), 3.69 (d, J = 12.7 Hz, 0.5H), 3.57-3.47 (m, |
| | 3.5H), 3.31 (d, J = 12.9 Hz, 1H), 3.17 (t, J = 10.5 Hz, 1H), 2.96 (dd, J = 8.0, |
| | 6.5 Hz, 2H), 2.72 (s, 3H), 2.17-1.63 (m, 4H), 1.55 (dd, J = 14.7, 7.4 Hz, 2H), |
| | 0.94 (t, J = 7.4 Hz, 3H). ¹⁹F NMR (376 MHz, Chloroform-d, 2 sets of |
| | rotamers) δ-109.19, -109.32. MS (ESI⁺): [M + H]⁺ 540.1/542.1 |
| 032 | Brown solid. ¹H NMR (400 MHz, Chloroform-d, 2 sets of rotamers) δ 8.36 & |
| | 8.36 (s, 1H), 7.48 (dd, J = 5.1, 2.1 Hz, 1H), 7.37 & 7.17 (dd, J = 8.5, 5.8 |
| | Hz, 1H), 7.25-7.19 (m, 1H), 7.11-7.03 (m, 1H), 4.64-4.58 (m, 1H), 4.22- |
| | 4.16 (m, 1H), 4.03-3.97 (m, 1H), 3.73 & 3.57 (d, J = 12.4 Hz, 1H), 3.33 (d, |
| | J = 12.8 Hz, 1H), 3.29-3.21 (m, 5H), 2.08-1.98 & 1.75-1.65 (m, 3H), 1.97- |
| | 1.84 (m, 1H), 1.17 (t, J = 7.1 Hz, 6H). ¹⁹F NMR (376 MHz, Chloroform-d, |
| | 2 sets of rotamers) δ-108.85, -108.98. (ESI⁺): [M + H]⁺ 515.0/517.0/519.0. |
| 033 | Off-white solid. ¹H NMR (400 MHz, Chloroform-d, 2 sets of rotamers) δ |
| | 8.13 (t, J = 2.2 Hz, 1H), 7.37 & 7.21 (dd, J = 8.5, 5.8 Hz, 1H), 7.24 (d, J = |
| | 2.1 Hz, 1H), 7.18-7.11 (m, 1H), 7.10-7.00 (m, 1H), 4.52 & 4.50 (d, J = 5.8 |
| | Hz, 1H), 4.38-4.33 (m, 1H), 4.18-4.13 (m, 1H), 4.04 & 4.03 (s, 3H), 3.62 & |
| | 3.46 (d, J = 12.6 Hz, 1H), 3.27-3.20 (m, 5H), 3.17-3.06 (m, 1H), 2.12-1.86 |
| | & 1.69-1.60 (m, 4H), 1.15 (t, J = 7.1 Hz, 6H). ¹⁹F NMR (376 MHz, |
| | Chloroform-d, 2 sets of rotamers) δ-109.07, -109.19. (ESI⁺): [M + H]⁺ |
| | 511.1/513.1 |
| 034 | Off-white solid. ¹H NMR (400 MHz, Chloroform-d, 2 sets of rotamers) δ |
| | 8.14 (t, J = 2.0 Hz, 1H), 7.49-7.30 & 7.26-7.23 (m, 6H), 7.25-6.93 (m, 3H), |
| | 5.47 (s, 2H), 4.50-4.46 & 4.45-4.43 (m, 1H), 4.40-4.35 (m, 1H), 4.17-4.12 |
| | (m, 1H), 3.58 & 3.39 (d, J = 12.5 Hz, 1H), 3.27-3.18 (m, 5H), 3.08 (t, J = |
| | 10.1 Hz, 1H), 2.05-1.83 & 1.66-1.59 (m, 4H), 1.16 (t, J = 7.1 Hz, 6H). ¹⁹F |
| | NMR (376 MHz, Chloroform-d, 2 sets of rotamers) δ-109.06, -109.19. |
| | (ESI⁺): [M + H]⁺ 587.1/589.1 |
| 035 | White solid. ¹H NMR (400 MHz, Chloroform-d, 2 sets of rotamers) δ 11.25 |
| | (s, 1H), 7.52-7.49 (m, 1H), 7.37 & 7.21 (dd, J = 8.5, 5.8 Hz, 1H), 7.17-7.12 |
| | (m, 1H), 7.11-7.01 (m, 1H), 6.76-6.74 (m, 1H), 4.67-4.54 (m, 2H), 4.48 (d, |
| | J = 13.2 Hz, 1H), 3.59 & 3.44 (d, J = 12.7 Hz, 1H), 3.28-3.19 (m, 5H), 3.09 |
| | (d, J = 12.7 Hz, 1H), 2.16-1.88 & 1.68-1.63 (m, 4H), 1.17 (t, J = 7.1 Hz, |
| | 6H). ¹⁹F NMR (376 MHz, Chloroform-d, 2 sets of rotamers) δ-108.97, |
| | -109.10. (ESI⁺): [M + H]⁺ 497.1/499.1. |
| 036 | White solid. ¹H NMR (400 MHz, Chloroform-d, 2 sets of rotamers) δ 7.55- |
| | 7.31 (m, 3H), 7.28-7.04 (m, 4H), 4.67 (d, J = 13.3 Hz, 1H), 4.14-3.61 (m, |
| | 3.5H), 3.44-3.37 (m, 0.5H), 3.35-3.24 (m, 1H), 2.99-2.90 (m, 2H), 2.71 (s, |
| | 3H), 2.37-1.94 (m, 4H), 1.58 (p, J = 7.2 Hz, 2H), 0.94 (t, J = 7.4 Hz, 3H). |
| | ¹⁹F NMR (376 MHz, Chloroform-d ) δ-108.6. MS (ESI⁺): [M + H]⁺ |
| | 496.5/498.3. |
| 037 | White solid. ¹H NMR (400 MHz, Chloroform-d, 2 sets of rotamers) δ 7.43- |
| | 7.28 (m, 4H), 7.25-7.18 (m, 2H), 7.16-7.08 (m, 3H), 7.08-6.99 (m, 1H), 6.88 |
| | (d, J = 8.5 Hz, 1H), 4.51-4.45 (m, 1H), 4.34 (s, 2H), 4.27-4.21 (m, 1H), |
| | 4.09-3.99 (m, 2H), 3.89 & 3.88 (s, 3H), 3.67-3.38 (m, 3H), 3.31-3.18 (m, |
| | 3H), 3.14-3.06 (m, 1H), 2.00-1.83 (m, 6H). ¹⁹F NMR (376 MHz, |
| | Chloroform-d, 2 sets of rotamers) δ-109.3, -109.4. MS |
| | (ESI⁺): [M + H]⁺ 614.2/616.1. |
| 038 | White solid. ¹H NMR (400 MHz, DMSO-d₆, multiple sets of rotamers) δ |
| | 7.63-7.40 (m, 3H), 7.37-7.28 (m, 1H), 7.25-7.10 (m, 2H), 4.55-4.35 (m, 2H), |
| | 4.12-3.30 (m, 7H), 3.30-2.70 (m, 5H), 2.63 & 2.62 (s, 3H), 1.91-1.72 (m, |
| | 2H), 0.95-0.70 (m, 3H). ¹⁹F NMR (376 MHz, DMSO-d₆, multiple sets of |
| | rotamers) δ-110.06, -110.07, -110.14, -219.73, -219.74, -219.75. MS |
| | (ESI⁺): [M + H]⁺ 516.1/518.1. |
| 039 | White solid. ¹H NMR (400 MHz, Chloroform-d, set of 2 rotamers) δ 7.40- |
| | 7.34 (m, 1.5H), 7.24-7.19 (m, 0.5H), 7.19-7.12 (m, 2H), 7.12-7.02 (m, |
| | 3H), 7.02-6.93 (m, 3H), 4.59-4.45 (m, 1H), 4.37-4.24 (m, 1H), 4.14- |
| | 4.02 (m, 1H), 3.98-3.93 (m, 3H), 3.92-3.83 (m, 2H), 3.72-3.62 (m, |
| | 0.5H), 3.55-3.49 (m, 0.5H), 3.35-3.24 (m, 1H), 3.24-3.07 (m, 1H), 2.46- |
| | 2.27 (m, 3H), 2.11-1.90 (m, 3.5H), 1.90-1.70 (m, 4H), 1.72-1.60 (m, |
| | 0.5H). ¹⁹F NMR (376 MHz, Chloroform-d, set of 2 rotamers) δ-109.20, -109.33, |
| | -116.42. MS (ESI⁺): [M + H]⁺ 616.2/618.2. |
| 040 | White solid. ¹H NMR (400 MHz, Chloroform-d, set of 2 rotamers) δ 7.54- |
| | 7.48 (m, 1H), 7.45-7.34 (m, 4H), 7.25-7.00 (m, 4H), 6.97 (d, J = 8.5 Hz, |
| | 1H), 4.59-4.46 (m, 1H), 4.36-4.26 (m, 1H), 4.13-4.03 (m, 1H), 3.98- |
| | 3.86 (m, 4.5H), 3.73-3.63 (m, 0.5H), 3.55-3.45 (m, 1H), 3.33-3.26 (m, |
| | 1H), 3.20-3.10 (m, 1H), 2.53-2.41 (m, 1H), 2.35 (t, J = 12.0 Hz, 2H), |
| | 2.11-1.72 (m, 7.5H), 1.69-1.61 (m, 0.5H). ¹⁹F NMR (376 MHz, |
| | Chloroform-d, set of 2 rotamers) δ-109.17, -109.31. MS |
| | (ESI⁺): [M + H]⁺ 623.3/625.2. |
| 041 | White solid. ¹H NMR (400 MHz, Chloroform-d, 2 sets of rotamers) δ 7.90- |
| | 7.86 (m, 1H), 7.84-7.82 (m, 1H), 7.40-7.32 (m, 3.5H), 7.21 (dd, J = |
| | 8.5, 2.4 Hz, 0.5H), 7.18-7.12 (m, 2H), 7.11-7.01 (m, 1H), 6.97 (d, J = |
| | 8.5 Hz, 1H), 4.55-4.49 (m, 1H), 4.33-4.28 (m, 1H), 4.10-4.04 (m, 1H), |
| | 3.97-3.86 (m, 8H), 3.72-3.48 (m, 1H), 3.33-3.26 (m, 1H), 3.18-3.11 |
| | (m, 1H), 2.52-2.42 (m, 1H), 2.38-2.30 (m, 2H), 2.09-1.78 (m, 7.5H), |
| | 1.68-1.61 (m, 0.5H). ¹⁹F NMR (376 MHz, Chloroform-d, 2 sets of |
| | rotamers) δ-109.3, -109.4. MS (ESI⁺): [M + H]⁺ 656.2, 658.2 [M + Na]⁺ |
| | 678.2/680.2. |
| 042 | White solid. ¹H NMR (400 MHz, Chloroform-d, 2 sets of rotamers) δ 7.98- |
| | 7.94 (m, 2H), 7.39-7.34 (m, 1.5H), 7.23-7.13 (m, 4.5H), 7.11-7.00 |
| | (m, 1H), 6.96 (d, J = 8.4 Hz, 1H), 4.55-4.49 (m, 1H), 4.33-4.28 (m, 1H), |
| | 4.09-4.05 (m, 1H), 3.95 & 3.94 (s, 4H), 3.90 (s, 4H), 3.69 (d, J = 12.8 Hz, |
| | 0.5H), 3.51 (d, J = 12.2 Hz, 0.5H), 3.33-3.26 (m, 1H), 3.18-3.10 (m, 1H), |
| | 2.52-2.43 (m, 1H), 2.39-2.29 (m, 2H), 2.09-1.78 (m, 7.5H), 1.68-1.60 |
| | (m, 0.5H). ¹⁹F NMR (376 MHz, Chloroform-d, 2 sets of rotamers) δ-109.2, |
| | -109.3. MS (ESI⁺): [M + H]⁺ 656.2/658.3. |
| 043 | White solid. ¹H NMR (400 MHz, Chloroform-d, set of 2 rotamers) δ 7.92 |
| | (d, J = 2.4 Hz, 1H), 7.73 (d, J = 1.7 Hz, 1H), 7.60-7.53 (m, 1H), 7.53- |
| | 7.44 (m, 1H), 7.44-7.33 (m, 2.5H), 7.26-7.14 (m, 2.5H), 7.14-7.01 (m, |
| | 2H), 6.99 (d, J = 8.5 Hz, 1H), 6.49 (t, J = 2.1 Hz, 1H), 4.59-4.47 (m, 1H), |
| | 4.33 (s, 1H), 4.10 (s, 1H), 4.01-3.87 (m, 5H), 3.75-3.52 (m, 1H), 3.33 (d, |
| | J = 12.9 Hz, 1H), 3.24-3.09 (m, 1H), 2.60-2.44 (m, 1H), 2.38 (t, J = 11.2 |
| | Hz, 2H), 2.18-1.78 (m, 7.5H), 1.74-1.62 (m, 0.5H). ¹⁹F NMR (376 MHz, |
| | Chloroform-d, set of 2 rotamers) δ-109.24, -109.37. MS |
| | (ESI⁺): [M + H]⁺ 664.3/666.3. |
| 044 | White solid. ¹H NMR (400 MHz, Chloroform-d, 2 sets of rotamers) δ 7.42-7.31 |
| | (m, 1.5H), 7.26-6.89 (m, 8.5H), 4.56-4.49 (m, 1H), 4.30 (d, J = |
| | 6.2 Hz, 1H), 4.15-4.04 (m, 1H), 3.94 (s, 1.5H), 3.93 (s, 1.5H), 3.69 (d, J = |
| | 12.3 Hz, 0.5H), 3.54-3.50 (m, 0.5H), 3.31 (d, J = 12.8 Hz, 1H), 3.21-3.11 |
| | (m, 1H), 3.09-2.89 (m, 4H), 2.82-2.68 (m, 4H), 2.10-1.84 (m, 5.5H), |
| | 1.69-1.61 (m, 0.5H), 1.06-0.98 (m, 1H), 0.95-0.89 (m, 1H). ¹⁹F NMR |
| | (376 MHz, Chloroform-d, 2 sets of rotamers) δ-109.23, -109.24, -109.37, |
| | -117.31, -117.33, -117.34, -117.35. MS (ESI⁺): [M + H]⁺ 657.2/659.0. |
| 045 | White solid. ¹H NMR (400 MHz, Chloroform-d, 2 sets of rotamers) δ 7.37 |
| | (dd, J = 8.5, 5.8 Hz, 0.5H), 7.32-7.29 (m, 1H), 7.24-7.12 (m, 1.5H), 7.12- |
| | 6.99 (m, 4H), 6.99-6.89 (m, 3H), 4.56-4.45 (m, 1H), 4.31-4.23 (m, |
| | 1H), 4.10-4.02 (m, 1H), 3.93 (s, 1.5H), 3.92 (s, 1.5H), 3.67 (d, J = 12.5 |
| | Hz, 0.5H), 3.49 (d, J = 11.2 Hz, 0.5H), 3.29 (d, J = 12.9 Hz, 1H), 3.18- |
| | 3.09 (m, 1H), 3.01-2.90 (m, 4H), 2.85 (q, J = 7.2 Hz, 1H), 2.58-2.32 (m, |
| | 6H), 2.10-1.82 (m, 3.5H), 1.66-1.59 (m, 0.5H), 1.18 (s, 1.5H), 1.16 (s, |
| | 1.5H). ¹⁹F NMR (376 MHz, Chloroform-d, 2 sets of rotamers) δ-109.25, |
| | -109.37, -117.13, -117.14. MS (ESI⁺): [M + H]⁺ 659.3/661.3. |
| 046 | White solid. ¹H NMR (400 MHz, Chloroform-d, multiple sets of rotamers) |
| | δ 7.39-7.31 (m, 1.5H), 7.23-7.01 (m, 5.5H), 6.98-6.90 (m, 3H), 4.54- |
| | 4.47 (m, 1H), 4.29-4.25 (m, 1H), 4.09-4.05 (m, 1H), 3.92 & 3.91 (s, 3H), |
| | 3.70-3.46 (m, 1H), 3.33-3.26 (m, 1H), 3.17-3.10 (m, 1H), 3.07-2.95 |
| | (m, 4H), 2.73-2.67 (m, 2H), 2.63-2.52 (m, 6H), 2.08-1.86 (m, 3.5H), |
| | 1.67-1.59 (m, 0.5H). ¹⁹F NMR (376 MHz, Chloroform-d, multiple sets of |
| | rotamers) δ-109.26, -109.39, -117.06, -117.07. MS |
| | (ESI⁺): [M + H]⁺ 645.3/647.3. |
| 047 | White solid. ¹H NMR (400 MHz, Chloroform-d, set of 2 rotamers) δ 7.39- |
| | 7.26 (m, 2.5H), 7.25-7.01 (m, 7.5H), 6.94-6.90 (m, 1H), 4.54-4.47 (m, |
| | 1H), 4.29-4.25 (m, 1H), 4.08-4.03 (s, 1H), 3.92 & 3.91 (s, 3H), 3.70- |
| | 3.47 (m, 1H), 3.32-3.26 (m, 1H), 3.17-3.10 (m, 1H), 2.99-2.82 (m, 5H), |
| | 2.56-2.39 (m, 6H), 2.08-1.87 (m, 3.5H), 1.66-1.58 (m, 0.5H), 1.20 & |
| | 1.19 (s, 3H). ¹⁹F NMR (376 MHz, Chloroform-d, set of 2 rotamers) |
| | δ-109.28, -109.40. MS (ESI⁺): [M + H]⁺ 641.3/643.3. |
| 048 | White solid. ¹H NMR (400 MHz, Chloroform-d, set of 2 rotamers) δ 7.42- |
| | 7.30 & 7.25-7.18 (m, 4H), 7.18-7.13 (m, 1H), 7.12-6.99 (m, 4H), 6.93 |
| | (dd, J = 8.5, 1.4 Hz, 1H), 4.51 (d, J = 12.9 Hz, 1H), 4.27 (d, J = 6.0 Hz, 1H), |
| | 4.06 (s, 1H), 3.92 & 3.91 (s, 3H), 3.67 & 3.49 (d, J = 12.5 Hz, 1H), 3.35- |
| | 3.24 (m, 1H), 3.13 (t, J = 10.1 Hz, 1H), 3.01 (s, 4H), 2.72-2.65 (m, 2H), |
| | 2.62-2.52 & 1.69-1.59 (m, 6H), 2.12-1.84 (m, 4H). ¹⁹F NMR (376 |
| | MHz, Chloroform-d, set of 2 rotamers) δ-109.26, -109.38. MS |
| | (ESI⁺): [M + H]⁺ 661.3/663.3. |
| 049 | White solid. ¹H NMR (400 MHz, Chloroform-d, 2 sets of rotamers) δ 7.41- |
| | 7.27 (m, 3.5H), 7.25-7.18 (m, 2H), 7.18-7.09 (m, 3H), 7.03 (t, J = 8.7 |
| | Hz, 0.5H), 6.96 (d, J = 8.5 Hz, 1H), 4.54-4.39 (m, 1H), 4.34-4.27 (m, |
| | 1H), 4.15-4.03 (m, 1H), 3.98-3.83 (m, 5H), 3.76-3.67 (m, 0.5H), 3.59- |
| | 3.50 (m, 0.5H), 3.30 (d, J = 12.8 Hz, 1H), 3.21-3.06 (m, 1H), 2.47-2.27 |
| | (m, 3H), 2.15-1.75 (m, 7.5H), 1.72-1.61 (m, 0.5H). ¹⁹F NMR (376 MHz, |
| | Chloroform-d, 2 sets of rotamers) δ-132.7 & -132.9 (d, JF-F = 21.0 Hz), |
| | -137.0 & -137.3 (d, J_{F-F} = 21.0 Hz), -185.87. MS (ESI⁺): [M + H]⁺ 616.1, |
| | 618.0 [M + Na]⁺ 638.2/640.1. |
| 050 | White solid. ¹H NMR (400 MHz, Chloroform-d, 2 sets of rotamers) δ 7.36- |
| | 7.26 (m, 2H), 7.26-7.21 (m, 2.5H), 7.21-7.15 (m, 1H), 7.15-7.10 (m, |
| | 3H), 7.07-7.00 (m, 0.5H), 6.94 & 6.92 (s, 1H), 4.51-4.43 (m, 1H), 4.30- |
| | 4.25 (m, 1H), 4.10-4.04 (m, 1H), 3.95-3.90 (m, 3H), 3.77-3.66 (m, |
| | 2.5H), 3.56-3.47 (m, 0.5H), 3.31-3.25 (m, 1H), 3.17-3.09 (m, 1H), 2.62- |
| | 2.54 (m, 2H), 2.24-2.14 (m, 2H), 2.09-1.84 (m, 3.5H), 1.81-1.70 (m, |
| | 2H), 1.67-1.61 (m, 0.5H), 1.60-1.57 (m, 1H), 1.55-1.51 (m, 2H), 1.40- |
| | 1.27 (m, 2H). ¹⁹F NMR (376 MHz, Chloroform-d, 2 sets of rotamers) δ- |
| | 132.7 & -132.9 (d, J_{F-F} = 21.0 Hz), -137.0 & -137.3 (d, J_{F-F} = 21.0 Hz), |
| | -185.87. MS (ESI⁺): [M + H]⁺ 644.2/646.2 |
| 051 | White solid. ¹H NMR (400 MHz, Chloroform-d, 2 sets of rotamers) δ 7.39- |
| | 7.34 (m, 1H), 7.34-7.29 (m, 0.5H), 7.29-7.27 (m, 0.5H), 7.26-7.19 |
| | (m, 2.5H), 7.16-7.12 (m, 1H), 7.07-7.00 (m, 0.5H), 6.95 (d, J = 8.5 Hz, |
| | 1H), 6.93-6.84 (m, 3H), 4.53-4.42 (m, 1H), 4.31-4.25 (m, 1H), 4.14- |
| | 4.04 (m, 1H), 3.97-3.88 (m, 3H), 3.73-3.66 (m, 0.5H), 3.56-3.46 (m, |
| | 0.5H), 3.34-3.27 (m, 1H), 3.27-3.20 (m, 4H), 3.19-3.08 (m, 5H), 2.13- |
| | 1.85 (m, 3.5H), 1.67-1.60 (m, 0.5H). ¹⁹F NMR (376 MHz, Chloroform- |
| | d, 2 sets of rotamers) δ-132.7 & -132.9 (d, J_{F-F} = 21.0 Hz), -137.0 & -137.3 |
| | (d, J_{F-F} = 21.0 Hz), -185.87. MS (ESI⁺): [M + H]⁺ 617.2/619.2 |
| 052 | White solid. ¹H NMR (400 MHz, Chloroform-d, 2 sets of rotamers) δ 7.34- |
| | 7.28 (m, 1.5H), 7.26-7.15 (m, 4H), 7.10-7.05 (m, 3H), 7.05-6.99 (m, |
| | 0.5H), 6.92 (d, J = 8.4 Hz, 1H), 4.49-4.42 (m, 1H), 4.29-4.23 (m, 1H), |
| | 4.08-4.01 (m, 1H), 3.95-3.88 (m, 3H), 3.76-3.65 (m, 2.5H), 3.55-3.47 |
| | (m, 0.5H), 3.31-3.24 (m, 1H), 3.16-3.08 (m, 1H), 2.53-2.47 (m, 2H), |
| | 2.20-2.11 (m, 2H), 2.07-1.83 (m, 3.5H), 1.72-1.60 (m, 2.5H), 1.46- |
| | 1.30 (m, 3H). ¹⁹F NMR (376 MHz, Chloroform-d, 2 sets of rotamers) δ- |
| | 132.7 & -132.9 (d, J_{F-F} = 21.0 Hz), -137.0 & -137.3 (d, J_{F-F} = 21.0 Hz), |
| | -185.87. MS (ESI⁺): [M + H]⁺ 630.2/632.3 |
| 053 | White solid. ¹H NMR (400 MHz, Chloroform-d, 2 sets of rotamers) δ 7.37- |
| | 7.27 (m, 3.5H), 7.24-7.17 (m, 2H), 7.13-7.06 (m, 3H), 7.06-6.97 (m, |
| | 0.5H), 6.97-6.89 (m, 1H), 6.31 (s, 1H), 4.52-4.42 (m, 1H), 4.30-4.22 |
| | (m, 1H), 4.11-4.02 (m, 1H), 3.97-3.87 (m, 3H), 3.72-3.64 (m, 0.5H), |
| | 3.57-3.46 (m, 0.5H), 3.32-3.24 (m, 1H), 3.17-3.06 (m, 3H), 2.97 (t, J = |
| | 6.1 Hz, 2H), 2.57 (t, J = 5.9 Hz, 2H), 2.45 (t, J = 5.8 Hz, 2H), 2.12-1.82 |
| | (m, 3.5H), 1.61 (s, 0.5H). ¹⁹F NMR (376 MHz, Chloroform-d, 2 sets of |
| | rotamers) δ-132.7 & -132.9 (d, J_{F-F} = 21.0 Hz), -137.0 & -137.3 (d, J_{F-F} = |
| | 21.0 Hz), -185.87. MS (ESI⁺): [M + H]⁺ 628.2, 630.2 [M + Na]⁺ 650.2/652.2. |
| 054 | White solid. ¹H NMR (400 MHz, Chloroform-d, 2 sets of rotamers) δ 7.42- |
| | 7.36 (m, 1H), 7.35-7.29 (m, 0.5H), 7.25-7.17 (m, 2H), 7.08-6.98 (m, |
| | 0.5H), 6.92-6.86 (m, 1H), 4.52-4.42 (m, 1H), 4.30-4.21 (m, 1H), 4.09- |
| | 4.01 (m, 1H), 3.94-3.88 (m, 3H), 3.72-3.65 (m, 0.5H), 3.57-3.49 (m, |
| | 0.5H), 3.33-3.25 (m, 1H), 3.16-3.08 (m, 1H), 2.92 (s, 3H), 2.08-1.83 |
| | (m, 3.5H), 1.67-1.60 (m, 0.5H), 1.32 (s, 9H). ¹⁹F NMR (376 MHz, |
| | Chloroform-d, 2 sets of rotamers) δ-132.7 & -132.9 (d, J_{F-F} = 21.0 Hz), |
| | -137.0 & -137.3 (d, J_{F-F} = 21.0 Hz). MS (ESI⁺): [M + H]⁺ 542.1, 544.1 |
| | [M + Na]⁺ 564.1/566.1. |
| 055 | White solid. ¹H NMR (400 MHz, , Chloroform-d, set of 2 rotamers) δ 7.41- |
| | 7.28 (m, 2.5H), 7.25-6.99 (m, 7.5H), 4.69 (td, J = 8.9, 3.1 Hz, 2H), 4.60- |
| | 4.50 (m, 1H), 4.46 (dd, J = 13.3, 4.0 Hz, 1H), 4.28 (s, 1H), 3.90 (d, J = |
| | 11.1 Hz, 2H), 3.76-3.44 (m, 1H), 3.39-3.19 (m, 3H), 3.19-2.98 (m, 1H), |
| | 2.53-2.29 (m, 3H), 2.16-1.62 (m, 8H). ¹⁹F NMR (376 MHz, Chloroform- |
| | d, 2 sets of rotamers) δ-109.16, -109.31. MS (ESI⁺): [M + H]⁺ 610.2/612.2. |
| 056 | White solid. ¹H NMR (400 MHz, Chloroform-d, set of 2 rotamers) δ 7.46- |
| | 7.18 (m, 2H), 7.17-6.94 (m, 7H), 4.76-4.65 (m, 2H), 4.58-4.41 (m, 2H), |
| | 4.30 (s, 1H), 3.91 (d, J = 11.5 Hz, 2H), 3.70-3.45 (m, 1H), 3.33-3.23 (m, |
| | 3H), 3.10 (dd, J = 12.9, 6.7 Hz, 1H), 2.53-2.32 (m, 3H), 2.19-1.74 (m, |
| | 7.5H), 1.72-1.62 (m, 0.5H). ¹⁹F NMR (376 MHz, Chloroform-d, 2 sets of |
| | rotamers) δ-109.14, -109.29, -116.43, -116.45. MS (ESI⁺): [M + H]⁺ |
| | 628.3/630.3. |
| 057 | White solid. ¹H NMR (400 MHz, Chloroform-d, set of 2 rotamers) δ 7.39- |
| | 7.33 (m, 0.5H), 7.28 (s, 1H), 7.23-7.18 (m, 0.5H), 7.16-6.98 (m, 3H), 4.72- |
| | 4.60 (m, 2H), 4.52-4.39 (m, 2H), 4.33 (s, 1H), 4.28-4.18 (m, 1H), 3.67- |
| | 3.57 (m, 0.5H), 3.52-3.42 (m, 0.5H), 3.24 (t, J = 8.8 Hz, 3H), 3.13-2.98 |
| | (m, 1H), 2.13-1.84 (m, 3.5H), 1.70-1.58 (m, 0.5H), 1.23 (s, 9H). ¹⁹F |
| | NMR (376 MHz, Chloroform-d, 2 sets of rotamers) δ-109.27, -109.39. MS |
| | (ESI⁺): [M + H]⁺ 522.2/524.2. |
| 058 | White solid. ¹H NMR (400 MHz, Chloroform-d, set of 2 rotamers) δ 7.40- |
| | 7.32 (m, 0.5H), 7.23-7.18 (m, 0.5H), 7.17-6.96 (m, 4H), 4.65 (td, J = 8.9, |
| | 2.6 Hz, 2H), 4.53-4.37 (m, 2H), 4.30-4.19 (m, 1H), 3.65-3.55 (m, 0.5H), |
| | 3.51-3.39 (m, 0.5H), 3.32-3.17 (m, 3H), 3.14-3.02 (m, 1H), 2.99-2.81 |
| | (m, 2H), 2.80 (s, 3H), 2.09-1.85 (m, 3.5H), 1.71-1.59 (m, 0.5H), 0.97- |
| | 0.80 (m, 1H), 0.62-0.47 (m, 2H), 0.22-0.08 (m, 2H). ¹⁹F NMR (376 MHz, |
| | Chloroform-d, 2 sets of rotamers) δ-109.22, -109.34. MS (ESI⁺): [M + H]⁺ |
| | 534.2/536.2. |
| 059 | White solid. ¹H NMR (400 MHz, Chloroform-d, set of 2 rotamers) δ 7.39 |
| | (dd, J = 8.5, 5.8 Hz, 0.5H), 7.23 (dd, J = 8.5, 2.5 Hz, 0.5H), 7.18-6.93 (m, |
| | 8H), 4.68 (td, J = 8.9, 5.0 Hz, 2H), 4.57-4.41 (m, 2H), 4.32 (s, 1H), 3.68- |
| | 3.39 (m, 1H), 3.32-3.19 (m, 3H), 3.17-2.97 (m, 5H), 2.79-2.68 (m, 2H), |
| | 2.68-2.53 (m, 6H), 2.16-1.88 (m, 3.5H), 1.72-1.60 (m, 0.5H). ¹⁹F NMR |
| | (376 MHz, Chloroform-d, 2 sets of rotamers) δ-109.20, -109.35, -117.04, |
| | -117.07. MS (ESI⁺): [M + H]⁺ 657.4/659.4 |
| 060 | White solid. ¹H NMR (400 MHz, Chloroform-d, set of 2 rotamers) δ 7.39 |
| | (dd, J = 8.5, 5.9 Hz, 0.5H), 7.34-7.20 (m, 2.5H), 7.20-7.01 (m, 5H), 6.99 |
| | (d, J = 1.7 Hz, 1H), 4.82-4.56 (m, 2H), 4.56-4.38 (m, 2H), 4.31 (s, 1H), |
| | 3.73-3.38 (m, 1H), 3.35-3.17 (m, 3H), 3.17-2.92 (m, 5H), 2.78-2.68 |
| | (m, 2H), 2.68-2.52 (m, 6H), 2.23-1.81 (m, 3.5H), 1.72-1.61 (m, 0.5H). |
| | ¹⁹F NMR (376 MHz, Chloroform-d, 2 sets of rotamers) δ-109.20, -109.35. |
| | MS (ESI⁺): [M + H]⁺ 673.4/675.4. |
| 061 | White solid. ¹H NMR (400 MHz, Chloroform-d, 2 sets of rotamers) δ 7.30 |
| | (t, J = 7.4 Hz, 3H), 7.23-7.12 (m, 5H), 7.03-6.98 (m, 1H), 4.73-4.65 |
| | (m, 2H), 4.54-4.48 (m, 1H), 4.46-4.38 (m, 1H), 4.30 (s, 1H), 3.89 (d, J = |
| | 11.4 Hz, 2H), 3.57 (dd, J = 71.0, 12.5 Hz, 1H), 3.32-3.21 (m, 3H), 3.11- |
| | 3.02 (m, 1H), 2.47-2.32 (m, 3H), 2.08-1.82 (m, 8H). ¹⁹F NMR (376 MHz, |
| | Chloroform-d, 2 sets of rotamers) δ-132.6 & -132.8 (d, J_{F-F}= 21 Hz), -136.9 |
| | & -137.3 (d, J_{F-F} = 21 Hz). MS (ESI⁺): [M + H]⁺ 628.2/630.2. |
| 062 | White solid. ¹H NMR (400 MHz, Chloroform-d, 2 sets of rotamers) δ 7.40- |
| | 7.35 (m, 1.5H), 7.26 (m, 0.5H), 7.25-7.19 (m, 3H), 7.19-7.10 (m, 4H), |
| | 7.10-7.03 (m, 1H), 7.03-6.97 (m, 1H), 4.68-4.50 (m, 1H), 3.95-3.79 |
| | (m, 1H), 3.77-3.67 (m, 2H), 3.67-3.58 (m, 1.5H), 3.53-3.44 (m, 0.5H), |
| | 3.37-3.25 (m, 1H), 3.25-3.15 (m, 1H), 2.58 (dd, J = 9.0, 6.8 Hz, 2H), |
| | 2.17 (td, J = 11.8, 2.5 Hz, 2H), 2.11-1.64 (m, 6H), 1.55 (dt, J = 9.5, 6.8 |
| | Hz, 2H), 1.41-1.06 (m, 4H). ¹⁹F NMR (376 MHz, Chloroform-d, 2 sets of |
| | rotamers) δ-108.65, -108.80. MS (ESI⁺): [M + H]⁺ 612.4/614.4. |
| 063 | White solid. ¹H NMR (400 MHz, Chloroform-d, 2 sets of rotamers) δ 7.42 |
| | & 7.40 (d, J = 2.1 Hz, 1H), 7.31 (dd, J = 8.5, 5.8 Hz, 0.5H), 7.27-7.20 (m, |
| | 3H), 7.18-7.10 (m, 2H), 7.10-7.04 (m, 2.5H), 7.04-6.94 (m, 2H), 6.80 |
| | (br s, 1H), 4.56 (d, J = 12.9 Hz, 1H), 3.82 (d, J = 11.6 Hz, 2H), 3.77-3.70 |
| | (m, 1H), 3.65-3.40 (m, 2H), 3.30-3.09 (m, 2H), 2.45-2.19 (m, 3H), 2.15- |
| | 1.85 (m, 3.5H), 1.86-1.61 (m, 4.5H). ¹⁹F NMR (376 MHz, Chloroform- |
| | d, 2 sets of rotamers δ-108.73, -108.87. MS (ESI⁺): [M + H]⁺ 584.1/586.1. |
| 064 | White solid. ¹H NMR (400 MHz, DMSO-d₆, 2 sets of rotamers) δ 10.47 (s, |
| | 1H), 7.66-6.89 (m, 10H), 4.49-3.98 (m, 3H), 3.44 (t, J = 12.2 Hz, 1H), |
| | 3.22-2.93 (m, 3H), 2.82 (s, 3H), 2.73-2.59 (m, 2H), 2.50-2.42 (m, 6H), |
| | 1.97-1.56 (m, 4H). ¹⁹F NMR (376 MHz, DMSO-d₆, 2 sets of rotamers) |
| | δ-110.08, -110.29. MS (ESI⁺): [M + H]⁺ 647.2/649.2. |
| 065 | Orange solid. ¹H NMR (400 MHz, Chloroform-d, set of 2 rotamers) δ 7.49- |
| | 7.45 (m, 1H), 7.40-7.35 (m, 0.5H), 7.32-7.28 (m, 1H), 7.25-7.14 (m, |
| | 1.5H), 7.13-7.03 (m, 4H), 7.01-6.94 (m, 2H), 6.83 (s, 1H), 4.67-4.60 |
| | (m, 1H), 3.93-3.85 (m, 2H), 3.83-3.77 (m, 1H), 3.69-3.46 (m, 2H), 3.32- |
| | 3.23 (m, 2H), 2.45-2.27 (m, 3H), 2.20-1.97 (m, 3.5H), 1.91-1.69 (m, |
| | 4.5H). ¹⁹F NMR (376 MHz, Chloroform-d, 2 sets of rotamers) δ-108.71, |
| | -108.85, -109.68, -116.35, -116.36. MS (ESI⁺): [M + H]⁺ 602.2/604.2. |
| 066 | White solid. ¹H NMR (400 MHz, Chloroform-d, set of 2 rotamers) δ 7.56- |
| | 7.28 (m, 5H), 7.26-7.02 (m, 6H), 4.65-4.57 (m, 1H), 4.21-4.15 (m, 1H), |
| | 4.00-3.87 (m, 3H), 3.78-3.71 (m, 0.5H), 3.62-3.56 (m, 0.5H), 3.39- |
| | 3.32 (m, 1H), 3.23 (t, J = 10.5 Hz, 1H), 2.50-2.33 (m, 3H), 2.12-1.78 (m, |
| | 8H). ¹⁹F NMR (376 MHz, Chloroform-d, set of 2 rotamers) δ-109.1, -109.2. |
| | (ESI⁺): [M + H]⁺ 602.1/604.2. |
| 067 | White solid. ¹H NMR (400 MHz, Chloroform-d, set of 2 rotamers) δ 7.41- |
| | 7.27 (m, 4.5H), 7.25-7.03 (m, 6.5H), 4.64-4.55 (m, 1H), 3.96-3.86 (m, |
| | 3H), 3.72-3.46 (m, 2H), 3.37-3.28 (m, 1H), 3.23 (t, J = 10.2 Hz, 1H), |
| | 2.51-2.25 (m, 6H), 2.14-1.76 (m, 7.5H), 1.72-1.62 (m, 0.5H). ¹⁹F NMR |
| | (376 MHz, Chloroform-d, set of 2 rotamers) δ-109.1, -109.3. |
| | (ESI⁺): [M + H]⁺ 582.2/584.2. |
| 068 | Yellow solid. ¹H NMR (400 MHz, Chloroform-d, 2 sets of rotamers) δ 7.40- |
| | 7.34 (m, 0.5H), 7.31 (s, 2H), 7.24-7.14 (m, 2.5H), 7.12-7.00 (m, 1H), |
| | 4.63-4.52 (m, 1H), 3.94-3.85 (m, 1H), 3.70-3.42 (m, 2H), 3.29 (d, J = |
| | 12.0 Hz, 1H), 3.25-3.17 (m, 1H), 2.92 (t, J = 7.3 Hz, 2H), 2.68 (s, 3H), |
| | 2.41 (d, J = 6.5 Hz, 3H), 2.10-1.90 (m, 3.5H), 1.70-1.63 (m, 0.5H), 1.55- |
| | 1.46 (m, 2H), 0.91 (t, J = 7.4 Hz, 3H). ¹⁹F NMR (376 MHz, Chloroform- |
| | d, 2 sets of rotamers) δ-109.17, -109.30. (ESI⁺): [M + H]⁺ 494.1/496.1. |
| 069 | Yellow solid. ¹H NMR (400 MHz, Chloroform-d, set of 2 rotamers) δ 7.88- |
| | 7.84 (m, 1H), 7.44-7.26 (m, 4H), 7.24-7.02 (m, 6H), 4.59-4.52 (m, |
| | 1H), 4.13-4.03 (m, 1H), 3.98-3.88 (m, 5H), 3.88-3.78 (m, 1H), 3.76- |
| | 3.55 (m, 1H), 3.37-3.29 (m, 1H), 3.22-3.13 (m, 1H), 2.47-2.34 (m, 3H), |
| | 2.09-1.78 (m, 8H). ¹⁹F NMR (376 MHz, Chloroform-d, 2 sets of rotamers) |
| | δ-109.04, -109.19, -110.03. MS (ESI⁺): [M + H]⁺ 626.2/628.2. |
| 070 | White solid. ¹H NMR (400 MHz, Chloroform-d, set of 2 rotamers) δ 7.64- |
| | 7.57 (m, 1H), 7.48-7.44 (m, 1H), 7.44-7.26 (m, 3.5H), 7.24 -7.03 (m, |
| | 5.5H), 4.95-4.80 (m, 2H), 4.64-4.57 (m, 1H), 3.97-3.87 (m, 3H), 3.75- |
| | 3.49 (m, 2H), 3.33-3.19 (m, 2H), 3.09-2.83 (m, 1H), 2.47-2.29 (m, |
| | 3H), 2.15-1.77 (m, 7.5H), 1.72-1.63 (m, 0.5H). ¹⁹F NMR (376 MHz, |
| | Chloroform-d, 2 sets of rotamers) δ-108.92, -109.04, -109.89. MS |
| | (ESI⁺): [M + H]⁺ 598.1/600.1. |
| 071 | ¹H NMR (400 MHz, Chloroform-d, 2 sets of rotamers) δ 12.85 (s, 1H), 8.36- |
| | 8.21 (m, 1H), 7.65-7.53 (m, 1H), 7.51-7.41 (m, 1H), 7.41-7.21 (m, |
| | 2.5H), 7.17-6.99 (m, 5.5H), 4.70-4.62 (m, 1H), 3.97-3.82 (m, 3H), 3.80- |
| | 3.48 (m, 2H), 3.34 (d, J = 13.4 Hz, 1H), 3.30-3.20 (m, 1H), 2.44-2.29 |
| | (m, 3H), 2.25-1.91 (m, 3H), 1.79 (dq, J = 16.9, 13.2 Hz, 5H). ¹⁹F NMR |
| | (376 MHz, Chloroform-d, 2 sets of rotamers) δ-108.22, -108.33, -109.07. |
| | MS (ESI⁺): [M + H]⁺ 612.1/613.7. |
| 072 | ¹H NMR (400 MHz, Chloroform-d, set of 2 rotamers) δ 9.08-8.66 (m, 1H), |
| | 7.62-7.29 (m, 5.5H), 7.26-7.05 (m, 5.5H), 4.71-4.60 (m, 1H), 3.99- |
| | 3.90 (m, 2H), 3.90-3.84 (m, 1H), 3.84-3.63 (m, 1H), 3.63-3.55 (m, 1H), |
| | 3.43 (d, J = 13.1 Hz, 1H), 3.34-3.21 (m, 1H), 2.50-2.06 (m, 6.5H), 1.98- |
| | 1.76 (m, 4.5H), 1.72-1.63 (m, 6H). ¹⁹F NMR (376 MHz, Chloroform-d, |
| | 2 sets of rotamers) δ-108.81, -108.91. MS (ESI⁺): [M + H]⁺ 626.2/628.2. |
| 073 | White solid. ¹H NMR (400 MHz, Chloroform-d, 2 sets of rotamers) δ 7.72- |
| | 7.28 (m, 4.5H), 7.25-6.99 (m, 6.5H), 4.63-4.53 (m, 1H), 4.17-4.06 |
| | (m, 1H), 3.99-3.83 (m, 3H), 3.74-3.44 (m, 3H), 3.40-3.09 (m, 2H), 2.54- |
| | 2.23 (m, 9H), 2.10-1.76 (m, 8H).¹⁹F NMR (376 MHz, Chloroform-d, 2 |
| | sets of rotamers) δ-109.16, -109.29. MS (ESI⁺): [M + H]⁺ 625.2/627.2. |
| 074 | White solid. ¹H NMR (400 MHz, Chloroform-d, 2 sets of rotamers) δ 7.58- |
| | 7.54 (m, 1H), 7.41-7.34 (m, 0.5H), 7.26-7.00 (m, 3.5H), 4.63-4.54 |
| | (m, 1H), 4.51 (s, 1H), 3.91-3.82 (m, 1H), 3.71-3.64 (m, 0.5H), 3.64- |
| | 3.55 (m, 1H), 3.54-3.48 (m, 0.5H), 3.34-3.27 (m, 1H), 3.27-3.16 (m, |
| | 1H), 2.49-2.42 (m, 3H), 2.12-1.85(m, 3.5H), 1.72-1.61 (m, 0.5H), 1.24 |
| | (s, 9H). ¹⁹F NMR (376 MHz, Chloroform-d, 2 sets of rotamers) δ-109.03, |
| | -109.16. MS (ESI⁺): [M + H]⁺ 528.0/530.0. |
| 075 | White solid. ¹H NMR (400 MHz, Chloroform-d, 2 sets of rotamers) δ 7.63- |
| | 7.58 (m, 1H), 7.43-7.28 (m, 1.5H), 7.25-7.00 (m, 2.5H), 4.64-4.55 |
| | (m, 2H), 4.16-4.09 (m, 1H), 3.95-3.85 (m, 1H), 3.77-3.70 (m, 0.5H), |
| | 3.62-3.55 (m, 0.5H), 3.38-3.30 (m, 1H), 3.27-3.16 (m, 1H), 2.13-1.82 |
| | (m, 3.5H), 1.71-1.63 (m, 0.5H), 1.26 (s, 9H). ¹⁹F NMR (376 MHz, |
| | Chloroform-d, 2 sets of rotamers) δ-109.00, -109.13. MS (ESI⁺): [M + H]⁺ |
| | 548.0/550.0. |
| 076 | White solid. ¹H NMR (400 MHz, Chloroform-d, 2 sets of rotamers) δ 7.36- |
| | 7.26 (m, 1.5H), 7.26-7.20 (m, 2H), 7.17-7.04 (m, 5.5H), 7.04-6.96 |
| | (m, 1H), 6.96-6.91 (m, 1H), 4.54-4.41 (m, 1H), 4.26-4.18 (m, 1H), 4.18- |
| | 4.14 (m, 2H), 4.04-3.94 (m, 1H), 3.94-3.76 (m, 4H), 3.65-3.37 (m, |
| | 1H), 3.24 (d, J = 13.0 Hz, 1H), 3.17-3.04 (m, 1H), 2.59 & 2.43 (t, J = 5.8 |
| | Hz, 1H), 2.39-2.21 (m, 3H), 2.02-1.68 (m, 7.5H), 1.64-1.55 (m, 0.5H). |
| | ¹⁹F NMR (376 MHz, Chloroform-d, 2 sets of rotamers) δ-109.09, -109.22. |
| | MS (ESI⁺): [M + H]⁺ 628.2/630.2. |
| 077 | White solid. ¹H NMR (400 MHz, Chloroform-d, 2 sets of rotamers) δ 7.43- |
| | 7.30 (m, 2H), 7.28-7.12 (m, 7H), 7.12-7.04 (m, 1H), 7.01 (dd, J = 8.5, |
| | 3.7 Hz, 1H), 4.62-4.53 (m, 1H), 4.34-4.27 (m, 1H), 4.24 (t, J = 4.5 Hz, |
| | 2H), 4.14-4.03 (m, 1H), 3.97 (dt, J = 12.3, 4.6 Hz, 2H), 3.80-3.72 (m, |
| | 2H), 3.70 (d, J = 12.8 Hz, 0.5H), 3.55 (d, J = 12.4 Hz, 0.5H), 3.34 (dd, J = |
| | 12.9, 5.6 Hz, 1H), 3.24-3.16 (m, 1H), 2.61 (dd, J = 9.1, 6.7 Hz, 2H), 2.22 |
| | (t, J = 11.6 Hz, 2H), 2.11-1.89 (m, 4H), 1.79 (d, J = 13.5 Hz, 3H), 1.71- |
| | 1.63 (m, 1H), 1.61-1.55 (m, 2H), 1.40-1.30 (m, 2H). ¹⁹F NMR (376 MHz, |
| | Chloroform-d, 2 sets of rotamers) δ-109.09, -109.19. MS (ESI⁺): [M + H]⁺ |
| | 656.4/658.4. |
| 078 | White solid. ¹H NMR (400 MHz, Chloroform-d ,2 sets of rotamers) δ 7.44- |
| | 7.29 (m, 2H), 7.25-6.94 (m, 8H), 4.60-4.50 (m, 1H), 4.32-4.17 (m, |
| | 3H), 4.10-3.87 (m, 3H), 3.73-3.41 (m, 2H), 3.38-2.39 (m, 13H), 2.15- |
| | 1.84 (m, 4H). ¹⁹F NMR (376 MHz, Chloroform-d, 2 sets of rotamers) δ- |
| | 109.14, -109.27. MS (ESI⁺): [M + H]⁺ 691.2/693.3. |
| 079 | Yellow solid. ¹H NMR (400 MHz, Chloroform-d, 2 sets of rotamers) δ 7.31 |
| | (dd, J = 8.5, 5.8 Hz, 1H), 7.23 (dd, J = 8.1, 1.7 Hz, 1H), 7.17-7.06 (m, 2H), |
| | 7.00 (dtd, J = 16.7, 8.2, 2.4 Hz, 1H), 6.84 (d, J = 8.1 Hz, 1H), 4.56-4.45 |
| | (m, 1H), 4.08-3.99 (m, 1H), 3.80 (s, 1H), 3.62-3.42 (m, 1H), 3.28-3.19 |
| | (m, 1H), 3.19-3.11 (m, 1H), 2.85 (t, J = 7.3 Hz, 2H), 2.61 (s, 3H), 2.28- |
| | 2.15 (m, 1H), 2.03-1.81 (m, 3H), 1.62-1.56 (m, 1H), 1.49-1.41 (m, 2H), |
| | 1.22-1.14 (m, 2H), 1.05-0.97 (m, 2H), 0.84 (t, J = 7.4 Hz, 3H). ¹⁹F NMR |
| | (376 MHz, Chloroform-d, 2 sets of rotamers) δ-109.17, -109.30. |
| | (ESI⁺): [M + H]⁺ 520.1/522.1. |
| 080 | Yellow solid. ¹H NMR (400 MHz, Chloroform-d, set of 2 rotamers) δ 7.38 |
| | & 7.25-7.00 (m, 7H), 7.35-7.27 (m, 3H), 6.94 (d, J = 8.1 Hz, 1H), 4.65- |
| | 4.55 (m, 1H), 4.12 (d, J = 5.9 Hz, 1H), 3.95-3.85 (m, 3H), 3.73-3.63 & |
| | 3.56-3.48 (m, 1H), 3.38-3.30 (m, 1H), 3.23 (t, J = 10.2 Hz, 1H), 2.49- |
| | 2.24 (m, 4H), 2.15-1.76 & 1.71-1.61 (m, 8H), 1.17-1.05 (m, 2H), 0.93- |
| | 0.70 (m, 2H). ¹⁹F NMR (376 MHz, Chloroform-d, set of 2 rotamers) δ- |
| | 109.14, -109.27. (ESI⁺): [M + H]⁺ 608.2/610.2. |
| 081 | White solid. ¹H NMR (400 MHz, DMSO-d₆ with one drop of DCL 37% in |
| | D₂O, 2 sets of rotamers) δ 9.35-9.25 (m, 1H), 8.11 (s, 1H), 7.77-7.66 (m, |
| | 1H), 7.64-7.21 (m, 8H), 7.20-7.10 (m, 3H), 4.24-4.12 (m, 1H), 4.00- |
| | 3.88 (m, 1H), 3.85-3.73 (m, 2H), 3.70-3.56 m, 1H), 3.45-3.33 (m, 1H), |
| | 3.27-3.18 (m, 0.5H), 3.18-3.10 (m, 0.5H), 2.99-2.82 (m, 1H), 2.41- |
| | 2.30 (m, 2H), 1.93-1.47 (m, 9H). ¹⁹F NMR (376 MHz, DMSO-d6 with one |
| | drop of DCL 37% in D₂O, 2 sets of rotamers) δ-108.96, -109.06. MS |
| | (ESI⁺): [M + H]⁺ 634.1/636.1. |
| 082 | White solid. ¹H NMR (400 MHz, Chloroform-d, 2 sets of rotamers) δ 7.90- |
| | 7.79 (m, 1H), 7.66-7.28 (m, 6H), 7.24-6.97 (m, 6H), 4.48-4.37 (m, |
| | 1H), 4.02-3.88 (m, 3H), 3.85-3.75 (m, 4H), 3.65-3.57 (m, 0.5H), 3.47- |
| | 3.40(m, 0.5H), 3.30-3.20 (m, 1H), 3.17-3.06 (m, 1H), 2.49-2.30 (m, |
| | 3H), 1.99-1.84 (m, 7H), 1.64-1.56 (m, 1H). ¹⁹F NMR (376 MHz, |
| | Chloroform-d, 2 sets of rotamers) δ-109.04, -109.24. MS (ESI⁺): [M + H]⁺ |
| | 648.2/650.2. |
| 083 | White solid. ¹H NMR (400 MHz, Chloroform-d, set of 2 rotamers) δ 7.39- |
| | 7.28 (m, 2H), 7.25-7.00 (m, 8H), 4.61-4.54 (m, 1H), 4.23-4.18 (m, 1H), |
| | 3.99-3.85 (m, 3H), 3.70-3.47 (m, 1H), 3.32-3.25 (m, 1H), 3.24-3.16 |
| | (m, 1H), 2.49-2.33 (m, 3H), 2.14-1.81 (m, 8H). ¹⁹F NMR (376 MHz, |
| | Chloroform-d, set of 2 rotamers) δ-108.84, -108.85, -108.98, -108.99, |
| | -135.08, -135.17. MS (ESI⁺): [M + H]⁺ 602.1/604.1. |
| 084 | Yellow solid. ¹H NMR (400 MHz, Chloroform-d, 2 sets of rotamers) δ 7.40- |
| | 7.33 (m, 0.5H), 7.21-6.96 (m, 8.5H), 6.17 (br s, 1H), 4.58 & 4.55 (s, 1H), |
| | 4.23-4.17 (m, 1H), 4.00-3.93 (m, 1H), 3.90-3.87 (s, 2H), 3.71-3.47 |
| | (m, 1H), 3.30-3.27 (s, 1H), 3.23-3.15 (m, 1H), 2.48-2.31 (m, 3H), 2.15- |
| | 3.93 (m, 3.5H), 1.92-1.69 (m, 4.5H). 19F NMR (376 MHz, Chloroform- |
| | d, 2 sets of rotamers) δ-108.83, -108.97, -116.29, -135.12, -135.19. MS |
| | (ESI⁺): [M + H]⁺ 620.1/622.1. |
| 085 | Yellow solid. ¹H NMR (400 MHz, Chloroform-d, 2 sets of rotamers) δ 7.40- |
| | 7.34 (m, 0.5H), 7.31 (d, J = 2.0 Hz, 1H), 7.22-7.01 (m, 3.5H), 6.27- |
| | 5.84 (br s, 1H), 4.57 & 4.54 (s, 1H), 4.40 (s, 1H), 4.14 (s, 1H), 3.90 (s, 1H), |
| | 3.70-3.44 (m, 1H), 3.29 & 3.26 (s, 1H), 3.24-3.15 (m, 1H), 2.11-1.92 |
| | (m, 3.5H), 1.74-1.67 (m, 0.5H), 1.23 (s, 9H). ¹⁹F NMR (376 MHz, |
| | Chloroform-d, 2 sets of rotamers) δ-108.92, -109.04, -135.28, -135.39. MS |
| | (ESI⁺): [M + H]⁺ 514.0/516.0. |
| 086 | White foam. ¹H NMR (400 MHz, Chloroform-d, 2 sets of rotamers) δ 7.40- |
| | 7.34 (m, 0.5H), 7.24-7.00 (m, 4.5H), 6.13 (br s, 1H), 4.57 & 4.55 (s, 1H), |
| | 4.17 (s, 1H), 3.93 (s, 1H), 3.69-3.45 (m, 1H), 3.28 & 3.24 (s, 1H), 3.22- |
| | 3.14 (m, 1H), 2.89 (d, J = 6.9, 2H), 2.81 (s, 3H), 2.13-1.92 (m, 3.5H), 1.73- |
| | 1.64 (m, 0.5H), 0.94-0.83 (m, 1H), 0.62-0.50 (m, 2H), 0.21-0.14 (m, |
| | 2H). ¹⁹F NMR (376 MHz, Chloroform-d, 2 sets of rotamers) δ-108.88, |
| | -109.02, -135.22, -135.32. MS (ESI⁺): [M + H]⁺ 526.0/528.0. |
| 087 | White solid. ¹H NMR (400 MHz, Chloroform-d, 2 sets of rotamers) δ 7.77 |
| | (s, 1H), 7.50-7.45 (m, 1H), 7.40-7.36 (m, 0.5H), 7.34-7.27 (m, 2H), |
| | 7.24-7.05 (m, 5.5H), 4.70 & 4.66 (s, 1H), 3.93 & 3.90 (s, 2H), 3.82-3.76 |
| | (m, 1H), 3.70-3.46 (m, 2H), 3.35-3.23 (m, 2H), 2.51-2.41 (m, 1H), 2.41- |
| | 2.33 (m, 2H), 2.21-2.02 (m, 3.5H), 1.97-1.72 (m, 4.5H). ¹⁹F NMR (376 |
| | MHz, Chloroform-d, 2 sets of rotamers) δ-62.68, -62.70, -108.46, -108.60. |
| | MS (ESI⁺): [M + H]⁺ 652.1/654.1. |
| 088 | White solid. ¹H NMR (400 MHz, Chloroform-d, 2 sets of rotamers) δ 7.76 |
| | (s, 1H), 7.49-7.44 (m, 1H), 7.41-7.36 (m, 0.5H), 7.25-7.16 (m, 1.5H), |
| | 7.14-7.04 (m, 3H), 7.02-6.95 (m, 2H), 4.69 & 4.66 (s, 1H), 3.93 & 3.89 |
| | (s, 2H), 3.82-3.75 (m, 1H), 3.70-3.46 (m, 2H), 3.34-3.25 (m, 2H), 2.49- |
| | 2.40 (m, 1H), 2.40-2.31 (m, 2H), 2.20-2.01 (m, 3.5H), 1.93-1.86 (m, |
| | 2H), 1.85-1.74 (m, 2.5H). ¹⁹F NMR (376 MHz, Chloroform-d, 2 sets of |
| | rotamers) δ-62.68, -62.70, -108.44, -108.57, -116.19, -116.20. MS |
| | (ESI⁺): [M + H]⁺ 670.1/672.1. |
| 089 | White solid. ¹H NMR (400 MHz, Chloroform-d, 2 sets of rotamers) δ 7.87 |
| | (s, 1H), 7.61-7.55 (m, 1H), 7.40-7.35 (m, 0.5H), 7.25-7.04 (m, 2.5H), |
| | 4.68 & 4.64 (s, 1H), 4.42 (s, 1H), 3.78-3.72 (m, 1H), 3.69-3.44 (m, 2H), |
| | 3.34-3.23 (m, 2H), 2.24-1.98 (m, 3.5H), 1.79-1.72 (m, 0.5H), 1.24 (s, |
| | 9H). ¹⁹F NMR (376 MHz, Chloroform-d, 2 sets of rotamers) δ-62.69, |
| | -62.71, -108.54, -108.67. MS (ESI⁺): [M + H]⁺ 564.0/566.0. |
| 090 | White solid. ¹H NMR (400 MHz, Chloroform-d, set of 2 rotamers) δ 7.39- |
| | 7.35 (m, 1H), 7.33-7.27 (m, 2H), 7.24-7.03 (m, 7H), 4.68-4.59 (m, 1H), |
| | 3.93-3.84 (m, 2H), 3.77-3.70 (m, 1H), 3.68-3.45 (m, 2H), 3.31-3.23 |
| | (m, 2H), 2.47-2.37 (m, 1H), 2.37-2.26 (m, 5H), 2.22-1.94 (m, 4H), 1.93- |
| | 1.66 (m, 5H). ¹⁹F NMR (376 MHz, Chloroform-d, set of 2 rotamers) |
| | -108.77, -108.91. MS (ESI⁺): [M + H]⁺ 598.2/600.2. |
| 091 | White solid. ¹H NMR (400 MHz, Chloroform-d, 2 sets of rotamers) δ 7.43- |
| | 7.35 (m, 1.5H), 7.26-7.15 (m, 2.5H), 7.14-6.95 (m, 6H), 4.66 & 4.63 |
| | (s, 1H), 3.90 & 3.87 (s, 2H), 3.77-3.69 (m, 1H), 3.69-3.45 (m, 2H), |
| | 3.33-3.23 (m, 2H), 2.47-2.37 (m, 1H), 2.37-2.27 (m, 5H), 2.23-1.95 (m, |
| | 4H), 1.92-1.67 (m, 3.5H), 1.70-1.60 (m, 0.5H). ¹⁹F NMR (376 MHz, |
| | Chloroform-d, 2 sets of rotamers) δ-108.75, -108.88, -116.38, -116.39. MS |
| | (ESI⁺): [M + H]⁺ 616.1/618.2. |
| 092 | White solid. ¹H NMR (400 MHz, Chloroform-d, 2 sets of rotamers) δ 7.42- |
| | 7.34 (m, 1.5H), 7.26-7.14 (m, 2.5H), 7.14-6.85 (m, 2H), 4.65 & 4.62 |
| | (s, 1H), 3.76-3.68 (m, 1H), 3.68-3.42 (m, 2H), 3.32-3.22 (m, 2H), 2.92- |
| | 2.82 (m, 2H), 2.79 (s, 3H), 2.30 (s, 3H), 2.20-1.92 (m, 3.5H), 1.76-1.66 |
| | (m, 0.5H), 0.95-0.80 (m, 1H), 0.60-0.50 (m, 2H), 0.23-0.13 (m, 2H). |
| | ¹⁹F NMR (376 MHz, Chloroform-d, 2 sets of rotamers) δ-108.81, -108.94. |
| | MS (ESI⁺): [M + H]⁺ 522.1/524.1. |
| 093 | White solid. ¹H NMR (400 MHz, Chloroform-d, 2 sets of rotamers) δ 7.48 |
| | (s, 1H), 7.43-7.37 (m, 0.5H), 7.32-7.17 (m, 2.5H), 7.15-6.90 (m, 2H), |
| | 4.66 & 4.63 (s, 1H), 4.35 (s, 1H), 3.76-3.68 (m, 1H), 3.68-3.40 (m, 2H), |
| | 3.35-3.20 (m, 2H), 2.32 (s, 3H), 2.25-1.95 (m, 3.5H), 1.80-1.68 (m, |
| | 0.5H), 1.24 (s, 9H). ¹⁹F NMR (376 MHz, Chloroform-d, 2 sets of rotamers) |
| | δ-108.84, -108.98. MS (ESI⁺): [M + H]⁺ 510.1/512.1. |
| 094 | Yellow solid. ¹H NMR (400 MHz, Chloroform-d, 2 sets of rotamers) δ 7.40- |
| | 7.36 (m, 1.5H), 7.32-7.27 (m, 2H), 7.24-7.04 (m, 6.5H), 4.66 & 4.62 |
| | (s, 1H), 3.91 & 3.88 (s, 2H), 3.77-3.70 (m, 1H), 3.68-3.45 (m, 2H), 3.32- |
| | 3.23 (m, 2H), 2.73 (q, J = 7.5 Hz, 2H), 2.48-2.37 (m, 1H), 2.37-2.27 |
| | (m, 2H), 2.21-1.93 (m, 3.5H), 1.92-1.72 (m, 4.5H), 1.26 (t, J = 7.5 Hz, |
| | 3H). ¹⁹F NMR (376 MHz, Chloroform-d, 2 sets of rotamers) 108.78, |
| | -108.91. MS (ESI⁺): [M + H]⁺ 612.2/614.2. |
| 095 | White solid. ¹H NMR (400 MHz, Chloroform-d, 2 sets of rotamers) δ 7.59 |
| | (d, J = 2.1 Hz, 1H), 7.43-7.16 (m, 3H), 7.15-7.00 (m, 1H), 6.56 (br s, |
| | 1H), 4.61 & 4.58 (s, 1H), 4.47 (s, 1H), 4.11 (s, 1H), 3.87 (s, 1H), 3.70-3.45 |
| | (m, 1H), 3.31 & 3.28 (s, 1H), 3.26-3.15 (m, 1H), 2.21-1.90 (m, 3.5H), |
| | 1.75-1.65 (m, 0.5H), 1.26 (s, 9H). ¹⁹F NMR (376 MHz, Chloroform-d, 2 |
| | sets of rotamers) δ-108.91, -109.04. MS (ESI⁺): [M + H]⁺ 530.1/532.1. |
| 096 | White solid. ¹H NMR (400 MHz, Chloroform-d, 2 sets of rotamers) δ 7.44 |
| | (d, J = 2.0 Hz, 1H), 7.41-7.35 (m, 0.5H), 7.25-7.02 (m, 3.5H), 6.54 (br |
| | s, 1H), 4.60 & 4.56 (s, 1H), 4.13 (s, 1H), 3.88 (s, 1H), 3.72-3.41 (m, 1H), |
| | 3.29 & 3.26 (s, 1H), 3.25-3.15 (m, 1H), 2.91 (d, J = 7.0 Hz, 2H), 2.83 (s, |
| | 3H), 2.23-1.85 (m, 3.5H), 1.75-1.65 (m, 0.5H), 0.95-0.83 (m, 1H), 0.62- |
| | 0.51 (m, 2H), 0.25-0.15 (m, 2H). ¹⁹F NMR (376 MHz, Chloroform-d, 2 |
| | sets of rotamers) δ-108.88, -109.02. MS (ESI⁺): [M + H]⁺ 542.1/544.1. |
| 097 | White solid. ¹H NMR (400 MHz, Chloroform-d, 2 sets of rotamers) δ 7.43 |
| | (d, J = 2.0 Hz, 1H), 7.42-7.35 (m, 0.5H), 7.26-7.13 (m, 1.5H), 7.13- |
| | 7.00 (m, 2H), 6.56 (br s, 1H), 4.85 & 4.73 (s, 1H), 4.60 & 4.57 (s, 1H), 4.13 |
| | (d, J = 6.1 Hz, 1H), 3.90 (s, 1H), 3.71-3.44 (m, 1H), 3.42-3.33 (m, 2H), |
| | 3.30 & 3.27 (s, 1H), 3.25-3.14 (m, 1H), 2.92-2.77 (m, 2H), 2.15-1.83 |
| | (m, 7.5H), 1.75-1.65 (m, 0.5H). ¹⁹F NMR (376 MHz, Chloroform-d, 2 sets |
| | of rotamers) δ-108.88, -109.01, -186.23. MS (ESI⁺): [M + H]⁺ 560.0/562.0. |
| 098 | White solid. ¹H NMR (400 MHz, Chloroform-d, 2 sets of rotamers) δ 7.49 |
| | (d, J = 2.0 Hz, 1H), 7.43-7.37 (m, 0.5H), 7.36-7.29 (m, 2H), 7.27-7.03 |
| | (m, 6.5H), 6.59 (br s, 1H), 4.62 & 4.59 (s, 1H), 4.17 (d, J = 5.6 Hz, 1H), |
| | 3.97-3.87 (m, 3H), 3.76-3.46 (m, 1H), 3.32 & 3.30 (s, 1H), 3.28-3.16 |
| | (m, 1H), 2.55-2.33 (m, 3H), 2.20-1.80 (m, 7.5H), 1.77-1.67 (m, 0.5H). |
| | ¹⁹F NMR (376 MHz, Chloroform-d, 2 sets of rotamers) δ-108.84, -108.99. |
| | MS (ESI⁺): [M + H]⁺ 618.1/620.1. |
| 099 | White solid. ¹H NMR (400 MHz, Chloroform-d, 2 sets of rotamers) δ 7.48 |
| | (d, J = 1.9 Hz, 1H), 7.43-7.37 (m, 0.5H), 7.27-7.05 (m, 5.5H), 7.05- |
| | 6.96 (m, 2H), 6.58 (br s, 1H), 4.62 & 4.59 (s, 1H), 4.17 (d, J = 6.0 Hz, 1H), |
| | 3.98-3.86 (m, 3H), 3.73-3.49 (m, 1H), 3.37-3.28 (m, 1H), 3.27-3.19 |
| | (m, 1H), 2.52-2.30 (m, 3H), 2.20-1.95 (m, 3.5H), 1.95-1.65 (m, 4.5H). |
| | ¹⁹F NMR (376 MHz, Chloroform-d, 2 sets of rotamers) δ-108.82, -108.96, |
| | -116.28. MS (ESI⁺): [M + H]⁺ 636.2/638.2. |
| 100 | White solid. ¹H NMR (400 MHz, Chloroform-d, 2 sets of rotamers) δ 7.58 |
| | (d, J = 2.1 Hz, 1H), 7.43-7.16 (m, 3H), 7.15-7.03 (m, 1H), 6.56 (br s, |
| | 1H), 4.61 & 4.58 (s, 1H), 4.29 (d, J = 7.6 Hz, 1H), 4.14 (s, 1H), 3.90 (s, 1H), |
| | 3.70-3.42 (m, 2H), 3.31 & 3.28 (s, 1H), 3.26-3.15 (m, 1H), 2.28-1.94 |
| | (m, 3.5H), 1.75-1.65 (m, 0.5H), 1.12 (d, J = 6.5 Hz, 6H). ¹⁹F NMR (376 |
| | MHz, Chloroform-d, 2 sets of rotamers) δ-108.89, -109.02. MS |
| | (ESI⁺): [M + H]⁺ 516.0/518.0. |
| 101 | White solid. ¹H NMR (400 MHz, Chloroform-d, 2 sets of rotamers) δ 7.58 |
| | (d, J = 2.0 Hz, 1H), 7.43-7.15 (m, 3H), 7.15-7.00 (m, 1H), 6.59 (br s, |
| | 1H), 4.91 (s, 1H), 4.61 & 4.58 (s, 1H), 4.14 (s, 1H), 3.91 & 3.88 (s, 1H), |
| | 3.71-3.45 (m, 1H), 3.32 & 3.29 (s, 1H), 3.26-3.17 (m, 1H), 2.19-1.92 |
| | (m, 3.5H), 1.75-1.65 (m, 0.5H), 1.24 (s, 3H), 0.83-0.77 (m, 2H), 0.55- |
| | 0.45 (m, 2H). ¹⁹F NMR (376 MHz, Chloroform-d, 2 sets of rotamers) δ- |
| | 108.87, -109.00. MS (ESI⁺): [M + H]⁺ 528.0/530.0. |
| 102 | White solid. ¹H NMR (400 MHz, Chloroform-d, 2 sets of rotamers) δ 7.58 |
| | (d, J = 2.0 Hz, 1H), 7.43-7.15 (m, 3H), 7.15-7.03 (m, 1H), 6.55 (br s, |
| | 1H), 4.74 (s, 1H), 4.61 & 4.58 (s, 1H), 4.14 (s, 1H), 3.89 (s, 1H), 3.72-3.46 |
| | (m, 1H), 3.32 & 3.28 (s, 1H), 3.25-3.17 (m, 1H), 2.25-1.95 (m, 5.5H), |
| | 1.94-1.87 (m, 2H), 1.82-1.65 (m, 2.5H), 1.40 (s, 3H). ¹⁹F NMR (376 |
| | MHz, Chloroform-d, 2 sets of rotamers) δ-108.89, -109.02. MS |
| | (ESI⁺): [M + H]⁺ 542.0/544.0. |
| 103 | White solid. ¹H NMR (400 MHz, Chloroform-d, 2 sets of rotamers) δ 7.62 |
| | (s, 1H), 7.45-7.32 (m, 1.5H), 7.26-7.15 (m, 2H), 7.15-7.00 (m, 1.5H), |
| | 4.88 (d, J = 3.8 Hz, 1H), 4.64-4.52 (m, 1H), 4.15 (s, 1H), 3.92 (s, 1H), 3.71- |
| | 3.47 (m, 1H), 3.33 & 3.30 (s, 1H), 3.36-3.14 (m, 1H), 2.23-1.94 (m, |
| | 3.5H), 1.76-1.66 (m, 6.5H). ¹⁹F NMR (376 MHz, Chloroform-d, 2 sets of |
| | rotamers) δ-108.93, -109.08. MS (ESI⁺): [M + H]⁺ 541.0/543.0. |
| 104 | White solid. ¹H NMR (400 MHz, Chloroform-d, 2 sets of rotamers) |
| | δ 7.59 (d, J = 2.1 Hz, 1H), 7.45-7.35 & 7.27-7.16 (m, 3H), 7.16-7.05 |
| | (m, 1H), 6.58 (s, 1H), 4.69-4.50 (m, 2H), 4.19-4.07 (m, 1H), 4.05-3.94 |
| | (m, 1H), 3.93-3.80 (m, 1H), 3.67 & 3.50 (d, J = 11.7 Hz, 1H), 3.30 (d, J = |
| | 13.0 Hz, 1H), 3.28-3.13 (m, 1H), 2.17-1.88 & 1.78-1.66 (m, 4H), 1.39 |
| | (d, J = 7.0 Hz, 3H). ¹⁹F NMR (376 MHz, Chloroform-d, 2 sets of rotamers) |
| | δ-77.35, -77.36, -77.37, -108.89, -109.01. MS (ESI⁺): [M + H]⁺ 570.0/572.0. |
| 105 | White solid. ¹H NMR (400 MHz, Chloroform-d, 2 sets of rotamers) δ 7.60 |
| | (d, J = 2.1 Hz, 1H), 7.43-7.36 & 7.27-7.16 (m, 3H), 7.15-7.04 (m, 1H), |
| | 6.59 (s, 1H), 4.80 (s, 1H), 4.60 (d, J = 13.3 Hz, 1H), 4.18-4.05 (m, 1H), |
| | 3.93-3.80 (m, 1H), 3.67 & 3.50 (d, J = 12.5 Hz, 1H), 3.30 (d, J = 13.1 Hz, |
| | 1H), 3.22 (dd, J = 12.4, 5.6 Hz, 1H), 2.24-1.91 & 1.75-1.67 (m, 4H), |
| | 1.46 (s, 6H). ¹⁹F NMR (376 MHz, Chloroform-d, 2 sets of rotamers) |
| | δ-82.05, -108.89, -109.02. MS (ESI⁺): [M + H]⁺ 583.9/585.9. |
| 106 | White solid. ¹H NMR (400 MHz, Chloroform-d, 2 sets of rotamers) δ 7.55 |
| | (d, J = 2.1 Hz, 1H), 7.42-7.38 & 7.28-7.16 (m, 3H), 7.10 (dtd, J = 16.6, |
| | 8.2, 2.4 Hz, 1H), 6.61 (s, 1H), 4.86 (d, J = 5.4 Hz, 2H), 4.68-4.54 (m, 1H), |
| | 4.35-4.25 (m, 2H), 4.15 (s, 1H), 3.91 (s, 1H), 3.72-3.63 & 3.54-3.44 |
| | (m, 1H), 3.35-3.16 (m, 2H), 2.54 (s, 3H), 2.21-1.95 & 1.83-1.68 (m, |
| | 7H). ¹⁹F NMR (376 MHz, Chloroform-d, 2 sets of rotamers) δ-108.87, |
| | -109.00. MS (ESI⁺): [M + H]⁺ 558.0/560.0. |
| 107 | White solid. ¹H NMR (400 MHz, Chloroform-d, 2 sets of rotamers) δ 7.48 |
| | (d, J = 2.0 Hz, 1H), 7.42-7.13 (m, 3H), 7.13-7.00 (m, 1H), 6.56 (br s, |
| | 1H), 4.61 & 4.56 (s, 1H), 4.08 (s, 1H), 3.85 (s, 1H), 3.70-3.45 (m, 1H), |
| | 3.30 & 3.27 (s, 1H), 3.25-3.16 (m, 1H), 2.93 (s, 3H), 2.15-1.90 (m, 3.5H), |
| | 1.75-1.65 (m, 0.5H), 1.36 (s, 9H). ¹⁹F NMR (376 MHz, Chloroform-d, 2 |
| | sets of rotamers) δ-108.92, -109.04. MS (ESI⁺): [M + H]⁺ 544.0/546.0. |
| 108 | White solid. ¹H NMR (400 MHz, Chloroform-d, 2 sets of rotamers δ 7.55 |
| | (s, 1H), 7.41-7.33 (m, 0.5H), 7.26-6.96 (m, 3.5H), 5.00 (s, 1H), 4.66 (d, |
| | J = 6.5 Hz, 2H), 4.58 (d, J = 13.3 Hz, 1H), 4.34 (d, J = 6.5 Hz, 2H), 4.12 (s, |
| | 1H), 3.89 (s, 1H), 3.65 (d, J = 12.6 Hz, 0.5H), 3.48 (d, J = 8.7 Hz, 0.5H), |
| | 3.23 (dd, J = 36.9, 11.9 Hz, 2H), 2.20-1.90 (m, 3.5H), 1.79-1.58 (m, |
| | 3.5H). ¹⁹F NMR (376 MHz, Chloroform-d, 4 sets of rotamers) δ-108.82, |
| | -108.85. MS (ESI⁺): [M + H]⁺ 543.9/545.9, [M + Na]⁺ 565.9/567.9. |
| 109 | White solid. ¹H NMR (400 MHz, Chloroform-d, 2 sets of rotamers) δ 7.46- |
| | 7.32 (m, 1.5H), 7.24-6.99 (m, 3.5H), 4.79-4.68 (m, 1H), 4.68-4.62 |
| | (m, 4H), 4.58 (d, J = 13.2 Hz, 1H), 4.14 (s, 1H), 3.90 (s, 1H), 3.70-3.45 |
| | (m, 1H), 3.32-3.14 (m, 2H), 2.81 (s, 3H), 2.13-1.92 (m, 3.5H), 1.76- |
| | 1.67 (m, 0.5H). ¹⁹F NMR (376 MHz, Chloroform-d, 4 sets of rotamers) |
| | δ-108.79, -108.92. MS (ESI⁺): [M + H]⁺ 543.9/545.9, [M+Na]+ 566.0/568.0. |
| 110 | White solid. ¹H NMR (400 MHz, Chloroform-d, 4 sets of rotamers) δ 7.82 |
| | (br s, 1H), 7.81-7.76 (m, 1H), 7.67-7.60 (m, 1H), 7.47-7.27 (m, 1H), |
| | 7.27-7.16 (m, 1H), 7.16-7.06 (m, 1H), 4.87-4.70 (m, 1H), 4.49 (s, 1H), |
| | 3.55-2.75 (m, 6H), 1.26 (s, 9H), 0.99-0.72 (m, 3H). ¹⁹F NMR (376 MHz, |
| | Chloroform-d, 4 sets of rotamers) δ-108.59, -108.62, -108.66, -108.70. MS |
| | (ESI⁺): [M + H]⁺ 518.0/520.0. |
| 111 | White solid. ¹H NMR (400 MHz, Chloroform-d, 4 sets of rotamers) δ 7.82 |
| | (br s, 1H), 7.81-7.76 (m, 1H), 7.67-7.60 (m, 1H), 7.47-7.27 (m, 1H), |
| | 7.27-7.16 (m, 1H), 7.16-7.06 (m, 1H), 4.87-4.70 (m, 1H), 4.49 (s, 1H), |
| | 3.55-2.75 (m, 6H), 1.26 (s, 9H), 0.99-0.72 (m, 3H). ¹⁹F NMR (376 MHz, |
| | Chloroform-d, 4 sets of rotamers) δ-108.59, -108.62, -108.66, -108.70. MS |
| | (ESI⁺): [M + H]⁺ 518.0/520.0. |
| 112 | White solid. ¹H NMR (400 MHz, Chloroform-d, 2 sets of rotamers) δ 7.63- |
| | 7.57 (m, 1H), 7.40-7.28 (m, 1.5H), 7.24-6.98 (m, 2.5H), 4.70-4.54 |
| | (m, 2H), 4.38-4.17 (m, 3H), 4.07-3.94 (m, 1H), 3.84-3.63 (m, 2.5H), |
| | 3.54 (d, J = 13.0 Hz, 0.5H), 3.36-3.15 (m, 2H), 2.12-1.81 (m, 3.5H), 1.74- |
| | 1.59 (m, 0.5H), 1.24 (s, 9H). ¹⁹F NMR (376 MHz, Chloroform-d, 2 sets of |
| | rotamers) δ-108.21, -108.95. MS (ESI⁺): [M + H]⁺ 574.0/576.0. |
| 113 | White solid. ¹H NMR (400 MHz, Chloroform-d, 2 sets of rotamers) δ 7.49- |
| | 7.44 (m, 1H), 7.40-7.33 (m, 0.5H), 7.24-6.98 (m, 3.5H), 4.63 (s, 1H), |
| | 4.54-4.45 (m, 1H), 4.42-4.34 (m, 1H), 4.24-4.06 (m, 3H), 3.76-3.69 |
| | (m, 2H), 3.69-3.62 (m, 0.5H), 3.54-3.44 (m, 0.5H), 3.44-3.35 (m, 3H), |
| | 3.33-3.22 (m, 1H), 3.14-3.04 (m, 1H), 2.16-1.81 (m, 3.5H), 1.67-1.58 |
| | (m, 0.5H), 1.31-1.15 (m, 9H). ¹⁹F NMR (376 MHz, Chloroform-d, 2 sets |
| | of rotamers) δ-109.07, -109.22. MS (ESI⁺): [M + H]⁺ 588.1/590.1. |
| 114 | White solid. ¹H NMR (400 MHz, Chloroform-d, 2 sets of rotamers) δ 7.46 |
| | (s, 1H), 7.37 (dd, J = 8.4, 5.9 Hz, 0.5H), 7.24-6.95 (m, 3.5H), 4.69 (s, 1H), |
| | 4.56-4.48 (m, 1H), 4.43-4.30 (m, 1H), 4.23-4.08 (m, 1H), 3.84 (s, 3H), |
| | 3.59 (d, J = 12.7 Hz, 0.5H), 3.44 (d, J = 12.4 Hz, 0.5H), 3.23 (d, J = 13.0 |
| | Hz, 1H), 3.20-3.07 (m, 1H), 2.09-1.82 (m, 3.5H), 1.70-1.60 (m, 0.5H), |
| | 1.37-1.12 (m, 9H). ¹⁹F NMR (376 MHz, Chloroform-d, 2 sets of rotamers) |
| | δ-108.99, -109.13. MS (ESI⁺): [M + H]⁺ 544.0/546.0. |
| 115 | White solid. ¹H NMR (400 MHz, Chloroform-d, 2 sets of rotamers) δ 7.53- |
| | 7.48 (m, 1H), 7.37 (dd, J = 8.5, 5.9 Hz, 1H), 7.30 (dd, J = 8.3, 1.9 Hz, 1H), |
| | 7.25-7.02 (m, 3H), 4.63-4.54 (m, 1H), 4.19-4.12 (m, 1H), 3.97-3.89 |
| | (m, 1H), 3.77-3.52 (m, 1H), 3.34 (d, J = 12.8 Hz, 1H), 3.21 (t, J = 10.6 Hz, |
| | 1H), 2.95 (t, J = 7.3 Hz, 2H), 2.71 (s, 3H), 2.12-1.86 (m, 4H), 1.73-1.59 |
| | (m, 1H), 1.55-1.49 (m, 1H), 0.92 (t, J = 7.4 Hz, 3H). ¹⁹F NMR (376 MHz, |
| | Chloroform-d, 2 sets of rotamers) δ-109.25, -109.36. (ESI⁺): [M + H]⁺ |
| | 514.1/516.1. |
| 116 | White solid. ¹H NMR (400 MHz, Chloroform-d, set of 2 rotamers) δ 7.40- |
| | 7.29 (m, 2.5H), 7.26-6.92 (m, 7.5H), 4.63-4.54 (m, 1H), 3.94-3.86 (m, |
| | 1H), 3.70-3.60 (m, 1.5H), 3.54-3.46 (m, 0.5H), 3.33-3.27 (m, 1H), 3.22 |
| | (t, J = 10.4 Hz, 1H), 3.08-2.94 (m, 4H), 2.77-2.66 (m, 2H), 2.64-2.52 |
| | (m, 6H), 2.49-2.35 (m, 3H), 2.13-1.87 (m, 3.5H), 1.71-1.61 (m, |
| | 0.5H). ¹⁹F NMR (376 MHz, Chloroform-d, set of 2 rotamers) δ-109.1, |
| | -109.3, -117.0. (ESI⁺): [M + H]⁺ 629.2/631.2. |
| 117 | Yellow solid. ¹H NMR (400 MHz, , Chloroform-d, set of 2 rotamers) δ 7.39- |
| | 7.28 (m, 2H), 7.24-7.18 (m, 2H), 7.18-7.13 (m, 2H), 7.13-6.98 (m, |
| | 2H), 6.84-6.78 (m, 2H), 6.03 (s, 2H), 4.53-4.40 (m, 2H), 4.25 (s, 1H), |
| | 3.89 (d, J = 12.0 Hz, 2H), 3.66-3.42 (m, 1H), 3.23 (d, J = 13.1 Hz, 1H), |
| | 3.07 (t, J = 11.0 Hz, 1H), 2.50-2.37 (m, 3H), 2.15-1.95 (m, 3H), 1.92- |
| | 1.79 (m, 4H), 1.71-1.62 (m, 1H). ¹⁹F NMR (376 MHz, Chloroform-d, 2 sets |
| | of rotamers) δ-108.97, -109.13. MS (ESI⁺): [M + H]⁺ 612.1/614.1. |
| 118 | White solid. ¹H NMR (400 MHz, Chloroform-d, 2 sets of rotamers) δ 7.43- |
| | 7.30 (m, 2.5H), 7.27-7.20 (m, 1.5H), 7.20-7.14 (m, 3H), 7.14-7.00 |
| | (m, 1H), 6.98 (d, J = 2.0 Hz, 1H), 6.81 (t, J = 2.5 Hz, 1H), 4.53 (d, J = 13.2 |
| | Hz, 1H), 4.45-4.37 (m, 2H), 4.36-4.27 (m, 3H), 4.09 (s, 1H), 3.90 (d, |
| | J = 11.6 Hz, 2H), 3.76-3.47 (m, 1H), 3.31 (d, J = 12.9 Hz, 1H), 3.22-3.07 |
| | (m, 1H), 2.58-2.29 (m, 3H), 2.17-1.60 (m, 8H). ¹⁹F NMR (376 MHz, |
| | Chloroform-d, 2 sets of rotamers δ-109.19, -109.32. MS (ESI⁺): [M + H]⁺ |
| | 626.2/628.2. |
| 119 | White solid. ¹H NMR (400 MHz, Chloroform-d, 2 sets of rotamers) δ 7.38- |
| | 7.27 (m, 2H), 7.23-6.99 (m, 8H), 4.51 (d, J = 14.0 Hz, 1H), 4.10 (s, 1H), |
| | 3.87 (d, J = 10.7 Hz, 3H), 3.70-3.23 (m, 4H), 3.19-2.94 (m, 6H), 2.50- |
| | 2.23 (m, 3H), 2.10-1.73 (m, 8H). ¹⁹F NMR (376 MHz, Chloroform-d, 2 |
| | sets of rotamers) δ-109.12, -109.29. MS (ESI⁺): [M + H]⁺ 623.2/625.2. |
| 120 | White solid. ¹H NMR (400 MHz, Chloroform-d, 2 sets of rotamers) δ 7.35- |
| | 7.28 (m, 2H), 7.23-7.18 (m, 2H), 7.14 (d, J = 8.8 Hz, 4H), 7.07-7.03 |
| | (m, 1H), 4.48 (d, J = 13.3 Hz, 1H), 4.11 (s, 1H), 3.88 (d, J = 11.8 Hz, 3H), |
| | 3.71-3.65 (m, 1H), 3.48 (dt, J = 18.5, 10.6 Hz, 3H), 3.29 (d, J = 13.0 Hz, |
| | 1H), 3.14 (t, J = 10.7 Hz, 1H), 3.07 (s, 3H), 3.02 (t, J = 8.8 Hz, 2H), 2.03- |
| | 1.79 (m, 9H). ¹⁹F NMR (376 MHz, Chloroform-d, 2 sets of rotamers) δ- |
| | 132.5 & -132.8 (d, JF-F = 21 Hz), -136.7 & -137.3 (d, JF-F= 21 Hz). MS |
| | (ESI⁺): [M + H]⁺ 641.2/643.2. |
| 121 | White solid. ¹H NMR (400 MHz, Chloroform-d, 2 sets of rotamers) δ 7.43- |
| | 7.29 (m, 3.5H), 7.26-7.20 (m, 1.5H), 7.20-6.99 (m, 6H), 4.60-4.40 |
| | (m, 2H), 4.26 (s, 1H), 3.93 (d, J = 11.6 Hz, 2H), 3.67 & 3.50 (d, J = 12.5 |
| | Hz, 1H), 3.35-3.22 (m, 1H), 3.16 (t, J = 10.6 Hz, 1H), 2.89 & 2.87 (s, 6H), |
| | 2.55-2.33 (m, 3H), 2.13-1.75 (m, 7.5H), 1.70-1.60 (m, 0.5H). ¹⁹F NMR |
| | (376 MHz, Chloroform-d, 2 sets of rotamers δ-109.22, -109.39. MS |
| | (ESI⁺): [M + H]⁺ 611.2/613.2. |
| 122 | White solid. ¹H NMR (400 MHz, Chloroform-d, 2 sets of rotamers) δ 7.34- |
| | 7.27 (m, 3H), 7.25-7.19 (m, 2H), 7.18-7.12 (m, 3H), 7.07-6.98 (m, |
| | 2H), 4.53-4.40 (m, 2H), 4.27 (s, 1H), 3.91 (d, J = 11.5 Hz, 2H), 3.70- |
| | 3.44 (m, 1H), 3.32-3.21 (m, 1H), 3.18-3.10 (m, 1H), 2.85 (s, 6H), 2.48- |
| | 2.32 (m, 3H), 2.08-1.78 (m, 8H). ¹⁹F NMR (376 MHz, Chloroform-d, 2 |
| | sets of rotamers) δ-132.6 & -132.9 (d, JF-F= 19 Hz), -136.9 & -137.3 (d, |
| | J_{F-F} = 19 Hz). MS (ESI⁺): [M + H]⁺ 629.3/631.3. |
| 123 | White solid. ¹H NMR (400 MHz, Chloroform-d, 2 sets of rotamers) δ 7.66- |
| | 7.42 (m, 2H), 7.40-7.22 (m, 4.5H), 7.22-7.12 (m, 3.5H), 7.09 (s, 1H), |
| | 6.68 & 6.66 (s, 1H), 4.35-3.77 (m, 5H), 3.77-3.62 (m, 2H), 3.60-3.45 |
| | (m, 4H), 3.29 (s, 3H), 3.22-2.95 (m, 2H), 2.40-2.15 (m, 3H), 1.95-1.50 |
| | (m, 8H). ¹⁹F NMR (376 MHz, DMSO-d6, 2 sets of rotamers) δ-110.0, |
| | -110.2. MS (ESI⁺): [M + H]⁺ 652.4/654.4. |
| 124 | White solid. ¹H NMR (400 MHz, Chloroform-d, set of 2 rotamers) δ 7.37 |
| | (dd, J = 8.5, 5.8 Hz, 0.5H), 7.33-7.26 (m, 2H), 7.25-7.01 (m, 5.5H), 6.95 |
| | (s, 1H), 6.83-6.77 (m, 1H), 6.11-5.96 (m, 1H), 4.64-4.50 (m, 1H), 3.96- |
| | 3.75 (m, 3H), 3.73-3.50 (m, 2H), 3.32 (d, J = 12.6 Hz, 1H), 3.21 (t, J = |
| | 9.7 Hz, 1H), 2.48-2.25 (m, 6H), 2.05-1.77 (m, 7.5H), 1.67-1.61 (m, |
| | 0.5H). ¹⁹F NMR (376 MHz, Chloroform-d, 2 sets of rotamers) δ-108.99, |
| | -109.13. MS (ESI⁺): [M + H]⁺ 598.3/600.3. |
| 125 | Yellow solid. ¹H NMR (400 MHz, Chloroform-d, 2 sets of rotamers) δ 7.74- |
| | 7.68 (m, 1H), 7.40-7.34 (m, 0.5H) 7.34-7.27 (m, 3.5H), 7.25-7.02 (m, |
| | 6H), 4.67-4.60 (m, 1H), 4.51-4.43 (m, 1H), 4.36-4.27 (m, 1H), 3.98- |
| | 3.90 (m, 2H), 3.77-3.57 (m, 1H), 3.34-3.22 (m, 2H), 2.52-2.41 (m, 3H), |
| | 2.08-1.81 (m, 7.5H), 1.75-1.70 (m, 0.5H). ¹⁹F NMR (376 MHz, |
| | Chloroform-d, 2 sets of rotamers) δ-108.72, -108.85. MS (ESI⁺): [M + H]⁺ |
| | 593.1/595.1 |
| 126 | White solid. ¹H NMR (400 MHz, Chloroform-d, 2 sets of rotamers) δ 7.42- |
| | 7.32 & 7.24-6.98 (m, 7H), 6.97-6.86 (m, 2H), 6.64-6.53 (m, 2H), 4.82- |
| | 4.77 (m, 1H), 4.52-4.42 (m, 1H), 4.28-4.21 & 4.20-4.13 (m, 1H), 4.04- |
| | 3.95 (m, 1H), 3.93 & 3.92 (s, 3H), 3.69-3.59 & 3.47-3.38 (m, 3H), 3.55- |
| | 3.48 (m, 1H), 3.31-3.21 (m, 2H), 3.15-3.05 (m, 1H), 2.12-2.04 (m, |
| | 2H), 2.02-1.80 & 1.61-1.57 (m, 4H). ¹⁹F NMR (376 MHz, Chloroform- |
| | d, 2 sets of rotamers) δ-109.32, -109.43. MS (ESI⁺): [M + H]⁺ 600.2/602.2. |
| 127 | White solid. ¹H NMR (400 MHz, Chloroform-d, 2 sets of rotamers) δ 7.42 |
| | (dd, J = 8.4, 2.0 Hz, 1H), 7.38-7.35 & 7.25-7.18 (m, 4H), 7.17-7.12 (m, |
| | 1H), 7.08 & 7.02 (td, J = 8.2, 2.5 Hz, 1H), 6.96 (d, J = 8.5 Hz, 1H), 6.81- |
| | 6.76 (m, 1H), 6.76-6.71 (m, 2H), 4.51 (dd, J = 13.3, 6.6 Hz, 1H), 4.33- |
| | 4.26 (m, 1H), 4.26-4.19 (m, 1H), 4.08-4.02 (m, 1H), 3.95 & 3.94 (s, 3H), |
| | 3.67 & 3.51 (d, J = 12.5 Hz, 1H), 3.48-3.40 (m, 1H), 3.37-3.27 (m, 2H), |
| | 3.21-3.07 (m, 3H), 2.73 & 2.72 (s, 3H), 2.09-2.00 (m, 2H), 2.00-1.85 |
| | & 1.65-1.59 (m, 4H). ¹⁹F NMR (376 MHz, Chloroform-d, 2 sets of |
| | rotamers) δ-109.24, -109.35. MS (ESI⁺): [M + H]⁺ 613.2/615.2. |
| 128 | White solid. ¹H NMR (400 MHz, Chloroform-d, 2 sets of rotamers) δ 7.44- |
| | 7.29 & 7.26-7.07 (m, 7H), 7.08-7.01 (m, 2H), 6.99 (d, J = 8.5 Hz, 1H), |
| | 4.69 (d, J = 10.1 Hz, 1H), 4.55 (d, J = 13.6 Hz, 1H), 4.32 (s, 1H), 4.11 (s, |
| | 1H), 3.97 & 3.96 (s, 3H), 3.70 & 3.53 (d, J = 11.8 Hz, 2H), 3.33 (d, J = 13.0 |
| | Hz, 1H), 3.17 (t, J = 10.1 Hz, 1H), 3.12-3.00 (m, 4H), 2.89-2.77 (m, 2H), |
| | 2.63-2.36 (m, 4H), 2.17-1.87 & 1.72-1.63 (m, 4H). ¹⁹F NMR (376 |
| | MHz, Chloroform-d, 2 sets of rotamers) δ-109.23, -109.35, -114.80, |
| | -114.81. MS (ESI⁺): [M + H]⁺ 661.3/663.3. |
| 129 | White solid. ¹H NMR (400 MHz, Chloroform-d, 2 sets of rotamers) δ 7.54- |
| | 7.41 (m, 1H), 7.35-7.28 (m, 0.5H), 7.28-7.26 (m, 1H), 7.25-7.17 (m, |
| | 0.5H), 7.09-6.97 (m, 1H), 6.90 (d, J = 8.5 Hz, 1H), 4.52-4.42 (m, 1H), |
| | 4.40-4.34 (m, 1H), 4.31-4.24 (m, 1H), 4.12-4.03 (m, 1H), 3.95-3.89 |
| | (m, 3H), 3.73-3.66 (m, 0.5H), 3.57-3.46 (m, 0.5H), 3.35-3.24 (m, 1H), |
| | 3.17-3.07 (m, 1H), 2.10-1.83 (m, 3.5H), 1.65-1.55 (m, 0.5H), 1.21 (s, |
| | 9H). ¹⁹F NMR (376 MHz, Chloroform-d, 2 sets of rotamers) δ-132.7 & |
| | -132.9 (d, J_{F-F} = 21.0 Hz), -137.0 & -137.3 (d, J_{F-F} = 21.0 Hz). MS |
| | (ESI⁺): [M + H]⁺ 528.1, 530.1 [M + Na]⁺ 550.1/552.1. |
| 130 | White solid. ¹H NMR (400 MHz, Chloroform-d, 2 sets of rotamers) δ 7.39- |
| | 7.32 (m, 0.5H), 7.23-7.17 (m, 0.5H), 7.16-6.94 (m, 4H), 4.90-4.74 |
| | (m, 0.5H), 4.74-4.61 (m, 2.5H), 4.53-4.38 (m, 2H), 4.34-4.19 (m, 1H), |
| | 3.69-3.57 (m, 0.5H), 3.52-3.40 (m, 0.5H), 3.39-3.16 (m, 5H), 3.13- |
| | 2.98 (m, 1H), 2.93-2.78 (m, 2H), 2.14-1.79 (m, 3.5H), 1.71-1.60 (m, |
| | 0.5H). ¹⁹F NMR (376 MHz, Chloroform-d, 2 sets of rotamers) δ-109.23, |
| | -109.36, -185.33. MS (ESI⁺): [M + H]⁺ 552.2/554.2. |
| 131 | Pink solid. ¹H NMR (400 MHz, Chloroform-d, 2 sets of rotamers) δ 7.50- |
| | 7.44 (m, 1H), 7.37 (dd, J = 8.5, 5.9 Hz, 0.5H), 7.24-6.95 (m, 3.5H), 6.55 |
| | (br s, 1H), 4.58 (d, J = 13.0 Hz, 1H), 4.15-4.05 (m, 1H), 3.97-3.82 (m, |
| | 1H), 3.80-3.60 (m, 2.5H), 3.55-3.40 (m, 3H), 3.30-3.15 (m, 3.5H), 2.17- |
| | 1.89 (m, 3.5H), 1.75-1.65 (m, 0.5H), 1.27 (s, 6H). ¹⁹F NMR (376 MHz, |
| | Chloroform-d, 2 sets of rotamers) δ-108.85, -108.98. MS (ESI⁺): [M + H]⁺ |
| | 572.0/574.0. |
| 132 | White solid. ¹H NMR (400 MHz, Chloroform-d, 2 sets of rotamers) δ 7.50- |
| | 7.45 (m, 1H), 7.41-7.34 (m, 0.5H), 7.26-7.14 (m, 2.5H), 7.14-7.00 |
| | (m, 1H), 4.66-4.61 (m, 1H), 3.92-3.79 (m, 1H), 3.72-3.65 (m, 0.5H), |
| | 3.65-3.56 (m, 1H), 3.55-3.48 (m, 0.5H), 3.36-3.28 (m, 1H), 3.27-3.17 |
| | (m, 1H), 3.01-2.91 (m, 3H), 2.52-2.37 (m, 3H), 2.12-1.84 (m, 3.5H), |
| | 1.71-1.66 (m, 0.5H), 1.35 (s, 9H). ¹⁹F NMR (376 MHz, Chloroform-d, 2 |
| | sets of rotamers) δ-109.05, -109.17. MS (ESI⁺): [M + H]⁺ 542.1/544.1. |
| 133 | White solid. ¹H NMR (400 MHz, Chloroform-d, 2 sets of rotamers) δ 7.55- |
| | 7.48 (m, 1H), 7.41-7.34 (m, 0.5H), 7.26-7.14 (m, 2.5H), 7.14-7.00 |
| | (m 1H), 4.67-4.55 (m, 1H), 4.16-4.08 (m, 1H), 3.95-3.85 (m, 1H), 3.77- |
| | 3.69 (m, 0.5H), 3.63-3.54 (m, 0.5H), 3.39-3.30 (m, 1H), 3.28-3.16 |
| | (m, 1H), 2.97 (s, 3H), 2.13-1.82 (m, 3.5H), 1.75-1.62 (m, 0.5H), 1.37 (s, |
| | 9H). ¹⁹F NMR (376 MHz, Chloroform-d, 2 sets of rotamers) δ-109.03, |
| | -109.16. MS (ESI⁺): [M + H]⁺ 562.0/564.0. |
| 134 | White solid. ¹H NMR (400 MHz, Chloroform-d) δ 7.55-7.46 (m, 1H), 7.40- |
| | 7.32 (m, 0.5H), 7.26-7.13 (m, 2.5H), 7.12-7.01 (m, 1H), 4.60 (s, 1H), |
| | 4.43-4.12 (m, 3H), 4.00 (d, J = 14.2 Hz, 1H), 3.85-3.73 (m, 2H), 3.70 (d, |
| | J = 12.9 Hz, 0.5H), 3.55 (d, J = 12.7 Hz, 0.5H), 3.42-3.29 (m, 1H), 3.21 |
| | (d, J = 12.9 Hz, 1H), 2.94 (s, 3H), 2.04-1.90 (m, 3.5H), 1.70-1.65 (m, |
| | 0.5H), 1.36 (s, 9H). ¹⁹F NMR (376 MHz, Chloroform-d, 2 sets of rotamers) |
| | δ-108.85, -108.96. MS (ESI⁺): [M + H]⁺ 588.1/590.1. |
| 135 | White solid. ¹H NMR (400 MHz, Chloroform-d) δ 7.43-7.36 (m, 1H), 7.24 |
| | (dd, J = 8.5, 2.5 Hz, 1H), 7.21-7.15 (m, 2H), 7.14-7.02 (m, 1H), 4.53 (d, |
| | J = 12.2 Hz, 1H), 4.41 (s, 1H), 4.21 (s, 1H), 4.19-4.13 (m, 2H), 3.78-3.72 |
| | (m, 2H), 3.69 (d, J = 12.7 Hz, 1H), 3.44 (d, 3H), 3.30 (d, J = 13.1 Hz, 1H), |
| | 3.18-3.08 (m, 1H), 2.97 (s, 3H), 2.04-1.89 (m, 3H), 1.69-1.62 (m, 1H), |
| | 1.38 (s, 9H). ¹⁹F NMR (376 MHz, Chloroform-d, 2 sets of rotamers) |
| | δ-109.10, -109.24. MS (ESI⁺): [M + H]⁺ 602.2/604.2. |
| 136 | White solid. ¹H NMR (400 MHz, Chloroform-d, set of 2 rotamers)δ 7.50- |
| | 7.29 (m, 6H), 7.24-6.95 (m, 4H), 5.09-4.93 (m, 2H), 4.45 (d, J = 13.1 |
| | Hz, 1H), 4.34 (s, 1H), 4.14 (s, 1H), 3.47 (d, J = 12.7 Hz, 0.5H), 3.26 (d, J = |
| | 12.2 Hz, 0.5H), 3.16 (dd, J = 13.4, 6.3 Hz, 1H), 3.03 (t, J = 10.5 Hz, 1H), |
| | 2.96 (s, 3H), 2.05-1.79 (m, 3.5H), 1.64-1.58 (m, 0.5H), 1.37 (s, 9H). ¹⁹F |
| | NMR (376 MHz, Chloroform-d, set of 2 rotamers) δ-109.07, -109.20. |
| | (ESI⁺): [M + H]⁺ 634.2/636.3. |
| 137 | White solid. ¹H NMR (400 MHz, Chloroform-d, set of 2 rotamers) δ 8.70 |
| | (s, 1H), 8.63 (d, J = 4.8 Hz, 1H), 7.84 (d, J = 7.9 Hz, 1H), 7.44-7.30 (m, |
| | 2.5H), 7.22-7.17 (m, 1.5H), 7.17-7.11 (m, 1H), 7.10-6.98 (m, 1H), 5.07 |
| | (s, 2H), 4.48 (d, J = 13.1 Hz, 1H), 4.31 (d, J = 5.7 Hz, 1H), 4.11 (s, 1H), |
| | 3.46 (d, J = 12.6 Hz, 0.5H), 3.28 (s, 0.5H), 3.21-3.04 (m, 2H), 2.96 (s, 3H), |
| | 2.04-1.78 (m, 3.5H), 1.70-1.56 (m, 0.5H), 1.37 (s, 9H). ¹⁹F NMR (376 |
| | MHz, Chloroform-d, set of 2 rotamers) δ-108.91, -109.08. (ESI⁺): [M + H]⁺ |
| | 635.2/637.3. |
| 138 | White solid. ¹H NMR (400 MHz, Chloroform-d, set of 2 rotamers) δ 8.66 |
| | (d, J = 5.0 Hz, 2H), 7.51-7.38 (m, 3H), 7.38-7.30 (m, 0.5H), 7.24-6.95 |
| | (m, 3.5H), 5.06 (s, 2H), 4.49 (d, J = 13.0 Hz, 1H), 4.31 (d, J = 5.7 Hz, 1H), |
| | 4.10 (s, 1H), 3.44 (d, J = 12.5 Hz, 0.5H), 3.24 (d, J = 12.4 Hz, 0.5H), 3.17- |
| | 3.04 (m, 2H), 2.97 (s, 3H), 2.04-1.79 (m, 3.5H), 1.70-1.56 (m, 0.5H), |
| | 1.37 (s, 9H). ¹⁹F NMR (376 MHz, Chloroform-d, set of 2 rotamers) |
| | δ-108.81, -108.97. (ESI⁺): [M + H]⁺ 635.2/637.3. |
| 139 | White solid. ¹H NMR (400 MHz, Chloroform-d, 2 sets of rotamers) δ 7.55 |
| | (d, J = 6.9 Hz, 1H), 7.30 (dd, J = 8.5, 5.8 Hz, 0.5H), 7.17-7.07 (m, 1.5H), |
| | 7.06-6.94 (m, 1H), 6.92-6.61 (m, 1H), 4.60-4.44 (m, 2H), 4.01-3.86 |
| | (m, 1H), 3.75-3.64 (m, 1H), 3.63-3.53 (m, 0.5H), 3.45-3.37 (m, 0.5H), |
| | 3.25-3.10 (m, 2H), 2.15-1.86 (m, 3.5H), 1.62 (d, J = 12.5 Hz, 0.5H), 1.17 |
| | (s, 9H). ¹⁹F NMR (376 MHz, Chloroform-d, 2 sets of rotamers) δ-108.94, |
| | -109.07, -124.05, -124.14. MS (ESI⁺): [M + H]⁺ 548.1/550.1. |

### Example 2: Biological Activity of the Compounds

The purpose of this experiment was to evaluate the GFRα1-RET activity of the compounds 001-139 according to the invention.

### Materials and Methods

The compounds were tested for their activating activity of Elk1 signaling using the previously developed reporter-gene-based system in cells expressing GFRα1-RET (MG87 murine fibroblast stably transfected with PathDetect Elk-1, GFRα1, and RET) disclosed in Sidorova, Y. A. et al.: “Persephin signaling through GFRα1: The potential for the treatment of Parkinson's disease.” Molecular and Cellular Neuroscience, July 2010, Vol. 44, pp. 223-232. DOI: 10.1016/j.men.2010.03.009. For ECso determination, a dose-response test was performed using 6 concentrations of each tested compound. Dose-response curves were fitted using the sigmoidal dose-response (variable slope) analysis in GraphPad Prism program (Graph Pad Inc) and ECso of agonist/activator activity was calculated. Dose-response experiments were all performed in duplicate, two times independently.

### Results

The results are presented on Table 4 below (* means “50 μM>EC₅₀>10 μM”, ** means “1 μM≤EC₅₀≤10 μM”, *** means “EC₅₀<1 μM”).

TABLE 4

| | | Lucif- |
|---|---|---|
| | | erase |
| | | Assay |
| Cpd | Name | EC₅₀ |
| 001 | 4-methoxy-N-methyl-N-propyl-3-[rac-(2S)-4-(2-chloro- | ** |
| | 4-fluoro-benzoyl)-2-methyl-piperazin-1- | |
| | yl]benzenesulfonamide | |
| 002 | N-tert-butyl-3-[(1S,5R)-3-(2-chloro-4-fluoro-benzoyl)- | ** |
| | 3,8-diazabicyclo[3.2.1]octan-8-yl]-4-methoxy- | |
| | benzenesulfonamide | |
| 003 | 3-[(1S,5R)-3-(2-chloro-4-fluoro-benzoyl)-3,8- | ** |
| | diazabicyclo[3.2.1]octan-8-yl]-4-methoxy-N-methyl-N- | |
| | propyl-benzenesulfonamide | |
| 004 | (2-chloro-4-fluoro-phenyl)-[(1S,5R)-8-(2-methoxy-5- | ** |
| | pyrrolidin-1-ylsulfonyl-phenyl)-3,8- | |
| | diazabicyclo[3.2.1]octan-3-yl]methanone | |
| 005 | 3-[(1S,5R)-3-(2-chloro-4-fluoro-benzoyl)-3,8- | *** |
| | diazabicyclo[3.2.1]octan-8-yl]-N-(cyclopropylmethyl)-4- | |
| | methoxy-N-methyl-benzenesulfonamide | |
| 006 | (2-chloro-4-fluoro-phenyl)-[(1S,5R)-8-[5-[(4-fluoro-1- | ** |
| | piperidyl)sulfonyl]-2-methoxy-phenyl]-3,8- | |
| | diazabicyclo[3.2.1]octan-3-yl]methanone | |
| 007 | (2-chloro-4-fluoro-phenyl)-[(1S,5R)-8-[2-methoxy-5-[[4- | *** |
| | (trifluoromethyl)-1-piperidyl]sulfonyl]phenyl]-3,8- | |
| | diazabicyclo[3.2.1]octan-3-yl]methanone | |
| 008 | [(1S,5R)-8-[5-[(4-benzylidene-1-piperidyl)sulfonyl]-2- | *** |
| | methoxy-phenyl]-3,8-diazabicyclo[3.2.1]octan-3-yl]-(2- | |
| | chloro-4-fluoro-phenyl)methanone | |
| 009 | (2-chloro-4-fluoro-phenyl)-[(1S,5R)-8-[2-methoxy-5-[(4- | ** |
| | phenyl-1-piperidyl)sulfonyl]phenyl]-3,8- | |
| | diazabicyclo[3.2.1]octan-3-yl]methanone | |
| 010 | (2-chloro-4-fluoro-phenyl)-[(1S,5R)-8-[2-methoxy-5-[[4- | *** |
| | (2-phenylethyl)-1-piperidyl]sulfonyl]phenyl]-3,8- | |
| | diazabicyclo[3.2.1]octan-3-yl]methanone | |
| 011 | [(1S,5R)-8-[5-[[(3RS)-3-benzyl-1-piperidyl]sulfonyl]-2- | *** |
| | methoxy-phenyl]-3,8-diazabicyclo[3.2.1]octan-3-yl]-(2- | |
| | chloro-4-fluoro-phenyl)methanone | |
| 012 | (2-chloro-4-fluoro-phenyl)-[(1S,5R)-8-[2-methoxy-5-(4- | *** |
| | phenylpiperazin-1-yl)sulfonyl-phenyl]-3,8- | |
| | diazabicyclo[3.2.1]octan-3-yl]methanone | |
| 013 | (2-chloro-4-fluoro-phenyl)-[(1S,5R)-8-[2-methoxy-5-(4- | ** |
| | pyrimidin-2-ylpiperazin-1-yl)sulfonyl-phenyl]-3,8- | |
| | diazabicyclo[3.2.1]octan-3-yl]methanone | |
| 014 | [(1S,5R)-8-[5-(4-benzylpiperazin-1-yl)sulfonyl-2- | ** |
| | methoxy-phenyl]-3,8-diazabicyclo[3.2.1]octan-3-yl]-(2- | |
| | chloro-4-fluoro-phenyl)methanone; ethane | |
| 015 | [(1S,5R)-8-[5-[[(3aS,7aR)-1,3,3a,4,5,6,7,7a- | *** |
| | octahydroisoindol-2-yl]sulfonyl]-2-methoxy-phenyl]- | |
| | 3,8-diazabicyclo[3.2.1]octan-3-yl]-(2-chloro-4-fluoro- | |
| | phenyl)methanone | |
| 016 | 4-methoxy-N-methyl-N-propyl-3-[rac-(2S)-4-[4-fluoro- | ** |
| | 2-(trifluoromethyl)benzoyl]-2- | |
| | (trifluoromethyl)piperazin-1-yl]benzenesulfonamide | |
| 017 | 4-methoxy-N-methyl-N-propyl-3-[rac-(2S)-2-ethyl-4-[4- | * |
| | fluoro-2-(trifluoromethyl)benzoyl]piperazin-1- | |
| | yl]benzenesulfonamide | |
| 018 | [4-fluoro-2-(trifluoromethyl)phenyl]-[rac-(3S)-4-[5-[(4- | ** |
| | benzyl-1-piperidyl)sulfonyl]-2-methoxy-phenyl]-3- | |
| | methyl-piperazin-1-yl]methanone | |
| 019 | (2-chloro-4-fluoro-phenyl)-[rac-(3S)-4-[5-[(4-benzyl-1- | *** |
| | piperidyl)sulfonyl]-2-methoxy-phenyl]-3-methyl- | |
| | piperazin-1-yl]methanone | |
| 020 | N-tert-butyl-4-methoxy-3-[rac-(2S)-4-(2-chloro-4- | * |
| | fluoro-benzoyl)-2-methyl-piperazin-1- | |
| | yl]benzenesulfonamide | |
| 021 | 4-methoxy-N-methyl-N-propyl-3-[rac-(2S)-4-[4-fluoro- | ** |
| | 2-(trifluoromethyl)benzoyl]-2-methyl-piperazin-1- | |
| | yl]benzenesulfonamide | |
| 022 | 4-methoxy-N-methyl-N-propyl-3-[rac-(2R)-4-[4-fluoro- | * |
| | 2-(trifluoromethyl)benzoyl]-2- | |
| | (methoxymethyl)piperazin-1-yl]benzenesulfonamide | |
| 023 | 3-[(1S,5R)-7-[4-fluoro-2-(trifluoromethyl)benzoyl]-3- | * |
| | oxa-7,9-diazabicyclo[3.3.1]nonan-9-yl]-4-methoxy-N- | |
| | methyl-N-propyl-benzenesulfonamide | |
| 024 | 3-[3-[4-fluoro-2-(trifluoromethyl)benzoyl]-3,8- | ** |
| | diazabicyclo[3.2.1]octan-8-yl]-4-methoxy-N-methyl-N- | |
| | propyl-benzenesulfonamide | |
| 025 | (2-chloro-4-fluoro-phenyl)-[rac-(1S,5S)-8-[5-[(4-benzyl- | ** |
| | 1-piperidyl)sulfonyl]-2-methoxy-phenyl]-3,8- | |
| | diazabicyclo[3.2.1]octan-3-yl]methanone | |
| 026 | 3-[(1S,5R)-3-(2-chloro-4-fluoro-benzoyl)-3,8- | ** |
| | diazabicyclo[3.2.1]octan-8-yl]-4-fluoro-N-methyl-N- | |
| | propyl-benzenesulfonamide | |
| 027 | 3-[(1S,5R)-3-(2-chloro-4-fluoro-benzoyl)-3,8- | ** |
| | diazabicyclo[3.2.1]octan-8-yl]-N-methyl-N-propyl-4- | |
| | (trifluoromethoxy)benzenesulfonamide | |
| 028 | 3-[(1S,5R)-3-(2-chloro-4-fluoro-benzoyl)-3,8- | ** |
| | diazabicyclo[3.2.1]octan-8-yl]-N-methyl-N-propyl- | |
| | benzenesulfonamide | |
| 029 | 3-[(1S,5R)-3-(2-chloro-4-fluoro-benzoyl)-3,8- | ** |
| | diazabicyclo[3.2.1]octan-8-yl]-4-ethoxy-N-methyl-N- | |
| | propyl-benzenesulfonamide | |
| 030 | 3-[(1S,5R)-3-(2-chloro-4-fluoro-benzoyl)-3,8- | ** |
| | diazabicyclo[3.2.1]octan-8-yl]-4-(cyclobutoxy)-N- | |
| | methyl-N-propyl-benzenesulfonamide | |
| 031 | 3-[(1S,5R)-3-(2-chloro-4-fluoro-benzoyl)-3,8- | ** |
| | diazabicyclo[3.2.1]octan-8-yl]-4-(methoxymethoxy)-N- | |
| | methyl-N-propyl-benzenesulfonamide | |
| 032 | 6-chloro-5-[(1S,5R)-3-(2-chloro-4-fluoro-benzoyl)-3,8- | ** |
| | diazabicyclo[3.2.1]octan-8-yl]-N,N-diethyl-pyridine-3- | |
| | sulfonamide | |
| 033 | 5-[(1S,5R)-3-(2-chloro-4-fluoro-benzoyl)-3,8- | ** |
| | diazabicyclo[3.2.1]octan-8-yl]-N,N-diethyl-6-methoxy- | |
| | pyridine-3-sulfonamide | |
| 034 | 6-benzyloxy-5-[(1S,5R)-3-(2-chloro-4-fluoro-benzoyl)- | *** |
| | 3,8-diazabicyclo[3.2.1]octan-8-yl]-N,N-diethyl-pyridine- | |
| | 3-sulfonamide | |
| 035 | 5-[(1S,5R)-3-(2-chloro-4-fluoro-benzoyl)-3,8- | * |
| | diazabicyclo[3.2.1]octan-8-yl]-N,N-diethyl-6-hydroxy- | |
| | pyridine-3-sulfonamide | |
| 036 | 3-[(1S,5R)-3-(2-chloro-4-fluoro-benzoyl)-3,8- | ** |
| | diazabicyclo[3.2.1]octan-8-yl]-4-hydroxy-N-methyl-N- | |
| | propyl-benzenesulfonamide | |
| 037 | [(1S,5R)-8-[5-[(3SR)-3-benzyloxypyrrolidin-1- | ** |
| | yl]sulfonyl-2-methoxy-phenyl]-3,8- | |
| | diazabicyclo[3.2.1]octan-3-yl]-(2-chloro-4-fluoro- | |
| | phenyl)methanone | |
| 038 | N-(3-fluoropropyl)-4-methoxy-N-methyl-3-[rac-(2S)-4- | ** |
| | (2-chloro-4-fluoro-benzoyl)-2-methyl-piperazin-1- | |
| | yl]benzenesulfonamide | |
| 039 | (2-chloro-4-fluoro-phenyl)-[(1S,5R)-8-[5-[[4-(4- | *** |
| | fluorophenyl)-1-piperidyl]sulfonyl]-2-methoxy-phenyl]- | |
| | 3,8-diazabicyclo[3.2.1]octan-3-yl]methanone | |
| 040 | 3-[1-[3-[(1S,5R)-3-(2-chloro-4-fluoro-benzoyl)-3,8- | *** |
| | diazabicyclo[3.2.1]octan-8-yl]-4-methoxy- | |
| | phenyl]sulfonyl-4-piperidyl]benzonitrile | |
| 041 | methyl 3-[1-[3-[(1S,5R)-3-(2-chloro-4-fluoro-benzoyl)- | *** |
| | 3,8-diazabicyclo[3.2.1]octan-8-yl]-4-methoxy- | |
| | phenyl]sulfonyl-4-piperidyl]benzoate | |
| 042 | methyl 4-[1-[3-[(1S,5R)-3-(2-chloro-4-fluoro-benzoyl)- | *** |
| | 3,8-diazabicyclo[3.2.1]octan-8-yl]-4-methoxy- | |
| | phenyl]sulfonyl-4-piperidyl]benzoate | |
| 043 | (2-chloro-4-fluoro-phenyl)-[(1S,5R)-8-[2-methoxy-5-[[4- | *** |
| | (3-pyrazol-1-ylphenyl)-1-piperidyl]sulfonyl]phenyl]-3,8- | |
| | diazabicyclo[3.2.1]octan-3-yl]methanone | |
| 044 | (2-chloro-4-fluoro-phenyl)-[(1S,5R)-8-[5-[4- | *** |
| | [(1SR,2RS)-2-(4-fluorophenyl)cyclopropyl]piperazin-1- | |
| | yl]sulfonyl-2-methoxy-phenyl]-3,8- | |
| | diazabicyclo[3.2.1]octan-3-yl]methanone | |
| 045 | (2-chloro-4-fluoro-phenyl)-[(1S,5R)-8-[5-[4-[(2RS)-2- | *** |
| | (4-fluorophenyl)propyl]piperazin-1-yl]sulfonyl-2- | |
| | methoxy-phenyl]-3,8-diazabicyclo[3.2.1]octan-3- | |
| | yl]methanone | |
| 046 | (2-chloro-4-fluoro-phenyl)-[(1S,5R)-8-[5-[4-[2-(4- | *** |
| | fluorophenyl)ethyl]piperazin-1-yl]sulfonyl-2-methoxy- | |
| | phenyl]-3,8-diazabicyclo[3.2.1]octan-3-yl]methanone | |
| 047 | (2-chloro-4-fluoro-phenyl)-[(1S,5R)-8-[2-methoxy-5-[4- | *** |
| | [(2RS)-2-phenylpropyl]piperazin-1-yl]sulfonyl-phenyl]- | |
| | 3,8-diazabicyclo[3.2.1]octan-3-yl]methanone | |
| 048 | (2-chloro-4-fluoro-phenyl)-[(1S,5R)-8-[5-[4-[2-(4- | *** |
| | chlorophenyl)ethyl]piperazin-1-yl]sulfonyl-2-methoxy- | |
| | phenyl]-3,8-diazabicyclo[3.2.1]octan-3-yl]methanone | |
| 049 | (2-chloro-4,5-difluoro-phenyl)-[(1S,5R)-8-[2-methoxy- | *** |
| | 5-[(4-phenyl-1-piperidyl)sulfonyl]phenyl]-3,8- | |
| | diazabicyclo[3.2.1]octan-3-yl]methanone | |
| 050 | (2-chloro-4,5-difluoro-phenyl)-[(1S,5R)-8-[2-methoxy- | *** |
| | 5-[[4-(2-phenylethyl)-1-piperidyl]sulfonyl]phenyl]-3,8- | |
| | diazabicyclo[3.2.1]octan-3-yl]methanone | |
| 051 | (2-chloro-4,5-difluoro-phenyl)-[(1S,5R)-8-[2-methoxy- | *** |
| | 5-(4-phenylpiperazin-1-yl)sulfonyl-phenyl]-3,8- | |
| | diazabicyclo[3.2.1]octan-3-yl]methanone | |
| 052 | [(1S,5R)-8-[5-[(3-benzyl-1-piperidyl)sulfonyl]-2- | *** |
| | methoxy-phenyl]-3,8-diazabicyclo[3.2.1]octan-3-yl]-(2- | |
| | chloro-4,5-difluoro-phenyl)methanone | |
| 053 | [(1S,5R)-8-[5-[(4-benzylidene-1-piperidyl)sulfonyl]-2- | *** |
| | methoxy-phenyl]-3,8-diazabicyclo[3.2.1]octan-3-yl]-(2- | |
| | chloro-4,5-difluoro-phenyl)methanone | |
| 054 | N-tert-butyl-3-[(1S,5R)-3-(2-chloro-4,5-difluoro- | ** |
| | benzoyl)-3,8-diazabicyclo[3.2.1]octan-8-yl]-4-methoxy- | |
| | N-methyl-benzenesulfonamide | |
| 055 | (2-chloro-4-fluoro-phenyl)-[(1S,5R)-8-[5-[(4-phenyl-1- | *** |
| | piperidyl)sulfonyl]-2,3-dihydrobenzofuran-7-yl]-3,8- | |
| | diazabicyclo[3.2.1]octan-3-yl]methanone | |
| 056 | (2-chloro-4-fluoro-phenyl)-[(1S,5R)-8-[5-[[4-(4- | *** |
| | fluorophenyl)-1-piperidyl]sulfonyl]-2,3- | |
| | dihydrobenzofuran-7-yl]-3,8-diazabicyclo[3.2.1]octan-3- | |
| | yl]methanone | |
| 057 | N-tert-butyl-7-[(1S,5R)-3-(2-chloro-4-fluoro-benzoyl)- | *** |
| | 3,8-diazabicyclo[3.2.1]octan-8-yl]-2,3- | |
| | dihydrobenzofuran-5-sulfonamide | |
| 058 | 7-[(1S,5R)-3-(2-chloro-4-fluoro-benzoyl)-3,8- | ** |
| | diazabicyclo[3.2.1]octan-8-yl]-N-(cyclopropylmethyl)- | |
| | N-methyl-2,3-dihydrobenzofuran-5-sulfonamide | |
| 059 | (2-chloro-4-fluoro-phenyl)-[(1S,5R)-8-[5-[4-[2-(4- | *** |
| | fluorophenyl)ethyl]piperazin-1-yl]sulfonyl-2,3- | |
| | dihydrobenzofuran-7-yl]-3,8-diazabicyclo[3.2.1]octan-3- | |
| | yl]methanone | |
| 060 | (2-chloro-4-fluoro-phenyl)-[(1S,5R)-8-[5-[4-[2-(4- | *** |
| | chlorophenyl)ethyl]piperazin-1-yl]sulfonyl-2,3- | |
| | dihydrobenzofuran-7-yl]-3,8-diazabicyclo[3.2.1]octan-3- | |
| | yl]methanone | |
| 061 | (2-chloro-4,5-difluoro-phenyl)-[(1S,5R)-8-[5-[(4-phenyl- | *** |
| | 1-piperidyl)sulfonyl]-2,3-dihydrobenzofuran-7-yl]-3,8- | |
| | diazabicyclo[3.2.1]octan-3-yl]methanone | |
| 062 | (2-chloro-4-fluoro-phenyl)-[(1S,5R)-8-[2-hydroxy-5-[[4- | *** |
| | (2-phenylethyl)-1-piperidyl]sulfonyl]phenyl]-3,8- | |
| | diazabicyclo[3.2.1]octan-3-yl]methanone | |
| 063 | (2-chloro-4-fluoro-phenyl)-[(1S,5R)-8-[2-hydroxy-5-[(4- | *** |
| | phenyl-1-piperidyl)sulfonyl]phenyl]-3,8- | |
| | diazabicyclo[3.2.1]octan-3-yl]methanone | |
| 064 | (2-chloro-4-fluoro-phenyl)-[(1S,5R)-8-[5-[4-[2-(4- | *** |
| | chlorophenyl)ethyl]piperazin-1-yl]sulfonyl-2-hydroxy- | |
| | phenyl]-3,8-diazabicyclo[3.2.1]octan-3-yl]methanone | |
| 065 | (2-chloro-4-fluoro-phenyl)-[(1S,5R)-8-[5-[[4-(4- | *** |
| | fluorophenyl)-1-piperidyl]sulfonyl]-2-hydroxy-phenyl]- | |
| | 3,8-diazabicyclo[3.2.1]octan-3-yl]methanone | |
| 066 | (2-chloro-4-fluoro-phenyl)-[(1S,5R)-8-[2-chloro-5-[(4- | *** |
| | phenyl-1-piperidyl)sulfonyl]phenyl]-3,8- | |
| | diazabicyclo[3.2.1]octan-3-yl]methanone | |
| 067 | (2-chloro-4-fluoro-phenyl)-[(1S,5R)-8-[2-methyl-5-[(4- | *** |
| | phenyl-1-piperidyl)sulfonyl]phenyl]-3,8- | |
| | diazabicyclo[3.2.1]octan-3-yl]methanone | |
| 068 | 3-[(1S,5R)-3-(2-chloro-4-fluoro-benzoyl)-3,8- | *** |
| | diazabicyclo[3.2.1]octan-8-yl]-N,4-dimethyl-N-propyl- | |
| | benzenesulfonamide | |
| 069 | methyl 2-[(1S,5R)-3-(2-chloro-4-fluoro-benzoyl)-3,8- | *** |
| | diazabicyclo[3.2.1]octan-8-yl]-4-[(4-phenyl-1- | |
| | piperidyl)sulfonyl]benzoate | |
| 070 | (2-chloro-4-fluoro-phenyl)-[(1S,5R)-8-[2- | *** |
| | (hydroxymethyl)-5-[(4-phenyl-1- | |
| | piperidyl)sulfonyl]phenyl]-3,8-diazabicyclo[3.2.1]octan- | |
| | 3-yl]methanone | |
| 071 | 2-[(1S,5R)-3-(2-chloro-4-fluoro-benzoyl)-3,8- | *** |
| | diazabicyclo[3.2.1]octan-8-yl]-4-[(4-phenyl-1- | |
| | piperidyl)sulfonyl]benzoic acid | |
| 072 | (2-chloro-4-fluoro-phenyl)-[(1S,5R)-8-[2-(1-hydroxy-1- | ** |
| | methyl-ethyl)-5-[(4-phenyl-1-piperidyl)sulfonyl]phenyl]- | |
| | 3,8-diazabicyclo[3.2.1]octan-3-yl]methanone | |
| 073 | (2-chloro-4-fluoro-phenyl)-[(1S,5R)-8-[2- | ** |
| | [(dimethylamino)methyl]-5-[(4-phenyl-1- | |
| | piperidyl)sulfonyl]phenyl]-3,8-diazabicyclo[3.2.1]octan- | |
| | 3-yl]methanone | |
| 074 | N-tert-butyl-3-chloro-5-[(1S,5R)-3-(2-chloro-4-fluoro- | *** |
| | benzoyl)-3,8-diazabicyclo[3.2.1]octan-8-yl]-4-methyl- | |
| | benzenesulfonamide | |
| 075 | N-tert-butyl-3,4-dichloro-5-[(1S,5R)-3-(2-chloro-4- | *** |
| | fluoro-benzoyl)-3,8-diazabicyclo[3.2.1]octan-8- | |
| | yl]benzenesulfonamide | |
| 076 | (2-chloro-4-fluoro-phenyl)-[(1S,5R)-8-[2-(2- | *** |
| | hydroxyethoxy)-5-[(4-phenyl-1- | |
| | piperidyl)sulfonyl]phenyl]-3,8-diazabicyclo[3.2.1]octan- | |
| | 3-yl]methanone | |
| 077 | (2-chloro-4-fluoro-phenyl)-[(1S,5R)-8-[2-(2- | *** |
| | hydroxyethoxy)-5-[[4-(2-phenylethyl)-1- | |
| | piperidyl]sulfonyl]phenyl]-3,8-diazabicyclo[3.2.1]octan- | |
| | 3-yl]methanone | |
| 078 | (2-chloro-4-fluoro-phenyl)-[(1S,5R)-8-[5-[4-[2-(4- | *** |
| | chlorophenyl)ethyl]piperazin-1-yl]sulfonyl-2-(2- | |
| | hydroxyethoxy)phenyl]-3,8-diazabicyclo[3.2.1]octan-3- | |
| | yl]methanone | |
| 079 | 3-[(1S,5R)-3-(2-chloro-4-fluoro-benzoyl)-3,8- | *** |
| | diazabicyclo[3.2.1]octan-8-yl]-4-cyclopropyl-N-methyl- | |
| | N-propyl-benzenesulfonamide | |
| 080 | (2-chloro-4-fluoro-phenyl)-[(1S,5R)-8-[2-cyclopropyl-5- | *** |
| | [(4-phenyl-1-piperidyl)sulfonyl]phenyl]-3,8- | |
| | diazabicyclo[3.2.1]octan-3-yl]methanone | |
| 081 | (2-chloro-4-fluoro-phenyl)-[(1S,5R)-8-[2-(1H-imidazol- | *** |
| | 4-yl)-5-[(4-phenyl-1-piperidyl)sulfonyl]phenyl]-3,8- | |
| | diazabicyclo[3.2.1]octan-3-yl]methanone | |
| 082 | (2-chloro-4-fluoro-phenyl)-[(1S,5R)-8-[2-(1- | ** |
| | methylimidazol-4-yl)-5-[(4-phenyl-1- | |
| | piperidyl)sulfonyl]phenyl]-3,8-diazabicyclo[3.2.1]octan- | |
| | 3-yl]methanone | |
| 083 | (2-chloro-4-fluoro-phenyl)-[(1S,5R)-8-[3-fluoro-2- | *** |
| | hydroxy-5-[(4-phenyl-1-piperidyl)sulfonyl]phenyl]-3,8- | |
| | diazabicyclo[3.2.1]octan-3-yl]methanone | |
| 084 | (2-chloro-4-fluoro-phenyl)-[(1S,5R)-8-[3-fluoro-5-[[4- | *** |
| | (4-fluorophenyl)-1-piperidyl]sulfonyl]-2-hydroxy- | |
| | phenyl]-3,8-diazabicyclo[3.2.1]octan-3-yl]methanone | |
| 085 | N-tert-butyl-3-[(1S,5R)-3-(2-chloro-4-fluoro-benzoyl)- | *** |
| | 3,8-diazabicyclo[3.2.1]octan-8-yl]-5-fluoro-4-hydroxy- | |
| | benzenesulfonamide | |
| 086 | 3-[(1S,5R)-3-(2-chloro-4-fluoro-benzoyl)-3,8- | *** |
| | diazabicyclo[3.2.1]octan-8-yl]-N-(cyclopropylmethyl)-5- | |
| | fluoro-4-hydroxy-N-methyl-benzenesulfonamide | |
| 087 | (2-chloro-4-fluoro-phenyl)-[(1S,5R)-8-[2-hydroxy-5-[(4- | *** |
| | phenyl-1-piperidyl)sulfonyl]-3-(trifluoromethyl)phenyl]- | |
| | 3,8-diazabicyclo[3.2.1]octan-3-yl]methanone | |
| 088 | (2-chloro-4-fluoro-phenyl)-[(1S,5R)-8-[5-[[4-(4- | *** |
| | fluorophenyl)-1-piperidyl]sulfonyl]-2-hydroxy-3- | |
| | (trifluoromethyl)phenyl]-3,8-diazabicyclo[3.2.1]octan-3- | |
| | yl]methanone | |
| 089 | N-tert-butyl-3-[(1S,5R)-3-(2-chloro-4-fluoro-benzoyl)- | ** |
| | 3,8-diazabicyclo[3.2.1]octan-8-yl]-4-hydroxy-5- | |
| | (trifluoromethyl)benzenesulfonamide | |
| 090 | (2-chloro-4-fluoro-phenyl)-[(1S,5R)-8-[2-hydroxy-3- | *** |
| | methyl-5-[(4-phenyl-1-piperidyl)sulfonyl]phenyl]-3,8- | |
| | diazabicyclo[3.2.1]octan-3-yl]methanone | |
| 091 | (2-chloro-4-fluoro-phenyl)-[(1S,5R)-8-[5-[[4-(4- | *** |
| | fluorophenyl)-1-piperidyl]sulfonyl]-2-hydroxy-3-methyl- | |
| | phenyl]-3,8-diazabicyclo[3.2.1]octan-3-yl]methanone | |
| 092 | 3-[(1S,5R)-3-(2-chloro-4-fluoro-benzoyl)-3,8- | *** |
| | diazabicyclo[3.2.1]octan-8-yl]-N-(cyclopropylmethyl)-4- | |
| | hydroxy-N,5-dimethyl-benzenesulfonamide | |
| 093 | N-tert-butyl-3-[(1S,5R)-3-(2-chloro-4-fluoro-benzoyl)- | *** |
| | 3,8-diazabicyclo[3.2.1]octan-8-yl]-4-hydroxy-5-methyl- | |
| | benzenesulfonamide | |
| 094 | (2-chloro-4-fluoro-phenyl)-[(1S,5R)-8-[3-ethyl-2- | *** |
| | hydroxy-5-[(4-phenyl-1-piperidyl)sulfonyl]phenyl]-3,8- | |
| | diazabicyclo[3.2.1]octan-3-yl]methanone | |
| 095 | N-tert-butyl-3-chloro-5-[(1S,5R)-3-(2-chloro-4-fluoro- | *** |
| | benzoyl)-3,8-diazabicyclo[3.2.1]octan-8-yl]-4-hydroxy- | |
| | benzenesulfonamide | |
| 096 | 3-chloro-5-[(1S,5R)-3-(2-chloro-4-fluoro-benzoyl)-3,8- | *** |
| | diazabicyclo[3.2.1]octan-8-yl]-N-(cyclopropylmethyl)-4- | |
| | hydroxy-N-methyl-benzenesulfonamide | |
| 097 | (2-chloro-4-fluoro-phenyl)-[(1S,5R)-8-[3-chloro-5-[(4- | *** |
| | fluoro-1-piperidyl)sulfonyl]-2-hydroxy-phenyl]-3,8- | |
| | diazabicyclo[3.2.1]octan-3-yl]methanone | |
| 098 | (2-chloro-4-fluoro-phenyl)-[(1S,5R)-8-[3-chloro-2- | *** |
| | hydroxy-5-[(4-phenyl-1-piperidyl)sulfonyl]phenyl]-3,8- | |
| | diazabicyclo[3.2.1]octan-3-yl]methanone | |
| 099 | (2-chloro-4-fluoro-phenyl)-[(1S,5R)-8-[3-chloro-5-[[4- | *** |
| | (4-fluorophenyl)-1-piperidyl]sulfonyl]-2-hydroxy- | |
| | phenyl]-3,8-diazabicyclo[3.2.1]octan-3-yl]methanone | |
| 100 | 3-chloro-5-[(1S,5R)-3-(2-chloro-4-fluoro-benzoyl)-3,8- | *** |
| | diazabicyclo[3.2.1]octan-8-yl]-4-hydroxy-N-isopropyl- | |
| | benzenesulfonamide | |
| 101 | 3-chloro-5-[(1S,5R)-3-(2-chloro-4-fluoro-benzoyl)-3,8- | ** |
| | diazabicyclo[3.2.1]octan-8-yl]-4-hydroxy-N-(1- | |
| | methylcyclopropyl)benzenesulfonamide | |
| 102 | 3-chloro-5-[(1S,5R)-3-(2-chloro-4-fluoro-benzoyl)-3,8- | *** |
| | diazabicyclo[3.2.1]octan-8-yl]-4-hydroxy-N-(1- | |
| | methylcyclobutyl)benzenesulfonamide | |
| 103 | 3-chloro-5-[(1S,5R)-3-(2-chloro-4-fluoro-benzoyl)-3,8- | ** |
| | diazabicyclo[3.2.1]octan-8-yl]-N-(1-cyano-1-methyl- | |
| | ethyl)-4-hydroxy-benzenesulfonamide | |
| 104 | 3-chloro-5-[(1S,5R)-3-(2-chloro-4-fluoro-benzoyl)-3,8- | ** |
| | diazabicyclo[3.2.1]octan-8-yl]-4-hydroxy-N-[(1SR)- | |
| | 2,2,2-trifluoro-1-methyl-ethyl]benzenesulfonamide | |
| 105 | 3-chloro-5-[(1S,5R)-3-(2-chloro-4-fluoro-benzoyl)-3,8- | *** |
| | diazabicyclo[3.2.1]octan-8-yl]-4-hydroxy-N-(2,2,2- | |
| | trifluoro-1,1-dimethyl-ethyl)benzenesulfonamide | |
| 106 | 3-chloro-5-[(1S,5R)-3-(2-chloro-4-fluoro-benzoyl)-3,8- | *** |
| | diazabicyclo[3.2.1]octan-8-yl]-4-hydroxy-N-methyl-N- | |
| | (3-methyloxetan-3-yl)benzenesulfonamide | |
| 107 | N-tert-butyl-3-chloro-5-[(1S,5R)-3-(2-chloro-4-fluoro- | *** |
| | benzoyl)-3,8-diazabicyclo[3.2.1]octan-8-yl]-4-hydroxy- | |
| | N-methyl-benzenesulfonamide | |
| 108 | 3-chloro-5-[(1S,5R)-3-(2-chloro-4-fluoro-benzoyl)-3,8- | ** |
| | diazabicyclo[3.2.1]octan-8-yl]-4-hydroxy-N-(3- | |
| | methyloxetan-3-yl)benzenesulfonamide | |
| 109 | 3-chloro-5-[(1S,5R)-3-(2-chloro-4-fluoro-benzoyl)-3,8- | ** |
| | diazabicyclo[3.2.1]octan-8-yl]-4-hydroxy-N-methyl-N- | |
| | (oxetan-3-yl)benzenesulfonamide | |
| 110 | N-tert-butyl-3-chloro-5-[(2S)-4-(2-chloro-4-fluoro- | *** |
| | benzoyl)-2-methyl-piperazin-1-yl]-4-hydroxy- | |
| | benzenesulfonamide | |
| 111 | N-tert-butyl-3-chloro-5-[(2R)-4-(2-chloro-4-fluoro- | *** |
| | benzoyl)-2-methyl-piperazin-1-yl]-4-hydroxy- | |
| | benzenesulfonamide | |
| 112 | N-tert-butyl-3-chloro-5-[(1S,5R)-3-(2-chloro-4-fluoro- | *** |
| | benzoyl)-3,8-diazabicyclo[3.2.1]octan-8-yl]-4-(2- | |
| | hydroxyethoxy)benzenesulfonamide | |
| 113 | N-tert-butyl-3-chloro-5-[(1S,5R)-3-(2-chloro-4-fluoro- | *** |
| | benzoyl)-3,8-diazabicyclo[3.2.1]octan-8-yl]-4-(2- | |
| | methoxyethoxy)benzenesulfonamide | |
| 114 | N-tert-butyl-3-chloro-5-[(1S,5R)-3-(2-chloro-4-fluoro- | *** |
| | benzoyl)-3,8-diazabicyclo[3.2.1]octan-8-yl]-4-methoxy- | |
| | benzenesulfonamide | |
| 115 | 4-chloro-3-[(1S,5R)-3-(2-chloro-4-fluoro-benzoyl)-3,8- | *** |
| | diazabicyclo[3.2.1]octan-8-yl]-N-methyl-N-propyl- | |
| | benzenesulfonamide | |
| 116 | (2-chloro-4-fluoro-phenyl)-[(1S,5R)-8-[5-[4-[2-(4- | *** |
| | fluorophenyl)ethyl]piperazin-1-yl]sulfonyl-2-methyl- | |
| | phenyl]-3,8-diazabicyclo[3.2.1]octan-3-yl]methanone | |
| 117 | (2-chloro-4-fluoro-phenyl)-[(1S,5R)-8-[6-[(4-phenyl-1- | *** |
| | piperidyl)sulfonyl]-1,3-benzodioxol-4-yl]-3,8- | |
| | diazabicyclo[3.2.1]octan-3-yl]methanone | |
| 118 | (2-chloro-4-fluoro-phenyl)-[(1S,5R)-8-[7-[(4-phenyl-1- | *** |
| | piperidyl)sulfonyl]-2,3-dihydro-1,4-benzodioxin-5-yl]- | |
| | 3,8-diazabicyclo[3.2.1]octan-3-yl]methanone | |
| 119 | (2-chloro-4-fluoro-phenyl)-[(1S,5R)-8-[1-methyl-5-[(4- | *** |
| | phenyl-1-piperidyl)sulfonyl]indolin-7-yl]-3,8- | |
| | diazabicyclo[3.2.1]octan-3-yl]methanone | |
| 120 | (2-chloro-4,5-difluoro-phenyl)-[(1S,5R)-8-[1-methyl-5- | *** |
| | [(4-phenyl-1-piperidyl)sulfonyl]indolin-7-yl]-3,8- | |
| | diazabicyclo[3.2.1]octan-3-yl]methanone | |
| 121 | (2-chloro-4-fluoro-phenyl)-[(1S,5R)-8-[2- | *** |
| | (dimethylamino)-5-[(4-phenyl-1- | |
| | piperidyl)sulfonyl]phenyl]-3,8-diazabicyclo[3.2.1]octan- | |
| | 3-yl]methanone | |
| 122 | (2-chloro-4,5-difluoro-phenyl)-[(1S,5R)-8-[2- | *** |
| | (dimethylamino)-5-[(4-phenyl-1- | |
| | piperidyl)sulfonyl]phenyl]-3,8-diazabicyclo[3.2.1]octan- | |
| | 3-yl]methanone | |
| 123 | (2-chloro-4-fluoro-phenyl)-[(1S,5R)-8-[2-[3- | *** |
| | (methylamino)azetidin-1-yl]-5-[(4-phenyl-1- | |
| | piperidyl)sulfonyl]phenyl]-3,8-diazabicyclo[3.2.1]octan- | |
| | 3-yl]methanone | |
| 124 | (2-chloro-4-fluoro-phenyl)-[(1S,5R)-8-[3-hydroxy-2- | *** |
| | methyl-5-[(4-phenyl-1-piperidyl)sulfonyl]phenyl]-3,8- | |
| | diazabicyclo[3.2.1]octan-3-yl]methanone | |
| 125 | 2-[(1R,5S)-3-(2-chloro-4-fluoro-benzoyl)-3,8- | *** |
| | diazabicyclo[3.2.1]octan-8-yl]-4-(4- | |
| | phenylcyclohexyl)sulfonyl-benzonitrile | |
| 126 | (2-chloro-4-fluoro-phenyl)-[(1S,5R)-8-[2-methoxy-5- | *** |
| | [(3SR)-3-phenoxypyrrolidin-1-yl]sulfonyl-phenyl]-3,8- | |
| | diazabicyclo[3.2.1]octan-3-yl]methanone | |
| 127 | (2-chloro-4-fluoro-phenyl)-[(1S,5R)-8-[2-methoxy-5- | *** |
| | [(3SR)-3-(N-methylanilino)pyrrolidin-1-yl]sulfonyl- | |
| | phenyl]-3,8-diazabicyclo[3.2.1]octan-3-yl]methanone | |
| 128 | (2-chloro-4-fluoro-phenyl)-[(1S,5R)-8-[5-[4-[(2RS)-2- | *** |
| | (4-fluorophenyl)-2-hydroxy-ethyl]piperazin-1- | |
| | yl]sulfonyl-2-methoxy-phenyl]-3,8- | |
| | diazabicyclo[3.2.1]octan-3-yl]methanone | |
| 129 | N-tert-butyl-3-[(1S,5R)-3-(2-chloro-4,5-difluoro- | *** |
| | benzoyl)-3,8-diazabicyclo[3.2.1]octan-8-yl]-4-methoxy- | |
| | benzenesulfonamide | |
| 130 | (2-chloro-4-fluoro-phenyl)-[(1S,5R)-8-[5-[(4-fluoro-1- | *** |
| | piperidyl)sulfonyl]-2,3-dihydrobenzofuran-7-yl]-3,8- | |
| | diazabicyclo[3.2.1]octan-3-yl]methanone | |
| 131 | [(1S,5R)-8-[3-chloro-5-(3,3-dimethylmorpholin-4- | *** |
| | yl)sulfonyl-2-hydroxy-phenyl]-3,8- | |
| | diazabicyclo[3.2.1]octan-3-yl]-(2-chloro-4-fluoro- | |
| | phenyl)methanone | |
| 132 | N-tert-butyl-3-chloro-5-[(1S,5R)-3-(2-chloro-4-fluoro- | *** |
| | benzoyl)-3,8-diazabicyclo[3.2.1]octan-8-yl]-N,4- | |
| | dimethyl-benzenesulfonamide | |
| 133 | N-tert-butyl-3,4-dichloro-5-[(1S,5R)-3-(2-chloro-4- | *** |
| | fluoro-benzoyl)-3,8-diazabicyclo[3.2.1]octan-8-yl]-N- | |
| | methyl-benzenesulfonamide | |
| 134 | N-tert-butyl-3-chloro-5-[(1S,5R)-3-(2-chloro-4-fluoro- | *** |
| | benzoyl)-3,8-diazabicyclo[3.2.1]octan-8-yl]-4-(2- | |
| | hydroxyethoxy)-N-methyl-benzenesulfonamide | |
| 135 | N-tert-butyl-3-chloro-5-[(1S,5R)-3-(2-chloro-4-fluoro- | *** |
| | benzoyl)-3,8-diazabicyclo[3.2.1]octan-8-yl]-4-(2- | |
| | methoxyethoxy)-N-methyl-benzenesulfonamide | |
| 136 | 4-benzyloxy-N-tert-butyl-3-chloro-5-[(1S,5R)-3-(2- | *** |
| | chloro-4-fluoro-benzoyl)-3,8-diazabicyclo[3.2.1]octan-8- | |
| | yl]-N-methyl-benzenesulfonamide | |
| 137 | N-tert-butyl-3-chloro-5-[(1S,5R)-3-(2-chloro-4-fluoro- | *** |
| | benzoyl)-3,8-diazabicyclo[3.2.1]octan-8-yl]-N-methyl-4- | |
| | (3-pyridylmethoxy)benzenesulfonamide | |
| 138 | N-tert-butyl-3-chloro-5-[(1S,5R)-3-(2-chloro-4-fluoro- | *** |
| | benzoyl)-3,8-diazabicyclo[3.2.1]octan-8-yl]-N-methyl-4- | |
| | (4-pyridylmethoxy)benzenesulfonamide | |
| 139 | N-tert-butyl-5-chloro-3-[(1S,5R)-3-(2-chloro-4-fluoro- | *** |
| | benzoyl)-3,8-diazabicyclo[3.2.1]octan-8-yl]-2-fluoro-4- | |
| | hydroxy-benzenesulfonamide | |

The above results clearly evidence that the tested compounds 001-139 have significant GFRα1-RET activity. Thus, the compounds of the invention are useful as neuroprotective and neurorestorative agents. The tested compounds 001-139 are well-representing the class of the compounds of formula (I).

1-15. (canceled)

16. A compound of formula (I) 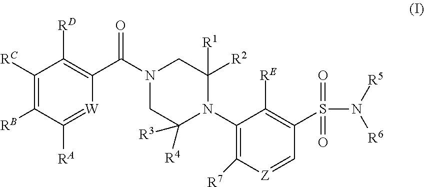
or a pharmaceutically acceptable salt and/or solvate thereof;
wherein:
W represents CH or N;
R^{A}, R^{B}, R^{C} and R^{D} each independently represents hydrogen, F, C, CH₃, CF₃, CHF₂, or CH₂F,
provided that at least one among R^{A}, R^{B}, R^{C} and R^{D} does not represent hydrogen;
R¹ represents (C₁-C₈) alkyl, wherein the alkyl is optionally substituted by at least one OH, (C₁-C₃) alkoxy, or F; and R², R³ and R⁴ represents hydrogen;
or R¹ and R⁴ form together —CH₂—O-CH₂— or —CH₂—CH₂—, wherein the —CH₂—CH₂— is optionally substituted by at least one F, OH, or OCH₃; and R² and R³ each represents hydrogen;
R⁷ represents hydrogen, OH, halogen, (C₁-C₈) alkyl, cycloalkyl, (C₁-C₈) alkyl-O—, cycloalkyl-O—, cycloalkyl-(C₁-C₈) alkyl-O—, heterocycloalkyl-O—, R¹¹O—(C₁-C₈) alkyl-O—, R¹¹R¹²N—(C₁-C₈) alkyl-O—, (R¹¹⁰) (R¹²)N—(C₁-C₈) alkyl-O—, R¹¹R 12N—(C₁-C₈) alkyl-, R¹¹O—(C₁-C₈) alkyl-, NR¹¹R¹², CN, CO₂H, CO₂R¹¹, CONH₂, CON(R¹¹) H, heterocycloalkyl, or heteroaryl; wherein R¹¹ and R¹² each independently represents hydrogen or (C₁-C₈) alkyl;
wherein the alkyl or cycloalkyl in R⁷ is optionally substituted by at least one F, Cl, OH, ═O, (C₁-C₈) alkyl, (C₁-C₈) alkyl-O—, heterocycloalkyl, aryl, or heteroaryl;
wherein the heterocycloalkyl, aryl or heteroaryl is optionally substituted by at least one F, Cl, (C₁-C₈) alkyl, CF₃, CHF₂, CH₂F, OCF₃, CN, OH, ═O,-0, (C₁-C₈) alkoxy, NR¹³R¹⁴, R¹³R¹⁴N—(C₁-C₈) alkyl-, R¹³O₂C (C₁-C₈) alkyl-, CO₂H, R¹³R¹⁴N—C(O)—, R¹³⁰—NR¹⁴, or (C₁-C₈) alkyl-CO₂—; wherein R¹³ and R¹⁴ each independently represents hydrogen or (C₁-C₈) alkyl;
Z represents C—H, C—R⁸ or N;
wherein R⁸ represents (C₁-C₄) alkyl, F, Cl, CF₃, CHF₂, CH₂F, OCF₃, CN, OH, or (C₁-C₄) alkoxy;
or Z represents C-R⁸ and R⁷ and R⁸ form together with the carbon atoms to which they are bound a cycloalkyl or heterocycloalkyl,
wherein the cycloalkyl or heterocycloalkyl is optionally substituted by at least one F, OH, ═O, →O, (C₁-C₈) alkyl, CF₃, HO₂C—CH₂—, (C₁-C₄) alkyl-CO₂—CH₂—, R¹⁵R¹⁶N—CH₂—, aryl, or aryl-(C₁-C₈) alkyl-, wherein R¹⁵ and R¹⁶ each independently represents hydrogen or (C₁-C₈) alkyl;
R⁵ represents hydrogen, (C₁-C₈) alkyl, CH₃ substituted by one to three (C₁-C₈) alkyl groups, cycloalkyl, heterocycloalkyl, aryl, or cycloalkyl-(C₁-C₈) alkyl;
wherein the alkyl or cycloalkyl in R⁵ is optionally substituted by at least one F, Cl, (C₁-C₈) alkyl, CF₃, OCF₃, CN, OH, ═O, (C₁-C₈) alkoxy, NR¹⁷R¹⁸, CO₂H, R¹⁷R¹⁸N—C(O)—, R¹⁷O—NR¹⁸—, heterocycloalkyl, aryl, or heteroaryl; wherein R¹⁷ and R¹⁸ each independently represents hydrogen or (C₁-C₈) alkyl;
wherein the heterocycloalkyl, aryl or heteroaryl is optionally substituted by at least one F, Cl, (C₁-C₈) alkyl, CF₃, CHF₂, CH₂F, OCF₃, CN, OH, ═O, →O, (C₁-C₈) alkoxy, NR¹⁹R²⁰, CO₂H, R¹⁹R²⁰N—C(O)—, R¹⁹—NR²⁰—, (C₁-C₈) alkyl-CO₂—, R¹⁹R²⁰N—(C₁-C₈) alkyl-, R¹⁹O²C—(C₁-C₈) alkyl-, heterocycloalkyl, heteroaryl, aryl, or aryl-(C₁-C₈) alkyl-; wherein R¹⁹ and R²⁰ each independently represents hydrogen or (C₁-C₈) alkyl;
R⁶ represents (C₁-C₈) alkyl, CH₃ substituted by one to three (C₁-C₈) alkyl groups, cycloalkyl, heterocycloalkyl, aryl, or cycloalkyl-(C₁-C₈) alkyl;
wherein the alkyl or cycloalkyl in R⁶ is optionally substituted by at least one F, Cl, (C₁-C₈) alkyl, CF₃, OCF₃, CN, OH, ═O, (C₁-C₈) alkoxy, NR²¹R²², CO₂H, R²¹R²²N—C(O)—, R²¹O—NR²², heterocycloalkyl, aryl, or heteroaryl; wherein R²¹ and R²² each independently represents hydrogen or (C₁-C₈) alkyl;
wherein the heterocycloalkyl, aryl or heteroaryl is optionally substituted by at least one F, Cl, (C₁-C₈) alkyl, CF₃, CHF₂, CH₂F, OCF₃, CN, OH, ═O, →O, (C₁-C₈) alkoxy, NR²³R²⁴, CO₂H, R²³R²⁴N—C(O)—, R²³O—NR²⁴—, (C₁-C₈) alkyl-CO₂—, R²³R²⁴N—(C₁-C₈) alkyl-, R²³O₂C—(C₁-C₅) alkyl-, heterocycloalkyl, heteroaryl, aryl, or aryl-(C₁-C₅) alkyl-; wherein R²³ and R²⁴ each independently represents hydrogen or (C₁-C₅) alkyl;
or R⁵ and R⁶ form together with the nitrogen atom to which they are bound an heterocycloalkyl,
wherein the heterocycloalkyl is optionally substituted by at least one F, Cl, (C₁-C₅) alkyl, CF₃, OCF₃, CN, OH, ═O, →O, (C₁-C₅) alkoxy, NR²⁵R²⁶, CO₂H, (C₁-C₅) alkyl-CO₂—, R²⁵R²⁶N—C(O)—, R²⁵O—NR²⁶—, R²⁵R²⁶N—(C₁-C₈) alkyl-, R²⁵O₂C—(C₁-C₅) alkyl-, cycloalkyl, heterocycloalkyl, aryl, heteroaryl, cycloalkyl-(C₁-C₅) alkyl-, heterocycloalkyl-(C₁-C₅) alkyl-, aryl-(C₁-C₅) alkyl-, heteroaryl-(C₁-C₅) alkyl-, aryl-cycloalkyl-, cycloalkyl-O—, heterocycloalkyl-O—, aryl-O—, heteroaryl-O—, cycloalkyl-(C₁-C₅) alkyl-O—, heterocycloalkyl-(C₁-C₅) alkyl-O—, aryl-(C₁-C₅) alkyl-O—, heteroaryl-(C₁-C₅) alkyl-O—, cycloalkyl-NR²⁵, heterocycloalkyl-NR²⁵-, aryl-NR²⁵_, heteroaryl-NR²⁵-, cycloalkyl-(C₁-C₅) alkyl-NR²⁵-, heterocycloalkyl-(C₁-C₅) alkyl-NR²⁵-, aryl-(C₁-C₈) alkyl-NR²⁵, heteroaryl-(C₁-C₅) alkyl-NR²⁵, benzylidene, heteroarylidene, aryl-(C₁-C₅) alkyl-ylidene-, or heteroaryl-(C₁-C₅) alkyl-ylidene-;
wherein R²⁵ and R²⁶ each independently represents hydrogen or (C₁-C₅) alkyl; and
wherein the heterocycloalkyl, aryl, heteroaryl, benzylidene or heteroarylidene is optionally substituted at least one F, Cl, (C₁-C₅) alkyl, CF₃, CHF₂, CH₂F, OCF₃, CN, OH, ═O,→O, (C₁-C₅) alkoxy, NR²⁷R²⁸, CO₂H, R²⁷R²⁸N—C(O)—, R²⁷O —NR²⁸, (C₁-C₅) alkyl-CO₂—, R²⁷R²⁸N—(C₁-C₅) alkyl-, R²⁷⁰²C—(C₁-C₅) alkyl-, heterocycloalkyl, heteroaryl, aryl, or aryl-(C₁-C₅) alkyl-; wherein R²⁷ and R²⁸ each independently represents hydrogen or (C₁-C₅) alkyl; and
R^{E} represents hydrogen, (C₁-C₃) alkyl or halogen.

17. The compound according to claim 16, wherein at least one among R^{A}, R^{B}, R^{C} and R^{D} represents hydrogen; and/or wherein R^{E} represents hydrogen.

18. The compound according to claim 17, wherein at least one among R^{A} and R^{C} represents hydrogen.

19. The compound according to claim 16, wherein at least one among R^{A}, R^{B} and R^{D} represents F or Cl.

20. The compound according to claim 19, wherein at least one among R^{B} and R^{D} represents F or Cl.

21. The compound according to claim 20, wherein R^{B} and R^{D} each independently represents F or Cl.

22. The compound according to claim 16, wherein R¹ and R⁴ form together —CH₂—O—CH₂—.

23. The compound according to claim 16, wherein R¹ and R⁴ form together —CH₂—CH₂—, wherein the —CH₂—CH₂— is optionally substituted by at least one F, OH, or OCH₃.

24. The compound according to claim 16, wherein R¹ represents methyl, ethyl, CF₃, or CH₃OCH₂—.

25. The compound according to claim 16, wherein R⁷ represents hydrogen, OH, or halogen; or wherein R⁷ represents (C₁-C₅) alkyl-O— or cycloalkyl-O—, wherein the alkyl or cycloalkyl is optionally substituted by at least one F, Cl, OH, (C₁-C₈) alkoxy, or aryl.

26. The compound according to claim 16, wherein R⁷ represents F, Cl, OCH₃, OCH₂CH₃, OCF₃, cyclobutyl-O—, HO—CH₂—CH₂—O—, CH₃O—CH₂—CH₂—O—, phenyl-CH₂—O—, 1H-imidazole-4-yl-, 1-methyl-imidazole-4-yl-, CH₃, CN, CO₂H, CH₂OH, C(CH₃)₂OH, CH₂N(CH₃)₂, cyclopropyl, cyclobutyl-O—, (4-pyridine) —CH₂—O—, (3-pyridine) —CH₂—O—, or benzyl-O—.

27. The compound according to claim 16, wherein Z represents C—R⁸, wherein R⁸ represents (C₁-C₄) alkyl, F, Cl, CF₃, CHF₂, CH₂F, OCF₃, CN, OH or (C₁-C₄) alkoxy.

28. The compound according to claim 16, wherein Z represents C—R⁸ and R⁷ and R⁸ form together with the carbon atoms to which they are bound an heterocycloalkyl;
wherein the heterocycloalkyl is optionally substituted by at least one F, OH, ═O, (C₁-C₅) alkyl, CF₃, HO₂C—CH₂—, (C₁-C₄) alkyl-CO₂—CH₂—, or R¹⁵R¹⁶N—CH₂—, wherein R¹⁵ and R¹⁶ each independently represents hydrogen or (C₁-C₈) alkyl.

29. The compound according to claim 16, wherein R⁵ represents hydrogen or (C₁-C₅) alkyl; and/or wherein R⁶ represents (C₁-C₈) alkyl, cycloalkyl, cycloalkyl-(C₁-C₅) alkyl-, or heterocycloalkyl, wherein the alkyl, cycloalkyl, or heterocycloalkyl in R⁶ is optionally substituted by at least one F, CN, or CF₃.

30. The compound according to claim 16, wherein R⁵ represents hydrogen, methyl, or ethyl; and/or wherein R⁶ represents methyl, ethyl, i-propyl, n-propyl, isobutyronitrile, 3-fluoro-n-propane, tert-butyl, cyclopropyl, 1-methylcyclopropyl, cyclobutyl, 1-methylcyclobutyl, cyclopropyl-CH₂—, (CN) (Me) 2C—, (F3C) C (Me) 2-, 3-methyloxetane-3-yl, or oxetane-3-yl.

31. The compound according to claim 16, wherein R⁵ and R⁶ form together with the nitrogen atom to which they are bound an heterocycloalkyl, wherein the heterocycloalkyl is optionally substituted by at least one F.

32. The compound according to claim 16, wherein R⁵ and R⁶ form together with the nitrogen atom to which they are bound a pyrrolidine, 3,3-dimethylmorpholine, 4-fluoropiperidine, 3-fluoropyrrolidine, 4-trifluoromethylpiperidine, 4-benzyl-piperidine, 3-benzylpyrrolidine, 3-benzyl-piperidine, 4-phenylpiperidine, 4-(4-fluorophenyl) piperidine, 4-benzylidenepiperidine, octahydro-1H-isoindole, 3-(benzyloxy) pyrrolidine, 3-phenoxypyrrolidine, N-methyl-N-phenyl-pyrrolidin-3-amine, 2-azabicyclo[2.2.1]heptane, 5-(4-methylpiperazin-1-yl)pyrimidine, 4-phenethylpiperidine, phenylpiperazine, 4-benzyl-piperazine, 3-(4-piperidyl)benzonitrile, methyl 3-(4-piperidyl)benzoate, methyl 4-(4-piperidyl)benzoate, 4-(3-pyrazol-1-ylphenyl) piperidine, 1-[(1R,2S)-2-(4-fluorophenyl)cyclopropyl]piperazine, 1-[2-(4-fluorophenyl) propyl]piperazine, 1-[2-(4-fluorophenyl)ethyl]piperazine 1-[2-(4-chlorophenyl)ethyl]piperazine, 1-(2-phenylpropyl) piperazine, or 1-(4-fluorophenyl)-2-piperazin-1-yl-ethanol.

33. The compound according to claim 16, wherein said compound is selected from the group consisting of:

| | |
|---|---|
| 001 | 4-methoxy-N-methyl-N-propyl-3-[rac-(2S)-4-(2-chloro-4-fluoro- |
| | benzoyl)-2-methyl-piperazin-1-yl]benzenesulfonamide |
| 002 | N-tert-butyl-3-[(1S,5R)-3-(2-chloro-4-fluoro-benzoyl)-3,8- |
| | diazabicyclo[3.2.1]octan-8-yl]-4-methoxy-benzenesulfonamide |
| 003 | 3-[(1S,5R)-3-(2-chloro-4-fluoro-benzoyl)-3,8- |
| | diazabicyclo[3.2.1]octan-8-yl]-4-methoxy-N-methyl-N-propyl- |
| | benzenesulfonamide |
| 004 | (2-chloro-4-fluoro-phenyl)-[(1S,5R)-8-(2-methoxy-5-pyrrolidin-1- |
| | ylsulfonyl-phenyl)-3,8-diazabicyclo[3.2.1]octan-3-yl]methanone |
| 005 | 3-[(1S,5R)-3-(2-chloro-4-fluoro-benzoyl)-3,8- |
| | diazabicyclo[3.2.1]octan-8-yl]-N-(cyclopropylmethyl)-4-methoxy-N- |
| | methyl-benzenesulfonamide |
| 006 | (2-chloro-4-fluoro-phenyl)-[(1S,5R)-8-[5-[(4-fluoro-1- |
| | piperidyl)sulfonyl]-2-methoxy-phenyl]-3,8-diazabicyclo[3.2.1]octan- |
| | 3-yl]methanone |
| 007 | (2-chloro-4-fluoro-phenyl)-[(1S,5R)-8-[2-methoxy-5-[4- |
| | (trifluoromethyl)-1-piperidyl]sulfonyl]phenyl]-3,8- |
| | diazabicyclo[3.2.1]octan-3-yl]methanone |
| 008 | [(1S,5R)-8-[5-[(4-benzylidene-1-piperidyl)sulfonyl]-2-methoxy- |
| | phenyl]-3,8-diazabicyclo[3.2.1]octan-3-yl]-(2-chloro-4-fluoro- |
| | phenyl)methanone |
| 009 | (2-chloro-4-fluoro-phenyl)-[(1S,5R)-8-[2-methoxy-5-[(4-phenyl-1- |
| | piperidyl)sulfonyl]phenyl]-3,8-diazabicyclo[3.2.1]octan-3- |
| | yl]methanone |
| 010 | (2-chloro-4-fluoro-phenyl)-[(1S,5R)-8-[2-methoxy-5-[[4-(2- |
| | phenylethyl)-1-piperidyl]sulfonyl]phenyl]-3,8- |
| | diazabicyclo[3.2.1]octan-3-yl]methanone |
| 011 | [(1S,5R)-8-[5-[[(3RS)-3-benzyl-1-piperidyl]sulfonyl]-2-methoxy- |
| | phenyl]-3,8-diazabicyclo[3.2.1]octan-3-yl]-(2-chloro-4-fluoro- |
| | phenyl)methanone |
| 012 | (2-chloro-4-fluoro-phenyl)-[(1S,5R)-8-[2-methoxy-5-(4- |
| | phenylpiperazin-1-yl)sulfonyl-phenyl]-3,8-diazabicyclo[3.2.1]octan- |
| | 3-yl]methanone |
| 013 | (2-chloro-4-fluoro-phenyl)-[(1S,5R)-8-[2-methoxy-5-(4-pyrimidin-2- |
| | ylpiperazin-1-yl)sulfonyl-phenyl]-3,8-diazabicyclo[3.2.1]octan-3- |
| | yl]methanone |
| 014 | [(1S,5R)-8-[5-(4-benzylpiperazin-1-yl)sulfonyl-2-methoxy-phenyl]- |
| | 3,8-diazabicyclo[3.2.1]octan-3-yl]-(2-chloro-4-fluoro- |
| | phenyl)methanone; ethane |
| 015 | [(1S,5R)-8-[5-[[(3aS,7aR)-1,3,3a,4,5,6,7,7a-octahydroisoindol-2- |
| | yl]sulfonyl]-2-methoxy-phenyl]-3,8-diazabicyclo[3.2.1]octan-3-yl]- |
| | (2-chloro-4-fluoro-phenyl)methanone |
| 016 | 4-methoxy-N-methyl-N-propyl-3-[rac-(2S)-4-[4-fluoro-2- |
| | (trifluoromethyl)benzoyl]-2-(trifluoromethyl)piperazin-1- |
| | yl]benzenesulfonamide |
| 017 | 4-methoxy-N-methyl-N-propyl-3-[rac-(2S)-2-ethyl-4-[4-fluoro-2- |
| | (trifluoromethyl)benzoyl]piperazin-1-yl]benzenesulfonamide |
| 018 | [4-fluoro-2-(trifluoromethyl)phenyl]-[rac-(3S)-4-[5-[(4-benzyl-1- |
| | piperidyl)sulfonyl]-2-methoxy-phenyl]-3-methyl-piperazin-1- |
| | yl]methanone |
| 019 | (2-chloro-4-fluoro-phenyl)-[rac-(3S)-4-[5-[(4-benzyl-1- |
| | piperidyl)sulfonyl]-2-methoxy-phenyl]-3-methyl-piperazin-1- |
| | yl]methanone |
| 020 | N-tert-butyl-4-methoxy-3-[rac-(2S)-4-(2-chloro-4-fluoro-benzoyl)-2- |
| | methyl-piperazin-1-yl]benzenesulfonamide |
| 021 | 4-methoxy-N-methyl-N-propyl-3-[rac-(2S)-4-[4-fluoro-2- |
| | (trifluoromethyl)benzoyl]-2-methyl-piperazin-1- |
| | yl]benzenesulfonamide |
| 022 | 4-methoxy-N-methyl-N-propyl-3-[rac-(2R)-4-[4-fluoro-2- |
| | (trifluoromethyl)benzoyl]-2-(methoxymethyl)piperazin-1- |
| | yl]benzenesulfonamide |
| 023 | 3-[(1S,5R)-7-[4-fluoro-2-(trifluoromethyl)benzoyl]-3-oxa-7,9- |
| | diazabicyclo[3.3.1]nonan-9-yl]-4-methoxy-N-methyl-N-propyl- |
| | benzenesulfonamide |
| 024 | 3-[3-[4-fluoro-2-(trifluoromethyl)benzoyl]-3,8- |
| | diazabicyclo[3.2.1]octan-8-yl]-4-methoxy-N-methyl-N-propyl- |
| | benzenesulfonamide |
| 025 | (2-chloro-4-fluoro-phenyl)-[rac-(1S,5S)-8-[5-[(4-benzyl-1- |
| | piperidyl)sulfonyl]-2-methoxy-phenyl]-3,8-diazabicyclo[3.2.1]octan- |
| | 3-yl]methanone |
| 026 | 3-[(1S,5R)-3-(2-chloro-4-fluoro-benzoyl)-3,8- |
| | diazabicyclo[3.2.1]octan-8-yl]-4-fluoro-N-methyl-N-propyl- |
| | benzenesulfonamide |
| 027 | 3-[(1S,5R)-3-(2-chloro-4-fluoro-benzoyl)-3,8- |
| | diazabicyclo[3.2.1]octan-8-yl]-N-methyl-N-propyl-4- |
| | (trifluoromethoxy)benzenesulfonamide |
| 028 | 3-[(1S,5R)-3-(2-chloro-4-fluoro-benzoyl)-3,8- |
| | diazabicyclo[3.2.1]octan-8-yl]-N-methyl-N-propyl- |
| | benzenesulfonamide |
| 029 | 3-[(1S,5R)-3-(2-chloro-4-fluoro-benzoyl)-3,8- |
| | diazabicyclo[3.2.1]octan-8-yl]-4-ethoxy-N-methyl-N-propyl- |
| | benzenesulfonamide |
| 030 | 3-[(1S,5R)-3-(2-chloro-4-fluoro-benzoyl)-3,8- |
| | diazabicyclo[3.2.1]octan-8-yl]-4-(cyclobutoxy)-N-methyl-N-propyl- |
| | benzenesulfonamide |
| 031 | 3-[(1S,5R)-3-(2-chloro-4-fluoro-benzoyl)-3,8- |
| | diazabicyclo[3.2.1]octan-8-yl]-4-(methoxymethoxy)-N-methyl-N- |
| | propyl-benzenesulfonamide |
| 032 | 6-chloro-5-[(1S,5R)-3-(2-chloro-4-fluoro-benzoyl)-3,8- |
| | diazabicyclo[3.2.1]octan-8-yl]-N,N-diethyl-pyridine-3-sulfonamide |
| 033 | 5-[(1S,5R)-3-(2-chloro-4-fluoro-benzoyl)-3,8- |
| | diazabicyclo[3.2.1]octan-8-yl]-N,N-diethyl-6-methoxy-pyridine-3- |
| | sulfonamide |
| 034 | 6-benzyloxy-5-[(1S,5R)-3-(2-chloro-4-fluoro-benzoyl)-3,8- |
| | diazabicyclo[3.2.1]octan-8-yl]-N,N-diethyl-pyridine-3-sulfonamide |
| 035 | 5-[(1S,5R)-3-(2-chloro-4-fluoro-benzoyl)-3,8- |
| | diazabicyclo[3.2.1]octan-8-yl]-N,N-diethyl-6-hydroxy-pyridine-3- |
| | sulfonamide |
| 036 | 3-[(1S,5R)-3-(2-chloro-4-fluoro-benzoyl)-3,8- |
| | diazabicyclo[3.2.1]octan-8-yl]-4-hydroxy-N-methyl-N-propyl- |
| | benzenesulfonamide |
| 037 | [(1S,5R)-8-[5-[(3SR)-3-benzyloxypyrrolidin-1-yl]sulfonyl-2- |
| | methoxy-phenyl]-3,8-diazabicyclo[3.2.1]octan-3-yl]-(2-chloro-4- |
| | fluoro-phenyl)methanone |
| 038 | N-(3-fluoropropyl)-4-methoxy-N-methyl-3-[rac-(2S)-4-(2-chloro-4- |
| | fluoro-benzoyl)-2-methyl-piperazin-1-yl]benzenesulfonamide |
| 039 | (2-chloro-4-fluoro-phenyl)-[(1S,5R)-8-[5-[[4-(4-fluorophenyl)-1- |
| | piperidyl]sulfonyl]-2-methoxy-phenyl]-3,8-diazabicyclo[3.2.1]octan- |
| | 3-yl]methanone |
| 040 | 3-[1-[3-[(1S,5R)-3-(2-chloro-4-fluoro-benzoyl)-3,8- |
| | diazabicyclo[3.2.1]octan-8-yl]-4-methoxy-phenyl]sulfonyl-4- |
| | piperidyl]benzonitrile |
| 041 | methyl 3-[1-[3-[(1S,5R)-3-(2-chloro-4-fluoro-benzoyl)-3,8- |
| | diazabicyclo[3.2.1]octan-8-yl]-4-methoxy-phenyl]sulfonyl-4- |
| | piperidyl]benzoate |
| 042 | methyl 4-[1-[3-[(1S,5R)-3-(2-chloro-4-fluoro-benzoyl)-3,8- |
| | diazabicyclo[3.2.1]octan-8-yl]-4-methoxy-phenyl]sulfonyl-4- |
| | piperidyl]benzoate |
| 043 | (2-chloro-4-fluoro-phenyl)-[(1S,5R)-8-[2-methoxy-5-[[4-(3-pyrazol- |
| | 1-ylphenyl)-1-piperidyl]sulfonyl]phenyl]-3,8- |
| | diazabicyclo[3.2.1]octan-3-yl]methanone |
| 044 | (2-chloro-4-fluoro-phenyl)-[(1S,5R)-8-[5-[4-[(1SR,2RS)-2-(4- |
| | fluorophenyl)cyclopropyl]piperazin-1-yl]sulfonyl-2-methoxy- |
| | phenyl]-3,8-diazabicyclo[3.2.1]octan-3-yl]methanone |
| 045 | (2-chloro-4-fluoro-phenyl)-[(1S,5R)-8-[5-[4-[(2RS)-2-(4- |
| | fluorophenyl)propyl]piperazin-1-yl]sulfonyl-2-methoxy-phenyl]-3,8- |
| | diazabicyclo[3.2.1]octan-3-yl]methanone |
| 046 | (2-chloro-4-fluoro-phenyl)-[(1S,5R)-8-[5-[4-[2-(4- |
| | fluorophenyl)ethyl]piperazin-1-yl]sulfonyl-2-methoxy-phenyl]-3,8- |
| | diazabicyclo[3.2.1]octan-3-yl]methanone |
| 047 | (2-chloro-4-fluoro-phenyl)-[(1S,5R)-8-[2-methoxy-5-[4-[(2RS)-2- |
| | phenylpropyl]piperazin-1-yl]sulfonyl-phenyl]-3,8- |
| | diazabicyclo[3.2.1]octan-3-yl]methanone |
| 048 | (2-chloro-4-fluoro-phenyl)-[(1S,5R)-8-[5-[4-[2-(4- |
| | chlorophenyl)ethyl]piperazin-1-yl]sulfonyl-2-methoxy-phenyl]-3,8- |
| | diazabicyclo[3.2.1]octan-3-yl]methanone |
| 049 | (2-chloro-4,5-difluoro-phenyl)-[(1S,5R)-8-[2-methoxy-5-[(4-phenyl- |
| | 1-piperidyl)sulfonyl]phenyl]-3,8-diazabicyclo[3.2.1]octan-3- |
| | yl]methanone |
| 050 | (2-chloro-4,5-difluoro-phenyl)-[(1S,5R)-8-[2-methoxy-5-[[4-(2- |
| | phenylethyl)-1-piperidyl]sulfonyl]phenyl]-3,8- |
| | diazabicyclo[3.2.1]octan-3-yl]methanone |
| 051 | (2-chloro-4,5-difluoro-phenyl)-[(1S,5R)-8-[2-methoxy-5-(4- |
| | phenylpiperazin-1-yl)sulfonyl-phenyl]-3,8-diazabicyclo[3.2.1]octan- |
| | 3-yl]methanone |
| 052 | [(1S,5R)-8-[5-[(3-benzyl-1-piperidyl)sulfonyl]-2-methoxy-phenyl]- |
| | 3,8-diazabicyclo[3.2.1]octan-3-yl]-(2-chloro-4,5-difluoro- |
| | phenyl)methanone |
| 053 | [(1S,5R)-8-[5-[(4-benzylidene-1-piperidyl)sulfonyl]-2-methoxy- |
| | phenyl]-3,8-diazabicyclo[3.2.1]octan-3-yl]-(2-chloro-4,5-difluoro- |
| | phenyl)methanone |
| 054 | N-tert-butyl-3-[(1S,5R)-3-(2-chloro-4,5-difluoro-benzoyl)-3,8- |
| | diazabicyclo[3.2.1]octan-8-yl]-4-methoxy-N-methyl- |
| | benzenesulfonamide |
| 055 | (2-chloro-4-fluoro-phenyl)-[(1S,5R)-8-[5-[(4-phenyl-1- |
| | piperidyl)sulfonyl]-2,3-dihydrobenzofuran-7-yl]-3,8- |
| | diazabicyclo[3.2.1]octan-3-yl]methanone |
| 056 | (2-chloro-4-fluoro-phenyl)-[(1S,5R)-8-[5-[4-(4-fluorophenyl)-1- |
| | piperidyl]sulfonyl]-2,3-dihydrobenzofuran-7-yl]-3,8- |
| | diazabicyclo[3.2.1]octan-3-yl]methanone |
| 057 | N-tert-butyl-7-[(1S,5R)-3-(2-chloro-4-fluoro-benzoyl)-3,8- |
| | diazabicyclo[3.2.1]octan-8-yl]-2,3-dihydrobenzofuran-5-sulfonamide |
| 058 | 7-[(1S,5R)-3-(2-chloro-4-fluoro-benzoyl)-3,8- |
| | diazabicyclo[3.2.1]octan-8-yl]-N-(cyclopropylmethyl)-N-methyl-2,3- |
| | dihydrobenzofuran-5-sulfonamide |
| 059 | (2-chloro-4-fluoro-phenyl)-[(1S,5R)-8-[5-[4-[2-(4- |
| | fluorophenyl)ethyl]piperazin-1-yl]sulfonyl-2,3-dihydrobenzofuran-7- |
| | yl]-3,8-diazabicyclo[3.2.1]octan-3-yl]methanone |
| 060 | (2-chloro-4-fluoro-phenyl)-[(1S,5R)-8-[5-[4-[2-(4- |
| | chlorophenyl)ethyl]piperazin-1-yl]sulfonyl-2,3-dihydrobenzofuran-7- |
| | yl]-3,8-diazabicyclo[3.2.1]octan-3-yl]methanone |
| 061 | (2-chloro-4,5-difluoro-phenyl)-[(1S,5R)-8-[5-[(4-phenyl-1- |
| | piperidyl)sulfonyl]-2,3-dihydrobenzofuran-7-yl]-3,8- |
| | diazabicyclo[3.2.1]octan-3-yl]methanone |
| 062 | (2-chloro-4-fluoro-phenyl)-[(1S,5R)-8-[2-hydroxy-5-[[4-(2- |
| | phenylethyl)-1-piperidyl]sulfonyl]phenyl]-3,8- |
| | diazabicyclo[3.2.1]octan-3-yl]methanone |
| 063 | (2-chloro-4-fluoro-phenyl)-[(1S,5R)-8-[2-hydroxy-5-[(4-phenyl-1- |
| | piperidyl)sulfonyl]phenyl]-3,8-diazabicyclo[3.2.1]octan-3- |
| | yl]methanone |
| 064 | (2-chloro-4-fluoro-phenyl)-[(1S,5R)-8-[5-[4-[2-(4- |
| | chlorophenyl)ethyl]piperazin-1-yl]sulfonyl-2-hydroxy-phenyl]-3,8- |
| | diazabicyclo[3.2.1]octan-3-yl]methanone |
| 065 | (2-chloro-4-fluoro-phenyl)-[(1S,5R)-8-[5-[[4-(4-fluorophenyl)-1- |
| | piperidyl]sulfonyl]-2-hydroxy-phenyl]-3,8-diazabicyclo[3.2.1]octan- |
| | 3-yl]methanone |
| 066 | (2-chloro-4-fluoro-phenyl)-[(1S,5R)-8-[2-chloro-5-[(4-phenyl-1- |
| | piperidyl)sulfonyl]phenyl]-3,8-diazabicyclo[3.2.1]octan-3- |
| | yl]methanone |
| 067 | (2-chloro-4-fluoro-phenyl)-[(1S,5R)-8-[2-methyl-5-[(4-phenyl-1- |
| | piperidyl)sulfonyl]phenyl]-3,8-diazabicyclo[3.2.1]octan-3- |
| | yl]methanone |
| 068 | 3-[(1S,5R)-3-(2-chloro-4-fluoro-benzoyl)-3,8- |
| | diazabicyclo[3.2.1]octan-8-yl]-N,4-dimethyl-N-propyl- |
| | benzenesulfonamide |
| 069 | methyl 2-[(1S,5R)-3-(2-chloro-4-fluoro-benzoyl)-3,8- |
| | diazabicyclo[3.2.1]octan-8-yl]-4-[(4-phenyl-1- |
| | piperidyl)sulfonyl]benzoate |
| 070 | (2-chloro-4-fluoro-phenyl)-[(1S,5R)-8-[2-(hydroxymethyl)-5-[(4- |
| | phenyl-1-piperidyl)sulfonyl]phenyl]-3,8-diazabicyclo[3.2.1]octan-3- |
| | yl]methanone |
| 071 | 2-[(1S,5R)-3-(2-chloro-4-fluoro-benzoyl)-3,8- |
| | diazabicyclo[3.2.1]octan-8-yl]-4-[(4-phenyl-1- |
| | piperidyl)sulfonyl]benzoic acid |
| 072 | (2-chloro-4-fluoro-phenyl)-[(1S,5R)-8-[2-(1-hydroxy-1-methyl- |
| | ethyl)-5-[(4-phenyl-1-piperidyl)sulfonyl]phenyl]-3,8- |
| | diazabicyclo[3.2.1]octan-3-yl]methanone |
| 073 | (2-chloro-4-fluoro-phenyl)-[(1S,5R)-8-[2-[(dimethylamino)methyl]- |
| | 5-[(4-phenyl-1-piperidyl)sulfonyl]phenyl]-3,8- |
| | diazabicyclo[3.2.1]octan-3-yl]methanone |
| 074 | N-tert-butyl-3-chloro-5-[(1S,5R)-3-(2-chloro-4-fluoro-benzoyl)-3,8- |
| | diazabicyclo[3.2.1]octan-8-yl]-4-methyl-benzenesulfonamide |
| 075 | N-tert-butyl-3,4-dichloro-5-[(1S,5R)-3-(2-chloro-4-fluoro-benzoyl)- |
| | 3,8-diazabicyclo[3.2.1]octan-8-yl]benzenesulfonamide |
| 076 | (2-chloro-4-fluoro-phenyl)-[(1S,5R)-8-[2-(2-hydroxyethoxy)-5-[(4- |
| | phenyl-1-piperidyl)sulfonyl]phenyl]-3,8-diazabicyclo[3.2.1]octan-3- |
| | yl]methanone |
| 077 | (2-chloro-4-fluoro-phenyl)-[(1S,5R)-8-[2-(2-hydroxyethoxy)-5-[[4- |
| | (2-phenylethyl)-1-piperidyl]sulfonyl]phenyl]-3,8- |
| | diazabicyclo[3.2.1]octan-3-yl]methanone |
| 078 | (2-chloro-4-fluoro-phenyl)-[(1S,5R)-8-[5-[4-[2-(4- |
| | chlorophenyl)ethyl]piperazin-1-yl]sulfonyl-2-(2- |
| | hydroxyethoxy)phenyl]-3,8-diazabicyclo[3.2.1]octan-3-yl]methanone |
| 079 | 3-[(1S,5R)-3-(2-chloro-4-fluoro-benzoyl)-3,8- |
| | diazabicyclo[3.2.1]octan-8-yl]-4-cyclopropyl-N-methyl-N-propyl- |
| | benzenesulfonamide |
| 080 | (2-chloro-4-fluoro-phenyl)-[(1S,5R)-8-[2-cyclopropyl-5-[(4-phenyl- |
| | 1-piperidyl)sulfonyl]phenyl]-3,8-diazabicyclo[3.2.1]octan-3- |
| | yl]methanone |
| 081 | (2-chloro-4-fluoro-phenyl)-[(1S,5R)-8-[2-(1H-imidazol-4-yl)-5-[(4- |
| | phenyl-1-piperidyl)sulfonyl]phenyl]-3,8-diazabicyclo[3.2.1]octan-3- |
| | yl]methanone |
| 082 | (2-chloro-4-fluoro-phenyl)-[(1S,5R)-8-[2-(1-methylimidazol-4-yl)-5- |
| | [(4-phenyl-1-piperidyl)sulfonyl]phenyl]-3,8- |
| | diazabicyclo[3.2.1]octan-3-yl]methanone |
| 083 | (2-chloro-4-fluoro-phenyl)-[(1S,5R)-8-[3-fluoro-2-hydroxy-5-[(4- |
| | phenyl-1-piperidyl)sulfonyl]phenyl]-3,8-diazabicyclo[3.2.1]octan-3- |
| | yl]methanone |
| 084 | (2-chloro-4-fluoro-phenyl)-[(1S,5R)-8-[3-fluoro-5-[[4-(4- |
| | fluorophenyl)-1-piperidyl]sulfonyl]-2-hydroxy-phenyl]-3,8- |
| | diazabicyclo[3.2.1]octan-3-yl]methanone |
| 085 | N-tert-butyl-3-[(1S,5R)-3-(2-chloro-4-fluoro-benzoyl)-3,8- |
| | diazabicyclo[3.2.1]octan-8-yl]-5-fluoro-4-hydroxy- |
| | benzenesulfonamide |
| 086 | 3-[(1S,5R)-3-(2-chloro-4-fluoro-benzoyl)-3,8- |
| | diazabicyclo[3.2.1]octan-8-yl]-N-(cyclopropylmethyl)-5-fluoro-4- |
| | hydroxy-N-methyl-benzenesulfonamide |
| 087 | (2-chloro-4-fluoro-phenyl)-[(1S,5R)-8-[2-hydroxy-5-[(4-phenyl-1- |
| | piperidyl)sulfonyl]-3-(trifluoromethyl)phenyl]-3,8- |
| | diazabicyclo[3.2.1]octan-3-yl]methanone |
| 088 | (2-chloro-4-fluoro-phenyl)-[(1S,5R)-8-[5-[4-(4-fluorophenyl)-1- |
| | piperidyl]sulfonyl]-2-hydroxy-3-(trifluoromethyl)phenyl]-3,8- |
| | diazabicyclo[3.2.1]octan-3-yl]methanone |
| 089 | N-tert-butyl-3-[(1S,5R)-3-(2-chloro-4-fluoro-benzoyl)-3,8- |
| | diazabicyclo[3.2.1]octan-8-yl]-4-hydroxy-5- |
| | (trifluoromethyl)benzenesulfonamide |
| 090 | (2-chloro-4-fluoro-phenyl)-[(1S,5R)-8-[2-hydroxy-3-methyl-5-[(4- |
| | phenyl-1-piperidyl)sulfonyl]phenyl]-3,8-diazabicyclo[3.2.1]octan-3- |
| | yl]methanone |
| 091 | (2-chloro-4-fluoro-phenyl)-[(1S,5R)-8-[5-[[4-(4-fluorophenyl)-1- |
| | piperidyl]sulfonyl]-2-hydroxy-3-methyl-phenyl]-3,8- |
| | diazabicyclo[3.2.1]octan-3-yl]methanone |
| 092 | 3-[(1S,5R)-3-(2-chloro-4-fluoro-benzoyl)-3,8- |
| | diazabicyclo[3.2.1]octan-8-yl]-N-(cyclopropylmethyl)-4-hydroxy- |
| | N,5-dimethyl-benzenesulfonamide |
| 093 | N-tert-butyl-3-[(1S,5R)-3-(2-chloro-4-fluoro-benzoyl)-3,8- |
| | diazabicyclo[3.2.1]octan-8-yl]-4-hydroxy-5-methyl- |
| | benzenesulfonamide |
| 094 | (2-chloro-4-fluoro-phenyl)-[(1S,5R)-8-[3-ethyl-2-hydroxy-5-[(4- |
| | phenyl-1-piperidyl)sulfonyl]phenyl]-3,8-diazabicyclo[3.2.1]octan-3- |
| | yl]methanone |
| 095 | N-tert-butyl-3-chloro-5-[(1S,5R)-3-(2-chloro-4-fluoro-benzoyl)-3,8- |
| | diazabicyclo[3.2.1]octan-8-yl]-4-hydroxy-benzenesulfonamide |
| 096 | 3-chloro-5-[(1S,5R)-3-(2-chloro-4-fluoro-benzoyl)-3,8- |
| | diazabicyclo[3.2.1]octan-8-yl]-N-(cyclopropylmethyl)-4-hydroxy-N- |
| | methyl-benzenesulfonamide |
| 097 | (2-chloro-4-fluoro-phenyl)-[(1S,5R)-8-[3-chloro-5-[(4-fluoro-1- |
| | piperidyl)sulfonyl]-2-hydroxy-phenyl]-3,8-diazabicyclo[3.2.1]octan- |
| | 3-yl]methanone |
| 098 | (2-chloro-4-fluoro-phenyl)-[(1S,5R)-8-[3-chloro-2-hydroxy-5-[(4- |
| | phenyl-1-piperidyl)sulfonyl]phenyl]-3,8-diazabicyclo[3.2.1]octan-3- |
| | yl]methanone |
| 099 | (2-chloro-4-fluoro-phenyl)-[(1S,5R)-8-[3-chloro-5-[[4-(4- |
| | fluorophenyl)-1-piperidyl]sulfonyl]-2-hydroxy-phenyl]-3,8- |
| | diazabicyclo[3.2.1]octan-3-yl]methanone |
| 100 | 3-chloro-5-[(1S,5R)-3-(2-chloro-4-fluoro-benzoyl)-3,8- |
| | diazabicyclo[3.2.1]octan-8-yl]-4-hydroxy-N-isopropyl- |
| | benzenesulfonamide |
| 101 | 3-chloro-5-[(1S,5R)-3-(2-chloro-4-fluoro-benzoyl)-3,8- |
| | diazabicyclo[3.2.1]octan-8-yl]-4-hydroxy-N-(1- |
| | methylcyclopropyl)benzenesulfonamide |
| 102 | 3-chloro-5-[(1S,5R)-3-(2-chloro-4-fluoro-benzoyl)-3,8- |
| | diazabicyclo[3.2.1]octan-8-yl]-4-hydroxy-N-(1- |
| | methylcyclobutyl)benzenesulfonamide |
| 103 | 3-chloro-5-[(1S,5R)-3-(2-chloro-4-fluoro-benzoyl)-3,8- |
| | diazabicyclo[3.2.1]octan-8-yl]-N-(1-cyano-1-methyl-ethyl)-4- |
| | hydroxy-benzenesulfonamide |
| 104 | 3-chloro-5-[(1S,5R)-3-(2-chloro-4-fluoro-benzoyl)-3,8- |
| | diazabicyclo[3.2.1]octan-8-yl]-4-hydroxy-N-[(1SR)-2,2,2-trifluoro-1- |
| | methyl-ethyl]benzenesulfonamide |
| 105 | 3-chloro-5-[(1S,5R)-3-(2-chloro-4-fluoro-benzoyl)-3,8- |
| | diazabicyclo[3.2.1]octan-8-yl]-4-hydroxy-N-(2,2,2-trifluoro-1,1- |
| | dimethyl-ethyl)benzenesulfonamide |
| 106 | 3-chloro-5-[(1S,5R)-3-(2-chloro-4-fluoro-benzoyl)-3,8- |
| | diazabicyclo[3.2.1]octan-8-yl]-4-hydroxy-N-methyl-N-(3- |
| | methyloxetan-3-yl)benzenesulfonamide |
| 107 | N-tert-butyl-3-chloro-5-[(1S,5R)-3-(2-chloro-4-fluoro-benzoyl)-3,8- |
| | diazabicyclo[3.2.1]octan-8-yl]-4-hydroxy-N-methyl- |
| | benzenesulfonamide |
| 108 | 3-chloro-5-[(1S,5R)-3-(2-chloro-4-fluoro-benzoyl)-3,8- |
| | diazabicyclo[3.2.1]octan-8-yl]-4-hydroxy-N-(3-methyloxetan-3- |
| | yl)benzenesulfonamide |
| 109 | 3-chloro-5-[(1S,5R)-3-(2-chloro-4-fluoro-benzoyl)-3,8- |
| | diazabicyclo[3.2.1]octan-8-yl]-4-hydroxy-N-methyl-N-(oxetan-3- |
| | yl)benzenesulfonamide |
| 110 | N-tert-butyl-3-chloro-5-[(2S)-4-(2-chloro-4-fluoro-benzoyl)-2- |
| | methyl-piperazin-1-yl]-4-hydroxy-benzenesulfonamide |
| 111 | N-tert-butyl-3-chloro-5-[(2R)-4-(2-chloro-4-fluoro-benzoyl)-2- |
| | methyl-piperazin-1-yl]-4-hydroxy-benzenesulfonamide |
| 112 | N-tert-butyl-3-chloro-5-[(1S,5R)-3-(2-chloro-4-fluoro-benzoyl)-3,8- |
| | diazabicyclo[3.2.1]octan-8-yl]-4-(2- |
| | hydroxyethoxy)benzenesulfonamide |
| 113 | N-tert-butyl-3-chloro-5-[(1S,5R)-3-(2-chloro-4-fluoro-benzoyl)-3,8- |
| | diazabicyclo[3.2.1]octan-8-yl]-4-(2- |
| | methoxyethoxy)benzenesulfonamide |
| 114 | N-tert-butyl-3-chloro-5-[(1S,5R)-3-(2-chloro-4-fluoro-benzoyl)-3,8- |
| | diazabicyclo[3.2.1]octan-8-yl]-4-methoxy-benzenesulfonamide |
| 115 | 4-chloro-3-[(1S,5R)-3-(2-chloro-4-fluoro-benzoyl)-3,8- |
| | diazabicyclo[3.2.1]octan-8-yl]-N-methyl-N-propyl- |
| | benzenesulfonamide |
| 116 | (2-chloro-4-fluoro-phenyl)-[(1S,5R)-8-[5-[4-[2-(4- |
| | fluorophenyl)ethyl]piperazin-1-yl]sulfonyl-2-methyl-phenyl]-3,8- |
| | diazabicyclo[3.2.1]octan-3-yl]methanone |
| 117 | (2-chloro-4-fluoro-phenyl)-[(1S,5R)-8-[6-[(4-phenyl-1- |
| | piperidyl)sulfonyl]-1,3-benzodioxol-4-yl]-3,8- |
| | diazabicyclo[3.2.1]octan-3-yl]methanone |
| 118 | (2-chloro-4-fluoro-phenyl)-[(1S,5R)-8-[7-[(4-phenyl-1- |
| | piperidyl)sulfonyl]-2,3-dihydro-1,4-benzodioxin-5-yl]-3,8- |
| | diazabicyclo[3.2.1]octan-3-yl]methanone |
| 119 | (2-chloro-4-fluoro-phenyl)-[(1S,5R)-8-[1-methyl-5-[(4-phenyl-1- |
| | piperidyl)sulfonyl]indolin-7-yl]-3,8-diazabicyclo[3.2.1]octan-3- |
| | yl]methanone |
| 120 | (2-chloro-4,5-difluoro-phenyl)-[(1S,5R)-8-[1-methyl-5-[(4-phenyl-1- |
| | piperidyl)sulfonyl]indolin-7-yl]-3,8-diazabicyclo[3.2.1]octan-3- |
| | yl]methanone |
| 121 | (2-chloro-4-fluoro-phenyl)-[(1S,5R)-8-[2-(dimethylamino)-5-[(4- |
| | phenyl-1-piperidyl)sulfonyl]phenyl]-3,8-diazabicyclo[3.2.1]octan-3- |
| | yl]methanone |
| 122 | (2-chloro-4,5-difluoro-phenyl)-[(1S,5R)-8-[2-(dimethylamino)-5-[(4- |
| | phenyl-1-piperidyl)sulfonyl]phenyl]-3,8-diazabicyclo[3.2.1]octan-3- |
| | yl]methanone |
| 123 | (2-chloro-4-fluoro-phenyl)-[(1S,5R)-8-[2-[3-(methylamino)azetidin- |
| | 1-yl]-5-[(4-phenyl-1-piperidyl)sulfonyl]phenyl]-3,8- |
| | diazabicyclo[3.2.1]octan-3-yl]methanone |
| 124 | (2-chloro-4-fluoro-phenyl)-[(1S,5R)-8-[3-hydroxy-2-methyl-5-[(4- |
| | phenyl-1-piperidyl)sulfonyl]phenyl]-3,8-diazabicyclo[3.2.1]octan-3- |
| | yl]methanone |
| 125 | 2-[(1R,5S)-3-(2-chloro-4-fluoro-benzoyl)-3,8- |
| | diazabicyclo[3.2.1]octan-8-yl]-4-(4-phenylcyclohexyl)sulfonyl- |
| | benzonitrile |
| 126 | (2-chloro-4-fluoro-phenyl)-[(1S,5R)-8-[2-methoxy-5-[(3SR)-3- |
| | phenoxypyrrolidin-1-yl]sulfonyl-phenyl]-3,8- |
| | diazabicyclo[3.2.1]octan-3-yl]methanone |
| 127 | (2-chloro-4-fluoro-phenyl)-[(1S,5R)-8-[2-methoxy-5-[(3SR)-3-(N- |
| | methylanilino)pyrrolidin-1-yl]sulfonyl-phenyl]-3,8- |
| | diazabicyclo[3.2.1]octan-3-yl]methanone |
| 128 | (2-chloro-4-fluoro-phenyl)-[(1S,5R)-8-[5-[4-[(2RS)-2-(4- |
| | fluorophenyl)-2-hydroxy-ethyl]piperazin-1-yl]sulfonyl-2-methoxy- |
| | phenyl]-3,8-diazabicyclo[3.2.1]octan-3-yl]methanone |
| 129 | N-tert-butyl-3-[(1S,5R)-3-(2-chloro-4,5-difluoro-benzoyl)-3,8- |
| | diazabicyclo[3.2.1]octan-8-yl]-4-methoxy-benzenesulfonamide |
| 130 | (2-chloro-4-fluoro-phenyl)-[(1S,5R)-8-[5-[(4-fluoro-1- |
| | piperidyl)sulfonyl]-2,3-dihydrobenzofuran-7-yl]-3,8- |
| | diazabicyclo[3.2.1]octan-3-yl]methanone |
| 131 | [(1S,5R)-8-[3-chloro-5-(3,3-dimethylmorpholin-4-yl)sulfonyl-2- |
| | hydroxy-phenyl]-3,8-diazabicyclo[3.2.1]octan-3-yl]-(2-chloro-4- |
| | fluoro-phenyl)methanone |
| 132 | N-tert-butyl-3-chloro-5-[(1S,5R)-3-(2-chloro-4-fluoro-benzoyl)-3,8- |
| | diazabicyclo[3.2.1]octan-8-yl]-N,4-dimethyl-benzenesulfonamide |
| 133 | N-tert-butyl-3,4-dichloro-5-[(1S,5R)-3-(2-chloro-4-fluoro-benzoyl)- |
| | 3,8-diazabicyclo[3.2.1]octan-8-yl]-N-methyl-benzenesulfonamide |
| 134 | N-tert-butyl-3-chloro-5-[(1S,5R)-3-(2-chloro-4-fluoro-benzoyl)-3,8- |
| | diazabicyclo[3.2.1]octan-8-yl]-4-(2-hydroxyethoxy)-N-methyl- |
| | benzenesulfonamide |
| 135 | N-tert-butyl-3-chloro-5-[(1S,5R)-3-(2-chloro-4-fluoro-benzoyl)-3,8- |
| | diazabicyclo[3.2.1]octan-8-yl]-4-(2-methoxyethoxy)-N-methyl- |
| | benzenesulfonamide |
| 136 | 4-benzyloxy-N-tert-butyl-3-chloro-5-[(1S,5R)-3-(2-chloro-4-fluoro- |
| | benzoyl)-3,8-diazabicyclo[3.2.1]octan-8-yl]-N-methyl- |
| | benzenesulfonamide |
| 137 | N-tert-butyl-3-chloro-5-[(1S,5R)-3-(2-chloro-4-fluoro-benzoyl)-3,8- |
| | diazabicyclo[3.2.1]octan-8-yl]-N-methyl-4-(3- |
| | pyridylmethoxy)benzenesulfonamide |
| 138 | N-tert-butyl-3-chloro-5-[(1S,5R)-3-(2-chloro-4-fluoro-benzoyl)-3,8- |
| | diazabicyclo[3.2.1]octan-8-yl]-N-methyl-4-(4- |
| | pyridylmethoxy)benzenesulfonamide |
| 139 | N-tert-butyl-5-chloro-3-[(1S,5R)-3-(2-chloro-4-fluoro-benzoyl)-3,8- |
| | diazabicyclo[3.2.1]octan-8-yl]-2-fluoro-4-hydroxy- |
| | benzenesulfonamide |

and pharmaceutically acceptable salts and/or solvates thereof.

34. A pharmaceutical composition comprising a compound according to claim 16 and at least one pharmaceutically acceptable carrier.

35. A process for manufacturing a compound according to claim 16, comprising a step of reacting:
a compound of formula (II) 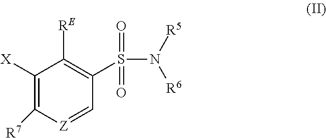
wherein
Z, R⁵, R⁶, R⁷ and R^{E} are as defined above, and
X represents halide or —CF₃SO₃,
with a compound of formula (III) 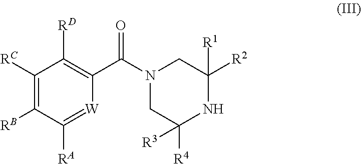
wherein W, R^{A}-R^{D} and R¹-R⁴ are as defined above,
in presence of a base and a metal catalyst;
thereby obtaining the compound of formula (I) or the pharmaceutically acceptable salt and/or solvate thereof.


SEQUENCE LISTING (2 sequences)

SEQ ID NO: 1 - protein, 465 aa, Homo sapiens
  SIGNAL 1..24
MFLATLYFALPLLDLLLSAEVSGGDRLDCVKASDQCLKEQSCSTKYRTLRQCVAGKETNFSLASGLEAKDECRSAMEALKQKSLYNCRCKRGMKKEKNCLRIYWSMYQSLQGNDLLEDSPYEPVNSRLSDIFRVVPFISDVFQQVEHIPKGNNCLDAAKACNLDDICKKYRSAYITPCTTSVSNDVCNRRKCHKALRQFFDKVPAKHSYGMLFCSCRDIACTERRRQTIVPVCSYEEREKPNCLNLQDSCKTNYICRSRLADFFTNCQPESRSVSSCLKENYADCLLAYSGLIGTVMTPNYIDSSSLSVAPWCDCSNSGNDLEECLKFLNFFKDNTCLKNAIQAFGNGSDVTVWQPAFPVQTTTATTTTALRVKNKPLGPAGSENEIPTHVLPPCANLQAQKLKSNVSGNTHLCISNGNYEKEGLGASSHITTKSMAAPPSCGLSPLLVLVVTALSTLLSLTETS

SEQ ID NO: 2 - protein, 1114 aa, Homo sapiens
  SIGNAL 1..28
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS